SEQUENCE LISTING (128 sequences)

SEQ ID NO: 1 - DNA, 345 bp, synthetic construct
  source 1..345: mol_type = other DNA
gccaccatggactacaaagacgatgacgacaagggcccggctgtggctgaggagcccctccaccgaccgtgagtttgggctgcatgactgcatggttgcatgaacacatattaatttcctccacttagttctacacctcattcattcattcagtgagtgtttctcgactactatgaataaaccgttatactccatgttgcgggcagaatggggatctggacagggaagcacagggcacgagttcaccaatggctgtcaagctacgctgcggccggcatggtcccagcctcctcgctggcgccggctgggcaacatgcttcggcatggcgaatgggacgcggccgc

SEQ ID NO: 2 - DNA, 312 bp, synthetic construct
  source 1..312: mol_type = other DNA
gccaccatggactacaaagacgatgacgacaagggcccggctgtggctgaggagcccctccaccgaccgtgagtttgggctgcatgactgcatggttgcatgaacacatattaatttcctccacttagttctacacctcattcattcattcagtgagtgtttctcgactactatgaataaaccgttatactccatgttgcgggcagaatggggatctggacagggaagcacagggcacgagttcaccaatggctgtcaagctacgctgcctgatgagtccgtgaggacgaaacgagtaagctcgtcgcagcg

SEQ ID NO: 3 - DNA, 324 bp, synthetic construct
  source 1..324: mol_type = other DNA
gccaccatggactacaaagacgatgacgacaagggcccggctgtggctgaggagcccctccaccgaccgtgagtttgggctgcatgactgcatggttgcatgaacacatattaatttcctccacttagttctacacctcattcattcattcagtgagtgtttctcgactactatgaataaaccgttatactccatgttgcgggcagaatggggatctggacagggaagcacagggcacgagttcaccaatggctgtcaagctacgctgcccgcctaacactgccaatgccggtcccaagcccggataaaaagtggagggggcgg

SEQ ID NO: 4 - DNA, 342 bp, synthetic construct
  source 1..342: mol_type = other DNA
gccaccatggactacaaagacgatgacgacaagagagccaagtggaggaagaagcgaatgcggcggtgagtttgggctgcatgactgcatggttgcatgaacacatattaatttccccagagttgcctttccctcccacattaaatcaaacgtccacataaagaatgaggtggtaaaatgaacaagcactacggttctatcgttctctgttctgttaaatcctggctccagggagaaaacactcaaacgtttttctcctaaagatcggccggcatggtcccagcctcctcgctggcgccggctgggcaacatgcttcggcatggcgaatgggacgcggccgc

SEQ ID NO: 5 - DNA, 309 bp, synthetic construct
  source 1..309: mol_type = other DNA
gccaccatggactacaaagacgatgacgacaagagagccaagtggaggaagaagcgaatgcggcggtgagtttgggctgcatgactgcatggttgcatgaacacatattaatttccccagagttgcctttccctcccacattaaatcaaacgtccacataaagaatgaggtggtaaaatgaacaagcactacggttctatcgttctctgttctgttaaatcctggctccagggagaaaacactcaaacgtttttctcctaaagatcctgatgagtccgtgaggacgaaacgagtaagctcgtcgatctt

SEQ ID NO: 6 - DNA, 321 bp, synthetic construct
  source 1..321: mol_type = other DNA
gccaccatggactacaaagacgatgacgacaagagagccaagtggaggaagaagcgaatgcggcggtgagtttgggctgcatgactgcatggttgcatgaacacatattaatttccccagagttgcctttccctcccacattaaatcaaacgtccacataaagaatgaggtggtaaaatgaacaagcactacggttctatcgttctctgttctgttaaatcctggctccagggagaaaacactcaaacgtttttctcctaaagatcccgcctaacactgccaatgccggtcccaagcccggataaaaagtggagggggcgg

SEQ ID NO: 7 - DNA, 351 bp, synthetic construct
  source 1..351: mol_type = other DNA
gccgccgccaccatggactacaaagacgatgacgacaagggcccggctgtggctgaggagcccctccaccgaccgtgagtttgggctgcatgactgcatggttgcatgaacacatattaatttcctccacttagttctacacctcattcattcattcagtgagtgtttctcgactactatgaataaaccgttatactccatgttgcgggcagaatggggatctggacagggaagcacagggcacgagttcaccaatggctgtcaagctacgctgcggccggcatggtcccagcctcctcgctggcgccggctgggcaacatgcttcggcatggcgaatgggacgcggccgc

SEQ ID NO: 8 - DNA, 51 bp, synthetic construct
  source 1..51: mol_type = other DNA
gtgagtttgggctgcatgactgcatggttgcatgaacacatattaatttcc

SEQ ID NO: 9 - DNA, 380 bp, synthetic construct
  source 1..380: mol_type = other DNA
agacccaagcttggtaccgagctcggatccggccaccatggactacaaagacgatgacgacaagggcccggctgtggctgaggagcccctccaccgaccgtgagtttgggctgcatgactgcatggttgcatgaacacatattaatttcctccacttagttctacacctcattcattcattcagtgagtgtttctcgactactatgaataaaccgttatactccatgttgcgggcagaatggggatctggacagggaagcacagggcacgagttcaccaatggctgtcaagctacgctgcccgcctaacactgccaatgccggtcccaagcccggataaaaagtggagggggcgggaattctgcagatatccatcacact

SEQ ID NO: 10 - DNA, 418 bp, synthetic construct
  source 1..418: mol_type = other DNA
agacccaagcttggtaccgagctcggatccggccaccatggactacaaagacgatgacgacaagggcccggctgtggctgaggagcccctccaccgaccgtgagtttgggctgcatgactgcatggttgcatgaacacatattaatttcctccacttagttctacacctcattcattcattcagtgagtgtttctcgactactatgaataaaccgttatactccatgttgcgggcagaatggggatctggacagggaagcacagggcacgagttcaccaatggctgtcaagctacgctgcgaaggtttttcttttcctgagaaaacaacacgtattgttttctcaggttttgctttttggcctttttctagcttaaaaaaaaaaaaagcaaaagaattctgcagatatccatcacact

SEQ ID NO: 11 - DNA, 418 bp, synthetic construct
  source 1..418: mol_type = other DNA
agacccaagcttggtaccgagctcggatccggccaccatggactacaaagacgatgacgacaagggcccggctgtggctgaggagcccctccaccgaccgtgagtttgggctgcatgactgcatggttgcatgaacacatattaatttcctccacttagttctacacctcattcattcattcagtgagtgtttctcgactactatgaataaaccgttatactccatgttgcgggcagaatggggatctggacagggaagcacagggcacgagttcaccaatggctgtcaagctacgctgcaggtgtttcttttactgagtgcagcccatggccgcactcaggttttgcttttcaccttcccatctgtgaaagagtgagcaggaaaaagcaaaagaattctgcagatatccatcacact

SEQ ID NO: 12 - DNA, 383 bp, synthetic construct
  source 1..383: mol_type = other DNA
agacccaagcttggtaccgagctcggatccggccaccatggactacaaagacgatgacgacaagggcccggctgtggctgaggagcccctccaccgaccgtgagtttgggctgcatgactgcatggttgcatgaacacatattaatttcctccacttagttctacacctcattcattcattcagtgagtgtttctcgactactatgaataaaccgttatactccatgttgcgggcagaatggggatctggacagggaagcacagggcacgagttcaccaatggctgtcaagctacgctgcaaaggtttttcttttcctgagaaatttctcaggttttgcttttaaaaaaaaagcaaaagaattctgcagatatccatcacact

SEQ ID NO: 13 - DNA, 473 bp, synthetic construct
  source 1..473: mol_type = other DNA
agacccaagcttggtaccgagctcggatccggccaccatggactacaaagacgatgacgacaagggcccggctgtggctgaggagcccctccaccgaccgtgagtttgggctgcatgactgcatggttgcatgaacacatattaatttcctccacttagttctacacctcattcattcattcagtgagtgtttctcgactactatgaataaaccgttatactccatgttgcgggcagaatggggatctggacagggaagcacagggcacgagttcaccaatggctgtcaagctacgctgcgaaggtttttcttttcctgagaaaacaacacgtattgttttctcaggttttgctttttggcctttttctagcttaaaaaaaaaaaaagcaaaaccgcctaacactgccaatgccggtcccaagcccggataaaaagtggagggggcgggaattctgcagatatccatcacact

SEQ ID NO: 14 - DNA, 473 bp, synthetic construct
  source 1..473: mol_type = other DNA
agacccaagcttggtaccgagctcggatccggccaccatggactacaaagacgatgacgacaagggcccggctgtggctgaggagcccctccaccgaccgtgagtttgggctgcatgactgcatggttgcatgaacacatattaatttcctccacttagttctacacctcattcattcattcagtgagtgtttctcgactactatgaataaaccgttatactccatgttgcgggcagaatggggatctggacagggaagcacagggcacgagttcaccaatggctgtcaagctacgctgcaggtgtttcttttactgagtgcagcccatggccgcactcaggttttgcttttcaccttcccatctgtgaaagagtgagcaggaaaaagcaaaaccgcctaacactgccaatgccggtcccaagcccggataaaaagtggagggggcgggaattctgcagatatccatcacact

SEQ ID NO: 15 - DNA, 438 bp, synthetic construct
  source 1..438: mol_type = other DNA
agacccaagcttggtaccgagctcggatccggccaccatggactacaaagacgatgacgacaagggcccggctgtggctgaggagcccctccaccgaccgtgagtttgggctgcatgactgcatggttgcatgaacacatattaatttcctccacttagttctacacctcattcattcattcagtgagtgtttctcgactactatgaataaaccgttatactccatgttgcgggcagaatggggatctggacagggaagcacagggcacgagttcaccaatggctgtcaagctacgctgcaaaggtttttcttttcctgagaaatttctcaggttttgcttttaaaaaaaaagcaaaaccgcctaacactgccaatgccggtcccaagcccggataaaaagtggagggggcgggaattctgcagatatccatcacact

SEQ ID NO: 16 - DNA, 473 bp, synthetic construct
  source 1..473: mol_type = other DNA
agacccaagcttggtaccgagctcggatccggccaccatggactacaaagacgatgacgacaagggcccggctgtggctgaggagcccctccaccgaccgtgagtttgggctgcatgactgcatggttgcatgaacacatattaatttcctccacttagttctacacctcattcattcattcagtgagtgtttctcgactactatgaataaaccgttatactccatgttgcgggcagaatggggatctggacagggaagcacagggcacgagttcaccaatggctgtcaagctacgctgcccgcctaacactgccaatgccggtcccaagcccggataaaaagtggagggggcgggaaggtttttcttttcctgagaaaacaacacgtattgttttctcaggttttgctttttggcctttttctagcttaaaaaaaaaaaaagcaaaagaattctgcagatatccatcacact

SEQ ID NO: 17 - DNA, 473 bp, synthetic construct
  source 1..473: mol_type = other DNA
agacccaagcttggtaccgagctcggatccggccaccatggactacaaagacgatgacgacaagggcccggctgtggctgaggagcccctccaccgaccgtgagtttgggctgcatgactgcatggttgcatgaacacatattaatttcctccacttagttctacacctcattcattcattcagtgagtgtttctcgactactatgaataaaccgttatactccatgttgcgggcagaatggggatctggacagggaagcacagggcacgagttcaccaatggctgtcaagctacgctgcccgcctaacactgccaatgccggtcccaagcccggataaaaagtggagggggcggaggtgtttcttttactgagtgcagcccatggccgcactcaggttttgcttttcaccttcccatctgtgaaagagtgagcaggaaaaagcaaaagaattctgcagatatccatcacact

SEQ ID NO: 18 - DNA, 438 bp, synthetic construct
  source 1..438: mol_type = other DNA
agacccaagcttggtaccgagctcggatccggccaccatggactacaaagacgatgacgacaagggcccggctgtggctgaggagcccctccaccgaccgtgagtttgggctgcatgactgcatggttgcatgaacacatattaatttcctccacttagttctacacctcattcattcattcagtgagtgtttctcgactactatgaataaaccgttatactccatgttgcgggcagaatggggatctggacagggaagcacagggcacgagttcaccaatggctgtcaagctacgctgcccgcctaacactgccaatgccggtcccaagcccggataaaaagtggagggggcggaaaggtttttcttttcctgagaaatttctcaggttttgcttttaaaaaaaaagcaaaagaattctgcagatatccatcacact

SEQ ID NO: 19 - DNA, 375 bp, synthetic construct
  source 1..375: mol_type = other DNA
agacccaagcttggtaccgagctcggatccggccaccatggactacaaagacgatgacgacaagggcccggctgtggctgaggagcccctccaccgaccgtgagtttgggctgcatgactgcatggttgcatgaacacatattaatttcctccacttagttctacacctcattcattcattcagtgagtgtttctcgactactatgaataaaccgttatactccatgttgcgggcagaatggggatctggacagggaagcacagggcacgagttcaccaatggctgtcaagctacgctgcaaacagagaagtcaaccagagaaacacacgttgtggtatattacctggtagaattctgcagatatccatcacact

SEQ ID NO: 20 - DNA, 377 bp, synthetic construct
  source 1..377: mol_type = other DNA
agacccaagcttggtaccgagctcggatccggccaccatggactacaaagacgatgacgacaagggcccggctgtggctgaggagcccctccaccgaccgtgagtttgggctgcatgactgcatggttgcatgaacacatattaatttcctccacttagttctacacctcattcattcattcagtgagtgtttctcgactactatgaataaaccgttatactccatgttgcgggcagaatggggatctggacagggaagcacagggcacgagttcaccaatggctgtcaagctacgctgccaacagcgaagcgcgccagggaaacacaccatgtgtggtatattatctggcagaattctgcagatatccatcacact

SEQ ID NO: 21 - DNA, 377 bp, synthetic construct
  source 1..377: mol_type = other DNA
agacccaagcttggtaccgagctcggatccggccaccatggactacaaagacgatgacgacaagggcccggctgtggctgaggagcccctccaccgaccgtgagtttgggctgcatgactgcatggttgcatgaacacatattaatttcctccacttagttctacacctcattcattcattcagtgagtgtttctcgactactatgaataaaccgttatactccatgttgcgggcagaatggggatctggacagggaagcacagggcacgagttcaccaatggctgtcaagctacgctgccaacagcgaagcggaacggcgaaacacaccttgtgtggtatattacccgttggaattctgcagatatccatcacact

SEQ ID NO: 22 - DNA, 501 bp, synthetic construct
  source 1..501: mol_type = other DNA
agacccaagcttggtaccgagctcggatccggccaccatggactacaaagacgatgacgacaagggcccggctgtggctgaggagcccctccaccgaccgtgagtttgggctgcatgactgcatggttgcatgaacacatattaatttcctccacttagttctacacctcattcattcattcagtgagtgtttctcgactactatgaataaaccgttatactccatgttgcgggcagaatggggatctggacagggaagcacagggcacgagttcaccaatggctgtcaagctacgctgcgggaaagcttgcgaagggcgtcgtcgccccgagcggtagtaagcagggaactcacctccaatttcagtactgaaattgtcgtagcagttgactactgttatgtgattggtagaggctaagtgacggtattggcgtaagtcagtattgcagcacagcacaagcccgcttgcgagaatgaattctgcagatatccatcacact

SEQ ID NO: 23 - DNA, 492 bp, synthetic construct
  source 1..492: mol_type = other DNA
agacccaagcttggtaccgagctcggatccggccaccatggactacaaagacgatgacgacaagggcccggctgtggctgaggagcccctccaccgaccgtgagtttgggctgcatgactgcatggttgcatgaacacatattaatttcctccacttagttctacacctcattcattcattcagtgagtgtttctcgactactatgaataaaccgttatactccatgttgcgggcagaatggggatctggacagggaagcacagggcacgagttcaccaatggctgtcaagctacgctgctaattatagcgcccgaactaagcgcccggaaaaaggcttagttgacgaggatggaggttatcgaattttcgggcggatgcctcccggctgagtgtgcagatcacagccgtaaggatttcttcaaaccaagggggtgactccttgaacaaagagaaatcacatgatctgaattctgcagatatccatcacact

SEQ ID NO: 24 - DNA, 401 bp, synthetic construct
  source 1..401: mol_type = other DNA
agacccaagcttggtaccgagctcggatccggccaccatggactacaaagacgatgacgacaagggcccggctgtggctgaggagcccctccaccgaccgtgagtttgggctgcatgactgcatggttgcatgaacacatattaatttcctccacttagttctacacctcattcattcattcagtgagtgtttctcgactactatgaataaaccgttatactccatgttgcgggcagaatggggatctggacagggaagcacagggcacgagttcaccaatggctgtcaagctacgctgcggacccgcaaggccgacggcatccgccgccgctggtgcaagtccagccgccccggggcgggcgctcatgggtaaacgaattctgcagatatccatcacact

SEQ ID NO: 25 - DNA, 403 bp, synthetic construct
  source 1..403: mol_type = other DNA
agacccaagcttggtaccgagctcggatccggccaccatggactacaaagacgatgacgacaagggcccggctgtggctgaggagcccctccaccgaccgtgagtttgggctgcatgactgcatggttgcatgaacacatattaatttcctccacttagttctacacctcattcattcattcagtgagtgtttctcgactactatgaataaaccgttatactccatgttgcgggcagaatggggatctggacagggaagcacagggcacgagttcaccaatggctgtcaagctacgctgcggacccgcaaggccgacggcatccgccgccgctggtgcaagtccagccgccccatggggcgggcgctcatgggtaaacgaattctgcagatatccatcacact

SEQ ID NO: 26 - DNA, 390 bp, synthetic construct
  source 1..390: mol_type = other DNA
agacccaagcttggtaccgagctcggatccggccaccatggactacaaagacgatgacgacaagggcccggctgtggctgaggagcccctccaccgaccgtgagtttgggctgcatgactgcatggttgcatgaacacatattaatttcctccacttagttctacacctcattcattcattcagtgagtgtttctcgactactatgaataaaccgttatactccatgttgcgggcagaatggggatctggacagggaagcacagggcacgagttcaccaatggctgtcaagctacgctgcggagccgttcgggcggctataaacagacctcaggcccgaagcgtggcggcgatccgccggtggtagaattctgcagatatccatcacact

SEQ ID NO: 27 - DNA, 405 bp, synthetic construct
  source 1..405: mol_type = other DNA
agacccaagcttggtaccgagctcggatccggccaccatggactacaaagacgatgacgacaagggcccggctgtggctgaggagcccctccaccgaccgtgagtttgggctgcatgactgcatggttgcatgaacacatattaatttcctccacttagttctacacctcattcattcattcagtgagtgtttctcgactactatgaataaaccgttatactccatgttgcgggcagaatggggatctggacagggaagcacagggcacgagttcaccaatggctgtcaagctacgctgccattcctcagaaaatgacaaacctgtggggcgtaagtagatatgtacatatctatgatcgtgcagacgttaaaatcaggtgaattctgcagatatccatcacact

SEQ ID NO: 28 - DNA, 474 bp, synthetic construct
  source 1..474: mol_type = other DNA
agacccaagcttggtaccgagctcggatccggccaccatggactacaaagacgatgacgacaagggcccggctgtggctgaggagcccctccaccgaccgtgagtttgggctgcatgactgcatggttgcatgaacacatattaatttcctccacttagttctacacctcattcattcattcagtgagtgtttctcgactactatgaataaaccgttatactccatgttgcgggcagaatggggatctggacagggaagcacagggcacgagttcaccaatggctgtcaagctacgctgcccgcctaacactgccaatgccggtcccaagcccggataaaaagtggagggggcgggtttctttttgggaatggactcttttgttcacgtaaacaaaagagtccttttgctttttggcctttttctagcttaaaaaaaaaaaaagcaaaagaattctgcagatatccatcacact

SEQ ID NO: 29 - DNA, 476 bp, synthetic construct
  source 1..476: mol_type = other DNA
agacccaagcttggtaccgagctcggatccggccaccatggactacaaagacgatgacgacaagggcccggctgtggctgaggagcccctccaccgaccgtgagtttgggctgcatgactgcatggttgcatgaacacatattaatttcctccacttagttctacacctcattcattcattcagtgagtgtttctcgactactatgaataaaccgttatactccatgttgcgggcagaatggggatctggacagggaagcacagggcacgagttcaccaatggctgtcaagctacgctgcccgcctaacactgccaatgccggtcccaagcccggataaaaagtggagggggcgggtttctttttgggaatggactcttttgttcacgtaaacaaaagagtcctttttgctttttggcctttttctagcttaaaaaaaaaaaaagcaaaaagaattctgcagatatccatcacact

SEQ ID NO: 30 - DNA, 476 bp, synthetic construct
  source 1..476: mol_type = other DNA
agacccaagcttggtaccgagctcggatccggccaccatggactacaaagacgatgacgacaagggcccggctgtggctgaggagcccctccaccgaccgtgagtttgggctgcatgactgcatggttgcatgaacacatattaatttcctccacttagttctacacctcattcattcattcagtgagtgtttctcgactactatgaataaaccgttatactccatgttgcgggcagaatggggatctggacagggaagcacagggcacgagttcaccaatggctgtcaagctacgctgcccgcctaacactgccaatgccggtcccaagcccggataaaaagtggagggggcgggtttctttttgggaatggactcttttgttcacgtaaacaaaagagtcctttttgctttttgcgctttttctcgcttaaaaaaaaaaaaagcaaaaagaattctgcagatatccatcacact

SEQ ID NO: 31 - DNA, 426 bp, synthetic construct
  source 1..426: mol_type = other DNA
agacccaagcttggtaccgagctcggatccggccaccatggactacaaagacgatgacgacaagggcccggctgtggctgaggagcccctccaccgaccgtgagtttgggctgcatgactgcatggttgcatgaacacatattaatttcctccacttagttctacacctcattcattcattcagtgagtgtttctcgactactatgaataaaccgttatactccatgttgcgggcagaatggggatctggacagggaagcacagggcacgagttcaccaatggctgtcaagctacgctgcccgcctaacactgccaatgccggtcccaagcccggataaaaagtggagggggcggttctcaaatatgagtttttacatgacaaagtttttaacgaggcagcgaattctgcagatatccatcacact

SEQ ID NO: 32 - DNA, 428 bp, synthetic construct
  source 1..428: mol_type = other DNA
agacccaagcttggtaccgagctcggatccggccaccatggactacaaagacgatgacgacaagggcccggctgtggctgaggagcccctccaccgaccgtgagtttgggctgcatgactgcatggttgcatgaacacatattaatttcctccacttagttctacacctcattcattcattcagtgagtgtttctcgactactatgaataaaccgttatactccatgttgcgggcagaatggggatctggacagggaagcacagggcacgagttcaccaatggctgtcaagctacgctgcccgcctaacactgccaatgccggtcccaagcccggataaaaagtggagggggcggggtacaaaacctgctggtgattttttacccaacaaataataaataaaagaattctgcagatatccatcacact

SEQ ID NO: 33 - DNA, 430 bp, synthetic construct
  source 1..430: mol_type = other DNA
agacccaagcttggtaccgagctcggatccggccaccatggactacaaagacgatgacgacaagggcccggctgtggctgaggagcccctccaccgaccgtgagtttgggctgcatgactgcatggttgcatgaacacatattaatttcctccacttagttctacacctcattcattcattcagtgagtgtttctcgactactatgaataaaccgttatactccatgttgcgggcagaatggggatctggacagggaagcacagggcacgagttcaccaatggctgtcaagctacgctgcccgcctaacactgccaatgccggtcccaagcccggataaaaagtggagggggcggtttccccaacctctgggttgggttttttctctttaaaatattcaataaaagaattctgcagatatccatcacact

SEQ ID NO: 34 - DNA, 428 bp, synthetic construct
  source 1..428: mol_type = other DNA
agacccaagcttggtaccgagctcggatccggccaccatggactacaaagacgatgacgacaagggcccggctgtggctgaggagcccctccaccgaccgtgagtttgggctgcatgactgcatggttgcatgaacacatattaatttcctccacttagttctacacctcattcattcattcagtgagtgtttctcgactactatgaataaaccgttatactccatgttgcgggcagaatggggatctggacagggaagcacagggcacgagttcaccaatggctgtcaagctacgctgcccgcctaacactgccaatgccggtcccaagcccggataaaaagtggagggggcggttctcacaagggtgagttttacctagacaggtttttaaccaggcaaccgaattctgcagatatccatcacact

SEQ ID NO: 35 - DNA, 465 bp, synthetic construct
  source 1..465: mol_type = other DNA
agacccaagcttggtaccgagctcggatccggccaccatggactacaaagacgatgacgacaagaggcctacatctccatggaggggtaccagtcctggacgctggctaacgggtggaacagccaggtgtactgcaccaaggaccagccacaggggtcccacttttggaaatcctctttcccaggtgagtttgggctgcatgactgcatggttgcatgaacacatattaatttcctaacaacaatggttaatttgattgattatcatagtttctcaaaccggcatcttaacaagcttgcaattttatggtgtggttattgggcacgttcatgaggacagtttctgcaaatgtcggttgtataaaaacaaacttacatacatagacccgcctaacactgccaatgccggtcccaagcccggataaaaagtggagggggcgggaattctgcagatatccatcacact

SEQ ID NO: 36 - DNA, 465 bp, synthetic construct
  source 1..465: mol_type = other DNA
agacccaagcttggtaccgagctcggatccggccaccatggactacaaagacgatgacgacaagaggcctacatctccatggaggggtaccagtcctggacgctggctaacgggtggaacagccaggtgtactgcaccaaggaccagccacaggggtcccacttttggaaatcctctttcccaggtgagtttgggctgcatgactgcatggttgcatgaacacatattaatttcccttaacaagcttgcaattttatggtgtggttattgggcacgttcatgaggacagtttctgcaaatgtcggttgtataaaaacaaacttacatacatagacacataccaactcaggtcttttgctcaaattttgatttcccgcgttcctatccgcctaacactgccaatgccggtcccaagcccggataaaaagtggagggggcgggaattctgcagatatccatcacact

SEQ ID NO: 37 - DNA, 465 bp, synthetic construct
  source 1..465: mol_type = other DNA
agacccaagcttggtaccgagctcggatccggccaccatggactacaaagacgatgacgacaagaggcctacatctccatggaggggtaccagtcctggacgctggctaacgggtggaacagccaggtgtactgcaccaaggaccagccacaggggtcccacttttggaaatcctctttcccaggtgagtttgggctgcatgactgcatggttgcatgaacacatattaatttcccaggggtgcaggaaatttttaaaaaatgccatgctgtttggtctttattatgtacatagacataacacttacctgttattaaagaaggattatttgcacttaacaacaatggttaatttgattgattatcatagtttctcaaaccggcatccgcctaacactgccaatgccggtcccaagcccggataaaaagtggagggggcgggaattctgcagatatccatcacact

SEQ ID NO: 38 - DNA, 465 bp, synthetic construct
  source 1..465: mol_type = other DNA
agacccaagcttggtaccgagctcggatccggccaccatggactacaaagacgatgacgacaagaggcctacatctccatggaggggtaccagtcctggacgctggctaacgggtggaacagccaggtgtactgcaccaaggaccagccacaggggtcccacttttggaaatcctctttcccaggtgagtttgggctgcatgactgcatggttgcatgaacacatattaatttcctgtacatagacataacacttacctgttattaaagaaggattatttgcacttaacaacaatggttaatttgattgattatcatagtttctcaaaccggcatcttaacaagcttgcaattttatggtgtggttattgggcacgttcatgaggccgcctaacactgccaatgccggtcccaagcccggataaaaagtggagggggcgggaattctgcagatatccatcacact

SEQ ID NO: 39 - DNA, 465 bp, synthetic construct
  source 1..465: mol_type = other DNA
agacccaagcttggtaccgagctcggatccggccaccatggactacaaagacgatgacgacaagaggcctacatctccatggaggggtaccagtcctggacgctggctaacgggtggaacagccaggtgtactgcaccaaggaccagccacaggggtcccacttttggaaatcctctttcccaggtgagtttgggctgcatgactgcatggttgcatgaacacatattaatttccttgaccaagtggagggtgctcttccagctcttgaacaggacctagagagttggatgtattagatgggcgtacgcagtatgtgcccagttgtatgattgtgcgttttcaaggaagggagtgtgcgtcgattcgttcagtatcgacagggggccgcctaacactgccaatgccggtcccaagcccggataaaaagtggagggggcgggaattctgcagatatccatcacact

SEQ ID NO: 40 - DNA, 1179 bp, synthetic construct
  source 1..1179: mol_type = other DNA
agacccaagcttggtaccgagctcggatccggccaccatggactacaaagacgatgacgacaaggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagaggcctacatctccatggaggggtaccagtcctggacgctggctaacgggtggaacagccaggtgtactgcaccaaggaccagccacaggggtcccacttttggaaatcctctttcccaggtgagtttgggctgcatgactgcatggttgcatgaacacatattaatttcccaggggtgcaggaaatttttaaaaaatgccatgctgtttggtctttattatgtacatagacataacacttacctgttattaaagaaggattatttgcacttaacaacaatggttaatttgattgattatcatagtttctcaaaccggcatccgcctaacactgccaatgccggtcccaagcccggataaaaagtggagggggcgggaattctgcagatatccatcacact

SEQ ID NO: 41 - DNA, 1488 bp, synthetic construct
  source 1..1488: mol_type = other DNA
agacccaagcttggtaccgagctcggatccggccaccatggactacaaagacgatgacgacaagaatttactgaccgtacaccaaaatttgcctgcattaccggtcgatgcaacgagtgatgaggttcgcaagaacctgatggacatgttcagggatcgccaggcgttttctgagcatacctggaaaatgcttctgtccgtttgccggtcgtgggcggcatggtgcaagttgaataaccggaaatggtttcccgcagaacctgaagatgttcgcgattatcttctatatcttcaggcgcgcggtctggcagtaaaaactatccagcaacatttgggccagctaaacatgcttcatcgtcggtccgggctgccacgaccaagtgacagcaatgctgtttcactggttatgcggcggatccgaaaagaaaacgttgatgccggtgaacgtgcaaaacaggctctagcgttcgaacgcactgatttcgaccaggttcgttcactcatggaaaatagcgatcgctgccaggatatacgtaatctggcatttctggggattgcttataacaccctgttacgtatagccgaaattgccaggatcagggttaaagatatctcacgtactgacggtgggagaatgttaatccatattggcagaacgaaaacgctggttagcaccgcaggtgtagagaaggcacttagcctgggggtaactaaactggtcgagcgatggatttccgtctctggtgtagctgatgatccgaataactacctgttttgccgggtcagaaaaaatggtgttgccgcgccatctgccaccagccagctatcaactcgcgccctggaagggatttttgaagcaactcatcgattgatttacggcgctaaggatgactctggtcagagatacctggcctggtctggacacagtgcccgtgtcggagccgcgcgagatatggcccgcgctggagtttcaataccggagatcatgcaagctggtggctggaccaatgtaaatattgtcatgaactatatccgtaacctggatagtgaaacaggggcaatggtgcgcctgctggaagatggcgataggcctacatctccatggaggggtaccagtcctggacgctggctaacgggtggaacagccaggtgtactgcaccaaggaccagccacaggggtcccacttttggaaatcctctttcccaggtgagtttgggctgcatgactgcatggttgcatgaacacatattaatttcccaggggtgcaggaaatttttaaaaaatgccatgctgtttggtctttattatgtacatagacataacacttacctgttattaaagaaggattatttgcacttaacaacaatggttaatttgattgattatcatagtttctcaaaccggcatccgcctaacactgccaatgccggtcccaagcccggataaaaagtggagggggcgggaattctgcagatatccatcacact

SEQ ID NO: 42 - DNA, 417 bp, synthetic construct
  source 1..417: mol_type = other DNA
agacccaagcttggtaccgagctcggatccggccaccatggactacaaagacgatgacgacaagatggcgacgaaggccgtgtgcgtgctgaagggcgacggcccagtgcagggcatcatcaattttgaacaaaaggtgagtttgggctgcatgactgcatggttgcatgaacacatattaatttccagagtttctgaacaaagaaaacagctgatttacctttacccctcacagtgaagtaaaaagcagccagacttgggagtggaaaaatctgtgtccaaatcccaggctgaccactttctagccagtgaacctccagcaagctgcttaactgctccgcctaacactgccaatgccggtcccaagcccggataaaaagtggagggggcgggaattctgcagatatccatcacact

SEQ ID NO: 43 - DNA, 417 bp, synthetic construct
  source 1..417: mol_type = other DNA
agacccaagcttggtaccgagctcggatccggccaccatggactacaaagacgatgacgacaagatggcgacgaaggccgtgtgcgtgctgaagggcgacggcccagtgcagggcatcatcaattttgaacaaaaggtgagtttgggctgcatgactgcatggttgcatgaacacatattaatttccctaggcctgtgtcctcaaaagggagatggtaatcttgttcccacactcaatgcacaagcaccctgtggccaggcttcatgattaaataacataaaaagcacctaatagagtggcaagcaaagtgagtacctacacatcagtgttagatacccgcctaacactgccaatgccggtcccaagcccggataaaaagtggagggggcgggaattctgcagatatccatcacact

SEQ ID NO: 44 - DNA, 417 bp, synthetic construct
  source 1..417: mol_type = other DNA
agacccaagcttggtaccgagctcggatccggccaccatggactacaaagacgatgacgacaagatggcgacgaaggccgtgtgcgtgctgaagggcgacggcccagtgcagggcatcatcaattttgaacaaaaggtgagtttgggctgcatgactgcatggttgcatgaacacatattaatttccttcataggctttgactattacagtaaataggtaatgtaagtacctatttatgtagtaaatacttttgaataggttatatttgtacatgttgaatatacatgtacatgtaaacgttttttattttgagacaagagtctcgctctgtcgcccccgcctaacactgccaatgccggtcccaagcccggataaaaagtggagggggcgggaattctgcagatatccatcacact

SEQ ID NO: 45 - DNA, 417 bp, synthetic construct
  source 1..417: mol_type = other DNA
agacccaagcttggtaccgagctcggatccggccaccatggactacaaagacgatgacgacaagatggcgacgaaggccgtgtgcgtgctgaagggcgacggcccagtgcagggcatcatcaattttgaacaaaaggtgagtttgggctgcatgactgcatggttgcatgaacacatattaatttccaggctggagtgcagtggcatgatctcagctcactgcaagctccgcctcccgggttcacgccattctcctgcctcagcctttttaacccaaaaggtgtatgtctcatcctacactctggagccccttgctttttctccacgcacaccctttccgcctaacactgccaatgccggtcccaagcccggataaaaagtggagggggcgggaattctgcagatatccatcacact

SEQ ID NO: 46 - DNA, 417 bp, synthetic construct
  source 1..417: mol_type = other DNA
agacccaagcttggtaccgagctcggatccggccaccatggactacaaagacgatgacgacaagatggcgacgaaggccgtgtgcgtgctgaagggcgacggcccagtgcagggcatcatcaattttgaacaaaaggtgagtttgggctgcatgactgcatggttgcatgaacacatattaatttccagtggaaaaatctgtgtccaaatcccaggctgaccactttctagccagtgaacctccagcaagctgcttaactgctctaggcctgtgtcctcaaaagggagatggtaatcttgttcccacactcaatgcacaagcaccctgtggccaggcccgcctaacactgccaatgccggtcccaagcccggataaaaagtggagggggcgggaattctgcagatatccatcacact

SEQ ID NO: 47 - DNA, 464 bp, synthetic construct
  source 1..464: mol_type = other DNA
agacccaagcttggtaccgagctcggatccggccaccatggactacaaagacgatgacgacaagcgcgtctccatctacagcacgcgccgcccggtgttggcgcgcacccacgtccagggccgcgtctacaacttcctcgagcgtcccaccggctggaaatgcttcgtttaccattttgcagtgtgagtttgggctgcatgactgcatggttgcatgaacacatattaatttccgcatcacggcctctgcccatcccacagatgcacctggggagtggaggggcccctggcctcagccccgcccccaggtaggaacagacagtgcttcgaaaacccagtcatccattagctggtaagcacacactagaggtaggtccgcacgagccgcctaacactgccaatgccggtcccaagcccggataaaaagtggagggggcgggaattctgcagatatccatcacact

SEQ ID NO: 48 - DNA, 464 bp, synthetic construct
  source 1..464: mol_type = other DNA
agacccaagcttggtaccgagctcggatccggccaccatggactacaaagacgatgacgacaagcgcgtctccatctacagcacgcgccgcccggtgttggcgcgcacccacgtccagggccgcgtctacaacttcctcgagcgtcccaccggctggaaatgcttcgtttaccattttgcagtgtgagtttgggctgcatgactgcatggttgcatgaacacatattaatttccggatgccgtcccgccacgggaagggacgaagcaccaacacatgctgccacgtggatgggtccgagaaacatggcgccaaggccagacacagacaggtgaccacctgctgaccacctgcagtgtggctacgccctggacagccagagccacccgcctaacactgccaatgccggtcccaagcccggataaaaagtggagggggcgggaattctgcagatatccatcacact

SEQ ID NO: 49 - DNA, 464 bp, synthetic construct
  source 1..464: mol_type = other DNA
agacccaagcttggtaccgagctcggatccggccaccatggactacaaagacgatgacgacaagcgcgtctccatctacagcacgcgccgcccggtgttggcgcgcacccacgtccagggccgcgtctacaacttcctcgagcgtcccaccggctggaaatgcttcgtttaccattttgcagtgtgagtttgggctgcatgactgcatggttgcatgaacacatattaatttccagaaacagaaagcaggctggtggctgccccagggcgggactgctgtgggcgtggggttttcttttggggtaattaaacgttctggatgtggatggtggtggtggctacacaactctgaacattctagagccacttagggccctagagccaccgcctaacactgccaatgccggtcccaagcccggataaaaagtggagggggcgggaattctgcagatatccatcacact

SEQ ID NO: 50 - DNA, 464 bp, synthetic construct
  source 1..464: mol_type = other DNA
agacccaagcttggtaccgagctcggatccggccaccatggactacaaagacgatgacgacaagcgcgtctccatctacagcacgcgccgcccggtgttggcgcgcacccacgtccagggccgcgtctacaacttcctcgagcgtcccaccggctggaaatgcttcgtttaccattttgcagtgtgagtttgggctgcatgactgcatggttgcatgaacacatattaatttcccaaatgaactgcagtgtgtaattacgtctctacaaagctgattttacaaatcacttccagagagcagtcaacgccccctgtccaggagttcagggcccagggcaggggtgttggtggtcacagcacagaagccagcccgccaggtgggggccgcctaacactgccaatgccggtcccaagcccggataaaaagtggagggggcgggaattctgcagatatccatcacact

SEQ ID NO: 51 - DNA, 464 bp, synthetic construct
  source 1..464: mol_type = other DNA
agacccaagcttggtaccgagctcggatccggccaccatggactacaaagacgatgacgacaagcgcgtctccatctacagcacgcgccgcccggtgttggcgcgcacccacgtccagggccgcgtctacaacttcctcgagcgtcccaccggctggaaatgcttcgtttaccattttgcagtgtgagtttgggctgcatgactgcatggttgcatgaacacatattaatttcctcctgacacctgcaccaagcgtctgcttcccagaggagcctccaggcggcaggctgtgggccgctctcctccagtttcacttcctcctttccaaggccccgaggccgagccacagaactcatgaaagggaaggcgcacagagccccacccccgcctaacactgccaatgccggtcccaagcccggataaaaagtggagggggcgggaattctgcagatatccatcacact

SEQ ID NO: 52 - DNA, 465 bp, synthetic construct
  source 1..465: mol_type = other DNA
agacccaagcttggtaccgagctcggatccggccaccatggactacaaagacgatgacgacaagaccaagtgggtaaactcgcacctggcccgggtcacgtgccgggtgggggacctgtacagcgacctccgggacggacgcaacctgctgaggctcctcgaggtgctctcgggcgaaattctggtgagtttgggctgcatgactgcatggttgcatgaacacatattaatttccctgtggagggacgggggcaaaatggcacgggactgtgggcttccaccttcttccccagccttcacagggcccagctttgcacaccttccccatgacctacctcaggaacacagacaggcacagccccagggctggagccaaagctgagctccgcctaacactgccaatgccggtcccaagcccggataaaaagtggagggggcgggaattctgcagatatccatcacact

SEQ ID NO: 53 - DNA, 465 bp, synthetic construct
  source 1..465: mol_type = other DNA
agacccaagcttggtaccgagctcggatccggccaccatggactacaaagacgatgacgacaagaccaagtgggtaaactcgcacctggcccgggtcacgtgccgggtgggggacctgtacagcgacctccgggacggacgcaacctgctgaggctcctcgaggtgctctcgggcgaaattctggtgagtttgggctgcatgactgcatggttgcatgaacacatattaatttcctacagatgaggcttgggcagattaagattcccaagggaattaattgcacccagagtaggtgggaaaagaaatgttttcaatggggccactgttcctgtctccatgaagcaatgctcctatgtaatctaagtggcaaaggcacttgaaatgccgcctaacactgccaatgccggtcccaagcccggataaaaagtggagggggcgggaattctgcagatatccatcacact

SEQ ID NO: 54 - DNA, 465 bp, synthetic construct
  source 1..465: mol_type = other DNA
agacccaagcttggtaccgagctcggatccggccaccatggactacaaagacgatgacgacaagaccaagtgggtaaactcgcacctggcccgggtcacgtgccgggtgggggacctgtacagcgacctccgggacggacgcaacctgctgaggctcctcgaggtgctctcgggcgaaattctggtgagtttgggctgcatgactgcatggttgcatgaacacatattaatttccaaaggcacttgaaatgaacccatcctctgagcagagggagccactgcttcccgccctgtgccgtcactctctctgagggctgttcttccagctggtccccttggacacttttctaaggcccccccacttcccttcatgaccacagctcacccgcctaacactgccaatgccggtcccaagcccggataaaaagtggagggggcgggaattctgcagatatccatcacact

SEQ ID NO: 55 - DNA, 465 bp, synthetic construct
  source 1..465: mol_type = other DNA
agacccaagcttggtaccgagctcggatccggccaccatggactacaaagacgatgacgacaagaccaagtgggtaaactcgcacctggcccgggtcacgtgccgggtgggggacctgtacagcgacctccgggacggacgcaacctgctgaggctcctcgaggtgctctcgggcgaaattctggtgagtttgggctgcatgactgcatggttgcatgaacacatattaatttccttccccagccttcacagggcccagctttgcacaccttccccatgacctacctcaggaacacagacaggcacagccccagggctggagccaaagctgagcttacagatgaggcttgggcagattaagattcccaagggaattaattgcaccccgcctaacactgccaatgccggtcccaagcccggataaaaagtggagggggcgggaattctgcagatatccatcacact

SEQ ID NO: 56 - DNA, 465 bp, synthetic construct
  source 1..465: mol_type = other DNA
agacccaagcttggtaccgagctcggatccggccaccatggactacaaagacgatgacgacaagaccaagtgggtaaactcgcacctggcccgggtcacgtgccgggtgggggacctgtacagcgacctccgggacggacgcaacctgctgaggctcctcgaggtgctctcgggcgaaattctggtgagtttgggctgcatgactgcatggttgcatgaacacatattaatttccctcaggaacacagacaggcacagccccagggctggagccaaagctgagcttacagatgaggcttgggcagattaagattcccaagggaattaattgcacccagagtaggtgggaaaagaaatgttttcaatggggccactgttcctgtctccgcctaacactgccaatgccggtcccaagcccggataaaaagtggagggggcgggaattctgcagatatccatcacact

SEQ ID NO: 57 - DNA, 399 bp, synthetic construct
  source 1..399: mol_type = other DNA
agacccaagcttggtaccgagctcggatccggccaccatggactacaaagacgatgacgacaagatggggagaaaaaagattcagattacgaggattatggatgaacgcaatcgacaggtgagtttgggctgcatgactgcatggttgcatgaacacatattaatttccaaaacaatggtgtgtagttgtcagagccctgggcactaacacattcagaaagaaagcaaataaatctaatattaatttctgcttatcatttgaaaatgtgtgatttctagcttgctgctgaatgtgagactctaaatgcaagatttaatgccgcctaacactgccaatgccggtcccaagcccggataaaaagtggagggggcgggaattctgcagatatccatcacact

SEQ ID NO: 58 - DNA, 399 bp, synthetic construct
  source 1..399: mol_type = other DNA
agacccaagcttggtaccgagctcggatccggccaccatggactacaaagacgatgacgacaagatggggagaaaaaagattcagattacgaggattatggatgaacgcaatcgacaggtgagtttgggctgcatgactgcatggttgcatgaacacatattaatttcctagggtattttaatgaaggtgaccagggaactggtcatgtttctttgagcaagttaagatttaaaaaaaaattaatttcagcatggttagcatatttcccaattaataattagtttggagcttgtttttgactatttgaagagtggacatccgcctaacactgccaatgccggtcccaagcccggataaaaagtggagggggcgggaattctgcagatatccatcacact

SEQ ID NO: 59 - DNA, 399 bp, synthetic construct
  source 1..399: mol_type = other DNA
agacccaagcttggtaccgagctcggatccggccaccatggactacaaagacgatgacgacaagatggggagaaaaaagattcagattacgaggattatggatgaacgcaatcgacaggtgagtttgggctgcatgactgcatggttgcatgaacacatattaatttccgacattaggaacttagcattttagatctgtgttcaaacaagtcttaaaattgtctagtctcatgtcatttaattgaaaacacttgaagggatatgactgtgatctcagacgatgttgtattctgttttctagaaacccaacagggcactgccgcctaacactgccaatgccggtcccaagcccggataaaaagtggagggggcgggaattctgcagatatccatcacact

SEQ ID NO: 60 - DNA, 399 bp, synthetic construct
  source 1..399: mol_type = other DNA
agacccaagcttggtaccgagctcggatccggccaccatggactacaaagacgatgacgacaagatggggagaaaaaagattcagattacgaggattatggatgaacgcaatcgacaggtgagtttgggctgcatgactgcatggttgcatgaacacatattaatttccggaagtccgttagccatcatatgcttagggttttggtttcattcttctgtgtatctttcttcttctctcaggtcagaaactgtattgttaatgccaccattttcttcttttaatctaatctattaccaagacccataattcctttcctcaccgcctaacactgccaatgccggtcccaagcccggataaaaagtggagggggcgggaattctgcagatatccatcacact

SEQ ID NO: 61 - DNA, 399 bp, synthetic construct
  source 1..399: mol_type = other DNA
agacccaagcttggtaccgagctcggatccggccaccatggactacaaagacgatgacgacaagatggggagaaaaaagattcagattacgaggattatggatgaacgcaatcgacaggtgagtttgggctgcatgactgcatggttgcatgaacacatattaatttccaaatatttctcaggttcacactatttcccaggcaactctatcactgatgtccttatcacttcaattttggacagcctagcagcttcctgtcctcgatgatctccctgcctggaatcattccttcctcaatttaactcaaactgcaaagccccgcctaacactgccaatgccggtcccaagcccggataaaaagtggagggggcgggaattctgcagatatccatcacact

SEQ ID NO: 62 - DNA, 465 bp, synthetic construct
  source 1..465: mol_type = other DNA
agacccaagcttggtaccgagctcggatccggccaccatggactacaaagacgatgacgacaagaaatatcggtgtctttggccgaagggactgcaacagttctttataatccctctgtaattagcccagaagaactcagagctgctatagaagacatgggatttgaggcttctgtagtgtctggtgagtttgggctgcatgactgcatggttgcatgaacacatattaatttccgaggtggggcagttagttcacatgtcacaatttaatcacatgaaaatctgctaaaggcctagaattgccactataagattttgatttggttaaaatatttaggacagtggctgccaaaaatatttacataggaaacacccccactttccaccgcctaacactgccaatgccggtcccaagcccggataaaaagtggagggggcgggaattctgcagatatccatcacact

SEQ ID NO: 63 - DNA, 465 bp, synthetic construct
  source 1..465: mol_type = other DNA
agacccaagcttggtaccgagctcggatccggccaccatggactacaaagacgatgacgacaagaaatatcggtgtctttggccgaagggactgcaacagttctttataatccctctgtaattagcccagaagaactcagagctgctatagaagacatgggatttgaggcttctgtagtgtctggtgagtttgggctgcatgactgcatggttgcatgaacacatattaatttccctcccaccgagcagcctcagcccctgggctccagtaactaccttgtccctcagccatcctggaggacagagctccctgctgttgttaatctctggggttcttcccattgcctatttggatttccagctctctaatacctttgtaaatagtccgcctaacactgccaatgccggtcccaagcccggataaaaagtggagggggcgggaattctgcagatatccatcacact

SEQ ID NO: 64 - DNA, 465 bp, synthetic construct
  source 1..465: mol_type = other DNA
agacccaagcttggtaccgagctcggatccggccaccatggactacaaagacgatgacgacaagaaatatcggtgtctttggccgaagggactgcaacagttctttataatccctctgtaattagcccagaagaactcagagctgctatagaagacatgggatttgaggcttctgtagtgtctggtgagtttgggctgcatgactgcatggttgcatgaacacatattaatttcccagccatcctggaggacagagctccctgctgttgttaatctctggggttcttcccattgcctatttggatttccagctctctaatacctttgtaaatagttctctatattaaatccttccactgagctaccccgtaacttcaccttatctccgcctaacactgccaatgccggtcccaagcccggataaaaagtggagggggcgggaattctgcagatatccatcacact

SEQ ID NO: 65 - DNA, 465 bp, synthetic construct
  source 1..465: mol_type = other DNA
agacccaagcttggtaccgagctcggatccggccaccatggactacaaagacgatgacgacaagaaatatcggtgtctttggccgaagggactgcaacagttctttataatccctctgtaattagcccagaagaactcagagctgctatagaagacatgggatttgaggcttctgtagtgtctggtgagtttgggctgcatgactgcatggttgcatgaacacatattaatttccttcccattgcctatttggatttccagctctctaatacctttgtaaatagttctctatattaaatccttccactgagctaccccgtaacttcaccttatctttcatttccttcaggaatacttactgcatgcggggtgctattgagtccaaccgcctaacactgccaatgccggtcccaagcccggataaaaagtggagggggcgggaattctgcagatatccatcacact

SEQ ID NO: 66 - DNA, 465 bp, synthetic construct
  source 1..465: mol_type = other DNA
agacccaagcttggtaccgagctcggatccggccaccatggactacaaagacgatgacgacaagaaatatcggtgtctttggccgaagggactgcaacagttctttataatccctctgtaattagcccagaagaactcagagctgctatagaagacatgggatttgaggcttctgtagtgtctggtgagtttgggctgcatgactgcatggttgcatgaacacatattaatttccttgaccaagtggagggtgctcttccagctcttgaacaggacctagagagttggatgtattagatgggcgtacgcagtatgtgcccagttgtatgattgtgcgttttcaaggaagggagtgtgcgtcgattcgttcagtatcgacagggggccgcctaacactgccaatgccggtcccaagcccggataaaaagtggagggggcgggaattctgcagatatccatcacact

SEQ ID NO: 67 - DNA, 465 bp, synthetic construct
  source 1..465: mol_type = other DNA
agacccaagcttggtaccgagctcggatccggccaccatggactacaaagacgatgacgacaagggggtggatcccgaagggtccatcctcgcagagccggaggagctgaaccagacggagcagacaacctacgaggtggaagggatcggctacgacttcatccccacggtgctcgatagaacggtgagtttgggctgcatgactgcatggttgcatgaacacatattaatttccgaccaagagggtgaacagcctcatggtggaccccgtgtctaggcggtaggaccccagaggtctcaccccagcctctccccctgggatcggcacgtctgcagactttccagttccaacacgttttgcagacagcaaaactgcccgccagccccgcctaacactgccaatgccggtcccaagcccggataaaaagtggagggggcgggaattctgcagatatccatcacact

SEQ ID NO: 68 - DNA, 465 bp, synthetic construct
  source 1..465: mol_type = other DNA
agacccaagcttggtaccgagctcggatccggccaccatggactacaaagacgatgacgacaagggggtggatcccgaagggtccatcctcgcagagccggaggagctgaaccagacggagcagacaacctacgaggtggaagggatcggctacgacttcatccccacggtgctcgatagaacggtgagtttgggctgcatgactgcatggttgcatgaacacatattaatttccaccccagaggtctcaccccagcctctccccctgggatcggcacgtctgcagactttccagttccaacacgttttgcagacagcaaaactgcccgccagccccactgagcatccgtgtgacccacaccggcccttcggtctctgctctcccccgcctaacactgccaatgccggtcccaagcccggataaaaagtggagggggcgggaattctgcagatatccatcacact

SEQ ID NO: 69 - DNA, 465 bp, synthetic construct
  source 1..465: mol_type = other DNA
agacccaagcttggtaccgagctcggatccggccaccatggactacaaagacgatgacgacaagggggtggatcccgaagggtccatcctcgcagagccggaggagctgaaccagacggagcagacaacctacgaggtggaagggatcggctacgacttcatccccacggtgctcgatagaacggtgagtttgggctgcatgactgcatggttgcatgaacacatattaatttccgactttccagttccaacacgttttgcagacagcaaaactgcccgccagccccactgagcatccgtgtgacccacaccggcccttcggtctctgctctccctcaggccaaagtcagctttttggctcttaacagacttttccggggccttaccgcctaacactgccaatgccggtcccaagcccggataaaaagtggagggggcgggaattctgcagatatccatcacact

SEQ ID NO: 70 - DNA, 465 bp, synthetic construct
  source 1..465: mol_type = other DNA
agacccaagcttggtaccgagctcggatccggccaccatggactacaaagacgatgacgacaagggggtggatcccgaagggtccatcctcgcagagccggaggagctgaaccagacggagcagacaacctacgaggtggaagggatcggctacgacttcatccccacggtgctcgatagaacggtgagtttgggctgcatgactgcatggttgcatgaacacatattaatttccccactgagcatccgtgtgacccacaccggcccttcggtctctgctctccctcaggccaaagtcagctttttggctcttaacagacttttccggggccttaattttcacacgttttccctgctccctgcctgtgccacctgggctacatccccgcctaacactgccaatgccggtcccaagcccggataaaaagtggagggggcgggaattctgcagatatccatcacact

SEQ ID NO: 71 - DNA, 465 bp, synthetic construct
  source 1..465: mol_type = other DNA
agacccaagcttggtaccgagctcggatccggccaccatggactacaaagacgatgacgacaagggggtggatcccgaagggtccatcctcgcagagccggaggagctgaaccagacggagcagacaacctacgaggtggaagggatcggctacgacttcatccccacggtgctcgatagaacggtgagtttgggctgcatgactgcatggttgcatgaacacatattaatttccttgaccaagtggagggtgctcttccagctcttgaacaggacctagagagttggatgtattagatgggcgtacgcagtatgtgcccagttgtatgattgtgcgttttcaaggaagggagtgtgcgtcgattcgttcagtatcgacagggggccgcctaacactgccaatgccggtcccaagcccggataaaaagtggagggggcgggaattctgcagatatccatcacact

SEQ ID NO: 72 - DNA, 456 bp, synthetic construct
  source 1..456: mol_type = other DNA
agacccaagcttggtaccgagctcggatccggccaccatggactacaaagacgatgacgacaagggcccggctgtggctgaggagcccctccaccgaccgtgagtttgggctgcatgactgcatggttgcatgaacacatattaatttcctccacttagttctacacctcattcattcattcagtgagtgtttctcgactactatgaataaaccgttatactccatgttgcgggcagaatggggatctggacagggaagcacagggcacgagttcaccaatggctgtcaagctacgctgcccgcctaacactgccaatgccggtcccaagcccggataaaaagtggagggggcggggccggcatggtcccagcctcctcgctggcgccggctgggcaacatgcttcggcatggcgaatgggacgcggccgcgaattctgcagatatccatcacact

SEQ ID NO: 73 - DNA, 456 bp, synthetic construct
  source 1..456: mol_type = other DNA
agacccaagcttggtaccgagctcggatccggccaccatggactacaaagacgatgacgacaagggcccggctgtggctgaggagcccctccaccgaccgtgagtttgggctgcatgactgcatggttgcatgaacacatattaatttcctccacttagttctacacctcattcattcattcagtgagtgtttctcgactactatgaataaaccgttatactccatgttgcgggcagaatggggatctggacagggaagcacagggcacgagttcaccaatggctgtcaagctacgctgcggccggcatggtcccagcctcctcgctggcgccggctgggcaacatgcttcggcatggcgaatgggacgcggccgcccgcctaacactgccaatgccggtcccaagcccggataaaaagtggagggggcgggaattctgcagatatccatcacact

SEQ ID NO: 74 - DNA, 380 bp, synthetic construct
  source 1..380: mol_type = other DNA
agacccaagcttggtaccgagctcggatccggccaccatggactacaaagacgatgacgacaagggcccggctgtggctgaggagcccctccaccgaccgtgagtttgggctgcatgactgcatggttgcatgaacacatattaatttccttcattcagtgagtgtttctcgactactatgaataaaccgttatactccatgttgcgggcagaatggggatctggacagggaagcacagggcacgagttcaccaatggctgtcaagctacgctgctcacagaaaaaacagatgatgttacccgcctaacactgccaatgccggtcccaagcccggataaaaagtggagggggcgggaattctgcagatatccatcacact

SEQ ID NO: 75 - DNA, 380 bp, synthetic construct
  source 1..380: mol_type = other DNA
agacccaagcttggtaccgagctcggatccggccaccatggactacaaagacgatgacgacaagggcccggctgtggctgaggagcccctccaccgaccgtgagtttgggctgcatgactgcatggttgcatgaacacatattaatttccaattaatatgtgttgaattacccactccacttagttctacacctcattcattcattcagtgagtgtttctcgactactatgaataaaccgttatactccatgttgcgggcagaatggggatctggacagggaagcacagggcacgagttcccgcctaacactgccaatgccggtcccaagcccggataaaaagtggagggggcgggaattctgcagatatccatcacact

SEQ ID NO: 76 - DNA, 380 bp, synthetic construct
  source 1..380: mol_type = other DNA
agacccaagcttggtaccgagctcggatccggccaccatggactacaaagacgatgacgacaagggcccggctgtggctgaggagcccctccaccgaccgtgagtttgggctgcatgactgcatggttgcatgaacacatattaatttcctctacacctcattcattcattcagtgagtgtttctcgactactatgaataaaccgttatactccatgttgcgggcagaatggggatctggacagggaagcacagggcacgagttcaccaatggctgtcaagctacgctgctcacagaaaaccgcctaacactgccaatgccggtcccaagcccggataaaaagtggagggggcgggaattctgcagatatccatcacact

SEQ ID NO: 77 - DNA, 380 bp, synthetic construct
  source 1..380: mol_type = other DNA
agacccaagcttggtaccgagctcggatccggccaccatggactacaaagacgatgacgacaagggcccggctgtggctgaggagcccctccaccgaccgtgagtttgggctgcatgactgcatggttgcatgaacacatattaatttcccactccacttagttctacacctcattcattcattcagtgagtgtttctcgactactatgaataaaccgttatactccatgttgcgggcagaatggggatctggacagggaagcacagggcacgagttcaccaatggctgtcaagctacgcccgcctaacactgccaatgccggtcccaagcccggataaaaagtggagggggcgggaattctgcagatatccatcacact

SEQ ID NO: 78 - DNA, 380 bp, synthetic construct
  source 1..380: mol_type = other DNA
agacccaagcttggtaccgagctcggatccggccaccatggactacaaagacgatgacgacaagggcccggctgtggctgaggagcccctccaccgaccgtgagtttgggctgcatgactgcatggttgcatgaacacatattaatttccacttagttctacacctcattcattcattcagtgagtgtttctcgactactatgaataaaccgttatactccatgttgcgggcagaatggggatctggacagggaagcacagggcacgagttcaccaatggctgtcaagctacgctgctcaccgcctaacactgccaatgccggtcccaagcccggataaaaagtggagggggcgggaattctgcagatatccatcacact

SEQ ID NO: 79 - DNA, 465 bp, synthetic construct
  source 1..465: mol_type = other DNA
agacccaagcttggtaccgagctcggatccggccaccatggactacaaagacgatgacgacaagcatcatccgtgaccggggacccatgtatgatgaccccaccctgcctgaaggctggacacggaagcttaagcaaaggaaatctggccgctctgctgggaagtatgatgtgtacttaattaagtgagtttgggctgcatgactgcatggttgcatgaacacatattaatttcccacaaggaacaattagaggctctccatagcaatgtcagagatagggcagagcggatggtggtgacaacgctctgacaaacgttactattgaacgagagtcacactgcctggctgcccctgaggacctgtcaccaaagccactcactcgtcccgcctaacactgccaatgccggtcccaagcccggataaaaagtggagggggcgggaattctgcagatatccatcacact

SEQ ID NO: 80 - DNA, 465 bp, synthetic construct
  source 1..465: mol_type = other DNA
agacccaagcttggtaccgagctcggatccggccaccatggactacaaagacgatgacgacaagcatcatccgtgaccggggacccatgtatgatgaccccaccctgcctgaaggctggacacggaagcttaagcaaaggaaatctggccgctctgctgggaagtatgatgtgtacttaattaagtgagtttgggctgcatgactgcatggttgcatgaacacatattaatttccggaacaattagaggctctccatagcaatgtcagagatagggcagagcggatggtggtgacaacgctctgacaaacgttactattgaacgagagtcacactgcctggctgcccctgaggacctgtcaccaaagccactcactcgtctgcctccgcctaacactgccaatgccggtcccaagcccggataaaaagtggagggggcgggaattctgcagatatccatcacact

SEQ ID NO: 81 - DNA, 465 bp, synthetic construct
  source 1..465: mol_type = other DNA
agacccaagcttggtaccgagctcggatccggccaccatggactacaaagacgatgacgacaagcatcatccgtgaccggggacccatgtatgatgaccccaccctgcctgaaggctggacacggaagcttaagcaaaggaaatctggccgctctgctgggaagtatgatgtgtacttaattaagtgagtttgggctgcatgactgcatggttgcatgaacacatattaatttccgaggctctccatagcaatgtcagagatagggcagagcggatggtggtgacaacgctctgacaaacgttactattgaacgagagtcacactgcctggctgcccctgaggacctgtcaccaaagccactcactcgtctgcctgccgctgaccccgcctaacactgccaatgccggtcccaagcccggataaaaagtggagggggcgggaattctgcagatatccatcacact

SEQ ID NO: 82 - DNA, 465 bp, synthetic construct
  source 1..465: mol_type = other DNA
agacccaagcttggtaccgagctcggatccggccaccatggactacaaagacgatgacgacaagcatcatccgtgaccggggacccatgtatgatgaccccaccctgcctgaaggctggacacggaagcttaagcaaaggaaatctggccgctctgctgggaagtatgatgtgtacttaattaagtgagtttgggctgcatgactgcatggttgcatgaacacatattaatttccaggcctctccaaagttcagcaaccaaagagtcaggcctggtacagaagggttgtcaggctgaggcccattctgctggttggtgcctgggcctgagtcaccatctagaacattcccaagtttgaactcaggtacggtgctcagtctctccaccgcctaacactgccaatgccggtcccaagcccggataaaaagtggagggggcgggaattctgcagatatccatcacact

SEQ ID NO: 83 - DNA, 465 bp, synthetic construct
  source 1..465: mol_type = other DNA
agacccaagcttggtaccgagctcggatccggccaccatggactacaaagacgatgacgacaagcatcatccgtgaccggggacccatgtatgatgaccccaccctgcctgaaggctggacacggaagcttaagcaaaggaaatctggccgctctgctgggaagtatgatgtgtacttaattaagtgagtttgggctgcatgactgcatggttgcatgaacacatattaatttcccagagatagggcagagcggatggtggtgacaacgctctgacaaacgttactattgaacgagagtcacactgcctggctgcccctgaggacctgtcaccaaagccactcactcgtctgcctgccgctgacccccccaggcctctccaaagtccgcctaacactgccaatgccggtcccaagcccggataaaaagtggagggggcgggaattctgcagatatccatcacact

SEQ ID NO: 84 - DNA, 541 bp, synthetic construct
  source 1..541: mol_type = other DNA
agacccaagcttggtaccgagctcggatccggccaccatggactacaaagacgatgacgacaagcatcatccgtgaccggggacccatgtatgatgaccccaccctgcctgaaggctggacacggaagcttaagcaaaggaaatctggccgctctgctgggaagtatgatgtgtacttaattaagtgagtttgggctgcatgactgcatggttgcatgaacacatattaatttccaattagaggctctccatagcaatgtcagagatagggcagagcggatggtggtgacaacgctctgacaaacgttactattgaacgagagtcacactgcctggctgcccctgaggacctgtcaccaaagccactcactcgtctgcctgccgcccgcctaacactgccaatgccggtcccaagcccggataaaaagtggagggggcggggccggcatggtcccagcctcctcgctggcgccggctgggcaacatgcttcggcatggcgaatgggacgcggccgcgaattctgcagatatccatcacact

SEQ ID NO: 85 - DNA, 541 bp, synthetic construct
  source 1..541: mol_type = other DNA
agacccaagcttggtaccgagctcggatccggccaccatggactacaaagacgatgacgacaagcatcatccgtgaccggggacccatgtatgatgaccccaccctgcctgaaggctggacacggaagcttaagcaaaggaaatctggccgctctgctgggaagtatgatgtgtacttaattaagtgagtttgggctgcatgactgcatggttgcatgaacacatattaatttccaattagaggctctccatagcaatgtcagagatagggcagagcggatggtggtgacaacgctctgacaaacgttactattgaacgagagtcacactgcctggctgcccctgaggacctgtcaccaaagccactcactcgtctgcctgccgcggccggcatggtcccagcctcctcgctggcgccggctgggcaacatgcttcggcatggcgaatgggacgcggccgcccgcctaacactgccaatgccggtcccaagcccggataaaaagtggagggggcgggaattctgcagatatccatcacact

SEQ ID NO: 86 - DNA, 605 bp, synthetic construct
  source 1..605: mol_type = other DNA
agacccaagcttggtaccgagctcggatccggccaccatggactacaaagacgatgacgacaaggcgaccctggaaaagctgatgaaggccttcgagtccctcaagtccttccagcagaatcaacagaataaccagcaaaatcagaaccaaaaccaacagaatcagcagaaccagccgccacctccaccaccgcctcctcccccacctcagcttcctcagccacctccacaggcacagcctctgctgcctcagccacagcctcccccaccacctcccccacctccacccggcccggctgtggctgaggagcccctgcaccgaccgtgagtttgggctgcatgactgcatggttgcatgaacacatattaatttcctccacttagttctacacctcattcattcattcagtgagtgtttctcgactactatgaataaaccgttatactccatgttgcgggcagaatggggatctggacagggaagcacagggcacgagttcaccaatggctgtcaagctacgctgcccgcctaacactgccaatgccggtcccaagcccggataaaaagtggagggggcgggaattctgcagatatccatcacact

SEQ ID NO: 87 - DNA, 626 bp, synthetic construct
  source 1..626: mol_type = other DNA
agacccaagcttggtaccgagctcggatccggccaccatggactacaaagacgatgacgacaaggcgaccctggaaaagctgatgaaggccttcgagtccctcaagtccttccagcagaatcaacagaataaccagcaaaatcagaaccaaaaccaacagaatcagcagaaccagccgccacctccaccaccgcctcctcccccacctcagcttcctcagccacctccacaggcacagcctctgctgcctcagccacagcctcccccaccacctcccccacctccacccggcccggctgtggctgaggagcccctgcaccgaccgtgagtttgggctgcatgactgcatggttgcatgaacacatattaatttcctccacttagttctacacctcattcattcattcagtgagtgtttctcgactactatgaataaaccgttatactccatgttgcgggcagaatggggatctggacagggaagcacagggcacgagttcaccaatggctgtcaagctacgctgcggccggcatggtcccagcctcctcgctggcgccggctgggcaacatgcttcggcatggcgaatgggacgcggccgcgaattctgcagatatccatcacact

SEQ ID NO: 88 - DNA, 605 bp, synthetic construct
  source 1..605: mol_type = other DNA
agacccaagcttggtaccgagctcggatccggccaccatggactacaaagacgatgacgacaaggcgaccctggaaaagctgatgaaggccttcgagtccctcaagtccttccagcagaatcaacagaataaccagcaaaatcagaaccaaaaccaacagaatcagcagaaccagccgccacctccaccaccgcctcctcccccacctcagcttcctcagccacctccacaggcacagcctctgctgcctcagccacagcctcccccaccacctcccccacctccacccggcccggctgtggctgaggagcccctgcaccgaccgtgagtttgggctgcatgactgcatggttgcatgaacacatattaatttccttgaccaagtggagggtgctcttccagctcttgaacaggacctagagagttggatgtattagatgggcgtacgcagtatgtgcccagttgtatgattgtgcgttttcaaggaagggagtgtgcgtcgattcgttcagtatcgacagggggccgcctaacactgccaatgccggtcccaagcccggataaaaagtggagggggcgggaattctgcagatatccatcacact

SEQ ID NO: 89 - DNA, 465 bp, synthetic construct
  source 1..465: mol_type = other DNA
agacccaagcttggtaccgagctcggatccggccaccatggactacaaagacgatgacgacaagcatcatccgtgaccggggacccatgtatgatgaccccaccctgcctgaaggctggacacggaagcttaagcaaaggaaatctggccgctctgctgggaagtatgatgtgtacttaattaagtgagtttgggctgcatgactgcatggttgcatgaacacatattaatttccgatagagggcctgctaccttgagaacttcccactcagggcagcgacctctgtacacggtcatttcaagcacacctggtctcagtgttcattgtttatgttccccccgaccccaccctgggcacatacattttcctgctccatgagggatcccgcctaacactgccaatgccggtcccaagcccggataaaaagtggagggggcgggaattctgcagatatccatcacact

SEQ ID NO: 90 - DNA, 465 bp, synthetic construct
  source 1..465: mol_type = other DNA
agacccaagcttggtaccgagctcggatccggccaccatggactacaaagacgatgacgacaagcatcatccgtgaccggggacccatgtatgatgaccccaccctgcctgaaggctggacacggaagcttaagcaaaggaaatctggccgctctgctgggaagtatgatgtgtacttaattaagtgagtttgggctgcatgactgcatggttgcatgaacacatattaatttccagataaagacttgaggtgtggagaggatagagggcctgctaccttgagaacttcccactcagggcagcgacctctgtacacggtcatttcaagcacacctggtctcagtgttcattgtttatgttccccccgaccccaccctgggcacatccgcctaacactgccaatgccggtcccaagcccggataaaaagtggagggggcgggaattctgcagatatccatcacact

SEQ ID NO: 91 - DNA, 465 bp, synthetic construct
  source 1..465: mol_type = other DNA
agacccaagcttggtaccgagctcggatccggccaccatggactacaaagacgatgacgacaagcatcatccgtgaccggggacccatgtatgatgaccccaccctgcctgaaggctggacacggaagcttaagcaaaggaaatctggccgctctgctgggaagtatgatgtgtacttaattaagtgagtttgggctgcatgactgcatggttgcatgaacacatattaatttccctgcttccgcagctattccatccccagataaagacttgaggtgtggagaggatagagggcctgctaccttgagaacttcccactcagggcagcgacctctgtacacggtcatttcaagcacacctggtctcagtgttcattgtttatgttccgcctaacactgccaatgccggtcccaagcccggataaaaagtggagggggcgggaattctgcagatatccatcacact

SEQ ID NO: 92 - DNA, 465 bp, synthetic construct
  source 1..465: mol_type = other DNA
agacccaagcttggtaccgagctcggatccggccaccatggactacaaagacgatgacgacaagcatcatccgtgaccggggacccatgtatgatgaccccaccctgcctgaaggctggacacggaagcttaagcaaaggaaatctggccgctctgctgggaagtatgatgtgtacttaattaagtgagtttgggctgcatgactgcatggttgcatgaacacatattaatttcctgaacccctagctctgcaagttcctctgcttccgcagctattccatccccagataaagacttgaggtgtggagaggatagagggcctgctaccttgagaacttcccactcagggcagcgacctctgtacacggtcatttcaagcacacctccgcctaacactgccaatgccggtcccaagcccggataaaaagtggagggggcgggaattctgcagatatccatcacact

SEQ ID NO: 93 - DNA, 465 bp, synthetic construct
  source 1..465: mol_type = other DNA
agacccaagcttggtaccgagctcggatccggccaccatggactacaaagacgatgacgacaagcatcatccgtgaccggggacccatgtatgatgaccccaccctgcctgaaggctggacacggaagcttaagcaaaggaaatctggccgctctgctgggaagtatgatgtgtacttaattaagtgagtttgggctgcatgactgcatggttgcatgaacacatattaatttcctattgaacgagagtcacactgcctggctgcccctgaggacctgtcaccaaagccactcactcgtctgcctgccgctgacccccccaggcctctccaaagttcagcaaccaaagagtcaggcctggtacagaagggttgtcaggctgaggcccgcctaacactgccaatgccggtcccaagcccggataaaaagtggagggggcgggaattctgcagatatccatcacact

SEQ ID NO: 94 - DNA, 465 bp, synthetic construct
  source 1..465: mol_type = other DNA
agacccaagcttggtaccgagctcggatccggccaccatggactacaaagacgatgacgacaagcatcatccgtgaccggggacccatgtatgatgaccccaccctgcctgaaggctggacacggaagcttaagcaaaggaaatctggccgctctgctgggaagtatgatgtgtacttaattaagtgagtttgggctgcatgactgcatggttgcatgaacacatattaatttccaattagaggctctccatagcaatgtcagagatagggcagagcggatggtggtgacaacgctctgacaaacgttactattgaacgagagtcacactgcctggctgcccctgaggacctgtcaccaaagccactcactcgtctgcctgccgcccgcctaacactgccaatgccggtcccaagcccggataaaaagtggagggggcgggaattctgcagatatccatcacact

SEQ ID NO: 95 - DNA, 325 bp, synthetic construct
  source 1..325: mol_type = other DNA
agacccaagcttggtaccgagctcggatccggccaccatggactacaaagacgatgacgacaagggcccggctgtggctgaggagcccctccaccgaccgtgagtttgggctgcatgactgcatggttgcatgaacacatattaatttcctccacttagttctacacctcattcattcattcagtgagtgtttctcgactactatgaataaaccgttatactccatgttgcgggcagaatggggatctggacagggaagcacagggcacgagttcaccaatggctgtcaagctacgctgcgaattctgcagatatccatcacact

SEQ ID NO: 96 - DNA, 523 bp, synthetic construct
  source 1..523: mol_type = other DNA
agacccaagcttggtaccgagctcggatccggccaccatggactacaaagacgatgacgacaagcatcatccgtgaccggggacccatgtatgatgaccccaccctgcctgaaggctggacacggaagcttaagcaaaggaaatctggccgctctgctgggaagtatgatgtgtacttaattaagtgagtttgggctgcatgactgcatggttgcatgaacacatattaatttccaattagaggctctccatagcaatgtcagagatagggcagagcggatggtggtgacaacgctctgacaaacgttactattgaacgagagtcacactgcctggctgcccctgaggacctgtcaccaaagccactcactcgtctgcctgccgcccgcctaacactgccaatgccggtcccaagcccggataaaaagtggagggggcggaaaggtttttcttttcctgagaaatttctcaggttttgcttttaaaaaaaaagcaaaagaattctgcagatatccatcacact

SEQ ID NO: 97 - DNA, 558 bp, synthetic construct
  source 1..558: mol_type = other DNA
agacccaagcttggtaccgagctcggatccggccaccatggactacaaagacgatgacgacaagcatcatccgtgaccggggacccatgtatgatgaccccaccctgcctgaaggctggacacggaagcttaagcaaaggaaatctggccgctctgctgggaagtatgatgtgtacttaattaagtgagtttgggctgcatgactgcatggttgcatgaacacatattaatttccaattagaggctctccatagcaatgtcagagatagggcagagcggatggtggtgacaacgctctgacaaacgttactattgaacgagagtcacactgcctggctgcccctgaggacctgtcaccaaagccactcactcgtctgcctgccgcccgcctaacactgccaatgccggtcccaagcccggataaaaagtggagggggcgggaaggtttttcttttcctgagaaaacaacacgtattgttttctcaggttttgctttttggcctttttctagcttaaaaaaaaaaaaagcaaaagaattctgcagatatccatcacact

SEQ ID NO: 98 - DNA, 558 bp, synthetic construct
  source 1..558: mol_type = other DNA
agacccaagcttggtaccgagctcggatccggccaccatggactacaaagacgatgacgacaagcatcatccgtgaccggggacccatgtatgatgaccccaccctgcctgaaggctggacacggaagcttaagcaaaggaaatctggccgctctgctgggaagtatgatgtgtacttaattaagtgagtttgggctgcatgactgcatggttgcatgaacacatattaatttccaattagaggctctccatagcaatgtcagagatagggcagagcggatggtggtgacaacgctctgacaaacgttactattgaacgagagtcacactgcctggctgcccctgaggacctgtcaccaaagccactcactcgtctgcctgccgcccgcctaacactgccaatgccggtcccaagcccggataaaaagtggagggggcggaggtgtttcttttactgagtgcagcccatggccgcactcaggttttgcttttcaccttcccatctgtgaaagagtgagcaggaaaaagcaaaagaattctgcagatatccatcacact

SEQ ID NO: 99 - DNA, 391 bp, synthetic construct
  source 1..391: mol_type = other DNA
agacccaagcttggtaccgagctcggatccggccaccatggactacaaagacgatgacgacaagggcccggctgtggctgaggagcccctccaccgaccgtgagtttgggctgcatgactgcatggttgcatgaacacatattaatttcctccacttagttctacacctcattcattcattcagtgagtgtttctcgactactatgaataaaccgttatactccatgttgcgggcagaatggggatctggacagggaagcacagggcacgagttcaccaatggctgtcaagctacgctgcataatttttgaccgcctaacactgccaatgccggtcccaagcccggataaaaagtggagggggcgggaattctgcagatatccatcacact

SEQ ID NO: 100 - DNA, 389 bp, synthetic construct
  source 1..389: mol_type = other DNA
agacccaagcttggtaccgagctcggatccggccaccatggactacaaagacgatgacgacaagggcccggctgtggctgaggagcccctccaccgaccgtgagtttgggctgcatgactgcatggttgcatgaacacatattaatttcctccacttagttctacacctcattcattcattcagtgagtgtttctcgactactatgaataaaccgttatactccatgttgcgggcagaatggggatctggacagggaagcacagggcacgagttcaccaatggctgtcaagctacgctgcaatttttgaccgcctaacactgccaatgccggtcccaagcccggataaaaagtggagggggcgggaattctgcagatatccatcacact

SEQ ID NO: 101 - DNA, 388 bp, synthetic construct
  source 1..388: mol_type = other DNA
agacccaagcttggtaccgagctcggatccggccaccatggactacaaagacgatgacgacaagggcccggctgtggctgaggagcccctccaccgaccgtgagtttgggctgcatgactgcatggttgcatgaacacatattaatttcctccacttagttctacacctcattcattcattcagtgagtgtttctcgactactatgaataaaccgttatactccatgttgcgggcagaatggggatctggacagggaagcacagggcacgagttcaccaatggctgtcaagctacgctgcatttgtggccgcctaacactgccaatgccggtcccaagcccggataaaaagtggagggggcgggaattctgcagatatccatcacact

SEQ ID NO: 102 - DNA, 391 bp, synthetic construct
  source 1..391: mol_type = other DNA
agacccaagcttggtaccgagctcggatccggccaccatggactacaaagacgatgacgacaagggcccggctgtggctgaggagcccctccaccgaccgtgagtttgggctgcatgactgcatggttgcatgaacacatattaatttcctccacttagttctacacctcattcattcattcagtgagtgtttctcgactactatgaataaaccgttatactccatgttgcgggcagaatggggatctggacagggaagcacagggcacgagttcaccaatggctgtcaagctacgctgcccgcctaacactgccaatgccggtcccaagcccggataaaaagtggagggggcggataatttttgagaattctgcagatatccatcacact

SEQ ID NO: 103 - DNA, 389 bp, synthetic construct
  source 1..389: mol_type = other DNA
agacccaagcttggtaccgagctcggatccggccaccatggactacaaagacgatgacgacaagggcccggctgtggctgaggagcccctccaccgaccgtgagtttgggctgcatgactgcatggttgcatgaacacatattaatttcctccacttagttctacacctcattcattcattcagtgagtgtttctcgactactatgaataaaccgttatactccatgttgcgggcagaatggggatctggacagggaagcacagggcacgagttcaccaatggctgtcaagctacgctgcccgcctaacactgccaatgccggtcccaagcccggataaaaagtggagggggcggaatttttgagaattctgcagatatccatcacact

SEQ ID NO: 104 - DNA, 388 bp, synthetic construct
  source 1..388: mol_type = other DNA
agacccaagcttggtaccgagctcggatccggccaccatggactacaaagacgatgacgacaagggcccggctgtggctgaggagcccctccaccgaccgtgagtttgggctgcatgactgcatggttgcatgaacacatattaatttcctccacttagttctacacctcattcattcattcagtgagtgtttctcgactactatgaataaaccgttatactccatgttgcgggcagaatggggatctggacagggaagcacagggcacgagttcaccaatggctgtcaagctacgctgcccgcctaacactgccaatgccggtcccaagcccggataaaaagtggagggggcggatttgtgggaattctgcagatatccatcacact

SEQ ID NO: 105 - DNA, 55 bp, synthetic construct
  source 1..55: mol_type = other DNA
ccgcctaacactgccaatgccggtcccaagcccggataaaaagtggagggggcgg

SEQ ID NO: 106 - DNA, 43 bp, synthetic construct
  source 1..43: mol_type = other DNA
  misc_difference 38..43: note = n is a, c, g, or t
ctgatgagtccgtgaggacgaaacgagtaagctcgtcnnnnnn

SEQ ID NO: 107 - DNA, 76 bp, synthetic construct
  source 1..76: mol_type = other DNA
ggccggcatggtcccagcctcctcgctggcgccggctgggcaacatgcttcggcatggcgaatgggacgcggccgc

SEQ ID NO: 108 - DNA, 50 bp, synthetic construct
  source 1..50: mol_type = other DNA
aaacagagaagtcaaccagagaaacacacgttgtggtatattacctggta

SEQ ID NO: 109 - DNA, 52 bp, synthetic construct
  source 1..52: mol_type = other DNA
caacagcgaagcgcgccagggaaacacaccatgtgtggtatattatctggca

SEQ ID NO: 110 - DNA, 52 bp, synthetic construct
  source 1..52: mol_type = other DNA
caacagcgaagcggaacggcgaaacacaccttgtgtggtatattacccgttg

SEQ ID NO: 111 - DNA, 176 bp, synthetic construct
  source 1..176: mol_type = other DNA
gggaaagcttgcgaagggcgtcgtcgccccgagcggtagtaagcagggaactcacctccaatttcagtactgaaattgtcgtagcagttgactactgttatgtgattggtagaggctaagtgacggtattggcgtaagtcagtattgcagcacagcacaagcccgcttgcgagaat

SEQ ID NO: 112 - DNA, 167 bp, synthetic construct
  source 1..167: mol_type = other DNA
taattatagcgcccgaactaagcgcccggaaaaaggcttagttgacgaggatggaggttatcgaattttcgggcggatgcctcccggctgagtgtgcagatcacagccgtaaggatttcttcaaaccaagggggtgactccttgaacaaagagaaatcacatgatct

SEQ ID NO: 113 - DNA, 76 bp, synthetic construct
  source 1..76: mol_type = other DNA
ggacccgcaaggccgacggcatccgccgccgctggtgcaagtccagccgccccggggcgggcgctcatgggtaaac

SEQ ID NO: 114 - DNA, 78 bp, synthetic construct
  source 1..78: mol_type = other DNA
ggacccgcaaggccgacggcatccgccgccgctggtgcaagtccagccgccccatggggcgggcgctcatgggtaaac

SEQ ID NO: 115 - DNA, 65 bp, synthetic construct
  source 1..65: mol_type = other DNA
ggagccgttcgggcggctataaacagacctcaggcccgaagcgtggcggcgatccgccggtggta

SEQ ID NO: 116 - DNA, 80 bp, synthetic construct
  source 1..80: mol_type = other DNA
cattcctcagaaaatgacaaacctgtggggcgtaagtagatatgtacatatctatgatcgtgcagacgttaaaatcaggt

SEQ ID NO: 117 - DNA, 463 bp, synthetic construct
  source 1..463: mol_type = other DNA
agacccaagcttggtaccgagctcggatccggccaccatggactacaaagacgatgacgacaagttgggtcttcccatggaaagctaccagccctgggcgctgcccaacggctggaacggccaaatgtactgccccaaagagcaggcgcagcctccccacctctggaagtccacacttcctggtgagtttgggctgcatgactgcatggttgcatgaacacatattaatttcctacacagggatcggaccccagccagggcataggcgacagctcgatctgagccacccgccttgcagcgcccggctgctctttgccacccgctgtacaatcctgtcttctgctaaagcctagagggtcagtggggaaggtagttagttctgaccgcctaacactgccaatgccggtcccaagcccggataaaaagtggagggggcgggaattctgcagatatccatcacact

SEQ ID NO: 118 - DNA, 463 bp, synthetic construct
  source 1..463: mol_type = other DNA
agacccaagcttggtaccgagctcggatccggccaccatggactacaaagacgatgacgacaagttgggtcttcccatggaaagctaccagccctgggcgctgcccaacggctggaacggccaaatgtactgccccaaagagcaggcgcagcctccccacctctggaagtccacacttcctggtgagtttgggctgcatgactgcatggttgcatgaacacatattaatttccccacccgccttgcagcgcccggctgctctttgccacccgctgtacaatcctgtcttctgctaaagcctagagggtcagtggggaaggtagttagttctgaactgaaatgaaatcacccagggctccagtgacttccccaacccggccatcccgcctaacactgccaatgccggtcccaagcccggataaaaagtggagggggcgggaattctgcagatatccatcacact

SEQ ID NO: 119 - DNA, 463 bp, synthetic construct
  source 1..463: mol_type = other DNA
agacccaagcttggtaccgagctcggatccggccaccatggactacaaagacgatgacgacaagttgggtcttcccatggaaagctaccagccctgggcgctgcccaacggctggaacggccaaatgtactgccccaaagagcaggcgcagcctccccacctctggaagtccacacttcctggtgagtttgggctgcatgactgcatggttgcatgaacacatattaatttcctgtcttctgctaaagcctagagggtcagtggggaaggtagttagttctgaactgaaatgaaatcacccagggctccagtgacttccccaacccggccatcctgcaggagcagcgcgtaggcagcctcgagtgatagcctggtccaacggcccgcctaacactgccaatgccggtcccaagcccggataaaaagtggagggggcgggaattctgcagatatccatcacact

SEQ ID NO: 120 - DNA, 463 bp, synthetic construct
  source 1..463: mol_type = other DNA
agacccaagcttggtaccgagctcggatccggccaccatggactacaaagacgatgacgacaagttgggtcttcccatggaaagctaccagccctgggcgctgcccaacggctggaacggccaaatgtactgccccaaagagcaggcgcagcctccccacctctggaagtccacacttcctggtgagtttgggctgcatgactgcatggttgcatgaacacatattaatttccactgaaatgaaatcacccagggctccagtgacttccccaacccggccatcctgcaggagcagcgcgtaggcagcctcgagtgatagcctggtccaacggcccacaccttagcgccaggctcaaggtacaacacttctgtgcctgcctcctccgcctaacactgccaatgccggtcccaagcccggataaaaagtggagggggcgggaattctgcagatatccatcacact

SEQ ID NO: 121 - DNA, 463 bp, synthetic construct
  source 1..463: mol_type = other DNA
agacccaagcttggtaccgagctcggatccggccaccatggactacaaagacgatgacgacaagttgggtcttcccatggaaagctaccagccctgggcgctgcccaacggctggaacggccaaatgtactgccccaaagagcaggcgcagcctccccacctctggaagtccacacttcctggtgagtttgggctgcatgactgcatggttgcatgaacacatattaatttccctgcaggagcagcgcgtaggcagcctcgagtgatagcctggtccaacggcccacaccttagcgccaggctcaaggtacaacacttctgtgcctgcctcctttctgggtgcccgtcccaatactcgaagcttctacactgaagccatttttccgcctaacactgccaatgccggtcccaagcccggataaaaagtggagggggcgggaattctgcagatatccatcacact

SEQ ID NO: 122 - DNA, 401 bp, synthetic construct
  source 1..401: mol_type = other DNA
agacccaagcttggtaccgagctcggatccggccaccatggactacaaagacgatgacgacaagggcccggctgtggctgaggagcccctccaccgaccgtgagtttgggctgcatgactgcatggttgcatgaacacatattaatttcctccacttagttctacacctcattcattcattcagtgagtgtttctcgactactatgaataaaccgttatactccatgttgcgggcagaatggggatctggacagggaagcacagggcacgagttcaccaatggctgtcaagctacgctgcggccggcatggtcccagcctcctcgctggcgccggctgggcaacatgcttcggcatggcgaatgggacgcggccgcgaattctgcagatatccatcacact

SEQ ID NO: 123 - DNA, 2473 bp, synthetic construct
  source 1..2473: mol_type = other DNA
agacccaagcttggtaccgagctcggatccggccaccatggactacaaagacgatgacgacaagctcttagaagagaaggtaaaaaaactttcagaacaattgggagttgaattaactagccctgttgctgcttctgaagagtttgaagatgaagaagaaagtcctgttaatttccccatttacatgacgaggattttgacagctttcaaagtggtgaggacactgaagactggttttggctttaccaatgtgactgcacaccaaaaatggaaattttcaagacctggcatcaggctcctttctgtcaaggcacagacagcacacattgtcctggaagatggaactaagatgaaaggttactcctttggccatccatcctctgttgctggtgaagtggtttttaatactggcctgggagggtacccagaagctattactgaccctgcctacaaaggacagattctcacaatggccaaccctattattgggaatggtggagctcctgatactactgctctggatgaactgggacttagcaaatatttggagtctaatggaatcaaggtttcaggtttgctggtgctggattatagtaaagactacaaccactggctggctaccaagagtttagggcaatggctacaggaagaaaaggttcctgcaatttatggagtggacacaagaatgctgactaaaataattcgggataagggtaccatgcttgggaagattgaatttgaaggtcagcctgtggattttgtggatccaaataaacagaatttgattgctgaggtttcaaccaaggatgtcaaagtgtacggcaaaggaaaccccacaaaagtggtagctgtagactgtgggattaaaaacaatgtaatccgcctgctagtaaagcgaggagctgaagtgcacttagttccctggaaccatgatttcaccaagatggagtatgatgggattttgatcgcgggaggaccggggaacccagctcttgcagaaccactaattcagaatgtcagaaagattttggagagtgatcgcaaggagccattgtttggaatcagtacaggaaacttaataacaggattggctgctggtgccaaaacctacaagatgtccatggccaacagagggcagaatcagcctgttttgaatatcacaaacaaacaggctttcattactgctcagaatcatggctatgccttggacaacaccctccctgctggctggaaaccactttttgtgaatgtcaacgatcaaacaaatgaggggattatgcatgagagcaaacccttcttcgctgtgcagttccacccagaggtcaccccggggccaatagacactgagtacctgtttgattcctttttctcactgataaagaaaggaaaagctaccaccattacatcagtcttaccgaagccagcactagttgcatctcgggttgaggtttccaaagtccttattctaggatcaggaggtctgtccattggtcaggctggagaatttgattactcaggatctcaagctgtaaaagccatgaaggaagaaaatgtcaaaactgttctgatgaacccaaacattgcatcagtccagaccaatgaggtgggcttaaagcaagcggatactgtctactttcttcccatcacccctcagtttgtcacagaggtcatcaaggcagaacagccagatgggttaattctgggcatgggtggccagacagctctgaactgtggagtggaactattcaagagaggtgtgctcaaggaatatggtgtgaaagtcctgggaacttcagttgagtccattatggctacggaagacaggcagctgttttcagataaactaaatgagatcaatgaaaagattgctccaagttttgcagtggaatcgattgaggatgcactgaaggcagcagacaccattggctacccagtgatgatccgttccgcctatgcactgggtgggttaggctcaggcatctgtcccaacagagagactttgatggacctcagcacaaaggcctttgctatgaccaaccaaattctggtggagaagtcagtgacggcccggctgtggctgaggagcccctccaccgaccgtgagtttgggctgcatgactgcatggttgcatgaacacatattaatttcctccacttagttctacacctcattcattcattcagtgagtgtttctcgactactatgaataaaccgttatactccatgttgcgggcagaatggggatctggacagggaagcacagggcacgagttcaccaatggctgtcaagctacgctgcccgcctaacactgccaatgccggtcccaagcccggataaaaagtggagggggcgggaaggtttttcttttcctgagaaaacaacacgtattgttttctcaggttttgctttttggcctttttctagcttaaaaaaaaaaaaagcaaaagaattctgcagatatccatcacact

SEQ ID NO: 124 - DNA, 5473 bp, synthetic construct
  source 1..5473: mol_type = other DNA
agacccaagcttggtaccgagctcggatccggccaccatggactacaaagacgatgacgacaagggatcaatccctgaccctagtttgccccttccaaatcaacttgagatcgctctaaggaaaattaaggagaacattcgaataattctagaaacacgggcaacttgcaaatcactagaagagaaactaaaagagaaagaatctgctttaaggttagcagaacaaaatatactgtcaagagacaaagtaatcaatgaactgaggcttcgattgcctgccactgcagaaagagaaaagctcatagctgagctaggcagaaaagagatggaaccaaaatctcaccacacattgaaaattgctcatcaaaccattgcaaacatgcaagcaaggttaaatcaaaaagaagaagtattaaagaagtatcaacgtcttctagaaaaagccagagaggagcaaagagaaattgtgaagaaacatgaggaagaccttcatattcttcatcacagattagaactacaggctgatagttcactaaataaattcaaacaaacggcttgggatttaatgaaacagtctcccactccagttcctaccaacaagcattttattcgtctggctgagatggaacagacagtagcagaacaagatgactctctttcctcactcttggtcaaactaaagaaagtatcacaagatttggagagacaaagagaaatcactgaattaaaagtaaaagaatttgaaaatatcaaattacagcttcaagaaaaccatgaagatgaagtgaaaaaagtaaaagcggaagtagaggatttaaagtatcttctggaccagtcacaaaaggagtcacagtgtttaaaatctgaacttcaggctcaaaaagaagcaaattcaagagctccaacaactacaatgagaaatctagtagaacggctaaagagccaattagccttgaaggagaaacaacagaaagcacttagtcgggcacttttagaactccgggcagaaatgacagcagctgctgaagaacgtattatttctgcaacttctcaaaaagaggcccatctcaatgttcaacaaatcgttgatcgacatactagagagctaaagacacaagttgaagatttaaatgaaaatcttttaaaattgaaagaagcacttaaaacaagtaaaaacagagaaaactcactaactgataatttgaatgacttaaataatgaactgcaaaagaaacaaaaagcctataataaaatacttagagagaaagaggaaattgatcaagagaatgatgaactgaaaaggcaaattaaaagactaaccagtggattacagggcaaacccctgacagataataaacaaagtctaattgaagaactccaaaggaaagttaaaaaactagagaaccaattagagggaaaggtggaggaagtagacctaaaacctatgaaagaaaagaatgctaaagaagaattaattaggtgggaagaaggtaaaaagtggcaagccaaaatagaaggaattcgaaacaagttaaaagagaaagagggggaagtctttactttaacaaagcagttgaatactttgaaggatctttttgccaaagccgataaagagaaacttactttgcagaggaaactaaaaacaactggcatgactgttgatcaggttttgggaatacgagctttggagtcagaaaaagaattggaagaattaaaaaagagaaatcttgacttagaaaatgatatattgtatatgagggcccaccaagctcttcctcgagattctgttgtagaagatttacatttacaaaatagatacctccaagaaaaacttcatgctttagaaaaacagttttcaaaggatacatattctaagccttcaatttcaggaatagagtcagatgatcattgtcagagagaacaggagcttcagaaggaaaacttgaagttgtcatctgaaaatattgaactgaaatttcagcttgaacaagcaaataaagatttgccaagattaaagaatcaagtcagagatttgaaggaaatgtgtgaatttcttaagaaagaaaaagcagaagttcagcggaaacttggccatgttagagggtctggtagaagtggaaagacaatcccagaactggaaaaaaccattggtttaatgaaaaaagtagttgaaaaagtccagagagaaaatgaacagttgaaaaaagcatcaggaatattgactagtgaaaaaatggctaatattgagcaggaaaatgaaaaattgaaggctgaattagaaaaacttaaagctcatcttgggcatcagttgagcatgcactatgaatccaagaccaaaggcacagaaaaaattattgctgaaaatgaaaggcttcgtaaagaacttaaaaaagaaactgatgctgcagagaaattacggatagcaaagaataatttagagatattaaatgagaagatgacagttcaactagaagagactggtaagagattgcagtttgcagaaagcagaggtccacagcttgaaggtgctgacagtaagagctggaaatccattgtggttacaagaatgtatgaaaccaagttaaaagaattggaaactgatattgccaaaaaaaatcaaagcattactgaccttaaacagcttgtaaaagaagcaacagagagagaacaaaaagttaacaaatacaatgaagaccttgaacaacagattaagattcttaaacatgttcctgaaggtgctgagacagagcaaggccttaaacgggagcttcaagttcttagattagctaatcatcagctggataaagagaaagcagaattaatccatcagatagaagctaacaaggaccaaagtggagctgaaagcaccatacctgatgctgatcaactaaaggaaaaaataaaagatctagagacacagctcaaaatgtcagatctagaaaagcagcatttgaaggaggaaataaagaagctgaaaaaagaactggaaaattttgatccttcattttttgaagaaattgaagatcttaagtataattacaaggaagaagtgaagaagaatattctcttagaagagaaggtaaaaaaactttcagaacaattgggagttgaattaactagccctgttgctgcttctgaagagtttgaagatgaagaagaaagtcctgttaatttccccatttacatgacgaggattttgacagctttcaaagtggtgaggacactgaagactggttttggctttaccaatgtgactgcacaccaaaaatggaaattttcaagacctggcatcaggctcctttctgtcaaggcacagacagcacacattgtcctggaagatggaactaagatgaaaggttactcctttggccatccatcctctgttgctggtgaagtggtttttaatactggcctgggagggtacccagaagctattactgaccctgcctacaaaggacagattctcacaatggccaaccctattattgggaatggtggagctcctgatactactgctctggatgaactgggacttagcaaatatttggagtctaatggaatcaaggtttcaggtttgctggtgctggattatagtaaagactacaaccactggctggctaccaagagtttagggcaatggctacaggaagaaaaggttcctgcaatttatggagtggacacaagaatgctgactaaaataattcgggataagggtaccatgcttgggaagattgaatttgaaggtcagcctgtggattttgtggatccaaataaacagaatttgattgctgaggtttcaaccaaggatgtcaaagtgtacggcaaaggaaaccccacaaaagtggtagctgtagactgtgggattaaaaacaatgtaatccgcctgctagtaaagcgaggagctgaagtgcacttagttccctggaaccatgatttcaccaagatggagtatgatgggattttgatcgcgggaggaccggggaacccagctcttgcagaaccactaattcagaatgtcagaaagattttggagagtgatcgcaaggagccattgtttggaatcagtacaggaaacttaataacaggattggctgctggtgccaaaacctacaagatgtccatggccaacagagggcagaatcagcctgttttgaatatcacaaacaaacaggctttcattactgctcagaatcatggctatgccttggacaacaccctccctgctggctggaaaccactttttgtgaatgtcaacgatcaaacaaatgaggggattatgcatgagagcaaacccttcttcgctgtgcagttccacccagaggtcaccccggggccaatagacactgagtacctgtttgattcctttttctcactgataaagaaaggaaaagctaccaccattacatcagtcttaccgaagccagcactagttgcatctcgggttgaggtttccaaagtccttattctaggatcaggaggtctgtccattggtcaggctggagaatttgattactcaggatctcaagctgtaaaagccatgaaggaagaaaatgtcaaaactgttctgatgaacccaaacattgcatcagtccagaccaatgaggtgggcttaaagcaagcggatactgtctactttcttcccatcacccctcagtttgtcacagaggtcatcaaggcagaacagccagatgggttaattctgggcatgggtggccagacagctctgaactgtggagtggaactattcaagagaggtgtgctcaaggaatatggtgtgaaagtcctgggaacttcagttgagtccattatggctacggaagacaggcagctgttttcagataaactaaatgagatcaatgaaaagattgctccaagttttgcagtggaatcgattgaggatgcactgaaggcagcagacaccattggctacccagtgatgatccgttccgcctatgcactgggtgggttaggctcaggcatctgtcccaacagagagactttgatggacctcagcacaaaggcctttgctatgaccaaccaaattctggtggagaagtcagtgacggcccggctgtggctgaggagcccctccaccgaccgtgagtttgggctgcatgactgcatggttgcatgaacacatattaatttcctccacttagttctacacctcattcattcattcagtgagtgtttctcgactactatgaataaaccgttatactccatgttgcgggcagaatggggatctggacagggaagcacagggcacgagttcaccaatggctgtcaagctacgctgcccgcctaacactgccaatgccggtcccaagcccggataaaaagtggagggggcgggaaggtttttcttttcctgagaaaacaacacgtattgttttctcaggttttgctttttggcctttttctagcttaaaaaaaaaaaaagcaaaagaattctgcagatatccatcacact

SEQ ID NO: 125 - DNA, 4473 bp, synthetic construct
  source 1..4473: mol_type = other DNA
agacccaagcttggtaccgagctcggatccggccaccatggactacaaagacgatgacgacaagatctcaatgttcaacaaatcgttgatcgacatactagagagctaaagacacaagttgaagatttaaatgaaaatcttttaaaattgaaagaagcacttaaaacaagtaaaaacagagaaaactcactaactgataatttgaatgacttaaataatgaactgcaaaagaaacaaaaagcctataataaaatacttagagagaaagaggaaattgatcaagagaatgatgaactgaaaaggcaaattaaaagactaaccagtggattacagggcaaacccctgacagataataaacaaagtctaattgaagaactccaaaggaaagttaaaaaactagagaaccaattagagggaaaggtggaggaagtagacctaaaacctatgaaagaaaagaatgctaaagaagaattaattaggtgggaagaaggtaaaaagtggcaagccaaaatagaaggaattcgaaacaagttaaaagagaaagagggggaagtctttactttaacaaagcagttgaatactttgaaggatctttttgccaaagccgataaagagaaacttactttgcagaggaaactaaaaacaactggcatgactgttgatcaggttttgggaatacgagctttggagtcagaaaaagaattggaagaattaaaaaagagaaatcttgacttagaaaatgatatattgtatatgagggcccaccaagctcttcctcgagattctgttgtagaagatttacatttacaaaatagatacctccaagaaaaacttcatgctttagaaaaacagttttcaaaggatacatattctaagccttcaatttcaggaatagagtcagatgatcattgtcagagagaacaggagcttcagaaggaaaacttgaagttgtcatctgaaaatattgaactgaaatttcagcttgaacaagcaaataaagatttgccaagattaaagaatcaagtcagagatttgaaggaaatgtgtgaatttcttaagaaagaaaaagcagaagttcagcggaaacttggccatgttagagggtctggtagaagtggaaagacaatcccagaactggaaaaaaccattggtttaatgaaaaaagtagttgaaaaagtccagagagaaaatgaacagttgaaaaaagcatcaggaatattgactagtgaaaaaatggctaatattgagcaggaaaatgaaaaattgaaggctgaattagaaaaacttaaagctcatcttgggcatcagttgagcatgcactatgaatccaagaccaaaggcacagaaaaaattattgctgaaaatgaaaggcttcgtaaagaacttaaaaaagaaactgatgctgcagagaaattacggatagcaaagaataatttagagatattaaatgagaagatgacagttcaactagaagagactggtaagagattgcagtttgcagaaagcagaggtccacagcttgaaggtgctgacagtaagagctggaaatccattgtggttacaagaatgtatgaaaccaagttaaaagaattggaaactgatattgccaaaaaaaatcaaagcattactgaccttaaacagcttgtaaaagaagcaacagagagagaacaaaaagttaacaaatacaatgaagaccttgaacaacagattaagattcttaaacatgttcctgaaggtgctgagacagagcaaggccttaaacgggagcttcaagttcttagattagctaatcatcagctggataaagagaaagcagaattaatccatcagatagaagctaacaaggaccaaagtggagctgaaagcaccatacctgatgctgatcaactaaaggaaaaaataaaagatctagagacacagctcaaaatgtcagatctagaaaagcagcatttgaaggaggaaataaagaagctgaaaaaagaactggaaaattttgatccttcattttttgaagaaattgaagatcttaagtataattacaaggaagaagtgaagaagaatattctcttagaagagaaggtaaaaaaactttcagaacaattgggagttgaattaactagccctgttgctgcttctgaagagtttgaagatgaagaagaaagtcctgttaatttccccatttacatgacgaggattttgacagctttcaaagtggtgaggacactgaagactggttttggctttaccaatgtgactgcacaccaaaaatggaaattttcaagacctggcatcaggctcctttctgtcaaggcacagacagcacacattgtcctggaagatggaactaagatgaaaggttactcctttggccatccatcctctgttgctggtgaagtggtttttaatactggcctgggagggtacccagaagctattactgaccctgcctacaaaggacagattctcacaatggccaaccctattattgggaatggtggagctcctgatactactgctctggatgaactgggacttagcaaatatttggagtctaatggaatcaaggtttcaggtttgctggtgctggattatagtaaagactacaaccactggctggctaccaagagtttagggcaatggctacaggaagaaaaggttcctgcaatttatggagtggacacaagaatgctgactaaaataattcgggataagggtaccatgcttgggaagattgaatttgaaggtcagcctgtggattttgtggatccaaataaacagaatttgattgctgaggtttcaaccaaggatgtcaaagtgtacggcaaaggaaaccccacaaaagtggtagctgtagactgtgggattaaaaacaatgtaatccgcctgctagtaaagcgaggagctgaagtgcacttagttccctggaaccatgatttcaccaagatggagtatgatgggattttgatcgcgggaggaccggggaacccagctcttgcagaaccactaattcagaatgtcagaaagattttggagagtgatcgcaaggagccattgtttggaatcagtacaggaaacttaataacaggattggctgctggtgccaaaacctacaagatgtccatggccaacagagggcagaatcagcctgttttgaatatcacaaacaaacaggctttcattactgctcagaatcatggctatgccttggacaacaccctccctgctggctggaaaccactttttgtgaatgtcaacgatcaaacaaatgaggggattatgcatgagagcaaacccttcttcgctgtgcagttccacccagaggtcaccccggggccaatagacactgagtacctgtttgattcctttttctcactgataaagaaaggaaaagctaccaccattacatcagtcttaccgaagccagcactagttgcatctcgggttgaggtttccaaagtccttattctaggatcaggaggtctgtccattggtcaggctggagaatttgattactcaggatctcaagctgtaaaagccatgaaggaagaaaatgtcaaaactgttctgatgaacccaaacattgcatcagtccagaccaatgaggtgggcttaaagcaagcggatactgtctactttcttcccatcacccctcagtttgtcacagaggtcatcaaggcagaacagccagatgggttaattctgggcatgggtggccagacagctctgaactgtggagtggaactattcaagagaggtgtgctcaaggaatatggtgtgaaagtcctgggaacttcagttgagtccattatggctacggaagacaggcagctgttttcagataaactaaatgagatcaatgaaaagattgctccaagttttgcagtggaatcgattgaggatgcactgaaggcagcagacaccattggctacccagtgatgatccgttccgcctatgcactgggtgggttaggctcaggcatctgtcccaacagagagactttgatggacctcagcacaaaggcctttgctatgaccaaccaaattctggtggagaagtcagtgacggcccggctgtggctgaggagcccctccaccgaccgtgagtttgggctgcatgactgcatggttgcatgaacacatattaatttcctccacttagttctacacctcattcattcattcagtgagtgtttctcgactactatgaataaaccgttatactccatgttgcgggcagaatggggatctggacagggaagcacagggcacgagttcaccaatggctgtcaagctacgctgcccgcctaacactgccaatgccggtcccaagcccggataaaaagtggagggggcgggaaggtttttcttttcctgagaaaacaacacgtattgttttctcaggttttgctttttggcctttttctagcttaaaaaaaaaaaaagcaaaagaattctgcagatatccatcacact

SEQ ID NO: 126 - DNA, 6473 bp, synthetic construct
  source 1..6473: mol_type = other DNA
agacccaagcttggtaccgagctcggatccggccaccatggactacaaagacgatgacgacaagaatcaatttggatgcacagaaggtggaacagatgttaagagatgaattagctgatagtgtgagcaaggcagtaagtgatgctgataggcaacggattctagaattagagaagaatgaaatggaactaaaagttgaagtgtcaaaactgagagagatttctgatattgccagaagacaagttgaaattttgaatgcacaacaacaatctagggacaaggaagtagagtccctcagaatgcaactgctagactatcaggcacagtctgatgaaaagtcgctcattgccaagttgcaccaacataatgtctctcttcaactgagtgaggctactgctcttggtaagttggagtcaattacatctaaactgcagaagatggaggcctacaacttgcgcttagagcagaaacttgatgaaaaagaacaggctctctattatgctcgtttggagggaagaaacagagcaaaacatctgcgccaaacaattcagtctctacgacgacagtttagtggagctttacccttggcacaacaggaaaagttctccaaaacaatgattcaactacaaaatgacaaacttaagataatgcaagaaatgaaaaattctcaacaagaacatagaaatatggagaacaaaacattggagatggaattaaaattaaagggcctggaagagttaataagcactttaaaggataccaaaggagcccaaaaggtaatcaactggcatatgaaaatagaagaacttcgtcttcaagaacttaaactaaatcgggaattagtcaaggataaagaagaaataaaatatttgaataacataatttctgaatatgaacgtacaatcagcagtcttgaagaagaaattgtgcaacagaacaagtttcatgaagaaagacaaatggcctgggatcaaagagaagttgacctggaacgccaactagacatttttgaccgtcagcaaaatgaaatactaaatgcggcacaaaagtttgaagaagctacaggatcaatccctgaccctagtttgccccttccaaatcaacttgagatcgctctaaggaaaattaaggagaacattcgaataattctagaaacacgggcaacttgcaaatcactagaagagaaactaaaagagaaagaatctgctttaaggttagcagaacaaaatatactgtcaagagacaaagtaatcaatgaactgaggcttcgattgcctgccactgcagaaagagaaaagctcatagctgagctaggcagaaaagagatggaaccaaaatctcaccacacattgaaaattgctcatcaaaccattgcaaacatgcaagcaaggttaaatcaaaaagaagaagtattaaagaagtatcaacgtcttctagaaaaagccagagaggagcaaagagaaattgtgaagaaacatgaggaagaccttcatattcttcatcacagattagaactacaggctgatagttcactaaataaattcaaacaaacggcttgggatttaatgaaacagtctcccactccagttcctaccaacaagcattttattcgtctggctgagatggaacagacagtagcagaacaagatgactctctttcctcactcttggtcaaactaaagaaagtatcacaagatttggagagacaaagagaaatcactgaattaaaagtaaaagaatttgaaaatatcaaattacagcttcaagaaaaccatgaagatgaagtgaaaaaagtaaaagcggaagtagaggatttaaagtatcttctggaccagtcacaaaaggagtcacagtgtttaaaatctgaacttcaggctcaaaaagaagcaaattcaagagctccaacaactacaatgagaaatctagtagaacggctaaagagccaattagccttgaaggagaaacaacagaaagcacttagtcgggcacttttagaactccgggcagaaatgacagcagctgctgaagaacgtattatttctgcaacttctcaaaaagaggcccatctcaatgttcaacaaatcgttgatcgacatactagagagctaaagacacaagttgaagatttaaatgaaaatcttttaaaattgaaagaagcacttaaaacaagtaaaaacagagaaaactcactaactgataatttgaatgacttaaataatgaactgcaaaagaaacaaaaagcctataataaaatacttagagagaaagaggaaattgatcaagagaatgatgaactgaaaaggcaaattaaaagactaaccagtggattacagggcaaacccctgacagataataaacaaagtctaattgaagaactccaaaggaaagttaaaaaactagagaaccaattagagggaaaggtggaggaagtagacctaaaacctatgaaagaaaagaatgctaaagaagaattaattaggtgggaagaaggtaaaaagtggcaagccaaaatagaaggaattcgaaacaagttaaaagagaaagagggggaagtctttactttaacaaagcagttgaatactttgaaggatctttttgccaaagccgataaagagaaacttactttgcagaggaaactaaaaacaactggcatgactgttgatcaggttttgggaatacgagctttggagtcagaaaaagaattggaagaattaaaaaagagaaatcttgacttagaaaatgatatattgtatatgagggcccaccaagctcttcctcgagattctgttgtagaagatttacatttacaaaatagatacctccaagaaaaacttcatgctttagaaaaacagttttcaaaggatacatattctaagccttcaatttcaggaatagagtcagatgatcattgtcagagagaacaggagcttcagaaggaaaacttgaagttgtcatctgaaaatattgaactgaaatttcagcttgaacaagcaaataaagatttgccaagattaaagaatcaagtcagagatttgaaggaaatgtgtgaatttcttaagaaagaaaaagcagaagttcagcggaaacttggccatgttagagggtctggtagaagtggaaagacaatcccagaactggaaaaaaccattggtttaatgaaaaaagtagttgaaaaagtccagagagaaaatgaacagttgaaaaaagcatcaggaatattgactagtgaaaaaatggctaatattgagcaggaaaatgaaaaattgaaggctgaattagaaaaacttaaagctcatcttgggcatcagttgagcatgcactatgaatccaagaccaaaggcacagaaaaaattattgctgaaaatgaaaggcttcgtaaagaacttaaaaaagaaactgatgctgcagagaaattacggatagcaaagaataatttagagatattaaatgagaagatgacagttcaactagaagagactggtaagagattgcagtttgcagaaagcagaggtccacagcttgaaggtgctgacagtaagagctggaaatccattgtggttacaagaatgtatgaaaccaagttaaaagaattggaaactgatattgccaaaaaaaatcaaagcattactgaccttaaacagcttgtaaaagaagcaacagagagagaacaaaaagttaacaaatacaatgaagaccttgaacaacagattaagattcttaaacatgttcctgaaggtgctgagacagagcaaggccttaaacgggagcttcaagttcttagattagctaatcatcagctggataaagagaaagcagaattaatccatcagatagaagctaacaaggaccaaagtggagctgaaagcaccatacctgatgctgatcaactaaaggaaaaaataaaagatctagagacacagctcaaaatgtcagatctagaaaagcagcatttgaaggaggaaataaagaagctgaaaaaagaactggaaaattttgatccttcattttttgaagaaattgaagatcttaagtataattacaaggaagaagtgaagaagaatattctcttagaagagaaggtaaaaaaactttcagaacaattgggagttgaattaactagccctgttgctgcttctgaagagtttgaagatgaagaagaaagtcctgttaatttccccatttacatgacgaggattttgacagctttcaaagtggtgaggacactgaagactggttttggctttaccaatgtgactgcacaccaaaaatggaaattttcaagacctggcatcaggctcctttctgtcaaggcacagacagcacacattgtcctggaagatggaactaagatgaaaggttactcctttggccatccatcctctgttgctggtgaagtggtttttaatactggcctgggagggtacccagaagctattactgaccctgcctacaaaggacagattctcacaatggccaaccctattattgggaatggtggagctcctgatactactgctctggatgaactgggacttagcaaatatttggagtctaatggaatcaaggtttcaggtttgctggtgctggattatagtaaagactacaaccactggctggctaccaagagtttagggcaatggctacaggaagaaaaggttcctgcaatttatggagtggacacaagaatgctgactaaaataattcgggataagggtaccatgcttgggaagattgaatttgaaggtcagcctgtggattttgtggatccaaataaacagaatttgattgctgaggtttcaaccaaggatgtcaaagtgtacggcaaaggaaaccccacaaaagtggtagctgtagactgtgggattaaaaacaatgtaatccgcctgctagtaaagcgaggagctgaagtgcacttagttccctggaaccatgatttcaccaagatggagtatgatgggattttgatcgcgggaggaccggggaacccagctcttgcagaaccactaattcagaatgtcagaaagattttggagagtgatcgcaaggagccattgtttggaatcagtacaggaaacttaataacaggattggctgctggtgccaaaacctacaagatgtccatggccaacagagggcagaatcagcctgttttgaatatcacaaacaaacaggctttcattactgctcagaatcatggctatgccttggacaacaccctccctgctggctggaaaccactttttgtgaatgtcaacgatcaaacaaatgaggggattatgcatgagagcaaacccttcttcgctgtgcagttccacccagaggtcaccccggggccaatagacactgagtacctgtttgattcctttttctcactgataaagaaaggaaaagctaccaccattacatcagtcttaccgaagccagcactagttgcatctcgggttgaggtttccaaagtccttattctaggatcaggaggtctgtccattggtcaggctggagaatttgattactcaggatctcaagctgtaaaagccatgaaggaagaaaatgtcaaaactgttctgatgaacccaaacattgcatcagtccagaccaatgaggtgggcttaaagcaagcggatactgtctactttcttcccatcacccctcagtttgtcacagaggtcatcaaggcagaacagccagatgggttaattctgggcatgggtggccagacagctctgaactgtggagtggaactattcaagagaggtgtgctcaaggaatatggtgtgaaagtcctgggaacttcagttgagtccattatggctacggaagacaggcagctgttttcagataaactaaatgagatcaatgaaaagattgctccaagttttgcagtggaatcgattgaggatgcactgaaggcagcagacaccattggctacccagtgatgatccgttccgcctatgcactgggtgggttaggctcaggcatctgtcccaacagagagactttgatggacctcagcacaaaggcctttgctatgaccaaccaaattctggtggagaagtcagtgacggcccggctgtggctgaggagcccctccaccgaccgtgagtttgggctgcatgactgcatggttgcatgaacacatattaatttcctccacttagttctacacctcattcattcattcagtgagtgtttctcgactactatgaataaaccgttatactccatgttgcgggcagaatggggatctggacagggaagcacagggcacgagttcaccaatggctgtcaagctacgctgcccgcctaacactgccaatgccggtcccaagcccggataaaaagtggagggggcgggaaggtttttcttttcctgagaaaacaacacgtattgttttctcaggttttgctttttggcctttttctagcttaaaaaaaaaaaaagcaaaagaattctgcagatatccatcacact

SEQ ID NO: 127 - DNA, 8473 bp, synthetic construct
  source 1..8473: mol_type = other DNA
agacccaagcttggtaccgagctcggatccggccaccatggactacaaagacgatgacgacaaggaattagttgaggctctgaagaggttaaaagattatgaatcgggagtatatggtttagaagatgctgtcgttgaaataaagaattgtaaaaaccaaattaaaataagagatcgagagattgaaatattaacaaaggaaatcaataaacttgaattgaagatcagtgatttccttgatgaaaatgaggcacttagagagcgtgtgggccttgaaccaaagacaatgattgatttaactgaatttagaaatagcaaacacttaaaacagcagcagtacagagctgaaaaccagattcttttgaaagagattgaaagtctagaggaagaacgacttgatctgaaaaaaaaaattcgtcaaatggctcaagaaagaggaaaaagaagtgcaacttcaggattaaccactgaggacctgaacctaactgaaaacatttctcaaggagatagaataagtgaaagaaaattggatttattgagcctcaaaaatatgagtgaagcacaatcaaagaatgaatttctttcaagagaactaattgaaaaagaaagagatttagaaaggagtaggacagtgatagccaaatttcagaataaattaaaagaattagttgaagaaaataagcaacttgaagaaggtatgaaagaaatattgcaagcaattaaggaaatgcagaaagatcctgatgttaaaggaggagaaacatctctaattatccctagccttgaaagactagttaatgctatagaatcaaagaatgcagaaggaatctttgatgcgagtctgcatttgaaagcccaagttgatcagcttaccggaagaaatgaagaattaagacaggagctcagggaatctcggaaagaggctataaattattcacagcagttggcaaaagctaatttaaagatagaccatcttgaaaaagaaactagtcttttacgacaatcagaaggatcaaatgttgtttttaaaggaattgacttacctgatgggatagcaccatctagtgccagtatcattaattctcagaatgaatatttaatacatttgttacaggaactagaaaataaagaaaaaaagttaaagaatttagaagattctcttgaagattacaacagaaaatttgctgtaattcgtcatcaacaaagtttgttgtataaagaatacctaagtgaaaaggagacctggaaaacagaatctaaaacaataaaagaggaaaagagaaaacttgaggatcaagtccaacaagatgctataaaagtaaaagaatataataatttgctcaatgctcttcagatggattcggatgaaatgaaaaaaatacttgcagaaaatagtaggaaaattactgttttgcaagtgaatgaaaaatcacttataaggcaatatacaaccttagtagaattggagcgacaacttagaaaagaaaatgagaagcaaaagaatgaattgttgtcaatggaggctgaagtttgtgaaaaaattgggtgtttgcaaagatttaaggaaatggccattttcaagattgcagctctccaaaaagttgtagataatagtgtttctttgtctgaactagaactggctaataaacagtacaatgaactgactgctaagtacagggacatcttgcaaaaagataatatgcttgttcaaagaacaagtaacttggaacacctggagtgtgaaaacatctccttaaaagaacaagtggagtctataaataaagaactggagattaccaaggaaaaacttcacactattgaacaagcctgggaacaggaaactaaattaggtaatgaatctagcatggataaggcaaagaaatcaataaccaacagtgacattgtttccatttcaaaaaaaataactatgctggaaatgaaggaattaaatgaaaggcagcgggctgaacattgtcaaaaaatgtatgaacacttacggacttcgttaaagcaaatggaggaacgtaattttgaattggaaaccaaatttgctgagcttaccaaaatcaatttggatgcacagaaggtggaacagatgttaagagatgaattagctgatagtgtgagcaaggcagtaagtgatgctgataggcaacggattctagaattagagaagaatgaaatggaactaaaagttgaagtgtcaaaactgagagagatttctgatattgccagaagacaagttgaaattttgaatgcacaacaacaatctagggacaaggaagtagagtccctcagaatgcaactgctagactatcaggcacagtctgatgaaaagtcgctcattgccaagttgcaccaacataatgtctctcttcaactgagtgaggctactgctcttggtaagttggagtcaattacatctaaactgcagaagatggaggcctacaacttgcgcttagagcagaaacttgatgaaaaagaacaggctctctattatgctcgtttggagggaagaaacagagcaaaacatctgcgccaaacaattcagtctctacgacgacagtttagtggagctttacccttggcacaacaggaaaagttctccaaaacaatgattcaactacaaaatgacaaacttaagataatgcaagaaatgaaaaattctcaacaagaacatagaaatatggagaacaaaacattggagatggaattaaaattaaagggcctggaagagttaataagcactttaaaggataccaaaggagcccaaaaggtaatcaactggcatatgaaaatagaagaacttcgtcttcaagaacttaaactaaatcgggaattagtcaaggataaagaagaaataaaatatttgaataacataatttctgaatatgaacgtacaatcagcagtcttgaagaagaaattgtgcaacagaacaagtttcatgaagaaagacaaatggcctgggatcaaagagaagttgacctggaacgccaactagacatttttgaccgtcagcaaaatgaaatactaaatgcggcacaaaagtttgaagaagctacaggatcaatccctgaccctagtttgccccttccaaatcaacttgagatcgctctaaggaaaattaaggagaacattcgaataattctagaaacacgggcaacttgcaaatcactagaagagaaactaaaagagaaagaatctgctttaaggttagcagaacaaaatatactgtcaagagacaaagtaatcaatgaactgaggcttcgattgcctgccactgcagaaagagaaaagctcatagctgagctaggcagaaaagagatggaaccaaaatctcaccacacattgaaaattgctcatcaaaccattgcaaacatgcaagcaaggttaaatcaaaaagaagaagtattaaagaagtatcaacgtcttctagaaaaagccagagaggagcaaagagaaattgtgaagaaacatgaggaagaccttcatattcttcatcacagattagaactacaggctgatagttcactaaataaattcaaacaaacggcttgggatttaatgaaacagtctcccactccagttcctaccaacaagcattttattcgtctggctgagatggaacagacagtagcagaacaagatgactctctttcctcactcttggtcaaactaaagaaagtatcacaagatttggagagacaaagagaaatcactgaattaaaagtaaaagaatttgaaaatatcaaattacagcttcaagaaaaccatgaagatgaagtgaaaaaagtaaaagcggaagtagaggatttaaagtatcttctggaccagtcacaaaaggagtcacagtgtttaaaatctgaacttcaggctcaaaaagaagcaaattcaagagctccaacaactacaatgagaaatctagtagaacggctaaagagccaattagccttgaaggagaaacaacagaaagcacttagtcgggcacttttagaactccgggcagaaatgacagcagctgctgaagaacgtattatttctgcaacttctcaaaaagaggcccatctcaatgttcaacaaatcgttgatcgacatactagagagctaaagacacaagttgaagatttaaatgaaaatcttttaaaattgaaagaagcacttaaaacaagtaaaaacagagaaaactcactaactgataatttgaatgacttaaataatgaactgcaaaagaaacaaaaagcctataataaaatacttagagagaaagaggaaattgatcaagagaatgatgaactgaaaaggcaaattaaaagactaaccagtggattacagggcaaacccctgacagataataaacaaagtctaattgaagaactccaaaggaaagttaaaaaactagagaaccaattagagggaaaggtggaggaagtagacctaaaacctatgaaagaaaagaatgctaaagaagaattaattaggtgggaagaaggtaaaaagtggcaagccaaaatagaaggaattcgaaacaagttaaaagagaaagagggggaagtctttactttaacaaagcagttgaatactttgaaggatctttttgccaaagccgataaagagaaacttactttgcagaggaaactaaaaacaactggcatgactgttgatcaggttttgggaatacgagctttggagtcagaaaaagaattggaagaattaaaaaagagaaatcttgacttagaaaatgatatattgtatatgagggcccaccaagctcttcctcgagattctgttgtagaagatttacatttacaaaatagatacctccaagaaaaacttcatgctttagaaaaacagttttcaaaggatacatattctaagccttcaatttcaggaatagagtcagatgatcattgtcagagagaacaggagcttcagaaggaaaacttgaagttgtcatctgaaaatattgaactgaaatttcagcttgaacaagcaaataaagatttgccaagattaaagaatcaagtcagagatttgaaggaaatgtgtgaatttcttaagaaagaaaaagcagaagttcagcggaaacttggccatgttagagggtctggtagaagtggaaagacaatcccagaactggaaaaaaccattggtttaatgaaaaaagtagttgaaaaagtccagagagaaaatgaacagttgaaaaaagcatcaggaatattgactagtgaaaaaatggctaatattgagcaggaaaatgaaaaattgaaggctgaattagaaaaacttaaagctcatcttgggcatcagttgagcatgcactatgaatccaagaccaaaggcacagaaaaaattattgctgaaaatgaaaggcttcgtaaagaacttaaaaaagaaactgatgctgcagagaaattacggatagcaaagaataatttagagatattaaatgagaagatgacagttcaactagaagagactggtaagagattgcagtttgcagaaagcagaggtccacagcttgaaggtgctgacagtaagagctggaaatccattgtggttacaagaatgtatgaaaccaagttaaaagaattggaaactgatattgccaaaaaaaatcaaagcattactgaccttaaacagcttgtaaaagaagcaacagagagagaacaaaaagttaacaaatacaatgaagaccttgaacaacagattaagattcttaaacatgttcctgaaggtgctgagacagagcaaggccttaaacgggagcttcaagttcttagattagctaatcatcagctggataaagagaaagcagaattaatccatcagatagaagctaacaaggaccaaagtggagctgaaagcaccatacctgatgctgatcaactaaaggaaaaaataaaagatctagagacacagctcaaaatgtcagatctagaaaagcagcatttgaaggaggaaataaagaagctgaaaaaagaactggaaaattttgatccttcattttttgaagaaattgaagatcttaagtataattacaaggaagaagtgaagaagaatattctcttagaagagaaggtaaaaaaactttcagaacaattgggagttgaattaactagccctgttgctgcttctgaagagtttgaagatgaagaagaaagtcctgttaatttccccatttacatgacgaggattttgacagctttcaaagtggtgaggacactgaagactggttttggctttaccaatgtgactgcacaccaaaaatggaaattttcaagacctggcatcaggctcctttctgtcaaggcacagacagcacacattgtcctggaagatggaactaagatgaaaggttactcctttggccatccatcctctgttgctggtgaagtggtttttaatactggcctgggagggtacccagaagctattactgaccctgcctacaaaggacagattctcacaatggccaaccctattattgggaatggtggagctcctgatactactgctctggatgaactgggacttagcaaatatttggagtctaatggaatcaaggtttcaggtttgctggtgctggattatagtaaagactacaaccactggctggctaccaagagtttagggcaatggctacaggaagaaaaggttcctgcaatttatggagtggacacaagaatgctgactaaaataattcgggataagggtaccatgcttgggaagattgaatttgaaggtcagcctgtggattttgtggatccaaataaacagaatttgattgctgaggtttcaaccaaggatgtcaaagtgtacggcaaaggaaaccccacaaaagtggtagctgtagactgtgggattaaaaacaatgtaatccgcctgctagtaaagcgaggagctgaagtgcacttagttccctggaaccatgatttcaccaagatggagtatgatgggattttgatcgcgggaggaccggggaacccagctcttgcagaaccactaattcagaatgtcagaaagattttggagagtgatcgcaaggagccattgtttggaatcagtacaggaaacttaataacaggattggctgctggtgccaaaacctacaagatgtccatggccaacagagggcagaatcagcctgttttgaatatcacaaacaaacaggctttcattactgctcagaatcatggctatgccttggacaacaccctccctgctggctggaaaccactttttgtgaatgtcaacgatcaaacaaatgaggggattatgcatgagagcaaacccttcttcgctgtgcagttccacccagaggtcaccccggggccaatagacactgagtacctgtttgattcctttttctcactgataaagaaaggaaaagctaccaccattacatcagtcttaccgaagccagcactagttgcatctcgggttgaggtttccaaagtccttattctaggatcaggaggtctgtccattggtcaggctggagaatttgattactcaggatctcaagctgtaaaagccatgaaggaagaaaatgtcaaaactgttctgatgaacccaaacattgcatcagtccagaccaatgaggtgggcttaaagcaagcggatactgtctactttcttcccatcacccctcagtttgtcacagaggtcatcaaggcagaacagccagatgggttaattctgggcatgggtggccagacagctctgaactgtggagtggaactattcaagagaggtgtgctcaaggaatatggtgtgaaagtcctgggaacttcagttgagtccattatggctacggaagacaggcagctgttttcagataaactaaatgagatcaatgaaaagattgctccaagttttgcagtggaatcgattgaggatgcactgaaggcagcagacaccattggctacccagtgatgatccgttccgcctatgcactgggtgggttaggctcaggcatctgtcccaacagagagactttgatggacctcagcacaaaggcctttgctatgaccaaccaaattctggtggagaagtcagtgacggcccggctgtggctgaggagcccctccaccgaccgtgagtttgggctgcatgactgcatggttgcatgaacacatattaatttcctccacttagttctacacctcattcattcattcagtgagtgtttctcgactactatgaataaaccgttatactccatgttgcgggcagaatggggatctggacagggaagcacagggcacgagttcaccaatggctgtcaagctacgctgcccgcctaacactgccaatgccggtcccaagcccggataaaaagtggagggggcgggaaggtttttcttttcctgagaaaacaacacgtattgttttctcaggttttgctttttggcctttttctagcttaaaaaaaaaaaaagcaaaagaattctgcagatatccatcacact

SEQ ID NO: 128 - DNA, 10473 bp, synthetic construct
  source 1..10473: mol_type = other DNA
agacccaagcttggtaccgagctcggatccggccaccatggactacaaagacgatgacgacaaggccatgaagatcgagtgccgcatcaccggcaccctgaacggcgtggagttcgagctggtgggcggcggagagggcacccccgagcagggccgcatgaccaacaagatgaagagcaccaaaggcgccctgaccttcagcccctacctgctgagccacgtgatgggctacggcttctaccacttcggcacctaccccagcggctacgagaaccccttcctgcacgccatcaacaacggcggctacaccaacacccgcatcgagaagtacgaggacggcggcgtgctgcacgtgagcttcagctaccgctacgaggccggccgcgtgatcggcgacttcaaggtggtgggcaccggcttccccgaggacagcgtgatcttcaccgacaagatcatccgcagcaacgccaccgtggagcacctgcaccccatgggcgataacgtgctggtgggcagcttcgcccgcaccttcagcctgcgcgacggcggctactacagcttcgtggtggacagccacatgcacttcaagagcgccatccaccccagcatcctgcagaacgggggccccatgttcgccttccgccgcgtggaggagctgcacagcaacaccgagctgggcatcgtggagtaccagcacgccttcaagacccccatcgccttcgccagatctcgagctcgaatgtcgacatgccacctaatataaactggaaagaaataatgaaagttgacccagatgacctgccccgtcaagaagaactggcagataatttattgatttccttatccaaggtggaagtaaatgagctaaaaagtgaaaagcaagaaaatgtgatacaccttttcagaattactcagtcactaatgaagatgaaagctcaagaagtggagctggctttggaagaagtagaaaaagctggagaagaacaagcaaaatttgaaaatcaattaaaaactaaagtaatgaaactggaaaatgaactggagatggctcagcagtctgcaggtggacgagatactcggtttttacgtaatgaaatttgccaacttgaaaaacaattagaacaaaaagatagagaattggaggacatggaaaaggagttggagaaagagaagaaagttaatgagcaattggctcttcgaaatgaggaggcagaaaatgaaaacagcaaattaagaagagagaacaaacgtctaaagaaaaagaatgaacaactttgtcaggatattattgactaccagaaacaaatagattcacagaaagaaacacttttatcaagaagaggggaagacagtgactaccgatcacagttgtctaaaaaaaactatgagcttatccaatatcttgatgaaattcagactttaacagaagctaatgagaaaattgaagttcagaatcaagaaatgagaaaaaatttagaagagtctgtacaggaaatggagaagatgactgatgaatataatagaatgaaagctattgtgcatcagacagataatgtaatagatcagttaaaaaaagaaaacgatcattatcaacttcaagtgcaggagcttacagatcttctgaaatcaaaaaatgaagaagatgatccaattatggtagctgtcaatgcaaaagtagaagaatggaagctaattttgtcttctaaagatgatgaaattattgagtatcagcaaatgttacataacctaagggagaaacttaagaatgctcagcttgatgctgataaaagtaatgttatggctctacagcagggtatacaggaacgagacagtcaaattaagatgctcaccgaacaagtagaacaatatacaaaagaaatggaaaagaatacttgtattattgaagatttgaaaaatgagctccaaagaaacaaaggtgcttcaaccctttctcaacagactcatatgaaaattcagtcaacgttagacattttaaaagagaaaactaaagaggctgagagaacagctgaactggctgaggctgatgctagggaaaaggataaagaattagttgaggctctgaagaggttaaaagattatgaatcgggagtatatggtttagaagatgctgtcgttgaaataaagaattgtaaaaaccaaattaaaataagagatcgagagattgaaatattaacaaaggaaatcaataaacttgaattgaagatcagtgatttccttgatgaaaatgaggcacttagagagcgtgtgggccttgaaccaaagacaatgattgatttaactgaatttagaaatagcaaacacttaaaacagcagcagtacagagctgaaaaccagattcttttgaaagagattgaaagtctagaggaagaacgacttgatctgaaaaaaaaaattcgtcaaatggctcaagaaagaggaaaaagaagtgcaacttcaggattaaccactgaggacctgaacctaactgaaaacatttctcaaggagatagaataagtgaaagaaaattggatttattgagcctcaaaaatatgagtgaagcacaatcaaagaatgaatttctttcaagagaactaattgaaaaagaaagagatttagaaaggagtaggacagtgatagccaaatttcagaataaattaaaagaattagttgaagaaaataagcaacttgaagaaggtatgaaagaaatattgcaagcaattaaggaaatgcagaaagatcctgatgttaaaggaggagaaacatctctaattatccctagccttgaaagactagttaatgctatagaatcaaagaatgcagaaggaatctttgatgcgagtctgcatttgaaagcccaagttgatcagcttaccggaagaaatgaagaattaagacaggagctcagggaatctcggaaagaggctataaattattcacagcagttggcaaaagctaatttaaagatagaccatcttgaaaaagaaactagtcttttacgacaatcagaaggatcaaatgttgtttttaaaggaattgacttacctgatgggatagcaccatctagtgccagtatcattaattctcagaatgaatatttaatacatttgttacaggaactagaaaataaagaaaaaaagttaaagaatttagaagattctcttgaagattacaacagaaaatttgctgtaattcgtcatcaacaaagtttgttgtataaagaatacctaagtgaaaaggagacctggaaaacagaatctaaaacaataaaagaggaaaagagaaaacttgaggatcaagtccaacaagatgctataaaagtaaaagaatataataatttgctcaatgctcttcagatggattcggatgaaatgaaaaaaatacttgcagaaaatagtaggaaaattactgttttgcaagtgaatgaaaaatcacttataaggcaatatacaaccttagtagaattggagcgacaacttagaaaagaaaatgagaagcaaaagaatgaattgttgtcaatggaggctgaagtttgtgaaaaaattgggtgtttgcaaagatttaaggaaatggccattttcaagattgcagctctccaaaaagttgtagataatagtgtttctttgtctgaactagaactggctaataaacagtacaatgaactgactgctaagtacagggacatcttgcaaaaagataatatgcttgttcaaagaacaagtaacttggaacacctggagtgtgaaaacatctccttaaaagaacaagtggagtctataaataaagaactggagattaccaaggaaaaacttcacactattgaacaagcctgggaacaggaaactaaattaggtaatgaatctagcatggataaggcaaagaaatcaataaccaacagtgacattgtttccatttcaaaaaaaataactatgctggaaatgaaggaattaaatgaaaggcagcgggctgaacattgtcaaaaaatgtatgaacacttacggacttcgttaaagcaaatggaggaacgtaattttgaattggaaaccaaatttgctgagcttaccaaaatcaatttggatgcacagaaggtggaacagatgttaagagatgaattagctgatagtgtgagcaaggcagtaagtgatgctgataggcaacggattctagaattagagaagaatgaaatggaactaaaagttgaagtgtcaaaactgagagagatttctgatattgccagaagacaagttgaaattttgaatgcacaacaacaatctagggacaaggaagtagagtccctcagaatgcaactgctagactatcaggcacagtctgatgaaaagtcgctcattgccaagttgcaccaacataatgtctctcttcaactgagtgaggctactgctcttggtaagttggagtcaattacatctaaactgcagaagatggaggcctacaacttgcgcttagagcagaaacttgatgaaaaagaacaggctctctattatgctcgtttggagggaagaaacagagcaaaacatctgcgccaaacaattcagtctctacgacgacagtttagtggagctttacccttggcacaacaggaaaagttctccaaaacaatgattcaactacaaaatgacaaacttaagataatgcaagaaatgaaaaattctcaacaagaacatagaaatatggagaacaaaacattggagatggaattaaaattaaagggcctggaagagttaataagcactttaaaggataccaaaggagcccaaaaggtaatcaactggcatatgaaaatagaagaacttcgtcttcaagaacttaaactaaatcgggaattagtcaaggataaagaagaaataaaatatttgaataacataatttctgaatatgaacgtacaatcagcagtcttgaagaagaaattgtgcaacagaacaagtttcatgaagaaagacaaatggcctgggatcaaagagaagttgacctggaacgccaactagacatttttgaccgtcagcaaaatgaaatactaaatgcggcacaaaagtttgaagaagctacaggatcaatccctgaccctagtttgccccttccaaatcaacttgagatcgctctaaggaaaattaaggagaacattcgaataattctagaaacacgggcaacttgcaaatcactagaagagaaactaaaagagaaagaatctgctttaaggttagcagaacaaaatatactgtcaagagacaaagtaatcaatgaactgaggcttcgattgcctgccactgcagaaagagaaaagctcatagctgagctaggcagaaaagagatggaaccaaaatctcaccacacattgaaaattgctcatcaaaccattgcaaacatgcaagcaaggttaaatcaaaaagaagaagtattaaagaagtatcaacgtcttctagaaaaagccagagaggagcaaagagaaattgtgaagaaacatgaggaagaccttcatattcttcatcacagattagaactacaggctgatagttcactaaataaattcaaacaaacggcttgggatttaatgaaacagtctcccactccagttcctaccaacaagcattttattcgtctggctgagatggaacagacagtagcagaacaagatgactctctttcctcactcttggtcaaactaaagaaagtatcacaagatttggagagacaaagagaaatcactgaattaaaagtaaaagaatttgaaaatatcaaattacagcttcaagaaaaccatgaagatgaagtgaaaaaagtaaaagcggaagtagaggatttaaagtatcttctggaccagtcacaaaaggagtcacagtgtttaaaatctgaacttcaggctcaaaaagaagcaaattcaagagctccaacaactacaatgagaaatctagtagaacggctaaagagccaattagccttgaaggagaaacaacagaaagcacttagtcgggcacttttagaactccgggcagaaatgacagcagctgctgaagaacgtattatttctgcaacttctcaaaaagaggcccatctcaatgttcaacaaatcgttgatcgacatactagagagctaaagacacaagttgaagatttaaatgaaaatcttttaaaattgaaagaagcacttaaaacaagtaaaaacagagaaaactcactaactgataatttgaatgacttaaataatgaactgcaaaagaaacaaaaagcctataataaaatacttagagagaaagaggaaattgatcaagagaatgatgaactgaaaaggcaaattaaaagactaaccagtggattacagggcaaacccctgacagataataaacaaagtctaattgaagaactccaaaggaaagttaaaaaactagagaaccaattagagggaaaggtggaggaagtagacctaaaacctatgaaagaaaagaatgctaaagaagaattaattaggtgggaagaaggtaaaaagtggcaagccaaaatagaaggaattcgaaacaagttaaaagagaaagagggggaagtctttactttaacaaagcagttgaatactttgaaggatctttttgccaaagccgataaagagaaacttactttgcagaggaaactaaaaacaactggcatgactgttgatcaggttttgggaatacgagctttggagtcagaaaaagaattggaagaattaaaaaagagaaatcttgacttagaaaatgatatattgtatatgagggcccaccaagctcttcctcgagattctgttgtagaagatttacatttacaaaatagatacctccaagaaaaacttcatgctttagaaaaacagttttcaaaggatacatattctaagccttcaatttcaggaatagagtcagatgatcattgtcagagagaacaggagcttcagaaggaaaacttgaagttgtcatctgaaaatattgaactgaaatttcagcttgaacaagcaaataaagatttgccaagattaaagaatcaagtcagagatttgaaggaaatgtgtgaatttcttaagaaagaaaaagcagaagttcagcggaaacttggccatgttagagggtctggtagaagtggaaagacaatcccagaactggaaaaaaccattggtttaatgaaaaaagtagttgaaaaagtccagagagaaaatgaacagttgaaaaaagcatcaggaatattgactagtgaaaaaatggctaatattgagcaggaaaatgaaaaattgaaggctgaattagaaaaacttaaagctcatcttgggcatcagttgagcatgcactatgaatccaagaccaaaggcacagaaaaaattattgctgaaaatgaaaggcttcgtaaagaacttaaaaaagaaactgatgctgcagagaaattacggatagcaaagaataatttagagatattaaatgagaagatgacagttcaactagaagagactggtaagagattgcagtttgcagaaagcagaggtccacagcttgaaggtgctgacagtaagagctggaaatccattgtggttacaagaatgtatgaaaccaagttaaaagaattggaaactgatattgccaaaaaaaatcaaagcattactgaccttaaacagcttgtaaaagaagcaacagagagagaacaaaaagttaacaaatacaatgaagaccttgaacaacagattaagattcttaaacatgttcctgaaggtgctgagacagagcaaggccttaaacgggagcttcaagttcttagattagctaatcatcagctggataaagagaaagcagaattaatccatcagatagaagctaacaaggaccaaagtggagctgaaagcaccatacctgatgctgatcaactaaaggaaaaaataaaagatctagagacacagctcaaaatgtcagatctagaaaagcagcatttgaaggaggaaataaagaagctgaaaaaagaactggaaaattttgatccttcattttttgaagaaattgaagatcttaagtataattacaaggaagaagtgaagaagaatattctcttagaagagaaggtaaaaaaactttcagaacaattgggagttgaattaactagccctgttgctgcttctgaagagtttgaagatgaagaagaaagtcctgttaatttccccatttacatgacgaggattttgacagctttcaaagtggtgaggacactgaagactggttttggctttaccaatgtgactgcacaccaaaaatggaaattttcaagacctggcatcaggctcctttctgtcaaggcacagacagcacacattgtcctggaagatggaactaagatgaaaggttactcctttggccatccatcctctgttgctggtgaagtggtttttaatactggcctgggagggtacccagaagctattactgaccctgcctacaaaggacagattctcacaatggccaaccctattattgggaatggtggagctcctgatactactgctctggatgaactgggacttagcaaatatttggagtctaatggaatcaaggtttcaggtttgctggtgctggattatagtaaagactacaaccactggctggctaccaagagtttagggcaatggctacaggaagaaaaggttcctgcaatttatggagtggacacaagaatgctgactaaaataattcgggataagggtaccatgcttgggaagattgaatttgaaggtcagcctgtggattttgtggatccaaataaacagaatttgattgctgaggtttcaaccaaggatgtcaaagtgtacggcaaaggaaaccccacaaaagtggtagctgtagactgtgggattaaaaacaatgtaatccgcctgctagtaaagcgaggagctgaagtgcacttagttccctggaaccatgatttcaccaagatggagtatgatgggattttgatcgcgggaggaccggggaacccagctcttgcagaaccactaattcagaatgtcagaaagattttggagagtgatcgcaaggagccattgtttggaatcagtacaggaaacttaataacaggattggctgctggtgccaaaacctacaagatgtccatggccaacagagggcagaatcagcctgttttgaatatcacaaacaaacaggctttcattactgctcagaatcatggctatgccttggacaacaccctccctgctggctggaaaccactttttgtgaatgtcaacgatcaaacaaatgaggggattatgcatgagagcaaacccttcttcgctgtgcagttccacccagaggtcaccccggggccaatagacactgagtacctgtttgattcctttttctcactgataaagaaaggaaaagctaccaccattacatcagtcttaccgaagccagcactagttgcatctcgggttgaggtttccaaagtccttattctaggatcaggaggtctgtccattggtcaggctggagaatttgattactcaggatctcaagctgtaaaagccatgaaggaagaaaatgtcaaaactgttctgatgaacccaaacattgcatcagtccagaccaatgaggtgggcttaaagcaagcggatactgtctactttcttcccatcacccctcagtttgtcacagaggtcatcaaggcagaacagccagatgggttaattctgggcatgggtggccagacagctctgaactgtggagtggaactattcaagagaggtgtgctcaaggaatatggtgtgaaagtcctgggaacttcagttgagtccattatggctacggaagacaggcagctgttttcagataaactaaatgagatcaatgaaaagattgctccaagttttgcagtggaatcgattgaggatgcactgaaggcagcagacaccattggctacccagtgatgatccgttccgcctatgcactgggtgggttaggctcaggcatctgtcccaacagagagactttgatggacctcagcacaaaggcctttgctatgaccaaccaaattctggtggagaagtcagtgacggcccggctgtggctgaggagcccctccaccgaccgtgagtttgggctgcatgactgcatggttgcatgaacacatattaatttcctccacttagttctacacctcattcattcattcagtgagtgtttctcgactactatgaataaaccgttatactccatgttgcgggcagaatggggatctggacagggaagcacagggcacgagttcaccaatggctgtcaagctacgctgcccgcctaacactgccaatgccggtcccaagcccggataaaaagtggagggggcgggaaggtttttcttttcctgagaaaacaacacgtattgttttctcaggttttgctttttggcctttttctagcttaaaaaaaaaaaaagcaaaagaattctgcagatatccatcacact

(19) United States
(12) Patent Application Publication
(10) Pub. No.: US 2025/0179494 A1
(43) Pub. Date: Jun. 5, 2025

(54) RIBOZYME-ENHANCED RNA TRANS-SPLICING

(71) Applicants: Massachusetts Institute of Technology, Cambridge, MA (US); The Brigham and Women's Hospital, Inc., Boston, MA (US); Beth Israel Deaconess Medical Center, Boston, MA (US)

(72) Inventors: Omar Abudayyeh, Cambridge, MA (US); Jonathan Gootenberg, Cambridge, MA (US); Cian Schmitt-Ulms, Cambridge, MA (US); Alisan Kayabolen, Cambridge, MA (US)

(73) Assignees: Massachusetts Institute of Institute of Technology, Cambridge, MA (US); The Brigham and Women's Hospital, Inc., Boston, MA (US); Beth Israel Deaconess Medical Center, Boston, MA (US)

(21) Appl. No.: 18/962,394

(22) Filed: Nov. 27, 2024

Related U.S. Application Data
(60) Provisional application No. 63/605,049, filed on Dec. 1, 2023.

(51) Int. Cl. C12N 15/113; C12N 15/86

(52) U.S. Cl. CPC C12N 15/113; C12N 15/86; C12N 2310/121; C12N 2750/14143

(57) ABSTRACT

The invention relates to compositions and methods for RNA editing. In particular, ribozymes can be utilized to enhance RNA trans-splicing.

### CROSS REFERENCE TO RELATED APPLICATION

This application claims the benefit under 35 U.S.C. § 119(e) of U.S. Provisional Application No. 63/605,049, filed Dec. 1, 2023, the entire contents of which is incorporated herein by reference.

### FEDERALLY SPONSORED RESEARCH

This invention was made with government support under EB031957 and under H0011857 awarded by the National Institutes of Health. The government has certain rights in the invention.

### REFERENCE TO AN ELECTRONIC SEQUENCE LISTING

The contents of the electronic sequence listing (M065670543US01-SEQ-EAS.xml; Size: 200,376 bytes; and Date of Creation: Nov. 26, 2024) is herein incorporated by reference in its entirety.

### FIELD OF THE INVENTION

The subject matter disclosed herein is generally related to systems, methods, and compositions for ribozyme-enhanced RNA trans-splicing.

### BACKGROUND OF THE INVENTION

Gene editing tools for programmable enzymatic modification of DNA, including base editors (Komor et al. 2016; Nishida et al. 2016; Gaudelli et al. 2017), prime editing (Anzalone et al. 2019), and insertion tools (Yarnall et al. 2023; Anzalone et al. 2022; Lampe et al. 2023), have made significant progress, but these tools are large and bulky, cannot install arbitrary edits, leaving many of the 141,342 known pathogenic mutations unaddressed, and have the risk of permanent off-targets (Zuo et al. 2019) and bystander edits (Fiumara et al. 2023), potentially limiting clinical utility. In contrast, RNA editors are typically simpler and easier to deliver to diverse tissues and have no risk of permanent off-targets (Vallecilli-Viejo et al. 2018; Katrekar et al. 2022; Ruchika & Nakamura 2022). However, mature RNA editing technologies are currently limited, and can only effect two base transitions, (A to I or C to U) (Cox et al. 2017; Abudayyeh et al. 2019; Merkle et al. 2019; Vogel et al. 2018; Fukuda et al. 2017; Wettengel et al. 2017; Montiel-Gonzalez et al. 2016; Vogel et al. 2014; Montiel-Gonzalez et al. 2013; Rees et al. 2018), leaving the ten other possible base transitions and transversions, as well as small or large insertions or deletions, completely unaddressed. Furthermore, both DNA and RNA editing approaches rely on large protein cargos, often precluding the use of common delivery vectors, such as adeno associated viruses (AAVs).

Alternatively, trans-splicing based RNA editing approaches can replace exons for flexible edits, but have been traditionally hampered by low efficiencies (Berger et al. 2016; Puttaraju et al. 1999; Liu et al. 2002; Wang et al. 2009; Coady & Lorson 2010; Coady et al. 2007; Berger et al. 2015; Rindt et al. 2012). As transversions, insertions, and deletions account for a majority of pathogenic mutations (Antonarakis & Cooper 2010), having the means to efficiently install these changes across any transcript in any cell type with viral or non-viral delivery is critical.

### SUMMARY OF THE INVENTION

The present invention relates, in part, to the discovery that single-component trans-splicing template polynucleotides comprising a ribozyme can be useful for RNA trans-splicing. In some aspects, for RNA trans-splicing, cleavage of the poly(A) tail of the trans-template by engineered ribozymes increases the splicing efficiency. The inventors further discovered that when using ribozymes, only the RNA trans-template is needed, with no exogenous proteins required, offering the simplest approach for programmable RNA writing. The invention relates, in some aspects, to the discovery that a protein-free, single trans-splicing template comprising a ribozyme can be harnessed for 5′ RNA trans-splicing and 3′ RNA trans-splicing. The invention relates, in some aspects to the use of more than one protein-free, single trans-splicing template comprising a ribozyme for simultaneous 5′ and 3′ RNA trans-splicing. The invention relates, in some aspects, to the use of a protein-free, single trans-splicing template comprising a ribozyme with, for instance, a Cas7-11 and gRNA for simultaneous 5′ and 3′ RNA trans-splicing.

Accordingly, aspects of the present disclosure provide non-naturally occurring, engineered compositions comprising: a trans-splicing template polynucleotide comprising: (a) an insertion sequence; (b) a 5′ splicing motif sequence; (c) optionally, a linker sequence; (d) a hybridization sequence; and (e) a nucleic acid sequence encoding a ribozyme.

In some embodiments, (a)-(e) are arranged 5′ to 3.

In some embodiments, the ribozyme is capable of cleaving RNA.

In some embodiments, the ribozyme is capable of cleaving DNA.

In some embodiments, the ribozyme is a self-cleaving ribozyme.

In some embodiments, the ribozyme is a naturally occurring ribozyme.

In some embodiments, the ribozyme is a synthetic ribozyme.

In some embodiments, the ribozyme is selected from Twister ribozyme, Hammerhead (HH) ribozyme, Hepatitis Delta Virus (HDV) ribozyme, Hairpin 1 ribozyme, Hairpin 2 ribozyme, Hairpin 3 ribozyme, Varkud Satellite ribozyme, glmS ribozyme, twister sister ribozyme, pistol ribozyme, and hatchet ribozyme.

In some embodiments, the trans-splicing template polynucleotide comprises a sequence that is at least 90% identical to one of SEQ ID NOs: 1-7, 9-104, or 117-128. In some embodiments, the trans-splicing template polynucleotide comprises a sequence of one of SEQ ID NOs: 1-7, 9-104, or 117-128.

In some embodiments, the insertion sequence is less than 1-2 kilobases, about 1-2 kilobases, or greater than 1-2 kilobases.

In some embodiments, the 5′ splicing motif is GURAGU.

In some embodiments, the linker is about 14 bp to 100 bp.

In some embodiments, the hybridization sequence is about 50 bp to 400 bp.

Further aspects of the present disclosure relate to cells comprising the trans-splicing template polynucleotides of the present disclosure.

In some embodiments, the cell is a prokaryotic cell or eukaryotic cell. In some embodiments, the eukaryotic cell is a mammalian cell or plant cell.

Further aspects of the present disclosure relate to methods of editing a target RNA sequence in a cell, the method comprising administering to the cell an effective amount of the trans-splicing template polynucleotide of the present disclosure.

In some embodiments, the methods of the present disclosure comprise delivering to the cell by a viral vector, optionally wherein the viral vector is Adeno-associated viral (AAV) vector, a virus, optionally wherein the virus is an Adenovirus, a lentivirus, a herpes simplex virus; and/or a lipid nanoparticle.

### F

Further aspects of the present disclosure relate to methods of editing a target RNA sequence via 3′ trans-splicing in a cell, the method comprising delivering to the cell a non-naturally occurring, engineered trans-splicing template polynucleotide comprising: (a) a nucleic acid sequence encoding a ribozyme; (b) a hybridization sequence; (c) a 3′ splicing motif sequence; (d) optionally, a linker sequence; and (e) an insertion sequence.

Further aspects of the present disclosure relate to methods of editing a target RNA sequence in a cell, the method comprising delivering to the cell: (i) a trans-splicing template polynucleotide comprising a nucleic acid sequence encoding a ribozyme, wherein the trans-splicing template polynucleotide hybridizes to at least a portion of the target RNA sequence; (ii) a polynucleotide encoding a Cas7-11 enzyme; and (iii) a polynucleotide encoding a Cas7-11 guide RNA sequence; causing cleavage and insertion steps to achieve editing of the target RNA sequence via simultaneous 5′ and 3′ trans-splicing.

Each of the limitations of the invention can encompass various embodiments of the invention. It is, therefore, anticipated that each of the limitations of the invention involving any one element or combinations of elements can be included in each aspect of the invention. This invention is not limited in its application to the details of construction and the arrangement of components set forth in the following description or illustrated in the drawings. The invention is capable of other embodiments and of being practiced or of being carried out in various ways. Also, the phraseology and terminology used herein is for the purpose of description and should not be regarded as limiting. The use of “including,” “comprising.” or “having.” “containing”, “involving”, and variations thereof herein, is meant to encompass the items listed thereafter and equivalents thereof as well as additional items.

### BRIEF DESCRIPTION OF DRAWINGS

The following drawings form part of the present specification and are included to further demonstrate certain aspects of the present disclosure, which can be better understood by reference to one or more of these drawings in combination with the detailed description of specific embodiments presented herein. The figures are illustrative only and are not required for enablement of the invention disclosed herein. In the drawings:

FIG. 1 shows a schematic for 5′ editing using an exemplary ribozyme (Twister) on an exemplary target and two possible outcomes for the mature mRNA (5′ spliced product or a cis spliced product).

FIGS. 2A-2C show the development of ribozyme-facilitated editing for efficient 5′ trans-splicing. HH=hammerhead ribozyme, HDV=Hepatitis delta virus ribozyme, Twister=Twister ribozyme. FIG. 2A shows a schematic of protein-free ribozyme-facilitated editing to liberate trans-templates of the poly(A) tail and enable trans-splicing based RNA writing. FIG. 2B shows the RNA editing rate of 5′ trans-splicing using ribozyme-facilitated editing on the endogenous HTT transcript with trans-templates cut by either DisCas7-11 or one of several ribozymes. DisCas7-11 targeting is compared to a non-targeting guide (NT). FIG. 2C shows the RNA editing rate of 5′ trans-splicing ribozyme-facilitated editing on the endogenous RPL41 transcript with trans-templates cut by either DisCas7-11 or one of several ribozymes. DisCas7-11 targeting is compared to a non-targeting guide (NT).

FIG. 3 shows evaluation of 5′ trans-splicing using ribozyme-facilitated editing on endogenous HTT transcript in iPSC-derived human neurons by AAV delivery of protein-free cargo with HDV ribozyme. Three different doses (1×10⁸, 3.3×10⁷, and 1×10⁷ genome copies, respectfully) of AAV for the delivery of a targeting cargo or a control GFP vector are compared.

FIG. 4 shows a schematic for simultaneous 5′ and 3′ trans-splicing using an exemplary ribozyme (Twister) and DisCas7-11 on an exemplary target (luciferase reporter target). Two possible outcomes for the mature mRNA are shown: non-functional product spliced into mRNA (left) and internally spliced functional G-luciferase spliced into mRNA (right).

FIG. 5 shows a schematic depicting the difference between 5′ editing using Cas7-11 to target the intron or pre-mRNA and the 5′ splicing with ribozymes strategy.

FIG. 6 shows a graph of HTT transcript editing rates with 5′ trans-splicing constructs using one of several ribozymes immediately downstream of the hybridization region. 5′ trans-splicing constructs tested were “Twister” (SEQ ID NO: 9), “Hairpin 1” (SEQ ID NO: 19). “Hairpin 2” (SEQ ID NO: 20). “Hairpin 3” (SEQ ID NO: 21), “Varkud Satellite” (SEQ ID NO: 22), “glmS” (SEQ ID NO: 23), “twister sister” (SEQ ID NO: 24), “twister sister AT insert” (SEQ ID NO: 25). “pistol” (SEQ ID NO: 26), and “hatchet” (SEQ ID NO: 27).

FIG. 7 shows a graph of HTT transcript editing rates with 5′ trans-splicing constructs, exploring different hybridization region positions and combinations of ribozymes. Hybridization constructs tested were “Hyb 1” (SEQ ID NO: 74), Hyb 2” (SEQ ID NO: 75), Hyb 3” (SEQ ID NO: 76), Hyb 4” (SEQ ID NO: 77), and “Hyb 5” (SEQ ID NO: 78). Combinations of ribozymes tested were constructs “Twister_HDV” (SEQ ID NO: 72) and “HDV_Twister” (SEQ ID NO: 73). “Normal HTT cargo” (SEQ ID NO: 9) and “Normal HTT cargo with terminator” (SEQ ID NO: 16) were used as controls.

FIG. 8 shows a graph of MECP2 transcript editing rates with 5′ trans-splicing constructs, exploring different hybridization region positions, combinations of ribozymes, and triple helix, or “terminator” RNA stability motif added to 3′ to the ribozyme. Hybridization constructs tested were “Hyb 1” (SEQ ID NO: 79), Hyb 2” (SEQ ID NO: 80), Hyb 3” (SEQ ID NO: 81), Hyb 4” (SEQ ID NO: 82), and Hyb 5” (SEQ ID NO: 83). Combinations of ribozymes tested were “Twister_HDV” (SEQ ID NO: 84) and “HDV_Twister” (SEQ ID NO: 85). Triple helix motifs 3′ to the ribozyme constructs tested were “Comp14 hyb 6” (SEQ ID NO: 18). “MALAT1 hyb 6” (SEQ NO: 16), and MENB hybrid 6” (SEQ ID NO: 17).

FIG. 9 shows a graph of HTT editing rates for 5′ trans-splicing constructs that have a triple helix, or “terminator” RNA stability motif added to one of three locations on the construct relative to the ribozyme: (1) 5′ to the ribozyme; (2) in place of the ribozyme; or (3) 3′ to the ribozyme. Constructs with a motif added 5′ to the ribozyme were “font_MALAT1” (SEQ ID NO: 13), “front_MENB” (SEQ ID NO: 14), and “front_Comp14” (SEQ ID NO: 15). Constructs with a motif in place of the ribozyme were “instead_MALAT1” (SEQ ID NO: 10). “instead_MENB” (SEQ ID NO: 11), and “instead_Comp14” (SEQ ID NO: 12). Constructs with a motif 3′ to the ribozyme were “back_MALAT1” (SEQ ID NO: 16), “back_MENB” (SEQ ID NO: 17), and “back_Comp14” (SEQ ID NO: 18). Construct “Normal Twister cargo” (SEQ ID NO: 9) was used as a control.

FIG. 10 shows a graph of HTT editing rates for various 5′ trans-splicing constructs that have one of several different “ENE” elements. Constructs shown: TWIFB1 (SEQ ID NO: 31), EHV2 (SEQ ID NO: 33), RRV (SEQ ID NO: 32), TCUP (SEQ ID NO: 34) and Terminator+Twister (SEQ ID NO: 16). Constructs JSG1 (SEQ ID NO: 28), JSG2 (SEQ ID NO: 29), JSG3 (SEQ ID NO: 30) are synthetic sequences that were designed by recoding the sequences encoding the triple helix structures. “Twister cargo” construct refers to SEQ ID NO: 9.

FIG. 11 shows a graph of HTT editing rates for various 5′ trans-splicing constructs that have one of several different “ENE” elements. Constructs “HTT_hyb_1” (SEQ ID NO: 74). “HTT_hyb_2” (SEQ ID NO: 75), “HTT_hyb_3” (SEQ ID NO: 76), “HTT_hyb_4” (SEQ ID NO: 77), and “HTT_hyb_5” (SEQ ID NO: 78) are synthetic constructs that were designed by varying the hybridization region. Constructs “Twister_HDV” (SEQ ID NO: 72) and “HDV_Twister” (SEQ ID NO: 73) tested two different ribozymes in tandem. “Twister cargo” construct refers to SEQ ID NO: 9.

FIG. 12 shows a graph of RNA editing rates for a first round of 5′ trans-splicing constructs targeting replacement of 5′ exons of HOXD13. Constructs tested “Cargo 1” (SEQ ID NO: 35), “Cargo 2” (SEQ ID NO: 36), Cargo 3” (SEQ ID NO: 37), “Cargo 4” (SEQ ID NO: 38), and “Cargo NT (non-targeting)” (SEQ ID NO: 39).

FIG. 13 shows a graph of RNA editing rates for 5′ trans-splicing constructs targeting replacement of 5′ exons of target gene HOXA13. Constructs tested “Cargo 1” (SEQ ID NO: 117), “Cargo 2” (SEQ ID NO: 118), Cargo 3” (SEQ ID NO: 119). “Cargo 4” (SEQ ID NO: 120), and “Cargo 5” (SEQ ID NO: 121).

FIG. 14 shows a graph of RNA editing rates for 5′ trans-splicing constructs targeting replacement of 5′ exons of target gene SOD1. Constructs tested “Cargo 1” (SEQ ID NO: 42). “Cargo 2” (SEQ ID NO: 43), Cargo 3” (SEQ ID NO: 44), “Cargo 4” (SEQ ID NO: 45), and “Cargo 5” (SEQ ID NO: 46).

FIG. 15 shows a graph of RNA editing rates for 5′ trans-splicing constructs targeting replacement of 5′ exons of target gene KCNQ1. Constructs tested “Cargo 1” (SEQ ID NO: 47), “Cargo 2” (SEQ ID NO: 48), Cargo 3” (SEQ ID NO: 49), “Cargo 4” (SEQ ID NO: 50), and “Cargo 5” (SEQ ID NO: 51).

FIG. 16 shows a graph of RNA editing rates for 5′ trans-splicing constructs targeting replacement of 5′ exons of target gene MEF2C. Constructs tested “Cargo 1” (SEQ ID NO: 57), “Cargo 2” (SEQ ID NO: 58), Cargo 3” (SEQ ID NO: 59), “Cargo 4” (SEQ ID NO: 60), and “Cargo 5” (SEQ ID NO: 61).

FIG. 17 shows a graph of RNA editing rates for 5′ trans-splicing constructs targeting replacement of 5′ exons of target gene SPTBN2. Constructs tested “Cargo 1” (SEQ ID NO: 52). “Cargo 2” (SEQ ID NO: 53). Cargo 3” (SEQ ID NO: 54), “Cargo 4” (SEQ ID NO: 55), and “Cargo 5” (SEQ ID NO: 56).

FIG. 18 shows a graph of RNA editing rates for 5′ trans-splicing constructs targeting replacement of 5′ exons of target gene ATP7B. Constructs tested “Cargo 1” (SEQ ID NO: 62), “Cargo 2” (SEQ ID NO: 63), Cargo 3” (SEQ ID NO: 64), “Cargo 4” (SEQ ID NO: 65), and “Cargo 5 (non-targeting)” (SEQ ID NO: 66).

FIG. 19 shows a graph of RNA editing rates for 5′ trans-splicing constructs targeting replacement of 5′ exons of target gene CBS. Constructs tested “Cargo 1” (SEQ ID NO: 67), “Cargo 2” (SEQ ID NO: 68). Cargo 3” (SEQ ID NO: 69), “Cargo 4” (SEQ TD NO: 70), and “Cargo 5 (non-targeting)” (SEQ ID NO: 71).

FIG. 20 shows a graph of RNA editing rates for 5′ trans-splicing constructs targeting replacement of 5′ exons of MECP2, which is related to Rett's Syndrome. Constructs tested “1” (SEQ ID NO: 89), “2” (SEQ ID NO: 90), “3” (SEQ ID NO: 91), “4” (SEQ ID NO: 92), “5” (SEQ ID NO: 93) and “6” (SEQ ID NO: 94). Constructs were tested at four different concentrations: 20 ng, 40 ng, 80 ng, and 160 ng.

FIG. 21 shows a graph of RNA editing rates for 5′ trans-splicing constructs inserting cargos ranging from 114-1167 bp for HOXD13. Constructs tested “Normal—144 bp” (SEQ ID NO: 35), “EGFP-858 bp” (SEQ ID NO: 40), “Cre—1167 bp” (SEQ ID NO: 41).

FIG. 22 shows a graph of RNA editing rates for 5′ trans-splicing constructs for AAV editing. Constructs tested were “regular cargo” (SEQ ID NO: 95), “regular cargo with HDV” (SEQ ID NO: 122), “regular cargo with twister” (SEQ ID NO: 9). “AAV HDV backbone” (SEQ ID NO: 87), “AAV Twister backbone” (SEQ ID NO: 86), and “AAV twister NT (non-targeting, un-transduced) backbone” (SEQ ID NO: 88).

FIG. 23 shows a graph of RNA editing rates for 5′ trans-splicing construct (SEQ ID NO: 9) for HTT via transfection into four different cell lines: Huh7, HeLa, A549, and HEK293.

FIG. 24 shows a graph of RNA editing rate for 5′ splicing for an editing AAV construct (PRECISE-R AAV2) of the HTT transcript in vitro in human iPSC derived neurons that were sorted for transduction. T=AAV targeting construct (SEQ ID NO: 9) and NT=non-targeting (un-transduced).

FIG. 25 shows a graph of RNA editing rates for 5′ trans-splicing construct (SEQ ID NO: 9) for HTT via transduction using AAV constructs packaged in AAV2/1 or AAV2/9 capsids.

FIG. 26 shows a graph of RNA editing rates for 5′ trans-splicing constructs for HIT via transduction using AAV constructs packaged in AAV2/1 or AAV2/9 capsids. Two batches of the same construct (SEQ ID NO: 9) tested are shown as “Twister 1” and “Twister 2”.

FIG. 27 shows a graph of initial messenger max editing rates of RNA versions of the 5′ trans-splicing construct (SEQ ID NO: 9) for HIT at different amounts and ratios.

FIG. 28 shows a schematic of survival motor neuron (SMN) protein dependent snRNP translocation to the nucleus.

FIG. 29 shows a graph of RNA editing rates of normal HIT constructs (“standard HIT cargo”, SEQ ID NO: 9) against versions generated with one of several SMN sequence motifs either 5′ to the ribozyme: “SMN1 before ribozyme” (SEQ ID NO: 99), “SMN2 before ribozyme” (SEQ ID NO: 100). “SMN3 before ribozyme” (SEQ ID NO: 101) or 3′ to the ribozyme: “SMN1 after ribozyme” (SEQ ID NO: 102), “SMN2 after ribozyme” (SEQ ID NO: 103), “SMN3 after ribozyme” (SEQ ID NO: 104). Editing rates are shown for constructs transfected at 40 ng and 80 ng.

FIG. 30 shows a graph of RNA editing rates of RNA versions of SMN containing 5′ trans-splicing constructs “SMN1” (SEQ ID NO: 99), “SMN2” (SEQ ID NO: 10)), “SMN3” (SEQ ID NO: 101) or 3′ to the ribozyme: “SMN4” (SEQ ID NO: 102), “SMN5” (SEQ ID NO: 103), “SMN6” (SEQ ID NO: 104) relative to the normal HIT twister construct (SEQ ID NO: 9) 24 h post transfection. Conditions for each construct were either tailed (addition of polyA) or untailed (no addition of polyA).

FIG. 31 shows a graph of RNA editing rates of an SMN motif construct (SEQ ID NO: 103) 24 h post transduction with the lipid nanoparticles. Various amounts RNA construct (100 ng, 150 ng, 200 ng, and 250 ng) were formulated in lipid nanoparticles and transduced on HEK293FT cells.

FIG. 32 shows a graph of RNA editing rates for 5′ trans-splicing constructs inserting cargos ranging from 62 bp-10 kb for HIT. Constructs tested “62 bp” (SEQ ID NO: 16), “2 kb” (SEQ ID NO: 123), “5 kb” (SEQ ID NO: 124), 4 bp” (SEQ ID NO: 125), “6 kb” (SEQ ID NO: 126), “8 kb” (SEQ ID NO: 127), and 10 kb (SEQ ID NO: 128).

### DETAILED DESCRIPTION

As described herein, it was surprisingly discovered that payload engineering could be combined with ribozymes to accomplish protein-free, high-efficiency RNA trans-splicing. The Examples unexpectedly show that the trans-splicing template polynucleotides of the present disclosure harnessed the catalytic properties of ribozymes and demonstrated efficiency in editing, e.g., editing of HTT exon 1 via AAV delivery. Surprisingly, for 5′ RNA trans-splicing, cleavage of the poly(A) tail increased RNA splicing efficiency when using the trans-splicing template polynucleotides of the present invention. Without wishing to be bound by any theory, it is contemplated that simply liberating the poly(A) tail from the trans-template via ribozymes allowed for efficient trans-splicing, presumably through nuclear retention of the trans-template and efficiency of the trans-template alone to serve as the 5′ donor without pre-mRNA cleavage. The inventors discovered that when using ribozymes, only the RNA trans-splicing template is necessary, with no exogenous proteins required. The Examples further unexpectedly show that the RNA trans-splicing template polynucleotides of the present disclosure can be modified in the hybridization region to increase editing efficiency, are capable of inserting cargos of various sizes, and are compatible with use in a variety of cell lines. Furthermore, the trans-splicing template polynucleotides of the present disclosure are shown to exhibit editing efficiency when packaged in AAV capsids and separately in lipid nanoparticles. The trans-splicing template polynucleotides of the present disclosure offers un unexpected and simplified approach for programmable RNA editing.

Unless defined otherwise, technical and scientific terms used herein have the same meaning as commonly understood by one of ordinary skill in the art to which this disclosure pertains.

As used herein, the singular forms “a”, “an,” and “the” include both singular and plural referents unless the context clearly dictates otherwise. Thus, for example, reference to “a cell” includes a plurality of such cells.

As used herein, the term “optional” or “optionally” means that the subsequent described event, circumstance or substituent may or may not occur, and that the description includes instances where the event or circumstance occurs and instances where it does not.

The recitation of numerical ranges by endpoints includes all numbers and fractions subsumed within the respective ranges, as well as the recited endpoints.

As used herein, the term “about” or “approximately” refers to a measurable value such as a parameter, an amount, a temporal duration, and the like, are meant to encompass variations of and from the specified value, such as variations of +/−10% or less, +/−5% or less, +/−1% or less, +/−0.5% or less, and +/−0.1% or less of and from the specified value, insofar such variations are appropriate to perform in the disclosure. It is to be understood that the value to which the modifier “about” or “approximately” refers is itself also specifically, and preferably, disclosed.

As used herein, the term “insertion sequence” may be used interchangeably with “cargo sequence”, “cargo”, “payload sequence”.

The determination of “percent identity” between two sequences (e.g., polypeptide or polynucleotides) can be accomplished using a mathematical algorithm. A specific, non-limiting example of a mathematical algorithm utilized for the comparison of two sequences is the algorithm of Karlin S & Altschul S F (1990) PNAS 87: 2264-2268, modified as in Karlin S & Altschul S F (1993) PNAS 90: 5873-5877, each of which is herein incorporated by reference in its entirety. Such an algorithm is incorporated into the NBLAST and XBLAST programs of Altschul S F et al., (1990) J Mol Biol 215: 403, which is herein incorporated by reference in its entirety. BLAST nucleotide searches can be performed with the NBLAST nucleotide program parameters set, e.g., for score=100, wordiength=12 to obtain nucleotide sequences homologous to a nucleic acid molecule described herein. BLAST protein searches can be performed with the XBLAST program parameters set. e.g., to score 50, wordlength=3 to obtain amino acid sequences homologous to a protein molecule described herein. To obtain gapped alignments for comparison purposes. Gapped BLAST can be utilized as described in Altschul S F et al., (1997) Nuc Acids Res 25: 3389-3402, which is herein incorporated by reference in its entirety. Alternatively, PSI BLAST can be used to perform an iterated search which detects distant relationships between molecules (Id.). When utilizing BLAST. Gapped BLAST, and PSI Blast programs, the default parameters of the respective programs (e.g., of XBLAST and NBLAST) can be used (See. e.g., National Center for Biotechnology Information (NCBI) on the worldwide web, ncbi.nlm.nih.gov). Another specific, non-limiting example of a mathematical algorithm utilized for the comparison of sequences is the algorithm of Myers and Miller, 1988. CABIOS 4:11-17, which is herein incorporated by reference in its entirety. Such an algorithm is incorporated in the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used. The percent identity between two sequences can be determined using techniques similar to those described above, with or without allowing gaps. In calculating percent identity, typically only exact matches are counted.

As used herein the term “pharmaceutical composition” means a composition that is suitable for administration to an animal, e.g., a human subject, and comprises a therapeutic agent and a pharmaceutically acceptable carrier or diluent. A “pharmaceutically acceptable carrier or diluent” means a substance for use in contact with the tissues of human beings and/or non-human animals without excessive toxicity, irritation, allergic response, or other problem or complication, commensurate with a reasonable therapeutic benefit/risk ratio.

The terms “polynucleotide,” “nucleic acid,” and “nucleic acid molecule” are used interchangeably herein and refer to a polymer of DNA or RNA. The nucleic acid molecule can be single-stranded or double-stranded; contain natural, non-natural, or altered nucleotides; and contain a natural, non-natural, or altered internucleotide linkage, such as a phosphoroamidate linkage or a phosphorothioate linkage, instead of the phosphodiester found between the nucleotides of an unmodified nucleic acid molecule. Nucleic acid molecules include, but are not limited to, all nucleic acid molecules which are obtained by any means available in the art, including, without limitation, recombinant means, e.g., the cloning of nucleic acid molecules from a recombinant library or a cell genome, using ordinary cloning technology and polymerase chain reaction, and the like, and by synthetic means. The skilled artisan will appreciate that, except where otherwise noted, nucleic acid sequences set forth in the instant application will recite thymidine (T) in a representative DNA sequence but where the sequence represents RNA (e.g., mRNA), the thymidines (Ts) would be substituted for uracils (Us). Thus, any of the RNA polynucleotides encoded by a DNA identified by a particular sequence identification number may also comprise the corresponding RNA (e.g., mRNA) sequence encoded by the DNA, where each thymidine (T) of the DNA sequence is substituted with uracil (U).

The terms “protein” and “polypeptide” are used interchangeably herein and refer to a polymer of at least two amino acids linked by a peptide bond.

As used herein, the term “RNA” or “RNA polynucleotide” refers to macromolecules that include multiple ribonucleotides that are polymerized via phosphodiester bonds. Ribonucleotides are nucleotides in which the sugar is ribose. RNA may contain modified nucleotides; and contain natural, non-natural, or altered internucleotide linkages, such as a phosphoroamidate linkage or a phosphorothioate linkage, instead of the phosphodiester found between the nucleotides of an unmodified nucleic acid molecule.

A “therapeutically effective amount” of a therapeutic agent (e.g., a composition or system described herein) refers to any amount of the therapeutic agent that, when used alone or in combination with another therapeutic agent, protects a subject against the onset of a disease or promotes disease regression evidenced by a decrease in severity of disease symptoms, an increase in frequency and duration of disease symptom-free periods, or a prevention of impairment or disability due to the disease affliction. The ability of a therapeutic agent to promote disease regression can be evaluated using a variety of methods known to the skilled practitioner, such as in human subjects during clinical trials, in animal model systems predictive of efficacy in humans, or by assaying the activity of the agent in in vitro assays.

As used herein, the terms “treat,” treating,” “treatment,” and the like refer to reducing or ameliorating a disease and/or symptom(s) associated therewith or obtaining a desired pharmacologic and/or physiologic effect. It will be appreciated that, although not precluded, treating a disease does not require that the disease, or symptom(s) associated therewith be completely eliminated. In some embodiments, the effect is therapeutic, i.e., without limitation, the effect partially or completely reduces, diminishes, abrogates, abates, alleviates, decreases the intensity of, or cures a disease and/or adverse symptom attributable to the disease. In some embodiments, the effect is preventative, i.e., the effect protects or prevents an occurrence or reoccurrence of a disease. To this end, the presently disclosed methods comprise administering a therapeutically effective amount of a compositions as described herein.

As used here, the term “trans-splicing template polynucleotide” may be used interchangeable with “trans-splicing template” or “trans-template”.

In some aspects, the present disclosure relates to non-naturally occurring, engineered compositions comprising a trans-splicing template polynucleotide comprising a ribozyme. In some embodiments the trans-splicing template polynucleotide comprises one or more or all of an insertion sequence; a 5′ splicing motif sequence; optionally, a linker sequence; a hybridization sequence; and a nucleic acid sequence encoding a ribozyme. In some aspects, the present disclosure relates to methods of editing target RNA using the trans-splicing template polynucleotides provided herein.

### Ribozymes

A ribozyme is a type of RNA molecule that possesses catalytic activity (e.g., self-splicing or self-cleaving RNAs). The catalytic reaction can be a self-splicing transesterification (to produce 3′-OH), hydrolysis (to produce 3′-OH), self-cleaving transesterification (to produce 2′,3′-cyclic phosphate), a peptidyl transfer (to produce a peptide bond), or a trans-splicing transesterification (to produce a 3′-OH). These reactions often rely on interactions between the phosphate backbone and the base of the nucleotide and cause drastic conformational changes. Metal ions, such as Mg⁺ or Mn²⁺, can facilitate the catalytic reactions but are not always necessary. Ribozymes can be engineered to cut an external substrate in ‘trans’, instead of their natural self-slicing reaction, while maintaining high precision, cleaving only a single bond. This can be done by changing the targeting sequence of a ribozyme to cleave and inactivate specific RNA sequences by relying on Watson-Crick base-pairing between sequences flanking the catalytic domain and sequences surrounding the cleavage site. In some embodiments, the ribozyme is a self-cleaving RNA. In some embodiments, the ribozyme cuts a target RNA in ‘trans’.

Ribozymes are typically 3 to 300 nucleotides in length. In some embodiments, the ribozyme is 3 to 10 nucleotides in length. In some embodiments, the ribozyme is 3 to 20 nucleotides in length. In some embodiments, the ribozyme is 3 to 30 nucleotides in length. In some embodiments, the ribozyme is 3 to 40 nucleotides in length. In some embodiments, the ribozyme is 3 to 50 nucleotides in length. In some embodiments, the ribozyme is 3 to 60 nucleotides in length. In some embodiments, the ribozyme is 3 to 70 nucleotides in length. In some embodiments, the ribozyme is 3 to 80 nucleotides in length. In some embodiments, the ribozyme is 3 to 90 nucleotides in length. In some embodiments, the ribozyme is 3 to 100 nucleotides in length. In some embodiments, the ribozyme is 3 to 110 nucleotides in length. In some embodiments, the ribozyme is 3 to 120 nucleotides in length. In some embodiments, the ribozyme is 3 to 130 nucleotides in length. In some embodiments, the ribozyme is 3 to 140 nucleotides in length. In some embodiments, the ribozyme is 3 to 150 nucleotides in length. In some embodiments, the ribozyme is 3 to 160 nucleotides in length. In some embodiments, the ribozyme is 3 to 170 nucleotides in length. In some embodiments, the ribozyme is 3 to 180 nucleotides in length. In some embodiments, the ribozyme is 3 to 190 nucleotides in length. In some embodiments, the ribozyme is 3 to 200 nucleotides in length. In some embodiments, the ribozyme is 3 to 210 nucleotides in length. In some embodiments, the ribozyme is 3 to 220 nucleotides in length. In some embodiments, the ribozyme is 3 to 230 nucleotides in length. In some embodiments, the ribozyme is 3 to 240 nucleotides in length. In some embodiments, the ribozyme is 3 to 250 nucleotides in length. In some embodiments, the ribozyme is 3 to 260 nucleotides in length. In some embodiments, the ribozyme is 3 to 270 nucleotides in length. In some embodiments, the ribozyme is 3 to 270 nucleotides in length. In some embodiments, the ribozyme is 3 to 280 nucleotides in length. In some embodiments, the ribozyme is 3 to 290 nucleotides in length. In some embodiments, the ribozyme is 3 to 300 nucleotides in length.

Ribozymes have been found naturally occurring in genomes of species from all kingdoms of life (e.g., eukaryotes, prokaryotes, in bacteriophages, viruses, viroids, and satellite viruses). In some embodiments, the ribozyme is a naturally occurring ribozyme.

Artificial ribozymes, also referred to as synthetic ribozymes, can be produced. e.g. by in vitro selection of RNAs originating from random-sequence RNAs that have self-cleaving properties. The use of single-stranded DNA molecules having ribozyme-like activity that have the ability to target and cleave single-stranded DNA (e.g., Deoxyribozymes or DNAzymes) is also contemplated. Non-limiting examples of deoxyribozymes include ribonucleases, that can catalyze a transesterification reaction and form a 2′3′-cyclic phosphate terminus and a 5′-hydroxyl terminus) and DNA ligases. Other non-limiting classes of deoxyribozymes include those that catalyze DNA phosphorylation, DNA adenylation, DNA deglycoslyation, porphyrin metalation, thymine dimer photoreversion, and DNA cleavage. In some embodiments, the ribozyme is an artificial ribozyme (e.g., a synthetic ribozyme).

Non-limiting examples of ribozymes include Group I introns, Group II introns, RNase P RNA, ribosomal RNAs, spliceosomal RNAs, GIRI branching ribozyme, gimSribozyme, Twister sister ribozyme, VS ribozyme, Pistol ribozyme. Hatchet ribozyme, Hammerhead ribozyme, hairpin ribozyme. Hepatitis delta virus (HDV) ribozyme, leadzyme, CPEB3 ribozyme, and Twister ribozyme. Other ribozymes known in the art are also contemplated for use herein.

In some embodiments, the ribozyme is Twister ribozyme. A non-limiting exemplary sequence for the Twister ribozyme is: CCGCCTAACACTGCCAATGCCGGTCCCAAGCCCGGATAAAAAGTGGAGGGGGCGG (SEQ ID NO: 105). In some embodiments, the ribozyme comprises a sequence that is 90% identical to SEQ ID NO: 105. In some embodiments, the ribozyme comprises SEQ ID NO: 105.

In some embodiments, the ribozyme is Hammerhead ribozyme. A non-limiting exemplary sequence for the Hammerhead ribozyme is: ctgatgagtcegtgaggacgaaacgagtaagctcgtc nnnnnn (SEQ ID NO: 106). In some embodiments, nnnnnn in SEQ ID NO: 106 is reverse complementary to the 6 bp diectly upstream (5′) of the ribozyme in a trans-splicing template polynucleotide. In some embodiments, the ribozyme comprises a sequence that is 90% identical to SEQ ID NO: 106. In some embodiments, the ribozyme comprises SEQ ID NO: 106.

In some embodiments, the ribozyme is Hepatitis delta virus (HDV) ribozyme. A non-limiting exemplary sequence for the HDV ribozyme is: ggccggcatggtcccagcctcctcgctggcgccggctgggcaacatgctteggcatggcgaatgggacGCGGCCGC (SEQ ID NO: 107).

In some embodiments, the ribozyme comprises a sequence that is 90% identical to SEQ ID NO: 107. In some embodiments, the ribozyme comprises SEQ ID NO: 107.

In some embodiments, the ribozyme is Hairpin 1 ribozyme. A non-limiting exemplary sequence for the Hairpin 1 ribozyme is: AAACAGAGAAGTCAACCAGAGAAACACACGTTGTGGTATATTACCTGGTA (SEQ ID NO: 108). In some embodiments, the ribozyme comprises a sequence that is 90% identical to SEQ ID NO: 108. In some embodiments, the ribozyme comprises SEQ ID NO: 108.

In some embodiments, the ribozyme is Hairpin 2 ribozyme. A non-limiting exemplary sequence for the Hairpin 2 ribozyme is: CAACAGCGAAGCGCGCCAGGGAAACACACCATGTGTGGTATATTATCTGGCA (SEQ ID NO: 109). In some embodiments, the ribozyme comprises a sequence that is 90% identical to SEQ ID NO: 109. In some embodiments, the ribozyme comprises SEQ ID NO: 109.

In some embodiments, the ribozyme is Hairpin 3 ribozyme. A non-limiting exemplary sequence for the Hairpin 3 ribozyme is: CAACAGCGAAGCGGAACGGCGAAACACACCTTGTGTGGTATATTACCCGTTG (SEQ ID NO: 110). In some embodiments, the ribozyme comprises a sequence that is 90% identical to SEQ ID NO: 110. In some embodiments, the ribozyme comprises SEQ ID NO: 110.

In some embodiments, the ribozyme is Varkud Satellite ribozyme. A non-limiting exemplary sequence for the Varkud Satellite ribozyme is: GGGAAAGCTTGCGAAGGGCGTCGTCGCCCCGAGCGGTAGTAAGCAGGGAACTCACC TCCAATTTCAGTACTGAAATGTCGTAGCAGTTGACTACTGTTATGTGATTGGTAGAGG CTAAGTGACGGTATTGGCGTAAGTCAGTATTGCAGCACAGCACAAGCCCGCTTGCGA GAAT (SEQ ID NO: 111). In some embodiments, the ribozyme comprises a sequence that is 90% identical to SEQ ID NO: 111. In some embodiments, the ribozyme comprises SEQ ID NO: 111.

In some embodiments, the ribozyme is gimS ribozyme. A non-limiting exemplary sequence for the gimS ribozyme is: TAATTATAGCGCCCGAACTAAGCGCCCGGAAAAAGGCTTAGTTGACGAGGATGGAGG TTATCGAAT1TTCGGGCGGATGCCTCCCGGCTGAGTGTGCAGATCACAGCCGTAAGGA TTTCTCAAACCAAGGGGGTGACTCCTTGAACAAAGAGAAATCACATGATCT (SEQ ID NO: 112). In some embodiments, the ribozyme comprises a sequence that is 90% identical to SEQ ID NO: 112. In some embodiments, the ribozyme comprises SEQ ID NO: 112.

In some emlxxliments, the ribozyme is twister sister ribozyme. A non-limiting exemplary sequence for the twister sister ribozyme is: GGACCCGCAAGGCCGACGGCATCCGCCGCCGCTGGTGCAAGTCCAGCCGCCCCGGG GCGGGCGCTCATGGGTAAAC (SEQ ID NO: 113). In some embodiments, the ribozyme comprises a sequence that is 90% identical to SEQ ID NO: 113. In some embodiments, the ribozyme comprises SEQ ID NO: 113.

In some embodiments, the ribozyme is twister sister ribozyme with an AT insert. A non-limiting exemplary sequence for the twister sister ribozyme with an AT insert is: GGACCCGCAAGGCCGACGGCATCCGCCGCCGCTGGTGCAAGTCCAGCCGCCCCATG GGGCGGGCGCTCATGGGTAAAC (SEQ ID NO: 114). In some embodiments, the ribozyme comprises a sequence that is 90% identical to SEQ ID NO: 114. In some embodiments, the ribozyme comprises SEQ ID NO: 114.

In some embodiments, the ribozyme is pistol ribozyme. A non-limiting exemplary sequence for the pistol ribozyme is: GGAGCCGTTCGGGCCGCTATAAACAGACCTCAGGCCCGAAGCGTGGCGGCGATCCG CCGGTGGTA (SEQ ID NO: 115). In some embodiments, the ribozyme comprises a sequence that is 90% identical to SEQ ID NO: 115. In some embodiments, the ribozyme comprises SEQ ID NO: 115, in some embodiments, the ribozyme is hatchet ribozyme. A non-limiting exemplary sequence for the hatchet ribozyme is: CAITCCTCAGAAAATGACAAACCTGTGGGGCGTAAGTAGATATGTACATATCTATGATC GTGCAGACGTTAAAATCAGGT (SEQ ID NO: 116). In some embodiments, the ribozyme comprises a sequence that is 90% identical to SEQ ID NO: 116. In some embodiments, the ribozyme comprises SEQ ID NO: 116.

### Trans-Splicing and Trans-Splicing Template Polynucleotide

Generally, trans-splicing relies on the recruitment of an RNA template to a pre-mRNA without any active targeting domains and involves competition with the cis target. The trans-splicing template polynucleotides of the present disclosure can help boost efficiency of the trans-splicing mechanism, at least in part by cleaving the poly(A) tail of the trans-splicing template, enabling any potential type of RNA edit, insertion (e.g., correction of a mutation, a transgene), deletion, or replacement to be incorporated into endogenous transcripts. This combination can be used, for example and without limitation, to edit a polynucleotide in a cell, treat or prevent a genetically inherited diseases, and engineering cells (e.g., CAR-T cells) via editing of a transgene.

The trans-splicing template polynucleotide disclosed herein can comprise one or more insertion sequences; one or more 3′ and/or 5′ splicing site sequences, optionally one or more linker sequences, one or more hybridization sequences, and one or more nucleic acid sequences encoding a ribozyme.

An insertion sequence is also referred to herein as a “cargo sequence”, “cargo”, or “payload sequence”. An insertion sequence is any sequence to be used for purposes of trans-splicing RNA such as an RNA sequence having an edit (e.g., mutation), insertion or deletion, relative to a naturally occurring RNA sequence. In some embodiments, an insertion sequence has one or more edits (e.g., mutations) relative to a naturally occurring RNA sequence. In some embodiments, an insertion sequence has one or more nucleotides inserted within the sequence relative to a naturally occurring RNA sequence. In some embodiments, an insertion sequence has one or more nucleotides removed (e.g., deleted) from the sequence relative to a naturally occurring RNA sequence. In some embodiments, an insertion sequence may comprise a correction of a mutation present in a naturally occurring RNA sequence. In some embodiments, an insertion sequence may be a transgene. In some embodiments, an insertion sequence may edit, insert, or delete one or more polynucleotides within an endogenous RNA transcript. In some embodiments, an insertion sequence may replace a portion of an endogenous RNA transcript. In some embodiments, the insertion sequence is a cargo sequence that replaces a desired exon during the trans-splicing mechanism. It should be understood that the insertion sequence can be designed according to desired results of the RNA trans-splicing event.

A variety of lengths of insertion sequences are contemplated for use with the trans-splicing template polynucleotides of the present invention. In some embodiments, the insertion sequence is less than 1-2 kilobases (kb). In non-limiting examples, the insertion sequence is less than 1 kb (e.g., the insertion sequence is at least 1 base pair (bp), at least 20 bp, at least 30 bp, at least 40 bp, at least 50 bp, at least 60 bp, at least 70 bp, at least 80 bp, at least 90 bp, at least 100 bp, at least 200 bp, at least 3M) bp, at least 400 bp, at least 500 bp, at least 600 bp, at least 700 bp, at least 800 bp, at least 900 bp). In some embodiments, the insertion sequence is about 1-2 kb. In some embodiments, the insertion sequence is about 1 kb. In some embodiments, the insertion sequence is about 2 kb. In some embodiments, the insertion sequence is greater than 1-2 kb. In non-limiting examples, the insertion sequence is at least 3 kb, at least 4 kb, at least 5 kb, at least 6 kb, at least 7 kb, at least 8 kb, at least 9 kb, or at least 10 kb. In some embodiments, the insertion sequence is greater than 3 kb. In some embodiments, the insertion sequence is about 3.7 kb.

The trans-splicing template polynucleotides may comprise a splicing motif. A splicing motif is a conserved or partially conserved nucleotide sequence that forms a boundary between an intron and an exon. A splicing motif can be located at the 5′ end of an intron (e.g., a 5′ splicing motif) or at the 3′ end of an intron (e.g., a 3′ splicing motif). A splicing motif (e.g., 5′ or 3) can be variable in length. For non-limiting examples, the length of a splicing motif is 2 or more nucleotides (e.g., at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, or at least 20 nucleotides). In some embodiments, the trans-splicing template polynucleotide comprises a 5′ splicing motif. In some embodiments, the length of a 5′ splicing motif is at least 6 nucleotides. In some embodiments, the 5′ splicing motif is the mammalian consensus sequence GURAGU. Other 5′ splicing motifs known in the art are contemplated for use herein. In some embodiments, the trans-splicing template polynucleotide comprises a 3′ splicing motif. In some embodiments, the length of the 3′ splicing motif is at least 14 nucleotides. 3′ splicing motifs known in the art are contemplated for use herein.

The trans-splicing template polynucleotide may, in some embodiments, comprise a linker sequence. In some embodiments a linker sequence may be operably linked to a 5′ spicing motif. In some embodiments, the linker sequence may be operably linked to a 3′ spicing motif. In non-limiting examples, a linker sequence may be at least 5 bp, at least 6 bp, at least 7 bp, at least 8 bp, at least 9 bp, at least 10 bp, at least 11 bp, at least 12 bp, at least 13 bp, at least 14 bp, at least 15 bp, at least 17 bp, at least 18 bp, at least 19 bp, or at least 20 bp. In some embodiments, a linker sequence may range from 14 bp to 100 bp. In some embodiments, the linker sequence is 14 bp to 20 bp. In some embodiments, the linker sequence is 14 bp to 30 bp. In some embodiments, the linker sequence is 14 bp to 40 bp. In some embodiments, the linker sequence is 14 bp to 50 bp. In some embodiments, the linker sequence is 14 bp to 60 bp. In some embodiments, the linker sequence is 14 bp to 70 bp. In some embodiments, the linker sequence is 14 bp to 80 bp. In some embodiments, the linker sequence is 14 bp to 90 bp. In some embodiments, the linker sequence is 14 bp to 100 bp.

A hybridization sequence is complementary to (e.g., hybridizes) to at least a portion of a target RNA sequence. For example, a hybridization sequence may bind to an intron or exon of the target RNA sequence (e.g., a premRNA sequence). As non-limiting examples, a hybridization sequence may be at least 10 bp, at least 20 bp, at least 30 bp, at least 40 bp, at least 50 bp, at least 60 bp, at least 70 bp, at least 80 bp, at least 90 bp, or at least 100 bp. In some embodiments, the hybridization sequence is at least 40 bp. In some embodiments, the hybridization sequence may range from 40 bp to 400 bp. In some embodiments, the hybridization sequence may range from 40 bp to 350 bp. In some embodiments, the hybridization sequence may range from 40 bp to 300 bp. In some embodiments, the hybridization sequence may range from 40 bp to 250 bp. In some embodiments, the hybridization sequence may range from 40 bp to 200 bp. In some embodiments, the hybridization sequence may range from 40 bp to 150 bp. In some embodiments, the hybridization sequence may range from 40 bp to 100 bp. In some embodiments, the hybridization sequence may range from 40 bp to 50 bp. In some embodiments, the hybridization sequence may range from 10 bp to 50 bp. In some embodiments, the hybridization sequence is 150 bp.

Trans-splicing template polynucleotides of the present invention can be delivered to a cell in a variety of mechanisms. Non-limiting examples of mechanisms for which a trans-splicing template polynucleotide can be delivered to a cell include by a viral vector, optionally wherein the viral vector is Adeno-associated viral (AAV) vector, by a virus, optionally wherein the virus is an Adenovirus, a lentivirus, a herpes simplex virus; and/or by a lipid nanoparticle. Other delivery mechanisms known in the art are also contemplated for use herein.

In some embodiments, the trans-splicing template polynucleotide is delivered in a single AAV. In some embodiments, the single AAV is a tissue specific AAV. Tissue specific AAV capsids known in the art are contemplated for use herein.

The trans-splicing template polynucleotides of the present invention may be used for RNA editing (e.g., pre-mRNA trans-splicing). In some embodiments, a trans-splicing template polynucleotide of the present disclosure may be used for 5′ trans-splicing. In some embodiments, a trans-splicing template polynucleotide of the present disclosure may be used for 3′ trans-splicing. In some embodiments, more than one trans-splicing template polynucleotides of the present disclosure may be used for simultaneous 5′ and 3′ splicing. In some embodiments, a trans-splicing template polynucleotide of the present disclosure may be used in combination with a Cas7-11/Cas7-11gRNA system for simultaneous 5′ and 3′ splicing. Compositions and methods relating to Cas7-1l/Cas7-11gRNAs suitable for use with the trans-splicing template polynucleotides of the present disclosure are described in U.S. application Ser. No. 18/455,380, U.S. application Ser. No. 18/322,675, and U.S. application Ser. No. 17/365,777 (US Publication No. US-2022/0073891A1), all of which are incorporated by reference herein in their entirety. Other Cas nuclease/Cas gRNA systems known in the art are also contemplated for use in combination with the trans-splicing template polynucleotides of the present disclosure to achieve simultaneous 5′ and 3′ splicing.

### Pharmaceutical Compositions

Pharmaceutical compositions described herein comprise at least one component of an editing system described herein (e.g., a trans-splicing template polynucleotide) and a pharmaceutically acceptable excipient (see, e.g., Remington's Pharmaceutical Sciences (1990) Mack Publishing Co., Easton, PA, the entire contents of which is incorporated by reference herein for all purposes).

In one aspect, also provided herein are methods of making pharmaceutical compositions described herein comprising providing at least one component of an editing system described herein (e.g., a trans-splicing template polynucleotide) and formulating it into a pharmaceutically acceptable composition by the addition of one or more pharmaceutically acceptable excipient. In some embodiments, the pharmaceutical composition comprises a single component described herein (e.g., a trans-splicing template polynucleotide). In some embodiments, the pharmaceutical composition comprises a plurality of the components described herein (e.g., one or more trans-splicing template polynucleotides, a Cas7-11, a Cas7-11 gRNA, etc.).

Acceptable excipients (e.g., carriers and stabilizers) are preferably nontoxic to recipients at the dosages and concentrations employed, and include buffers such as phosphate, citrate, or other organic acids; antioxidants including ascorbic acid or methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; or m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, or other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN™, PLURONICS™ or polyethylene glycol (PEG).

A pharmaceutical composition may be formulated for any route of administration to a subject. The skilled person knows the various possibilities to administer a pharmaceutical composition described herein in order to deliver the editing system or composition to a target cell. Non-limiting embodiments include parenteral administration, such as intramuscular, intradermal, subcutaneous, transcutaneous, or mucosal administration. In one embodiment, the pharmaceutical composition is formulated for intravenous administration. In one embodiment, the pharmaceutical composition is formulated for administration by intramuscular, intradermal, or subcutaneous injection. Injectables can be prepared in conventional forms, either as liquid solutions or suspensions. The injectables can contain one or more excipients. Exemplary excipients include, for example, water, saline, dextrose, glycerol, or ethanol. In addition, if desired, the pharmaceutical compositions to be administered can also contain minor amounts of non-toxic auxiliary substances such as wetting or emulsifying agents, pH buffering agents, stabilizers, solubility enhancers, or other such agents, such as for example, sodium acetate, sorbitan monolaurate, triethanolamine oleate or cyclodextrins. In some embodiments, the pharmaceutical composition is formulated in a single dose. In some embodiments, the pharmaceutical compositions if formulated as a multi-dose.

Pharmaceutically acceptable excipients (e.g., carriers) used in the parenteral preparations described herein include for example, aqueous vehicles, nonaqueous vehicles, antimicrobial agents, isotonic agents, buffers, antioxidants, local anesthetics, suspending and dispersing agents, emulsifying agents, sequestering or chelating agents or other pharmaceutically acceptable substances. Examples of aqueous vehicles, which can be incorporated in one or more of the formulations described herein, include sodium chloride injection, Ringer's injection, isotonic dextrose injection, sterile water injection, dextrose or lactated Ringer's injection. Nonaqueous parenteral vehicles, which can be incorporated in one or more of the formulations described herein, include fixed oils of vegetable origin, cottonseed oil, corn oil, sesame oil or peanut oil. Antimicrobial agents in bacteriostatic or fungistatic concentrations can be added to the parenteral preparations described herein and packaged in multiple-dose containers, which include phenols or cresols, mercurials, benzyl alcohol, chlorobutanol, methyl and propyl p-hydroxybenzoic acid esters, thimerosal, benzalkonium chloride or benzethonium chloride. Isotonic agents, which can be incorporated in one or more of the formulations described herein, include sodium chloride or dextrose. Buffers, which can be incorporated in one or more of the formulations described herein, include phosphate or citrate. Antioxidants, which can be incorporated in one or more of the formulations described herein, include sodium bisulfate. Local anesthetics, which can be incorporated in one or more of the formulations described herein, include procaine hydrochloride. Suspending and dispersing agents, which can be incorporated in one or more of the formulations described herein, include sodium carboxymethylcelluose, hydroxypropyl methylcellulose or polyvinylpyrrolidone. Emulsifying agents, which can be incorporated in one or more of the formulations described herein, include Polysorbate 80 (TWEEN® 80). A sequestering or chelating agent of metal ions, which can be incorporated in one or more of the formulations described herein, is EDTA. Pharmaceutical carriers, which can be incorporated in one or more of the formulations described herein, also include ethyl alcohol, polyethylene glycol or propylene glycol for water miscible vehicles; or sodium hydroxide, hydrochloric acid, citric acid or lactic acid for pH adjustment.

The precise dose to be employed in a pharmaceutical composition will also depend on the route of administration, and the seriousness of the condition caused by it, and should be decided according to the judgment of the practitioner and each subject's circumstances. For example, effective doses may also vary depending upon means of administration, target site, physiological state of the subject (including age, body weight, and health), other medications administered, or whether therapy is prophylactic or therapeutic. Therapeutic dosages are preferably titrated to optimize safety and efficacy.

### Kits

Also provided herein are kits comprising at least one pharmaceutical composition described herein. In addition, the kit may comprise a liquid vehicle for solubilizing or diluting, and/or technical instructions. The technical instructions of the kit may contain information about administration and dosage and subject groups. In some embodiments, the kit contains a single container comprising a single pharmaceutical composition described herein. In some embodiments, the kit comprises at least two separate containers, each comprising a different pharmaceutical composition described herein (e.g., a first container comprising a pharmaceutical composition comprising one component of an editing system described herein, e.g., a trans-splicing template polynucleotide described herein, and a second container comprising a second pharmaceutical composition comprising a second component of an editing system described herein, e.g., a polynucleotide encoding a Cas7-11 and/or a Cas7-11 gRNA).

### Methods of Use

Provided herein are various methods of using the editing systems, compositions, pharmaceutical compositions described herein and any one or more of the components thereof (e.g., a trans-splicing template polynucleotide).

In one aspect, provided herein are methods of editing a target polynucleotide, the method comprising contacting the target polynucleotide with an editing system, composition, pharmaceutical composition, or any component thereof (e.g., a trans-splicing template polynucleotide). In some embodiments, the target polynucleotide is or is within a gene. In some embodiments, the target polynucleotide is or is within a genome.

In one aspect, provided herein are methods of editing a target polynucleotide within a cell, the method comprising introducing into the cell an editing system, composition, pharmaceutical composition, or any component thereof (e.g., a trans-splicing template polynucleotide). In some embodiments, the target polynucleotide is or is within a gene. In some embodiments, the target polynucleotide is or is within a genome.

In one aspect, provided herein are methods of editing a target polynucleotide within a cell in a subject, the method comprising administering to the subject an editing system, composition, pharmaceutical composition, or any component thereof (e.g., a trans-splicing template polynucleotide), in an amount sufficient to deliver the editing system, composition, pharmaceutical composition, or component to a cell in the subject. In some embodiments, the target polynucleotide is or is within a gene. In some embodiments, the target polynucleotide is or is within a genome.

In one aspect, provided herein are methods of delivering an editing system, composition, pharmaceutical composition, or any component thereof (e.g., a trans-splicing template polynucleotide) to a cell comprising contacting the cell with the editing system, composition, pharmaceutical composition, or component thereof, in an amount sufficient to deliver the editing system, composition, pharmaceutical composition, or any component thereof to the cell.

In one aspect, provided herein are methods of delivering an editing system, composition, pharmaceutical composition, or any component thereof (e.g., a trans-splicing template polynucleotide) to a subject, the method comprising administering the editing system, composition, pharmaceutical composition, or component thereof to the subject.

In one aspect, provided herein are methods of delivering an editing system, composition, pharmaceutical composition, or any component thereof (e.g., a trans-splicing template polynucleotide) to a cell in a subject, the method comprising administering the editing system, composition, pharmaceutical composition, or component thereof to the subject, in an amount sufficient to deliver the editing system, composition, pharmaceutical composition, or component to a cell in the subject.

In one aspect, provided herein are methods of treating a subject diagnosed with or suspected of having a disease associated with a genetic mutation comprising administering a composition or system described herein to the subject in an amount sufficient to correct the genetic mutation. Exemplary diseases associated with a genetic mutation, include, but are not limited to cystic fibrosis, muscular dystrophy, hemochromatosis, Tay-Sachs, Huntington disease, Congenital Deafness, Sickle cell anemia. Familial hypercholesterolemia, adenosine deaminase (ADA) deficiency, X-linked SCID (X-SCID), and Wiskott-Aldrich syndrome (WAS).

In some embodiments, the genetic mutation is in one of the following genes: HOXA13, HOXD13, SOD1, KCNQ1, SPTBN2, MEF2C, ATP7B, CBS, GBA, BTK, ADA, CNGB3, CNGA3, ATF6, GNAT2, ABCA1, ABCA7, APOE, CETP, LIPC, MMP9, PLTP, VTN, ABCA4, MFSD8, TLR3, TLR4, ERCC6, HMCNI, HTRA1, MCDR4, MCDR5, ARMS2, C2, C3, CFB, CFH, JAG1, NOTCH2, CACNA1F, SERPINA1, TTR, GSN, B2M, APOA2, APOA1, OSMR, ELP4, PAX6, ARG, ASL, PCfX2, FOXC1, BBS1, BBS10, BBS2, BBS9, MKKS, MKS1, BBS4, BBS7, TTC8, ARL6, BBS5, BBS12, TRIM32, CEP290, ADIPORI, BBIPL, CEP19, IFT27, LZTFL1, DMD, BESTI, HBB, CYP4V2, AMACR, CYP7B1, HSD3B7, AKRIDI, OPNISW, NR2F1, RLBP1, RGS9, RGS9BP, PROMI, PRPH2, GUCY2D, CACD, CHM, ALAD, ASS1, SLC25A13, OTC, ACADVL, ETFDH, TMEM67, CC2D2A, RPGRIP1L, KCNV2, CRX, GUCA1A, CERKL, CDHR1, PDE6C, TTLL5, RPGR, CEP78, C21orf2, C80RF37, RPGRIP1, ADAM9, POCIB, PITPNM3, RAB28, CACNA2D4, AIPL1, UNCI19, PDE6H, OPNILW, RIMS1, CNNM4, IFT81, RAX2, RDH5, SEMA4A, CORD17, PDE6B, GRKI, SAG, RHO, CABP4, GNB3, SLC24A1, GNAT1, GRM6, TRPMI, LRIT3, TGFBI, TACSTD2, KRT12, OVOL2, CPSI, UGT1A1, UGT1A9, UGTIA8, UGT1A7, UGT1A6, UGT1A5, UGT1A4, CFTR, DLD, EFEMP1, ABCC2, ZNF408, LRP5, FZD4, TSPAN12, EVR3, APOB, SLC2A2, LOC106627981, GBA1, NR2E3, OAT, SLC40A1, F8, F9, UROD, CPOX, HFE, JH, LDLR, EPHXI, TJP2, BAAT, NBAS, LARS1, HAMP, HJV, RSI, ADAMTS18, LRAT, RPE65, LCA5, MERTK, GDF6, RD3, CCT2, CLUAPI, DTHDI, NMNATI, SPATA7, IFil40, IMPDH1, OTX2, RDH12, TULP1, CRB, MT-ND4, MT-ND), MT-ND6, BCKDHA, BCKDHB, DBT, MMAB, ARSB, GUSB, NAGS, NPC1, NPC2, NDP, OPAl, OPA3, OPA4, OPA5, RTN4IP1, TMEM126A, OPA6, OPA8, ACO2, PAH, PRKCSH, SEC63, GAA, UROS, PPOX, HPX, HMOX1, HMBS, MIR223, CYPiBI, LTBP2, AGXT, ATP8B1, ABCB11, ABCB4, FECH, ALAS2, PRPF31, RP1, EYS, TOPORS, USH2A, CNGA1, C2ORF71, RP2, KLHL7, ORF1, RP6, RP24, RP34, ROMI, ADGRA3, AGBLS, AHR, ARHGEF18, CA4, CLCCI, DHDDS, EMCI, FAM161A, HGSNAT, HK1, IDH3B, KIAA1549, KIZ, MAK, NEURODI, NRL, PDE6A, PDE6G, PRCD, PRPF3, PRPF4, PRPF6, PRPF8, RBP3, REEP6, SAMD11, SLC7A14, SNRNP200, SPP2, ZNF513, NEK2, NEK4, NXNL1, OFDl, RP1L1, RP22, RP29, RP32, RP63, RP9, RGR, POMGNTIL DHX38, ARL3, COL2A1, SLCOIBI, SLCOIB3, KCNJ13, TIMP3, ELOVL4, TFR2, FAH, HPD, MYO7A, CDH23, PCDH15, DFNB31, GPR98, USH1C, USH1G, CIB2, CLRN1. HARS, ABHD12. ADGRVt, ARSG, CEP250, IMPG1, IMPG2, VCAN, G6PC1, ATP7B, HIT, STAT3, PABPC1, PPIB, TOP2A, SHANK3, USFK, gLuc, and RPL41.

In some embodiments, the genetically inherited disease is selected from the group consisting of Meier-Gorlin syndrome; Seckel syndrome 4; Joubert syndrome 5; Leber congenital amaurosis 10; Charcot-Marie-Tooth disease, type 2; leukoencephalopathy; Usher syndrome, type 2C; spinocerebellar ataxia 28; glycogen storage disease type III; primary hyperoxaluria, type 1; long QT syndrome 2; Sjǒgren-Larsson syndrome; hereditary fructosuria; neuroblastoma; amyotrophic lateral sclerosis type 9; Kallmann syndrome 1; limb-girdle muscular dystrophy, type 2L; familial adenomatous polyposis 1; familial type 3 hyperlipoproteinemia; Alzheimer's disease, type 1; metachromatic leukodystrophy; cancer; Uveitis; SCA1; SCA2; FUS-Amyotrophic Lateral Sclerosis (ALS); MAPT-Frontotemporal Dementia (FTD); Myotonic Dystrophy Type 1 (DM1); Diabetic Retinopathy (DR/DME); Oculopharyngeal Muscular Dystrophy (OPMD); SCA8; C9ORF72-Amyotrophic Lateral Sclerosis (ALS); SOD1-Amyotrophic Lateral Sclerosis (ALS); Spinal Cord Injury (targets: mTOR, PTEN, KLF6/7, SOXI 1, KCC2, and growth factors); SCA6; SCA3 (Machado-Joseph Disease); Multiple system Atrophy (MSA); Treatment-resistant Hypertension; Myotonic Dystrophy Type 2 (DM2); Fragile X-associated Tremor Ataxia Syndrome (FXTAS); West Syndrome with ARX Mutation; Age-related Macular Degeneration (AMD)/Geographic Atrophy (GA); C90RF72-Frontotemporal Dementia (FTD); Facioscapulohumeral Muscular Dystrophy (FSHD); Fragile X Syndrome (FXS); Huntington's Disease; Glaucoma; Acromegaly; Achromatopsia (total color blindness); Ullrich congenital muscular dystrophy; Hereditary myopathy with lactic acidosis; X-linked spondyloepiphyseal dysplasia tarda; Neuropathic pain (Target: CPEB); Persistent Inflammation and injury pain (Target: PABP); Neuropathic pain (Target: miR-30c-5p); Neuropathic pain (Target: miR-195); Friedreich's Ataxia; Uncontrolled gout; Inflammatory pain (Target: Nay1.7 and Nav1.8); Choroideremia; Focal epilepsy; Alpha-1 Antitrypsin deficiency (AATD); Androgen Insensitivity Syndrome; Opioid-induced hyperalgesia (Target: Raf-1); Neurofibromatosis type 1; Stargardt's Disease; Dravet Syndrome; Retinitis Pigmentosa; and Parkinson's Disease.

### Additional Embodiments

Aspects of the present disclosure provide non-naturally occurring, engineered compositions comprising: a trans-splicing template polynucleotide comprising: (a) an insertion sequence; (b) a 5′ splicing motif sequence; (c) optionally, a linker sequence; (d) a hybridization sequence; and (e) a nucleic acid sequence encoding a ribozyme.

In some embodiments. (a)-(e) are arranged 5′ to 3′.

In some embodiments, the ribozyme is capable of cleaving RNA.

In some embodiments, the ribozyme is capable of cleaving DNA.

In some embodiments, the ribozyme is a self-cleaving ribozyme.

In some embodiments, the ribozyme is a naturally occurring ribozyme.

In some embodiments, the ribozyme is a synthetic ribozyme.

In some embodiments, the ribozyme is selected from Twister ribozyme, Hammerhead (HH) ribozyme, Hepatitis Delta Virus (HDV) ribozyme, Hairpin 1 ribozyme, Hairpin 2 ribozyme, Hairpin 3 ribozyme, Varkud Satellite ribozyme, glmS ribozyme, twister sister ribozyme, pistol ribozyme, and hatchet ribozyme.

In some embodiments, the ribozyme is Twister ribozyme.

In some embodiments, the ribozyme is Hepatitis Delta Virus (HDV) ribozyme.

In some embodiments, the trans-splicing template polynucleotide comprises a sequence that is at least 90% identical to one of SEQ ID NOs: 1-7, 9-104, or 117-128. In some embodiments, the trans-splicing template polynucleotide comprises a sequence of one of SEQ ID NOs: 1-7, 9-104, or 17-128.

In some embodiments, the insertion sequence is less than 1-2 kilobases. In some embodiments, the insertion sequence is about 1-2 kilobases. In some embodiments, the insertion sequence is greater than 1-2 kilobases. In some embodiments, the insertion sequence is 62 base pairs (bp). In some embodiments, the insertion sequence is 2 kilobases (kb). In some embodiments, the insertion sequence is 4 kb. In some embodiments, the insertion sequence is 6 kb. In some embodiments, the insertion sequence is 8 kb. In some embodiments, the insertion sequence is 10 kb.

In some embodiments, the 5′ splicing motif is GURAGU.

In some embodiments, the linker is about 14 bp to 100 bp. In some embodiments, the linker is about 50 bp.

In some embodiments, the hybridization sequence is about 50 bp to 400 bp.

In some embodiments, the hybridization sequence is 150 bp.

Further aspects of the present disclosure relate to cells comprising the trans-splicing template polynucleotides of the present disclosure.

In some embodiments, the cell is a prokaryotic cell. In some embodiments, the cell is a eukaryotic cell. In some embodiments, the eukaryotic cell is a mammalian cell. In some embodiments, the mammalian cell is a human cell. In some embodiments, the mammalian cell is a rodent cell. In some embodiments, the eukaryotic cell is a plant cell.

Further aspects of the present disclosure relate to methods of editing a target RNA sequence in a cell, the method comprising delivering to the cell a trans-splicing template polynucleotide comprising a nucleic acid sequence encoding a ribozyme, wherein the trans-splicing template polynucleotide hybridizes to at least a portion of the target RNA sequence, causing cleavage and insertion steps to achieve editing of the target RNA sequence via 5′ trans-splicing. In some embodiments, the ribozyme cleaves the poly(A) tail of the trans-splicing template polynucleotide in the cell.

Further aspects of the present disclosure relate to methods of altering 5′ splicing of a pre-mRNA in a cell comprising administering to the cell an effective amount of the trans-splicing template polynucleotides of the present disclosure. In some embodiments, the ribozyme cleaves the poly(A) tail of the trans-splicing template polynucleotide in the cell.

Further aspects of the present disclosure relate to methods of generating specific cuts in a target RNA in a cell comprising administering to the cell an effective amount of the trans-splicing template polynucleotides of the present disclosure. In some embodiments, the ribozyme cleaves the poly(A) tail of the trans-splicing template polynucleotide in the cell.

Further aspects of the present disclosure relate to methods of editing a target RNA sequence via 5′ trans-splicing in a cell, the method comprising delivering to the cell a non-naturally occurring, engineered trans-splicing template polynucleotide comprising: (a) an insertion sequence; (b) a 5′ splicing motif sequence; (c) optionally, a linker sequence; (d) a hybridization sequence; and (c) a nucleic acid encoding a ribozyme. In some embodiments, the ribozyme cleaves the poly(A) tail of the trans-splicing template polynucleotide in the cell.

In some embodiments, the methods of the present disclosure comprise delivering to the cell by a viral vector, optionally wherein the viral vector is Adeno-associated viral (AAV) vector, a virus, optionally wherein the virus is an Adenovirus, a lentivirus, a herpes simplex virus; and/or a lipid nanoparticle. In some embodiments, the delivering to the cell occurs in vivo.

In some embodiments, the cell is a eukaryotic cell. In some embodiments, the eukaryotic cell is a mammalian cell. In some embodiments, the mammalian cell is a human cell. In some embodiments, the mammalian cell is a rodent cell. In some embodiments, the eukaryotic cell is a plant cell.

Further aspects of the present disclosure relate to methods of editing a target RNA sequence in a cell, the method comprising delivering to the cell a trans-splicing template polynucleotide comprising a nucleic acid sequence encoding a ribozyme, wherein the trans-splicing template polynucleotide hybridizes to at least a portion of the target RNA sequence, causing cleavage and insertion steps to achieve editing of the target RNA sequence via 3′ trans-splicing.

Further aspects of the present disclosure relate to methods of editing a target RNA sequence via 3′ trans-splicing in a cell, the method comprising delivering to the cell a non-naturally occurring, engineered trans-splicing template polynucleotide comprising: (a) a nucleic acid sequence encoding a ribozyme; (b) a hybridization sequence; (c) a 3′ splicing motif sequence; (d) optionally, a linker sequence; and (e) an insertion sequence.

Further aspects of the present disclosure relate to methods of editing a target RNA sequence in a cell, the method comprising delivering to the cell: (i) a trans-splicing template polynucleotide comprising a nucleic acid sequence encoding a ribozyme, wherein the trans-splicing template polynucleotide hybridizes to at least a portion of the target RNA sequence; (ii) a polynucleotide encoding a Cas7-11 enzyme; and (iii) a polynucleotide encoding a Cas7-11 guide RNA sequence; causing cleavage and insertion steps to achieve editing of the target RNA sequence via simultaneous 5′ and 3′ trans-splicing.

Further aspects of the present disclosure relate to methods of editing a target RNA sequence via simultaneous 5′ and 3′ trans-splicing in a cell, the method comprising delivering to the cell: (i) a non-naturally occurring, engineered trans-splicing template polynucleotide comprising: (a) an insertion sequence; (b) a 5′ splicing motif sequence; (c) optionally, a linker sequence; (d) a hybridization sequence; and (e) a nucleic acid encoding a ribozyme; (ii) a polynucleotide encoding a Cas7-11 enzyme; and (iii) a polynucleotide encoding a Cas7-11 guide RNA sequence.

### EXAMPLES

### Example 1: Ribozyme-Facilitated Editing for Efficient 5′ Trans-Splicing

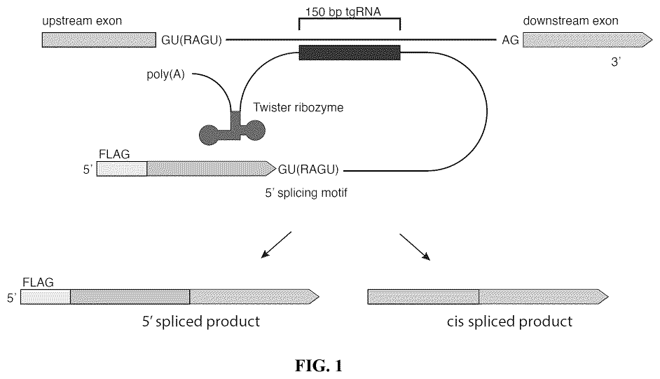

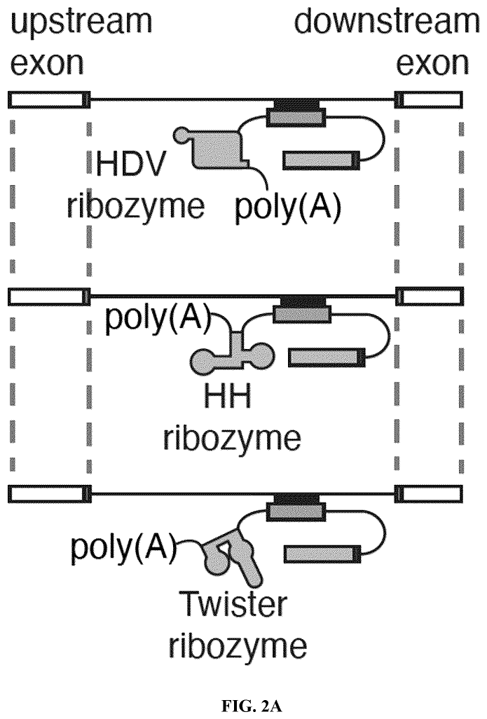

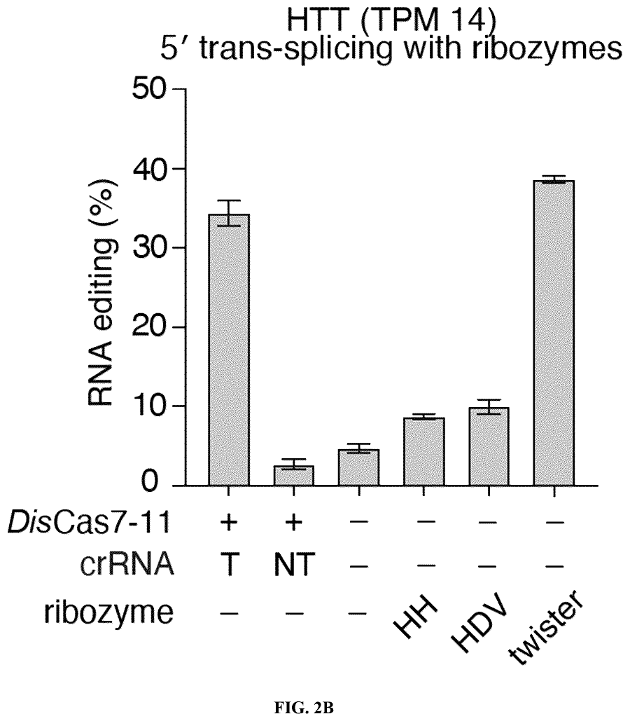

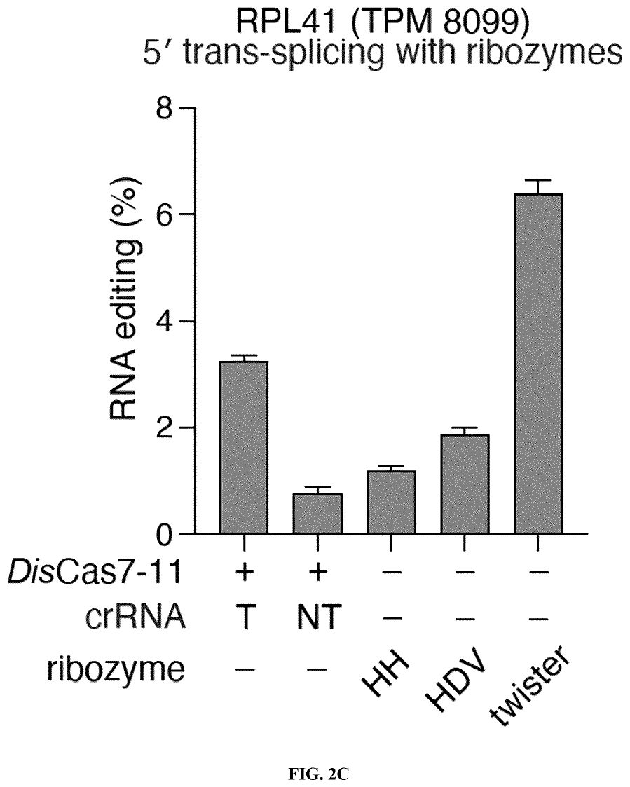

To overcome inefficiencies with existing trans-splicing-based RNA editing approaches, it was hypothesized that precise cleavage of pre-mRNAs could separate downstream cis exons and bias composition towards the trans-splicing template and increase trans-splicing efficiency. As ribozymes are catalytically active RNA molecules capable of specific ribonucleolytic self-cleavage, it was hypothesized that ribozymes could be used to precisely cleave pre-mRNAs and generate specific cuts in target RNAs (FIG. 1). To evaluate this hypothesis, a set of trans-templates was designed using a panel of ribozymes, including Hepatitis Delta Virus (HDV), Hammerhead (HH), or Twister ribozyme (FIG. 2A, Table 1). Each 5′ splicing construct (e.g., each trans-splicing template) was designed with the following components: 1) an insertion sequence (e.g., a cargo/payload sequence); 2) a GURAGU 5′ splicing motif sequence+linker sequence (SEQ ID NO: 8); 3) a hybridization sequence; and a sequence encoding a ribozyme.

TABLE 1
Exemplary trans-splicing template (e.g., Cargo) construct sequences.

| SEQ ID | | |
|---|---|---|
| NO: | Description | Sequence |
| 1 | HTT HDV | GCCACCATGGACTACAAAGACGATGACGACAAGggcccggctgtggctgaggagccCc |
| | ribozyme cargo | tCcaccgaccGTGAGTttgggcTGCATGacTGCATGgTTGCATGaacacaTATTAATttcctc |
| | | cacttagttctacacctcattcattcattcagtgagtgtttctcgactactatgaataaaccgttatactccatgttgcgggcaga |
| | | atcgggatctggacagggaagcacagggcacgagttcaccaatggctgtcaagctacgctgcggccggcatggtccca |
| | | gcctcctcgctcgcgccggctgggcaacatgcttcggcatcgcgaatcggacGCGGCCGC |
| 2 | HTT HH | GCCACCATGGACTACAAAGACGATGACGACAAGggcccggctgtggctgaggagccCc |
| | ribozyme cargo | CCcaccgaccGTGAGTttgggcTGCATGacTGCATGgCTGCATGaacacaTATTAATttcctc |
| | | cacttagttctacacctcattcattcattcagtgagtgtttctcgactactatgaataaaccgttatactccatgttgcgggcaga |
| | | atggggatctggacagcgaagcacagggcacgagttcaccaatggctgtcaagctacgctgcctgatgagtccgtgagg |
| | | acgaaacgagtaagctcgtcGCAGCG |
| 3 | HTT twister | GCCACCATGGACTACAAAGACGATGACGACAAGggcccggctgtggctgaggagccCc |
| | ribozyme cargo | tCcaccgaccGTGAGTttgggcTGCATGacTGCATGgtTGCATGaacacaTATTAATttcctc |
| | | cacttagttctacacctcattcattcattcagtgagtgtttctcgactactatgaataaaccgttatactccatgttgcgggcaga |
| | | atggggatctggacagggaagcacagggcacgagttcaccaatggctgtcaagctacgctgcCCGCCTAACAC |
| | | TGCCAATGCCGGTCCCAAGCCCGGATAAAAAGTGGAGGGGGGGG |
| 4 | RPL41 HDV | GCCACCATGGACTACAAAGACGATGACGACAAGagagccaagtggaggaagaagcgaa |
| | ribozyme cargo | tccgGCgGTGAGTttgggcTGCATGacTGCATGgtTGCATGaacacaTATTAATttccCC |
| | | AGAGTTGCCTTTCCCTCCCACATTAAATCAAACGTCCACATAAAGAATGA |
| | | GGTGGTAAAATGAACAAGCACTACGGTTCTATCGTTCTCTGTTCTGTTAAA |
| | | TCCTGGCTCCAGGGAGAAAACACTCAAACGTTTTTCTCCTAAAGATCggccg |
| | | gcaggtcccagcctcctcgctggcgccggctgggcascatgcttcggcatggcgaatgggacGCGGCCGC |
| 5 | RPL41 HH | GCCACCATGGACTACAAAGACGATGACGACAAGagagccaagtggaggaagttagcgaa |
| | ribozyme cargo | tgcgGCgGTGAGTttgggcTGCATGacTGCATGgtTGCATGaacacaTATTAATttccCC |
| | | AGAGTTGCCTTTCCCTCCCACATTAAATCAAACGTCCACATAAAGAATGA |
| | | GGTGGTAAAATGAACAAGCACTACGGTTCTATCGTTCTCTGTTCTGTTAAA |
| | | TCCTGGCTCCAGGGAGAAAACACTCAAACGTTTTTCTCCTAAAGATCctgatg |
| | | agtccctgaggacgaaacgagtaagctcctcgatctt |
| 6 | RPL41 twister | GCCACCATGGACTACAAAGACGATGACGACAAGagagccaagtggaggangaagcgaa |
| | ribozyme cargo | tgcgGCgGTGAGTttgggcTGCATGacTGCATGgTGCATGaacacaTATTAATttccCC |
| | | AGAGTTGCCTTTCCCTCCCACATTAAATCAAACGTCCACATAAAGAATGA |
| | | GGTGGTAAAATGAACAAGCACTACGGTTCTATGGTTCTCTGTTCTGTTAAA |
| | | TCCTGGCTCCAGGGAGAAAACACTCAAACGTTTTTCTCCTAAAGATCCCG |
| | | CCTAACACTGCCAATGCCGGTGCCAAGCCCGGATAAAAAGTGGAGGGGG |
| | | CGG |
| 7 | HTT AAV | gccgccGCCACCATGGACTACAAAGACGATGACGACAAGggcccggctgtggctgagg |
| | backbone HDV | agccCctCcaccgaccGTGAGTttgggcTGCATGacTGCATGgtTGCATGaacacaTATTA |
| | ribozyme cargo | ATttcctccacttagttctacacctcattcattcattcagtgagtgtttctcgactactatgaataaaccgttatactccatgttgc |
| | | cggcagaatggggatctggacagggaagcacagggcacgagttcaccaatggctgtcaagctacgctgcggccggcat |
| | | ggtcccagcctcctcgctgccgccggctgggcaacatgcttcggcatggcgaatcgcacGCGGCCGC |
| 8 | GURAGU 5′ | GTGAGTttgggcTGCATGacTGCATGgtTGCATGaacacaTATTAATttcc |
| | splicing motif + | |
| | linker sequence | |

Using these designs, 5′ trans-splicing was piloted on the HTT exon 1, where triplet expansions cause Huntington's Disease pathology, replacing the endogenous exon 1 with a new copy of the exon carrying no CAG repeats and being only 68 nucleotides in length. Using HH or HDV resulted in ˜8-12% editing, while 5′ trans-splicing was improved ˜10-fold (˜35-58% trans-splicing efficiency) using Twister (FIG. 2B). After demonstrating the principle of ribozyme-facilitated editing on HTT, the constructs were tested on RPL41 pre-mRNA transcripts (FIG. 2C). The data similarly showed that Twister and HDV ribozymes performed the best, producing a range of 2-7% RNA editing.

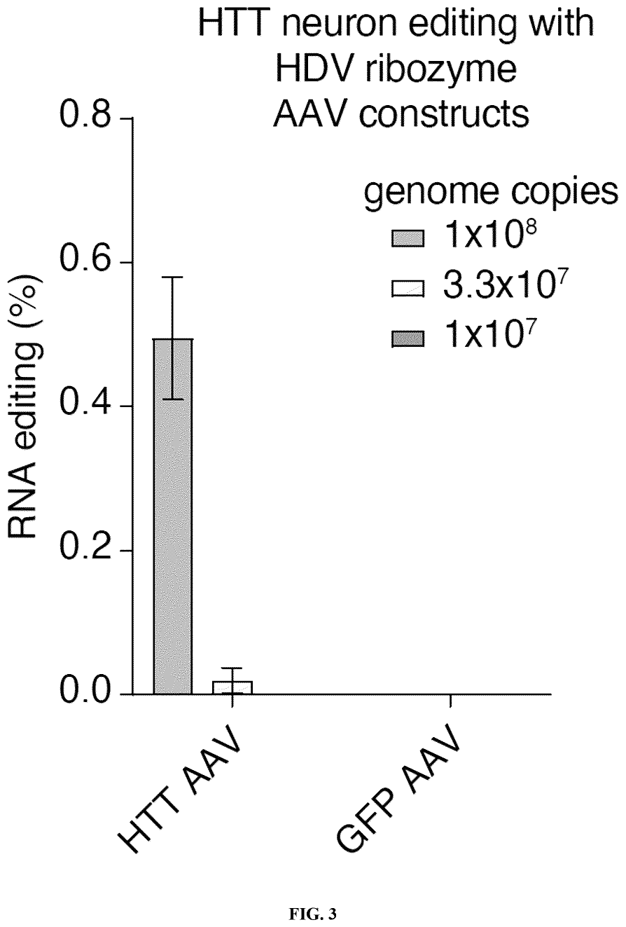

Finally, these constructs were evaluated for their suitability for efficient editing via AAV delivery. For this experiment, the HTT trans-splicing Twister ribozyme construct was packaged into AAV8 vectors. Human iPSC-derived neurons were transduced with these AAVs at three different doses. The results demonstrated that ribozyme-facilitated editing could achieve˜0.5% RNA editing efficiency (FIG. 3).

Taken together, the high activity of ribozyme-facilitated editing enabled editing in non-dividing neurons and via AAV delivery demonstrate that payload engineering and ribozymes can be combined for protein-free, high-efficiency trans-splicing.

### Methods

Cloning of cargo constructs: Constructs were ordered as eBlock Gene Fragments from Integrated DNA Technologies (San Diego, CA, USA) and cloned by Gibson Assembly. A pcDNA3.1-mCardinal cloning backbone (Addgene #513111) was digested using Fermentas FD BamHI and Fermentas FD EcoRI (Thermo Fisher Scientific, FD0054, FD0274) with 10× FastDigest Buffer, for a reaction containing 2-5 μg of each enzyme in a total reaction size of 20 pI, in UltraPure water. Digestions were incubated for 1 hour at 37° C., and then diluted 1:5 with UltraPure water before loading on E-gel EX 2% Agarose gels (Invitrogen, G401002). Backbones were purified using the Monarch DNA Gel Extraction Kit (New England Biolabs, Ipswich. MA, USA) and assembled directly with the eBlock constructs at a 1:3 molar ratio of backbone:insert, using 50 ng of backbone and 2.5 μL of HiFi DNA Assembly Mix (New England Biolabs, Ipswich, MA, USA) in a total 5 μL reaction size. Assembly mixes were incubated for 1 hour at 50° C. Post incubation, assembled products were diluted 1:1 with UltraPure water. 2 μL of product was transformed into One Shot™ StbI3′m Chemically Competent E. coli cells, then plated on Agarose plates with 100 μg ampicillin for overnight growth at 37° C. Single clones were plated into 1 mL of TB media containing 100 μg/mL ampicillin in 2 mL 96-well plates and grown overnight in a 37° C. rotating shaker. Plasmid DNA was purified from cells using a QIAprep 96 Plus Miniprep Kit (Qiagen, Hilden Germany) and EconoSpin® Miniprep Filter plates (Epoch Life Science, Fort Bend Count, TX, USA). Purified plasmids were prepared for sequencing using a Tn5 transposase and tagmentation, and sequenced using an illumina MiSeq (Illumina, San Diego, CA, USA). Correct clones were verified using Geneious Prime.

HEK cell culture: HEK293FT cells (Invitrogen, R70007) were cultured in Dulbecco's modified Eagle medium with high glucose, sodium pyruvate, and GlutaMAX™ (Thermo Fisher Scientific, 35050079) and supplemented with 10% (vol/vol) fetal bovine serum (FBS) and 1× penicillin-streptomycin (Thermo Fisher Scientific, 35050079). Cells were maintained at 37° and 5% CO₂ throughout all experiments.

Neuron cell culture: iPSC-derived neurons were generated according to the approach outlined in Tian et al. 2019 “CRISPR Interference-Based Platform for Multimodal Genetic Screens in Human iPSC-Derived Neurons”. Neurons were plated on black-well 96-well plates for all experiment and maintained in differentiation media consisting of Dulbecco's modified Eagle medium supplemented with 0.5× Neurobasal-A, 1× NEAA, 0.5× GlutaMAX™, 0.5× B27-CA, 10 ng/mL NT-3, 10 ng/mL BDNF, 1 μg/mL mouse laminin, and 2 μg/mL doxycycline.

AAV production and transduction: AAV constructs were produced in HEK293FT cells cultured in T225 flasks by transfection of 1:1:1 molar ratios of helper plasmid, capsid, and transfer plasmids (per construct), totaling 90 μg of DNA. PEI was used for all transfections, and media was changed on transfected cells 4-6 hours post transfection. 48 hours post-transfection, the supernatant from transfected flasks was collected and briefly centrifuged at 1000×g for 5 minute to pellet cell debris. Clarified supernatant was then passed through a 0.45 μm syringe filter before centrifuging through 100 kDa MWCO Amicon filters to concentrate (Millipore Sigma, UFC910024). Two washes with PBS were also performed in an Amicon filter. The concentrated AAV was collected and a small fraction was used to estimate viral genome titers by qPCR as follows: first, a DNAse digestion was performed using 1 μL of concentrated AAV, 2 μL of DNase I buffer, and 0.5 μl of DNase I (New England Biolabs, M0303S) in UltraPure water for a total reaction volume of 20 μL. The DNase digestion as incubated at 37° for 1 hour, then at 75° for 15 minutes. The Proteinase K treatment was incubated at 50° for 30 minutes, then at 98° for 10 minutes. qPCR was performed using custom primers binding within the intra-ITR CMV promoter of all constructs, using Fast SYBR Green Master Mix (Applied Biosystems, 4385612). 0.2 μL each of 50 μM forward and reverse primers were combined with 4 μL of the treated sample, along with 10 μL of 2× SYBR Green mix in UltraPure water for a total reaction volume of 2 μL. A serial dilution was prepared for each transfer plasmid from 1:10 to 1:10” and SYBR reactions were set up for each dilution. Reactions were run on the CFX384 Touch Real-Time PCR System (BioRad, Hercules, CA, USA). Copy numbers were calculated according to the standard curves generated by the serial dilution reactions.

### Example 2: Simultaneous 5′ and 3′ Trans-Splicing of RNA (Prophetic)

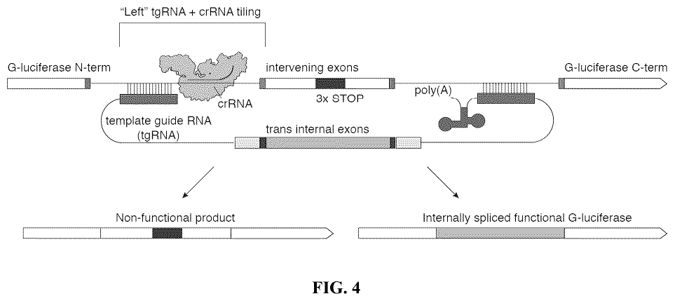

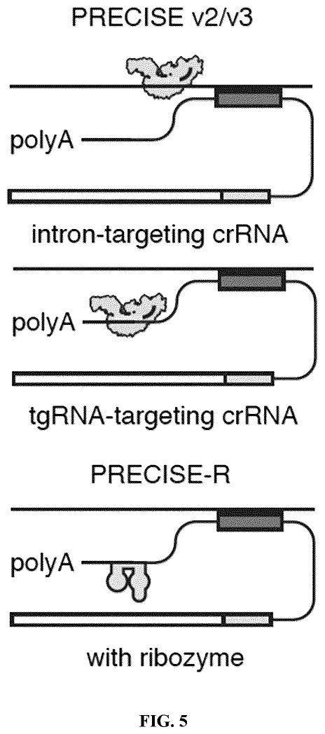

Simultaneous 5′ and 3′ trans-splicing of RNA would allow for “internal” trans-splicing, where an internal exon within a precursor mRNA is replaced independently of flanking exons. This strategy differs from other approaches that replace an internal exon and all 5′ or 3′ sequence, as it allows for a small cargo construct and would be capable of replacing exons in situations where including the entire upstream or downstream sequence isn't feasible (i.e., due to size constrains of delivery). Constructs can be generated with both 5′ and 3′ hybridization regions (FIG. 4). The upstream (5′) side of the cargo undergoes a 3′ splicing event, while the downstream (3′) side of the cargo undergoes a 5′ splicing event. The cargos can be designed such that the 3′ and 5′ events are both assisted by ribozymes, or where either the 3′ or 5′ event is assisted by ribozymes and the other by a DisCas7-11 construct.

In particular, the 5′ splicing event was shown in Example 1 to be relatively efficient after attaching a ribozyme sequence behind the hybridization region. Constructs will be tested on a fluorescence reporter as well as on endogenous transcript (e.g., STAT3, PPIB, TOP2A, PABPC1), where an internal exon within a pre-mRNA is replaced. These constructs will have an upstream 3′ splicing event assisted by DisCas7-11 cleavage of the targeted intron, and a downstream 5′ splicing event. The downstream splicing hybridization could have one of several potential ribozymes trailing it (e.g., HH, HDV, or Twister) to remove the poly(A) tail. Multiple cargos with different hybridization regions (e.g., size, binding position, linker length) will be generated to evaluate the design rules.

### Example 3: Ribozyme-Facilitated Editing for Efficient 3′ Trans-Splicing (Prophetic)

Cargos can also be generated to facilitate 3′ splicing events or replace 5′ exons on mRNA in situations where the first exon or portion of only the 5′ ends of genes need to be edited. To edit the upstream sequence of mRNA, constructs can have the opposite arrangement of cargo elements (e.g., ribozyme—hybridization—insertion sequence). The upstream splicing hybridization could have one of several potential ribozymes trailing it (e.g., HH, HDV, or Twister) to remove the poly(A) tail.

Constructs will be tested on a fluorescence reporter as well as on endogenous transcripts (e.g., STAT3, PPIB, TOP2A, PABPC1), where a 5′ exon within a pre-mRNA is replaced. Multiple cargos with different hybridization region (e.g., size, binding position, linker length) will be generated to evaluate the design rules.

The constructs will also be evaluated for their suitability for efficient editing via AAV or lentiviral delivery. Methods for AAV delivery have been discussed in Example 1. For lentiviral delivery, lentiviruses can be produced in HEK293FT cells cultured in T225 flasks by cotransfection of packaging plasmid (e.g., psPAX2, Addgene #12260), envelope plasmid (e.g., VSV-G, Addgene #8454), and transfer plasmid using polyethylene imine. Media will be changed 6-8 hours after transfection with fresh D10. Media containing lentiviruses will be harvested after 48 hours of transfection, briefly centrifuged at 1000×g for 5 minutes to pellet cell debris and filtered through vacuum filters (e.g., 0.45 μm vacuum filters), then ultracentrifuged for 2 hours at 120,000×g and resuspended in PBS. Cells (e.g., HEK293FT cells, hHD fibroblasts, and iPSC-derived neurons) can be plated and infected with concentrated lentiviruses in DMEM 10% FBS, in the presence of polybrene, to evaluate editing efficiency via lentiviral delivery.

### Example 4: Additional Engineering Approaches for Ribozyme-Facilitated 5′ Editing

To further assess the capabilities of the 5′ splicing construct (e.g., trans-splicing template) described in Example 1, which showed that ribozymes could be used to precisely cleave pre-mRNAs and generate specific cuts in target RNAs, additional trans-splicing templates were designed using different engineering approaches. All construct sequences are provided in Table 2.

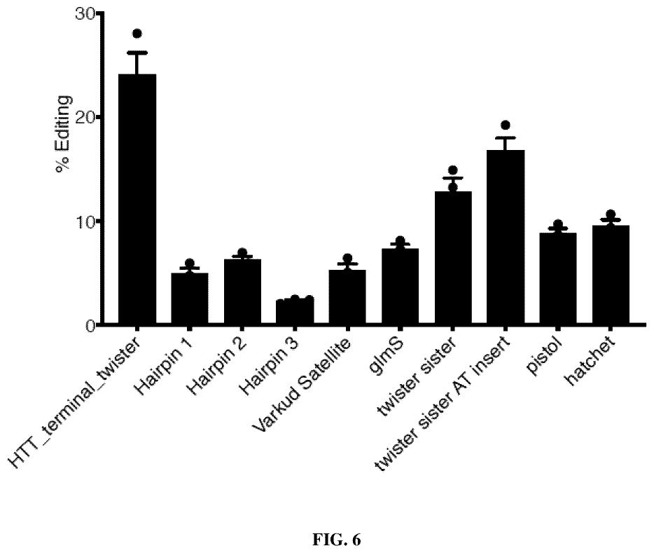

First, additional trans-splicing templates were designed using a panel of ribozymes with representatives of most of the known ribozyme families. Ribozymes analyzed were Twister, Hairpin 1, Hairpin 2. Hairpin 3. Varkud Satellite, glmS, twister sister, twister sister AT insert, pistol, and hatchet (FIG. 6). Each 5′ splicing construct (e.g., each trans-splicing template) was designed with the following components: 1) an insertion sequence (e.g., a cargo/payload sequence); 2) a GURAGU 5′ splicing motif sequence+linker sequence (SEQ ID NO: 8); 3) a hybridization sequence; and a sequence encoding a ribozyme. The editing efficiencies shown in FIG. 6 demonstrate the 5′ splicing construct (e.g., trans-splicing template) is compatible with representatives of most of the known ribozyme families.

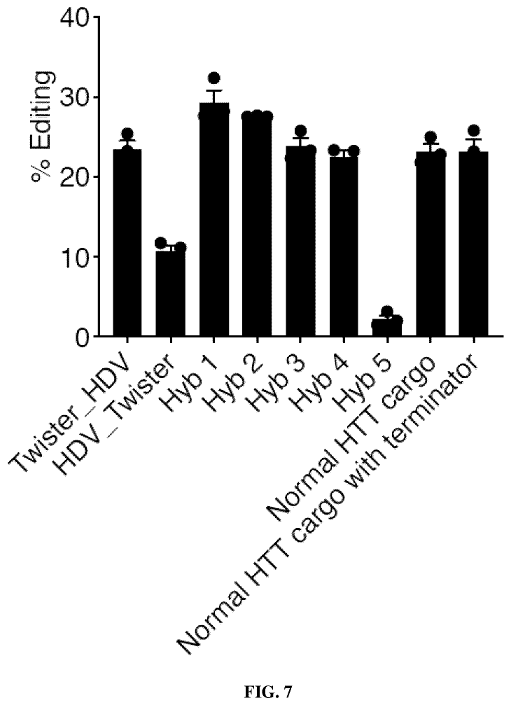

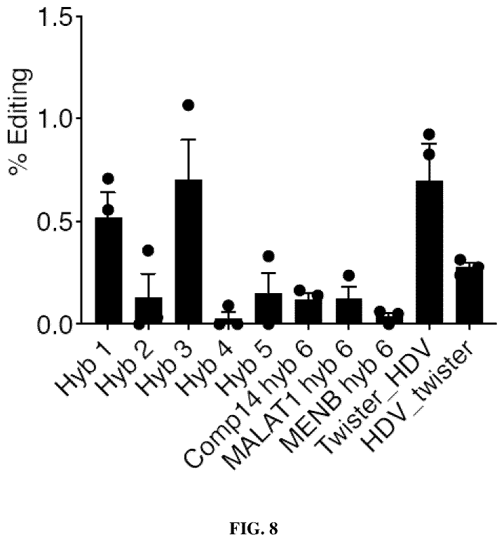

Editing efficiency is strongly dependent on identifying the best hybridization regions for the cargos. Therefore, additional 5′ splicing constructs (e.g., trans-splicing templates) were generated with marginally different hybridization (e.g., binding) regions. HTT transcript editing rates (FIG. 7) and MECP2 transcript editing rates (FIG. 8) were tested with 5′ trans-splicing constructs comprising different hybridization regions. Several of these new hybridization regions (“Hyb 1” and “Hyb 2”) exhibited increased editing efficiency relative to the “Normal HTT cargo” comprising the twister ribozyme (FIG. 7). The combination of multiple ribozymes in tandem (Twister_HDV” and HDV_Twister”) was also evaluated relative to the “Normal HTT cargo” comprising the twister ribozyme (FIGS. 7 and 8).

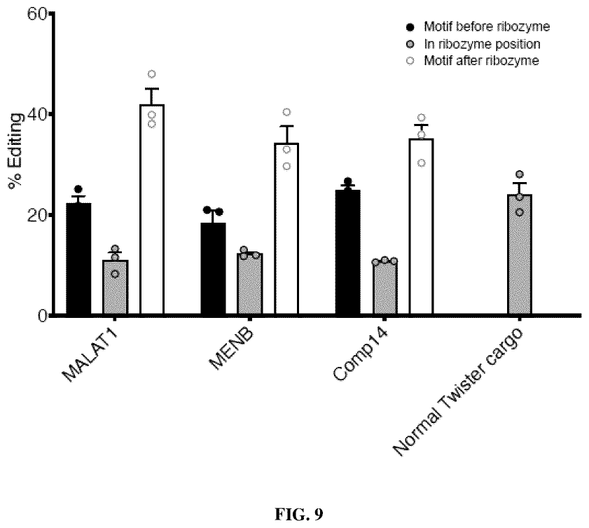

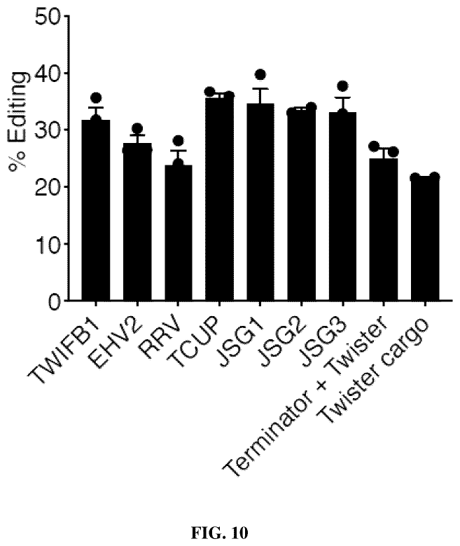

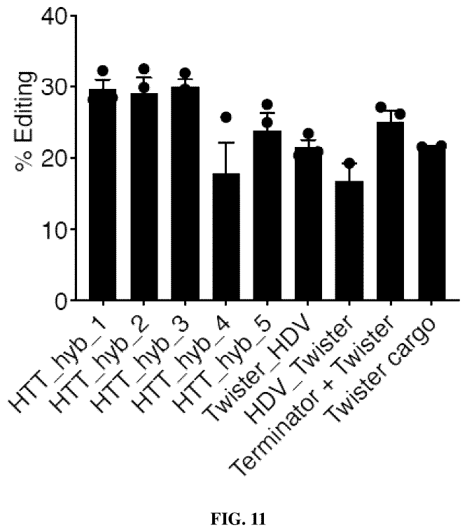

Triple helix sequence motifs form a secondary structure that has been shown to affect several features related to transcript stability. Therefore, additional 5′ splicing constructs (e.g., trans-splicing templates) were generated with triple helix sequence motifs in place of the ribozyme, 5′ to the ribozyme, or 3′ to the ribozyme. HTT editing rates for said constructs were subsequently evaluated (FIG. 9) and surprisingly showed that the most efficient approach is placing the triple helix 3′ of the ribozyme. Additional 5′ splicing constructs (e.g., trans-splicing templates) were designed to have one of several different element for nuclear expression (“ENE”) elements that were recoded based on known sequences. HTT editing rates for said constructs were subsequently evaluated (FIG. 10). Additional 5′ splicing constructs (HTT_hyb_1, HTT_hyb_2, HTT_hyb_3, HTT_hyb_4, HTT_hyb_5) were designed with different hybridization (e.g., binding) regions and HTT editing rates for said constructs were subsequently evaluated (FIG. 11). Without wishing to be bound by theory, incorporating the ENE elements in the cargo likely improves the stability of the construct, which resulted in higher editing over time.

### Example 5: Ribozyme-Facilitated 5′ Editing of Target Genes

To further assess the capabilities of the 5′ splicing construct (e.g., trans-splicing template) described in the preceding Examples, which showed that ribozymes could be used to precisely cleave pre-mRNAs and generate specific cuts in target RNAs, trans-splicing templates were designed using ribozyme Twister and different hybridization regions to target replacement of 5′ exons within several targets associated with genetic disorders (HOXD13, HOXA13, SOD1, KCNQ1, SPTBN2, ATP7B, CBS, MEF2C, and MECP2). Each experimental 5′ splicing construct (e.g., each trans-splicing template) was designed with the following components: 1) an insertion sequence (e.g., a cargo/payload sequence); 2) a GURAGU 5′ splicing motif sequence+linker sequence (SEQ ID NO: 8); 3) a hybridization sequence; and a sequence encoding a Twister ribozyme. All construct sequences are provided in Table 2.

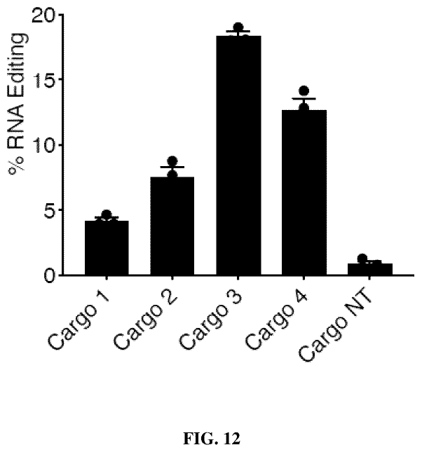

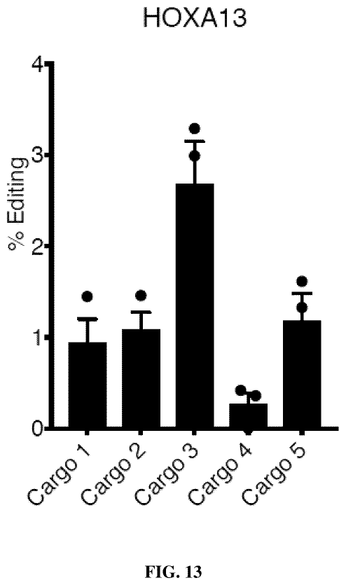

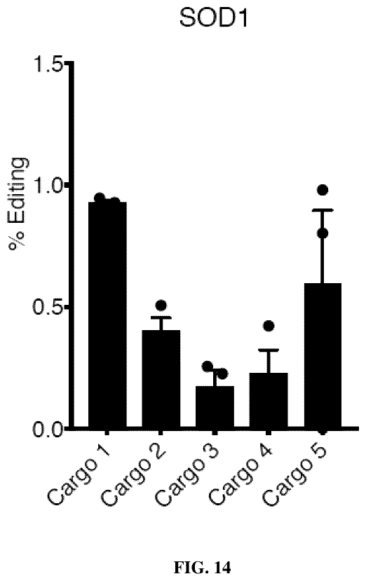

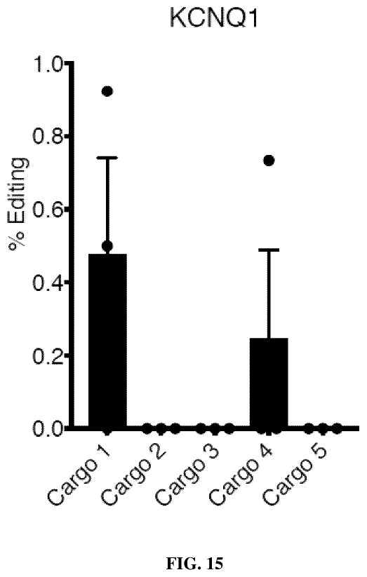

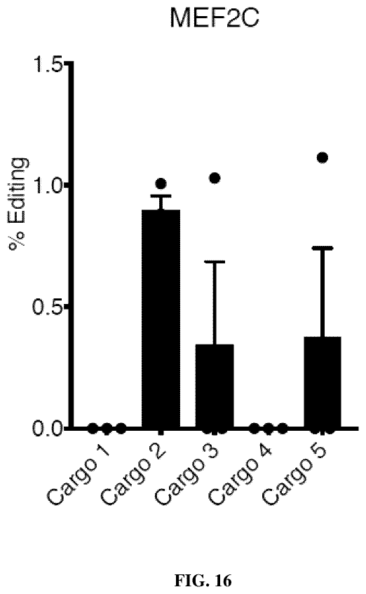

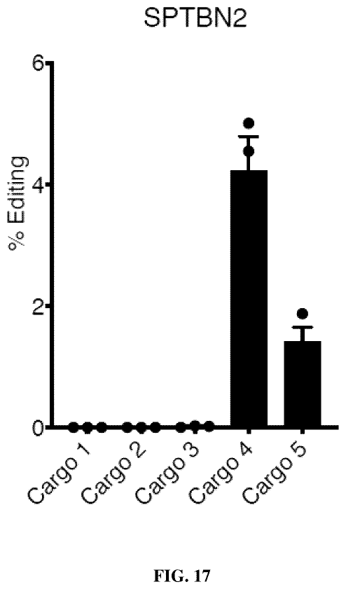

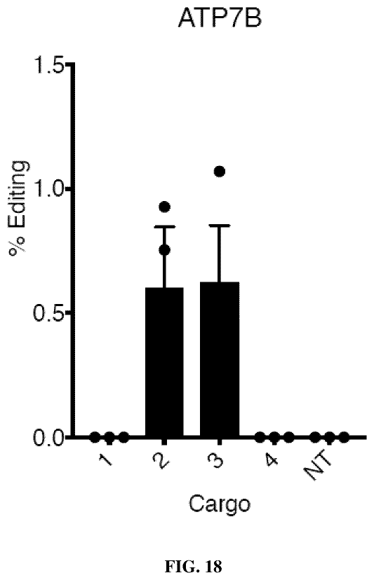

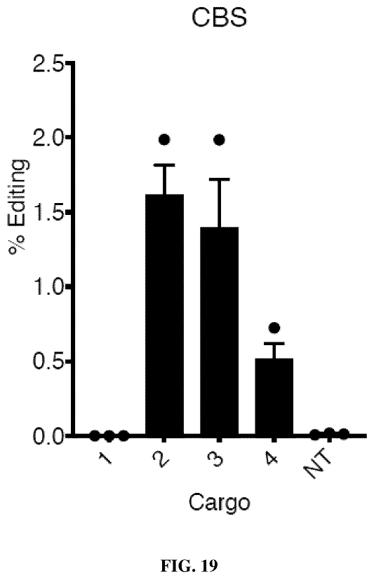

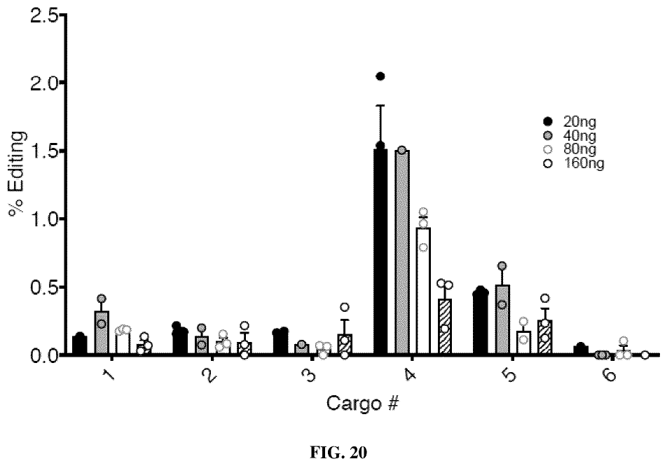

Editing rates for constructs targeting replacement of 5′ exons of HOXD13 are shown in FIG. 12. Editing rates for constructs targeting replacement of 5′ exons of HOXA13 are shown in FIG. 13. Editing rates for constructs targeting replacement of 5′ exons of SOD1 are shown in FIG. 14. Editing rates for constructs targeting replacement of 5′ exons of KCNQ1 are shown in FIG. 15. Editing rates for constructs targeting replacement of 5′ exons of MEF2C are shown in FIG. 16. Editing rates for constructs targeting replacement of 5′ exons of SPTBN2 are shown in FIG. 17. Editing rates for constructs targeting replacement of 5′ exons of ATP7B are shown in FIG. 18. Editing rates for constructs targeting replacement of 5′ exons of CBS are shown in FIG. 19. Editing rates for constructs targeting replacement of 5′ exons of MECP2 that were delivered at four different concentrations (20 ng, 40 ng, 80 ng, and 160 ng) are shown in FIG. 20. The data surprisingly demonstrate the capability of 5′ splicing constructs (e.g., trans-splicing templates) comprising ribozymes to replace 5′ exons in different targets. Such data exemplifies the utility of the ribozyme editing strategy described by the disclosure in the context of editing genes associated with genetic disorders.

### Example 6: Ribozyme-Facilitated 5′ Editing Insertion Size

The capability of the 5′ splicing constructs (e.g., trans-splicing templates) described in the preceding Examples to insert cargo/payloads of different sizes was assessed as follows. Experimental 5′ splicing constructs (e.g., trans-splicing templates) were designed with the following components: 1) an insertion sequence (e.g., a cargo/payload sequence); 2) a GURAGU 5′ splicing motif sequence+linker sequence (SEQ ID NO: 8); 3) a hybridization sequence; and a sequence encoding a Twister ribozyme. All constructs were transfected at a 96-well scale on HEK293FT cells in DMEM 10% FBS. Transfections were carried out using 80 g of cargo plasmid alone using Lipofectamine 3000. RNA was harvested 3 days post-transfection and reverse transcribed using the RevertAid cDNA prep kit with gene specific reverse transcription primers binding within the wildtype downstream exons. Two rounds of PCR were run using amplicon primers binding upstream and downstream of the targeted splicing junction and loaded on an Illumina MiSeq for sequencing. Following sequencing, raw reads were analyzed by searching for counts of the wildtype and trans-product splicing junctions and a percentage was calculated.

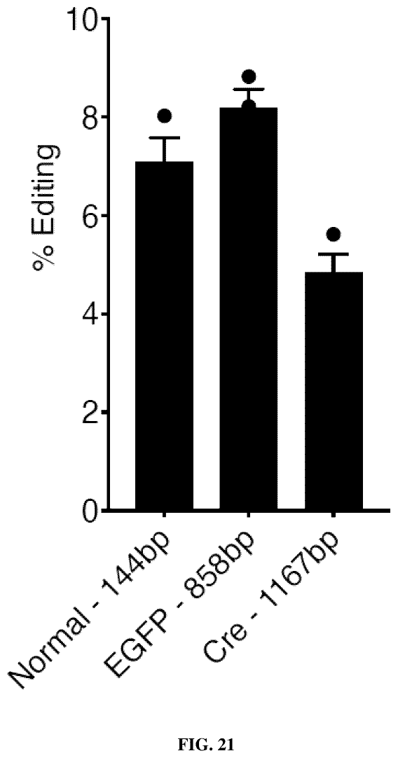

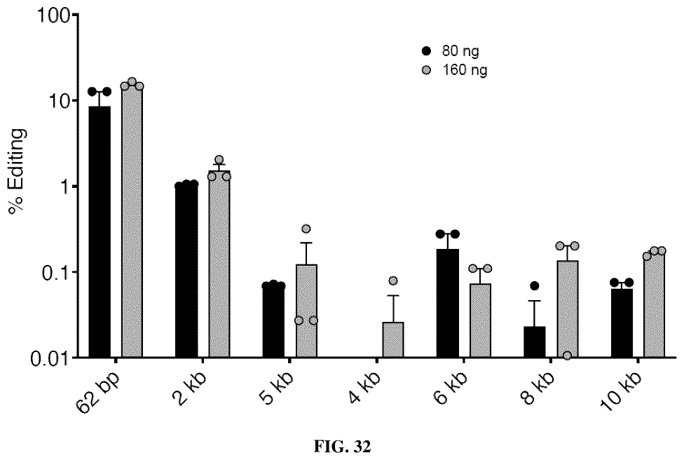

All construct sequences are provided in Table 2. Editing rates of three constructs, which comprised insertion sequences of 114 base pairs, 858 base pairs, or 1167 base pairs in length, respectively, for HOXD13 target are shown in FIG. 21. Editing rates of six constructs, which comprised insertion sequences of 62 base pairs, 2 kilobases, 5 kilobases, 4 kilobases, 6 kilobases, 8 kilobases, and 10 kilobases in length, respectively, for HTT target are shown in FIG. 32. The data surprisingly demonstrate the capability of 5′ splicing constructs (e.g., trans-splicing templates) comprising ribozymes to insert cargos of various sizes, including constructs up to 10 kilobases long, into targets.

### Example 7: Ribozyme-Facilitated 5′AAV Editing

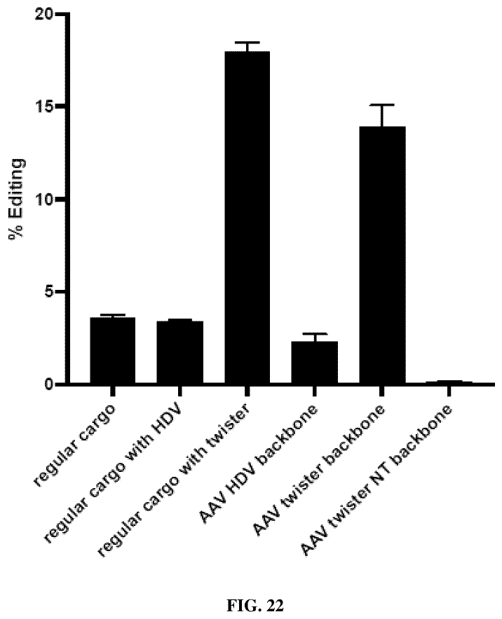

The capability of the 5′ splicing constructs (e.g., trans-splicing templates) described in the preceding Examples to be delivered via AAV backbone was assessed as follows. Experimental 5′ splicing constructs (e.g., trans-splicing templates) were designed with the following components: 1) an insertion sequence encoding “regular cargo” or “AAV”; 2) a GURAGU 5′ splicing motif sequence+linker sequence (SEQ ID NO: 8); 3) a hybridization sequence; and a sequence encoding either HDV ribozyme or Twister ribozyme. All constructs were transfected at a 96-well scale on HEK293FT cells in DMEM 10% FBS. Transfections were carried out using 10-60 ng of cargo plasmid alone using Lipofectamine 300. RNA was harvested 3 days post-transfection and reverse transcribed using the RevertAid cDNA prep kit with gene specific reverse transcription primers binding within the wildtype downstream exons. Two rounds of PCR were run using amplicon primers binding upstream and downstream of the targeted splicing junction and loaded on an Illumina MiSeq for sequencing. Following sequencing, raw reads were analyzed by searching for counts of the wildtype and trans-product splicing junctions and a percentage was calculated. All construct sequences are provided in Table 2. HTT editing rates for constructs comparing regular cargos relative to AAV backbones are shown in FIG. 22. This data demonstrates efficient editing rates for the twister ribozyme based AAV backbone construct.

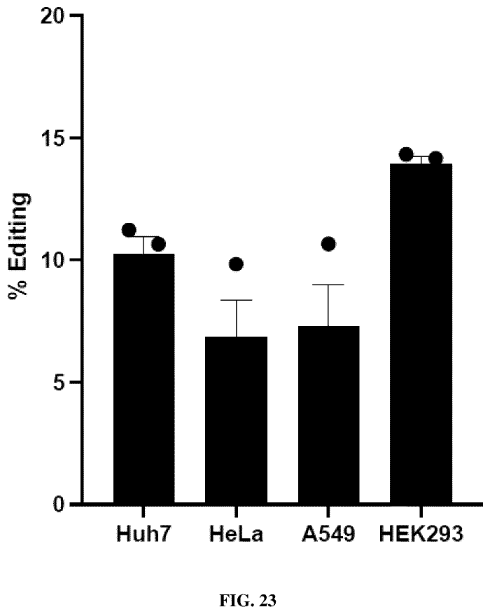

The capability of 5′ splicing constructs (e.g., trans-splicing templates) to edit HTT transcripts in cell lines other than HEK293FT cells were assessed as follows. All constructs were transfected at a 96-well scale on HEK293FT. Huh7. HeLa, or A549 cells in DMEM 10% FBS. Transfections were carried out using 10-60 ng of cargo plasmid alone using Lipofectamine 3000. RNA was harvested 3 days post-transfection and reverse transcribed using the RevertAid cDNA prep kit with gene specific reverse transcription primers binding within the wildtype downstream exons. Two rounds of PCR were run using amplicon primers binding upstream and downstream of the targeted splicing junction and loaded on an Illumina MiSeq for sequencing. Following sequencing, raw reads were analyzed by searching for counts of the wildtype and trans-product splicing junctions and a percentage was calculated. HTT editing rates for 5′ splicing construct (e.g., trans-splicing template) comprising Twister ribozyme transfected into Huh7. HeLa, A549, and HEK293 cell lines are shown in FIG. 23. This data demonstrates that the constructs could be used in cell types other than HEK293FT cells and is generally applicable to a range of cell lines frequently used for research.

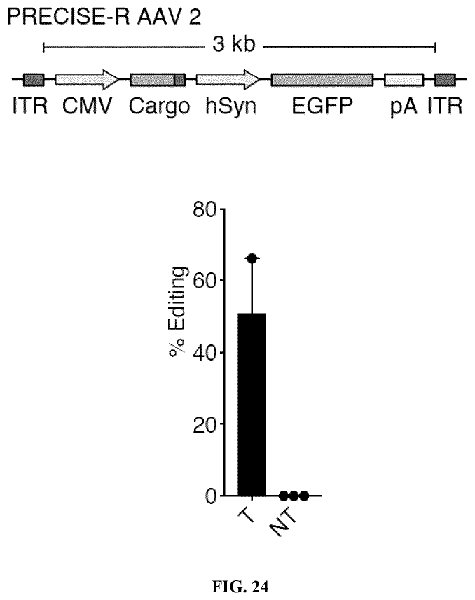

The capability of 5′ splicing constructs (e.g., trans-splicing templates) to edit target transcripts in vitro in human iPSC derived neurons was assessed by transducing human iPSC derived neurons with 5′ splicing constructs packaged into AAV vectors (PRECISE-R AAV 2). Editing of the HTT transcript using the 5′ splicing constructs (e.g., trans-splicing templates) is shown in FIG. 24. This experiment demonstrates in vin the potential to use the ribozyme approach for therapeutic correction of the HTT transcript.

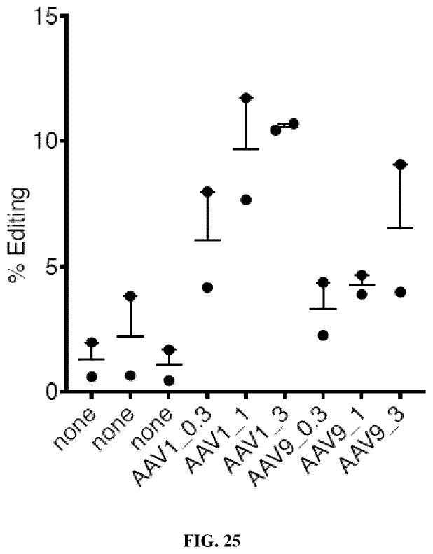

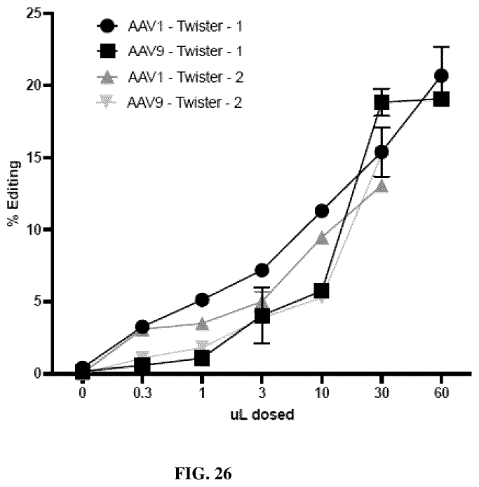

The capability of 5′ splicing constructs (e.g., trans-splicing templates) to edit target transcripts via transduction using AAV constructs packaged into AAV2/1 or AAV2/9 capsids was assessed. All constructs were transduced at a 96-well scale on HEK293FT cells in DMEM 10% FBS. Transfections were carried out using 10-60 ng of cargo plasmid alone using Lipofectamine 3000. RNA was harvested 3 days post-transfection and reverse transcribed using the RevertAid cDNA prep kit with gene specific reverse transcription primers binding within the wildtype downstream exons. Two rounds of PCR were run using amplicon primers binding upstream and downstream of the targeted splicing junction and loaded on an Illumina MiSeq for sequencing. Following sequencing, raw reads were analyzed by searching for counts of the wildtype and trans-product splicing junctions and a percentage was calculated. HTT editing rates for 5′ splicing constructs (e.g., trans-splicing templates) packaged into AAV2/1 or AAV2/9 capsids delivered at three different viral concentrations (0.3 ng, 1 ng, 3 ng) is shown in FIG. 25. HTT editing rates for 5′ splicing constructs (e.g., trans-splicing templates) containing “Twister 1” or “Twister 2” packaged into AAV1 or AAV9 capsids with increasing concentrations of virus is shown in FIG. 26. These data demonstrates that the 5′ splicing constructs function with the AAV capsid delivery mode in a dose-dependent manner.

### Example 8: Ribozyme-Facilitated 5′ RNA and Lipid Nanoparticle Editing

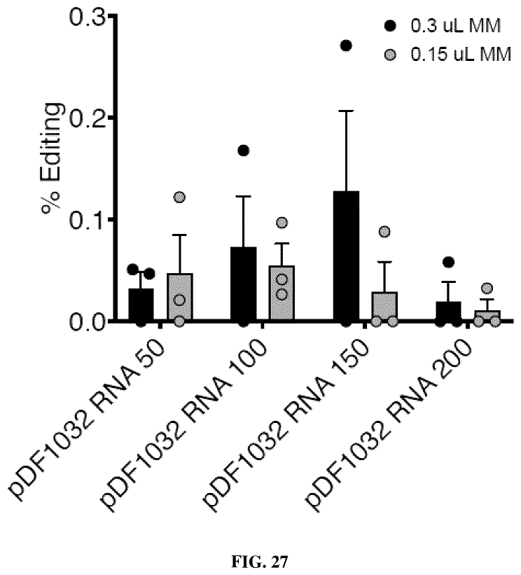

The capability of RNA versions of the 5′ splicing construct (e.g., trans-splicing template) to edit the HTT transcript was assessed. All constructs were transduced at a 96-well scale on HEK293FT cells in DMEM 10% PBS. Transfections were carried out using 50-200 ng of cargo plasmid alone using MessengerMax. RNA was harvested 3 days post-transfection and reverse transcribed using the RevertAid cDNA prep kit with gene specific reverse transcription primers binding within the wildtype downstream exons. Two rounds of PCR were run using amplicon primers binding upstream and downstream of the targeted splicing junction and loaded on an Illumina MiSeq for sequencing. Following sequencing, raw reads were analyzed by searching for counts of the wildtype and trans-product splicing junctions and a percentage was calculated. Messengermax editing rates of an RNA version of the 5′ splicing construct (e.g., trans-splicing template) comprising Twister at different amounts (50 ng, 100 ng, 150 ng, and 200 ng) is shown in FIG. 27. This data demonstrates that while inefficient, RNA editing is possible with RNA versions of the 5′ splicing constructs described herein.

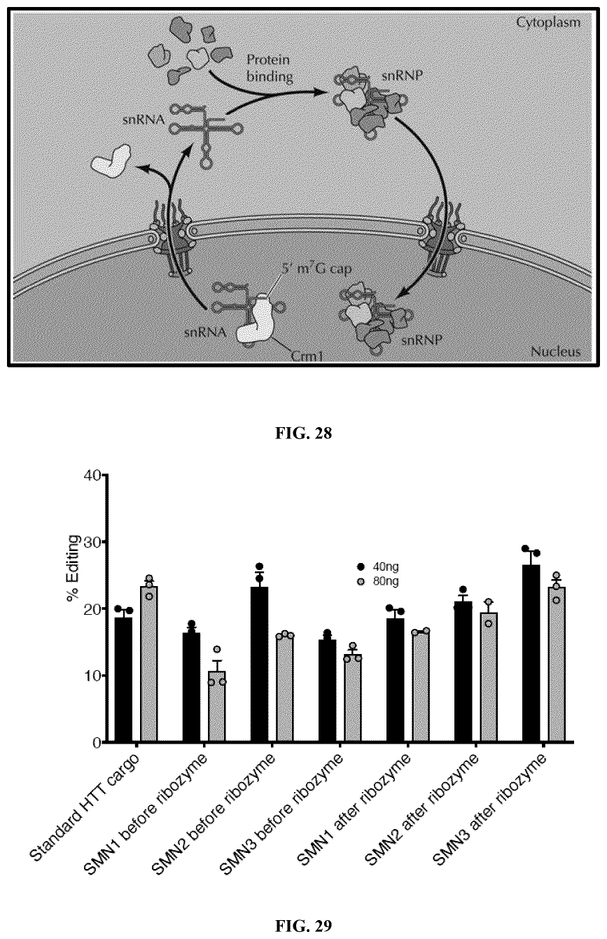

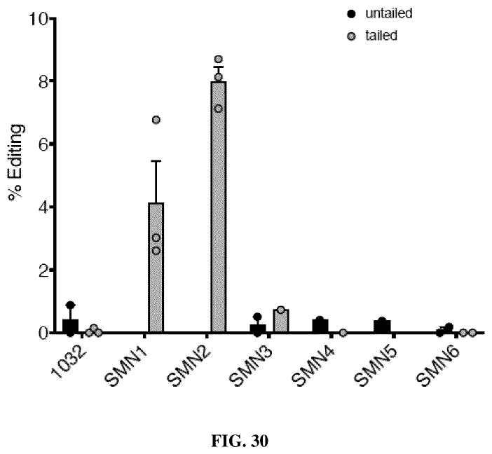

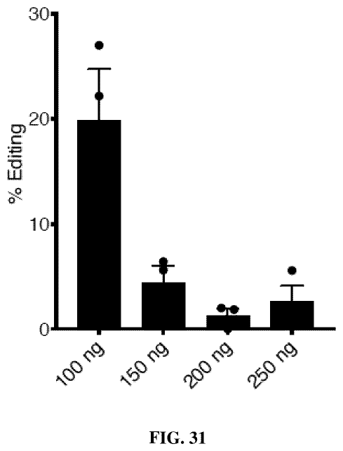

To assess whether the low RNA editing rates observed by messengermax transfection of the RNA (FIG. 27) were due to subcellular localization of the RNA to the cytoplasm, a revised assay was designed utilizing SMN motifs. SMN proteins are known to translocate snRNAs to the nucleus, where splicing occurs (FIG. 28). Therefore, RNA versions of the 5′ splicing construct (e.g., trans-splicing template) tagged with a sequence motif believed to recruit SMN proteins were generated. All construct sequences are provided in Table 2. Editing rates of normal 5′ splicing construct (e.g., trans-splicing template) relative to versions generated with one of several SMN sequence motifs either 5′ (“before”) or 3′ (“after”) to the ribozyme (Twister) are shown in (FIG. 29). Editing rates of the new SMN motif constructs against the normal 5′ splicing construct (e.g., trans-splicing template) with twister was further assessed 24 h post transfection (FIG. 30). Editing rates of the new SMN motif construct 24 h post transduction with lipid nanoparticles is shown in FIG. 31. This data demonstrates that lipid nanoparticle delivery is compatible with the 5′ splicing constructs described herein.

TABLE 2
Exemplary sequences associated with the disclosure

| SEQ ID | | |
|---|---|---|
| NO: | Name | Sequence 5′ to 3′ |
| 9 | pDF1032 | agacccaagcttggtaccgagctcggatccgGCCACCATGGACTACAAAGACGATGACGACAAGggc |
| | HTT | ccggctgtggctgaggagccCctCcaccgaccGTGAGTttgggcTGCATGacTGCATGgcTGCATGaacaca |
| | twister | TATTAATtcctccacttagttctacacctcattcattcattcagtgagtgtttctcgactactatgaataaaccgttatactccatgttgc |
| | | gggcagaatggggatctggacagggaagcacagggcacgagttcaccaatggctgtcaagctacgctgcCCGCCTAACA |
| | | CTGCCAATGCCGGTGCCAAGCCCGGATAAAAAGTGGAGGGGGGGGgaattctgcagatatc |
| | | catcacact |
| 10 | instead_ | agacccaagcttggtaccgagctcggatccgGCCACCATGGACTACAAAGACGATGACGACAAGggc |
| | MALAT | ccggctgtggctgaggagccCctCcaccgaccGTGAGTttgggcTGCATGacTGCATGgtTGCATGaacaca |
| | 1 | TATTAATttcctccacttagttctacacctcattcattcattcagtgagtgtttctcgactactatgaataaaccgttatactccatcttgc |
| | | gggcagaatggggatctggacagggaagcacagggcacgagttcaccaatggctgtcaagctacgctgcgaaggtttttcttttcctg |
| | | agaaaacaacacgtattgttttctctggttttgctttttggcctttttctagcttaaaaaaaaaaaaagcaaaagaattctgcagatatccatc |
| | | acact |
| 11 | instead_ | agacccaagcttggtaccgagctcggatccgGCCACCATGGACTACAAAGACGATGACGACAAGggc |
| | MENB | ccggctgtggctgaggagccCcCCcaccgaccGTGAGTttgggcTGCATGacTGCATGgtTGCATGaacaca |
| | | TATTAATttcctccacttagttctacacctcattcattcattcagtgagtgtttctcgactactatgaataaaccgttatactccatgttgc |
| | | gggcagaatggggatctggacagggaagcacagggcacgagttcaccaatggctgtcaagctacgctgcAGGTGTTTCTT |
| | | TTACTGAGTGCAGCCCATGGCCGCACTCAGGTTTTGCTTTTCACCTTCCCATCTGT |
| | | GAAAGAGTGAGCAGGAAAAAGCAAAAgaattctgcagatatccatcacact |
| 12 | instead_ | agacccaagcttagtaccgagctcggatccgGCCACCATGGACTACAAAGACGATGACGACAAGggc |
| | Comp14 | ccggctgtggctgaggagccCctCcaccgaccGTGAGTtttgggcTGCATGacTGCATGgtTGCATGaacaca |
| | | TATTAATttcctccacttagttctacacctcattcattcattcagtgagtgtttctcgactactatgaaaacccttatactccatgttgc |
| | | gggcagaatggggatctggacagggaagcacagggcacgagttcaccaatggctgtcaagctgctgcAAAGGTTTTT |
| | | CTTTTCCTGAGAAATTTCTCAGGTTTTGCTTTTAAAAAAAAAGCAAAAgaattctgcagat |
| | | atccatcacact |
| 13 | front_ | agacccaagcttggtaccgagctcggatccgGCCACCATGGACTACAAAGACGATGACGACAAGggc |
| | MALAT1 | ccggctgtggctgaggagccCctCcaccgaccGTGAGTttgggcTGCATGacTGCATGgCTGCATGaacaca |
| | | TATTAATttcctccacttagttctacacctcattcattcattcagtgagtgtttctcgactactatgaataaaccgttatactccatgttgc |
| | | gggcagaatggggatctggacagggaagcacagggcacgagttcaccaatggctgtcaagctacgctgcgaaggtttttcttttcctg |
| | | agaaaacaacacgtattgttttctcaggttttgctttttggcctttttctagcttaaaaaaaaaaaaagcaaaaCCGCCTAACACT |
| | | GCCAATGCCGGTCCCAAGCCCGGATAAAAAGTGGAGGGGGCGGgaattctgcagatatccat |
| | | cacact |
| 14 | front_ | agacccaagcttggtaccgagctcggatccgGCCACCATGGACTACAAAGACGATGACGACAAGggc |
| | MENB | ccggctgtggctgaggagccCctCcaccgaccGTGAGTttgggcTGCATGacTGCATGgCTGCATGaacaca |
| | | TATTAATtcctccacttagttctacacctcattattcagtgagtgtttctcgactactatgaataaaccgttatactccatgttgc |
| | | gggcagaatggggatctggacagcgaagcacagggcacgagttcaccaatggctgtcaagctagctgcAGGTGTTTCTT |
| | | TTACTGAGTGCAGCCCATGGCCGCACTCAGGTTTTGCTTTTCACCTTGCCATCTGT |
| | | GAAAGAGTGAGCAGGAAAAAGCAAAACCGCCTAACACTGCCAATGCCGGTCCCA |
| | | AGCCGGGATAAAAAGTGGAGGGGGCGGgaattctccagatatccatcacact |
| 15 | front_ | agacccaagcttggtaccgagctcgatccgGCCACCATGGACTACAAAGACGATGACGACAAGggc |
| | Comp14 | ccggctgtggctgaggagccCctCcaccgaccGTGAGTttgggcTGCATGacTGCATGgCTGCATGaacaca |
| | | TATTAATttcctccacttagttctacacctcattcattcattcagtgagtgtttctcgactactatgaataaacccttatactccatgttgc |
| | | gggcagaatggggatctggacagggaagcacagggcacgagttcaccaatggctgtcaagctacgctgcAAAGGTTTTT |
| | | CTTTTCCTGAGAAATTTCTCAGGTTTTGCTTTTAAAAAAAAAGCAAAACCGGCTAA |
| | | CACTGCCAATGCCGGTCCCAAGCCCGGATAAAAAGTGGAGGGGGGGGgaattctgcaga |
| | | tatccatcacact |
| 16 | back_ | agacccaagcttggtaccgagctcggatccgGCCACCATGGACTACAAAGACGATGACGACAAGggc |
| | MALAT1 | ccggctgtggctgaggagccCctCcaccgaccGTGAGTttgggcTGCATGacTGCATGgtTGCATGaacaca |
| | | TATTAATtcctccacagttctacacctcattcattcattcagtgagtgtttctcgactactatgaataaacccttatactccatgttgc |
| | | gggcagaatggggatctggacagggaagcacagggcacgagttcaccaatggctgtcaagctacgctgcCCGCCTAACA |
| | | CTGCCAATGCCGGTCCCAAGCCCGGATAAAAAGTGGAGGGGGGGGgaaggtttttcttttcct |
| | | gagaaaacaacacgtattgttttctcaggttttgctttttggcctttttctagcttaaaaaaaaaaaaagcaaaagaattctgcagatatccat |
| | | cacact |
| 17 | back_ | agacccaagcttggtaccgagctcggatccgGCCACCATGGACTACAAAGACGATGACGACAAGggc |
| | MENB | ccggctgtggctgaggagccCctCcaccgaccGTGAGTttgggcTGCATGacTGCATGgTTGCATGaacaca |
| | | TATTAATttcctccacttagttctacacctcattcattcattcagtgagtgtttctcgactactatgaataaaccgttatactccatgttgc |
| | | gggcagaatggggatctggacagggaagcacagggcacgagttcaccaatggctgtcaagctacgctgcCCGCCTAACA |
| | | CTGCCAATGCCGGTCCCAAGCCCGGATAAAAAGTGGAGGGGGGGGAGGTGTTTCT |
| | | TTTACTGAGTGCAGCCCATGGCCGCACTCAGGTTTTGCTTTTCACCTTCCCATCTG |
| | | TGAAAGAGTGAGCAGGAAAAAGCAAAA |
| 18 | back_ | agacccaagcttagtaccgagctcggatcccGCCACCATGGACTACAAAGACGATGACGACAAGggc |
| | Comp14 | ccggctgtagctgaggagccCctCcaccgaccGTGAGTttgggcTGCATGacTGCATGgCTGCATGaacaca |
| | | TATTAATttcctccacttagttctacacctcattcattcattcagtgagtgtttctcgactactatgaataaaccgttatactccatgttgc |
| | | gggcagaatggggatctggacagggaagcacagggcacgagttcaccaatggctgtcaagctacgctgcCCGCCTAACA |
| | | CTGCCAATGCCGGTCCCAAGCCCGGATAAAAAGTGGAGGGGGCGGAAAGGTTTT |
| | | TCTTTTCCTGAGAAATTTCTCAGGTTTTGCTTTTAAAAAAAAAGCAAAAgaattctgcag |
| | | atatccatcacact |
| 19 | Hairpin 1 | agacccaagcttggtaccgagctcggatccgGCCACCATGGACTACAAAGACGATGACGACAAGggc |
| | | ccggctgtggctgaggagccCctCcaccgaccGTGAGTttgggcTGCATGaTGCATGgTTGCATGaacaca |
| | | TATTAATttcctccacttagttctacacctcattcattcattcagtgagtgtttctcgactactatgaataaaccgttatactccatgttgc |
| | | gggcagaatggggatctggacagggaagcacagggcacgagttcaccaatggctgtcaagctacgctgcAAACAGAGAA |
| | | GTCAACCAGAGAAACACACGTTGTGGTATATTACCTGGTAgaattctggatatccatcacact |
| 20 | Hairpin 2 | agacccaagcttggtaccgagctcggatcccGCCACCATGGACTACAAAGACGATGACGACAAGggc |
| | | ccggctgtagctgaggagccCctCcaccgaccGTGAGTttgggcTGCATGacTGCATGgTTGCATGaacaca |
| | | TATTAATttcctccacttagttctacacctcattcattcattcagtgagtgtttctcgactactatgaataaaccgttatactccatgttgc |
| | | gggcagaatggggatctggacagggaagcacagggcacgagttcaccaatggctgtcaagctacgctgcCAACAGCGAA |
| | | GCGCGCCAGGGAAACACACCATGTGTGGTATATTATCTGGCAgaattctgcagatatccatcaca |
| 21 | Hairpin 3 | agacccaagcttggtaccgagctcggatccgGCCACCATGGACTACAAAGACGATGACGACAAGggc |
| | | ccggctgtggctgaggagccCcCcaccgaccGTGAGTttgggcTGCATGacTGCATGgCTGCATGaacaca |
| | | TATTAATttcctccacttagttctacacctcattcattcattcagtgagtgtttctcgactactatgaataaaccgttatactccatgttgc |
| | | gcgcagaatggggatctggacagggaagcacagggcacgagttcaccaatggctgtcaagctacgctgcCAACAGCGAA |
| | | GCGGAACGGCGAAACACACCTTGTGTGGTATATTACCCGTTGgaattctgcagatccatcaca |
| 22 | Varkud | agacccaagcttggtaccgagctcggatccgGCCACCATGGACTACAAAGACGATGACGACAAGggc |
| | Satellite | ccggctgtggctgaggagccCcCCcaccgaccGTGAGTttgggcTGCATGacTGCATGgCTGCATGaacaca |
| | | TATTAAT&tcctccacttagttctacacctcattcatcattcagtgagtgtttctcgactactatgaataaacccttatactccatgttgc |
| | | gggcagaatggggatctggacagggaagcacagggcacgagttcaccaatggctgtcaagctacgctgcGGGAAAGCTT |
| | | GCGAAGGGCGTCGTCGCCCCGAGCGGTAGTAAGCAGGGAACTCACCTCCAATTTC |
| | | AGTACTGAAATTGTCGTAGCAGTTGACTACTGTTATGTGATTGGTAGAGGCTAAGT |
| | | GACGGTATTGGCGTAAGTCAGTATTGCAGCACAGCACAAGCCCGCTTGCGAGAAT |
| | | gaattctgcagatatccatcacact |
| 23 | glms | agacccaagcttggtaccgagctcggatccgGCCACCATGGACTACAAAGACGATGACGACAAGggC |
| | | ccggctgtggctgaggagccCctCcaccgaccGTGAGTttgggcTGCATCacTGCATGgtTGCATGaacaca |
| | | TATTAATttcctccacttagttctacacctcattcatttcattcagtgagtgttttctcgactactatgaataaaccgttatactccatgttgc |
| | | gggcagaatggggatctggacagggaagcacagggcacgagttcaccaatggctgtcaagctacgctgcTAATTATAGCG |
| | | CCCGAACTAAGCGCGCGGAAAAAGGCTTAGTTGACGAGGATGGAGGTTATCGAAT |
| | | TTTCGGGGGGATGCCTCCCGGCTGAGTGTGCAGATCACAGCCGTAAGGATTTCTTC |
| | | AAACCAAGGGGGTGACTCCTTGAACAAAGAGAAATCACATGATCTgaattctgcagatatc |
| | | catcacact |
| 24 | twister | agacccaagcttggtaccgagctcggatccgGCCACCATGGACTACAAAGACGATGACGACAAGggc |
| | sister | ccggctgtggctgaggagccCctCcaccgaccGTGAGTttgggcTGCATGacTGCATGgtTGCATGaacaca |
| | | TATTAATttcctccacttagttctacacctcattcattcattcagtgagtgtttctcgactactatgaataaaccgttatactccatgttgc |
| | | gggcagaatggggatctggacagggaagcacagggcacgagttcaccaatggctgtcaagctacgctgcGGACCCGCAA |
| | | GGCCGACGGCATCCGCCGCCGCTGGTGCAAGTCCAGCCGCCCCGGGGGGGGCGC |
| | | TCATGGGTAAACgaattctgcagatatccatcacact |
| 25 | twister | agacccaagcttggtaccgagctcggatccgGCCACCATGGACTACAAAGACGATGACGACAAGggc |
| | sister AT | ccggctgtggctgaggagccCctCcaccgaccGTGAGTttgggcTGCATGacTGCATGgtTGCATGaacaca |
| | insert | TATTAATttcctccacttagttctacacctcattcattcattcagtgagtgtttctcgactactatgaataaaccgttatactccatgttgc |
| | | gcgcagaatggggatctggacagggaagcacagggcacgagttcaccaatggctgtcaagctacgctgcGGACCCGCAA |
| | | GGCCGACGGCATCCGCCGCCGCTGGTGCAAGTCCAGCCGCCCCATGGGGGGGGC |
| | | GCTCATGGGTAAACgaattctgcagatatccatcacact |
| 26 | pistol | agacccaagcttcgtaccgagctcggGCCACCATGGACTACAAAGACGATGACGACAAGggc |
| | | ccggctgtggctgaggagccCctCcaccgaccGTGAGTttgggcTGCATGacTGCATGgCTGCATGaacaca |
| | | TATTAATttcctccacttagttctacacctcattcattcattcagtgagtgtttctcgactactatgaataaaccgttatactccatgttgc |
| | | gcgcagaatggggatctggacagggaagcacagggcacgagttcaccaatggctgtcaagctacgctgcGGAGCCGTTC |
| | | GGGCGGCTATAAACAGACCTCAGGCCCGAAGCGTGGCGGCGATCCGCCGGTGGT |
| | | Agaattctgcagatatccatcacact |
| 27 | hatcher | agacccaagcttcgtaccgagctcggatccgGCCACCATGGACTACAAAGACGATGACGACAAGggc |
| | | ccggctgtggctgaggagccCctCcaccgaccGTGAGTttgggcTGCATGacTGCATGgTTGCATGaacaca |
| | | TATTAATttcctccacttagttctacacctcattcattcattcagtgagtgtttctcgactactatgaataaaccgttatactccatgttgc |
| | | gcgcagaatggggatctggacagggaagcacagggcacgagttcaccaatggctgtcaagctacgctgcCATTCCTCAG |
| | | AAAATGACAAACCTGTGGGGCGTAAGTAGATATGTAGATATCTATGATCGTGCAGA |
| | | CGTTAAAATCAGGTgaattctgcagatatccatcacact |
| 28 | JSG1 | agacccaagcttggtaccgagctcggatccgGCCACCATGGACTACAAAGACGATGACGACAAGggc |
| | (synthetic | ccggctgtggctgaggagccCcCcaccgaccGTGAGTttgggcTGCATGacTGCATGgCTGCATGaacaca |
| | terminator) | TATTAATttcctccacttagttctacacctcattcattcattcagtgagtgtttctcgactactatgaataaaccgttatactccatgttgc |
| | | gggcagaatggggatctggacagcagggcacgagttcaccaatggctgtcaagctacgctgcCCGCCTAACA |
| | | CTGCCAATGCCGGTCCCAAGCCCGGATAAAAAGTGGAGGGGGGGGgtttctttttgggaatg |
| | | aaaaaaaaaaaagcaaaagaattctgcagatatcc |
| | | atcacact |
| 29 | JSG2 | agacccaagcttggtaccgagctcggatccgGCCACCATGGACTACAAAGACGATGACGACAAGggc |
| | (synthetic | ccggctgtggctgaggagccCctCcaccgaccGTGAGTttgggcTGCATGacTGCATGgtTGCATGaacaca |
| | terminator) | TATTAAT@cctccacttagttctacacctcattcattcattcagtgagtgtttctcgactactatgaataaaccgttatactccatgttgc |
| | | gggcagaatggggatctggacagggaagcacagggcacgagttcaccaatggctgtcaagctacgctgcCCGCCTAACA |
| | | CTGCCAATGCCGGTCCCAAGCCCGGATAAAAAGTGGAGGGGGCGGgtttctttttgggaatg |
| | | catcacact |
| 30 | JSG3 | agacccaagcttggtaccgagctcggatccgGCCACCATGGACTACAAAGACGATGACGACAAGggc |
| | (synthetic | ccggctgtggctgaggagccCctCcaccgaccGTGAGTttgggcTGCATGacTGCATGgTTGCATGaacaca |
| | terminator) | TATTAATttcctccacttagttctacacctcattcattcattcagtgagtgtttctcgactactatgaataaaccgttatactccatgttgc |
| | | gggcagaatggggatctggacagggaagcacagggcacgagttcaccaatggctgtcaagctacgctgcCCGCCTAACA |
| | | CTGCCAATGCCGGTGCCAAGCCCGGATAAAAAGTGGAGGGGGCGGgtttctttttgggaatg |
| | | gactcttttgttcacgtaaacaaaagactttttgctttttgcgctttttctcgcttaaaaacaaaga;ttctgcagatatc |
| | | catcacact |
| 31 | TW1FB1_ | agacccaagcttcgtaccgagctcggatccgGCCACCATGGACTACAAAGACGATGACGACAAGggc |
| | Osa | ccggctgtggctgaggagccCctCcaccgaccGTGAGTttgggcTGCATGacTGCATGgCTGCATGaacaca |
| | | TATTAATttcctccacttagttctacacctcattcattcattcagtgagtgtttctcgactactatgaataaaccgttatactccatgttgc |
| | | gggcagaatggggatctggacagggaagcacagggcacgagttcaccaatggctgtcaagctacgctgcCCGCCTAACA |
| | | CTGCCAATGCCGGTCCCAAGCCCGGATAAAAAGTGGAGGGGGCGGTTCTCAAATA |
| | | TGAGTTTTTACATGACAAAGTTTTTAACGAGGCAGCgaattctgcagatatccatcacact |
| 32 | RRV | agacccgcttggtacctcctcggatccgGCCACCATGGACTACAAAGACGATGACGACAAGggc |
| | | ccggctgtygctgaggccCctCcaccgaccGTGAGTttgggcTGCATGacTGCATGgCTGCATGaacaca |
| | | TATTAATatcctccacttagttctacacctcattcattcattcagagtgtttctcgactactatgaataaacccttatactccatgttgc |
| | | gggcagaatggggatctggacagggaagcacaggagttcaccaatggctgtcaagctacgctgcCCGCCTAACA |
| | | CTGCCAATGCCGGTCCCAAGCCCGGATAAAAAGTGGAGGGGGGGGGGTACAAAA |
| | | CCTGCTGGTGATTTTTTACCCAACAA |
| 33 | EHV2 | agacccaagcttggtaccgagctcggatcGCACCATGGACTACAAAGACGATGACGACAAGggc |
| | | ccggctgtggctgaggagccCctCcaccgaccGTGAGTttgggcTGCATGacTGCATGgtTGCATGaacaca |
| | | TATTAATttcctccacttagttctacacctcattcattcattcagtgagtgtttctcgactactatgaataaaccgttatactccatgttgc |
| | | gggcagaatgcggatctggacagcgaagcacagttcaccaatggctgtcaagctacCTAACA |
| | | CTGCCAATGCCGGTCCCAAGCCCGGATAAAAAGTGGAGGGGGGGGTTTCCCCAAC |
| | | CTCTGGGTTGGGTTTTTTCTCTTTAAAATATTCAATAAAAgaattctgcagatatccatcacact |
| 34 | TCUP_ | agacccaagcttggtaccgagctcggatccgGCCACCATGGACTACAAAGACGATGACGACAAGggc |
| | Zma | ccggctgtggctgaggagccCctCcaccgaccGTGAGTttgggcTGCATGacTGCATGgTTGCATGaacaca |
| | | TATTAATttccctcacattcagtgagtgtttctcgactactatgaataaacccttatactccatgttgc |
| | | gggcagaatggggatctggacagggaagcacagggcacgagttcaccaatggctgtcaagctagctgcCCGCCTAACA |
| | | CTGCCAATGCCGGTGCGACGATAAAAAGTGGAGGGGGGGGTTCTCACAA |
| | | GGGTGAGTTTTACCTAGACAGGTTTTTAACCAGGCAACCgaattctgcagatatccatcacact |
| 35 | HOXD13_ | agacccaagcttggtaccagctcgGCCACCATGGACTACAAAGACGATGACGACAAGagg |
| | 1 | cctacatctccatggaggtcgctggctaacgggtggaacagccaggtgtactgcaccaaggaccagccacag |
| | | gcgtcccacttttggaaatcctctttcccagGTGAGTttgggcTGCATGacTGCATGgtTGCATGaacacaTATT |
| | | AATttccTAACAACAATGGTTAATTTGATTGATTATCATAGTTTCTCAAACCGGCATCT |
| | | TAACAAGCTTGCAATTTTATGGTGTGGTTATTGGGCACGTTCATGAGGACAGTTTC |
| | | TGCAAATGTCGGTTGTATAAAAACAAACTTACATACATAGACCCGCCTAACACTGC |
| | | CAATGCCGGTCCCAAGCCCGGATAAAAAGTGGAGGGGGCGGgaattctgcagatatccatcac |
| | | act |
| 36 | HOXD13_ | agacccaagcttggtaccgagctcggatccgGCCACCATGGACTACAAAGACGATGACGACAAGagg |
| | 2 | cctacatctccatggaggggtaccagtcctggacgctggcmacgggggaacagccagptgtactgcaccaaggaccagccacag |
| | | gggtcccacttttggaaatcctctttcccagGTGAGTttgggcTGCATGacTGCATGgtTGCATGaacacaTATT |
| | | AATaccCTTAACAAGCTTGCAATTTTATGGTGTGGTTATTGGGCACGTTCATGAGGA |
| | | CAGTTTCTGCAAATGTCGGTTGTATAAAAACAAACTTACATACATAGACACATACC |
| | | AACTCAGGTCTTTTGCTCAAATTTTGATTTCCCGCGTTCCTATCCGCCTAACACTGC |
| | | CAATGCCGGTGCCAAGCCCGGATAAAAAGTGGAGGGGGGGGgaattctgcagatatccatcac |
| | | act |
| 37 | HOXD13_ | agacccaagcttggtaccgagctcggatcccGCCACCATGGACTACAAAGACGATGACGACAAGagg |
| | 3 | cctacatctccatggaggggtaccagtcctggacgctggctaacgggtggaacagccaggtgtactgcaccaaggttccagccacag |
| | | gggtcccacttttggaaatcctctttcccagGTGAGTttgggcTGCATGacTGCATGgtTGCATGaacacaTATT |
| | | AATHccCAGGGGTGCAGGAAATTTTTAAAAAATGCCATGCTGTTTGGTCTTTATTAT |
| | | GTACATAGACATAACACTTACCTGTTATTAAAGAAGGATTATTTGCACTTAACAACA |
| | | ATGGTTAATTTGATTGATTATCATAGTTTCTCAAACCGGCATGCGCCTAACACTGCC |
| | | AATGCCGGTCCCAAGCCCGGATAAAAAGTGGAGGGGGGGGgaattctgcagatatccatcacac |
| | | t |
| 38 | HOXD13_ | agacccaagcttggtaccgagctcggatccgGCCACCATGGACTACAAAGACGATGACGACAAGagg |
| | 4 | cctacatctccatggaggggtaccagtcctggacgctggctaacgggtggaacagccaggtgtactgcaccaaggaccagccacag |
| | | gggtcccacttttggaaatcctctttcccagGTGAGTttgggcTGCATGacTGCATGgtTGCATGaacacaTATT |
| | | AATttccTGTACATAGACATAACACTTACCTGTTATTAAAGAAGGATTATTTGCACTTA |
| | | ACAACAATGGTTAATTTGATTGATTATCATAGTTTCTCAAACCGGCATCTTAACAAG |
| | | CTTGCAATTTTATGGTGTGGTTATTGGGCACGTTCATGAGGCCGCCTAACACTGCC |
| | | AATGCCGGTCCCAAGCCCGGATAAAAAGTGGAGGGGGCGGgaattctgcagatatccatcacac |
| 39 | HOXD13_ | agacccaagcttcgtaccgagctcggatccgGCCACCATGGACTACAAAGACGATGACGACAAGagg |
| | 5 | cctacatctccatggaggggtaccagtcctggacgctggctaacgcgtggaacagccaggtgtactgcaccaaggaccagccacag |
| | | gggtcccacttttggaaatcctctttcccagGTGAGTttgggcTGCATGacTGCATGgtTGCATGaacacaTATT |
| | | AATttccTTGACCAAGTGGAGGGTGCTCTTCCAGCTCTTGAACAGGACCTAGAGAGT |
| | | TGGATGTATTAGATGGGCGTACGCAgTATGTGCCCAGTTGTATGATTGTGGGTTTTC |
| | | AAGGAAGGGAGTGTGCGTCGATTCGTTCAGTATCGACAgGGGGCCGCCTAACACT |
| | | GCCAATGCCGGTCCCAAGCCCGGATAAAAAGTGGAGGGGGGGGgaattctgcagatatccat |
| | | cacact |
| 40 | HOXD13 | agacccaagcttggtaccgctcggatccgGCCACCATGGACTACAAAGACGATGACGACAAGgtg |
| | EGFP | agcaagggcgaggaccggggtggtgcccatcctggtcgagctggacggcgacgtaaacggccacaagttcagcgtgt |
| | insert | ccggcttgccacctacggcaagctgaccctgaagttcatctgcaccaccggcaagctgcccgtgccctggcc |
| | | caccctcgtgaccaccctgacctacggcgtgcagtgcttcagccgctaccccgaccacatgaagcagcacgacttcttcaagtccgcc |
| | | atgcccgaaggctacgtccaggagcgcaccatcttcttcaaggacgacggcaactacaagacccgcgccgaggtgaagttcgagg |
| | | gcgacaccctggtgaaccgcatcgagctgaagggcatcgacttcaaggaggacggcaacatcctggggcacaagctggagtacaa |
| | | ctacaacagccacaacgtctatatcatggccgacaagcagaagaacggcatcaaggtgaacttcaagatccgccacaacatcgagg |
| | | acggcagcgtgcagctcgccgaccactaccagcagaacaccccatcggcgacggccccgtgctgctgcccgacaaccactacct |
| | | gagcacccagtccgccctgagcaaagaccccaacgagaagcgcgatcacatggtcctgctggagttcgtgaccgccgcccggatc |
| | | actcctcggcatggacgagctgtacaagaggcctacatctccatggaggggtaccagtcctggacgctggctaacgggtcgaacagc |
| | | caggtgtactgcacccaggggtcccacttttggaaatcctctttcccagGTGAGTagggcTGCATGac |
| | | TGCATGgtTGCATGaacacaTATTAATttccCAGGGGTGCAGGAAATTTTTAAAAAATGCC |
| | | ATGCTGTTTGGTCTTTATTATGTACATAGACATAACACTTACCTGTTATTAAAGAAG |
| | | GATTATTTGCACTTAACAACAATGGTTAATTTGATTGATTATCATAGTTTCTCAAACC |
| | | GGCATCCGCCTAACACTGCCAATGCCGGTCCCAAGCCCGGATAAAAAGTGGAGGG |
| | | GGCGGgaattctgtccatcacact |
| 41 | HOXD13 | agacccaagcttcgtaccgagctcggatccgGCCACCATGGACTACAAAGACGATGACGACAAGaat |
| | Cre | tactgaccgtacaccaaaatttgcctgcattaccggtcgtttgcaacgagtgatgaggttcgcaagaacctgatggacatgttcagggat |
| | insert | cgccaggcgttttctgagcatacctggaaaatgcttctgtccgtttgccggtcgtgggggcatggtgcaagttgaataaccggaaatg |
| | | gttttcccgcagaacctgaagatgttcgcgattatcttctatatcttcaggcgcgcggtctggcagtaaaaactatccagcaacatttgggc |
| | | cagctaaacatgcttcatcgtcggtccgggctgccacgaccaagtgacagcaatgtgtttcactggttatgcggcggatccgaaaag |
| | | aaaacgttgaacgcaaaacaggctctagcgttcgaacgcactgatttcgaccaggttcgttcactcatggaaaatagc |
| | | gatcgctgccaggatatacgtaatctggcatttctggggattgcttataacaccctgttacgtatagccgaaattgccaggatcagggtta |
| | | aagatatctcacgtactgacgggggagaatgttaatccatattggcagaacgaaaacgctggttagcaccgcaggtgtagagaaggc |
| | | acttagcctgggggtaactaaactggtcgagcgatggatttccgtctctggtgtagctgatgatccgaataactacctgttttgccgggtc |
| | | agaaaaaatggtgttgccgcgccatctgccaccagccagctatcaactcgcgccctggaagggatttttgaagcaactcatcgattgat |
| | | tacggcgctaaggatgactctggtcagagatacctggcctggtctggacacagtgcccgtgtcggagccgcgcgagatatggcccg |
| | | cgctggagtttcaataccggagatcatgtaagctggggctggaccaatgtaaatattgtcatgaactatatccgtaacctggatagtga |
| | | aacaggggcaatggtgcgcctgctggaagatggcgataggcctacatctccatggaggggtaccagtcctggacgctggctaacgg |
| | | gtggaacagccaggtgtactgcaccaaggaccagccacaggggtcccacttttggaaatcctctttcccagGTGAGTttgggcT |
| | | GCATGacTGCATGgTTGCATGaacacaTATTAATttccCAGGGGTGCAGGAAATTTTTAAAA |
| | | AATGCCATGCTGTTTGGTCTTTATTATGTACATAGACATAACACTTACCTGTTATTAA |
| | | AGAAGGATTATTTGCACTTAACAACAATGGTTAATTTGATTGATTATCATAGTTTCT |
| | | CAAACCGGCATCCGCCTAACACTGCCAATGCCGGTCCCAAGCCCGGATAAAAAGT |
| | | GGAGGGGGGGGgaattctgcagatatccatcacact |
| 42 | SOD1_1 | agacccaagcttggtaccgagctcggatccgCCCACCATGGACTACAAAGACGATGACGACAAGatg |
| | | gcgacgaaggccptgtgcgtgctgaagggcgacggcccagtgcagggcatcatcaatttTGAACAAAAGGTGAGTE |
| | | gggcTGCATGacTGCATGgtTGCATGaacacaTATTAATttccAGAGTTTCTGAACAAAGAA |
| | | AACAGCTGATTTACCTTTACCCCTCACAGTGAAGTAAAAAGCAGCCAGACTTGGG |
| | | AGTGGAAAAATCTGTGTCCAAATCCCAGGCTGACCACTTTCTAGCCAGTGAACCT |
| | | CCAGCAAGCTGCTTAACTGCTCCGCCTAACACTGCCAATGCCGGTCCCAAGCGCG |
| | | GATAAAAAGTGGAGGGGGGGGgaattctgcagatatccatcacact |
| 43 | SOD1_2 | agacccaagcttggtaccgagctcggatcccGCCACCATGGACTACAAAGACGATGACGACAAGatg |
| | | gcgacgaaggccgtgtgcgtgctgaagggcgacggcccagtgcagggcatcatcaattTGAACAAAAGGTGAGTtt |
| | | gggcTGCATGacTGCATGgCTGCATGaacacaTATTAATttccCTAGGCCTGTGTCCTCAAAA |
| | | GGGAGATGGTAATCTTGTTCCCACACTCAATGCACAAGCACCCTGTGGCCAGGCT |
| | | TCATGATTAAATAACATAAAAAGCACCTAATAGAGTGGCAAGCAAAGTGAGTACCT |
| | | ACACATCAGTGTTAGATACCCGCCTAACACTGCCAATGCCGGTCCCAAGCCGGGAT |
| | | AAAAAGTGGAGGGGGGGGgaattctgcagatatccatcacact |
| 44 | SOD1_3 | agacccaagcttagtaccgagctcggatccgGCCACCATGGACTACAAAGACGATGACGACAAGatg |
| | | gcgacgaaggccgtgtgcgtgctgttagggcgacggcccagtgcagggcatcatcaattTGAACAAAAGGTGAGTH |
| | | gggcTGCATGacTGCATGgTGCATGaacacaTATTAATtccTTCATAGGCTTTGACTATTAC |
| | | AGTAAATAGGTAATGTAAGTACCTATTTATGTAGTAAATACTTTTGAATAGGTTATAT |
| | | TTGTACATGTTGAATATACATGTACATGTAAACGTTTTTTATTTTGAGACAAGAGTC |
| | | TCGCTCTGTCGCCCCCGCCTAACACTGCCAATGCCGGTCCCAAGCCCGGATAAAA |
| | | AGTGGAGGGGGGGGgaattctccagatatccatcacact |
| 45 | SOD1_4 | agacccaagcttcgtaccgagctcggatccgGCCACCATGGACTACAAAGACGATGACGACAAGag |
| | | gcgacgaaggccgtgtgcgtgctgaagggcgacggcccagtgcagggcatcatcaattTGAACAAAAGGTGAGTH |
| | | gggcTGCATGacTGCATGgtTGCATGaacacaTATTAATttccAGGCTGGAGTGCAGTGGCAT |
| | | GATCTCAGCTCACTGCAAGCTCCGCCTCCCGGGTTCACGCCATTCTCCTGCCTCAG |
| | | CCTTTTTAACCCAAAAGGTGTATGTCTCATCCTACACTCTGGAGCCCCTTGCTTTTT |
| | | CTCCACGCACACCCTTTCCGCCTAACACTGCCAATGCCGGTCCCAAGCCCGGATA |
| | | AAAAGTGGAGGGGGGGGgaattctgcagatatccatcacact |
| 46 | SOD1_5 | agacccaagcttggtaccgagctcggatccgCCCACCATGGACTACAAAGACGATGACGACAAGatg |
| | | gcgacgaaggccgtgtgcgtgctgaagggcgacggcccagtgcagggcatcatcaattTGAACAAAAGGTGAGTU |
| | | cggCTGCATGacTGCATGgtTGCATGaacacaTATTAATttccAGTGGAAAAATGTGTGTCCA |
| | | AATCCCAGGCTGACCACTTTCTAGCCAGTGAACCTCCAGCAAGCTGCTTAACTGC |
| | | TCTAGGCCTGTGTCCTCAAAAGGGAGATGGTAATCTTGTTCCCACACTCAATGCAC |
| | | AAGCACCCTGTGGCCAGGCCCGCCTAACACTGCCAATGCCGGTCCCAAGCCCGGA |
| | | TAAAAAGTGGAGGGGGGGGgaattctgcagatatccatcacact |
| 47 | KCNQ1_ | agacccaagcttggtaccgagctcggatccgGCCACCATGGACTACAAAGACGATGACGACAAGcgc |
| | 1 | gtctccatctacagcacgcgccgcccggtgttggcgcgcacccacgtccagggccgcgtctttcaacttcctcgagcgtcccaccggc. |
| | | tggaaatgcttcgtttaccATTTTGCAGTGTGAGTtgggcTGCATGacTGCATGgCTGCATGaacacaT |
| | | ATTAATttccGCATCACGGGCTCTGCCCATCCCACAGATGCACCTGGGGAGTGGAGG |
| | | GGCCCCTGGCCTCAGCCCCGCCCCCAGGTAGGAACAGACAGTGCTTCGAAAACC |
| | | CAGTCATCCATTAGCTGGTAAGCACACACTAGAGGTAGGTGCGCACGAGCCGCCT |
| | | AACACTGCCAATGCCGGTCCCAAGCCGGGATAAAAAGTGGAGGGGGGGGgaattctgc |
| | | agatatccatcacact |
| 48 | KCNQ1_ | agacccaagcttggtaccgagctcggatccgGCCACCATGGACTACAAAGACGATGACGACAAGcgc |
| | 2 | gtctccatctacagcacgcgccgcccgctgttggcgcgcacccacgtccagggccgcgtctacactcttcctcgagcgtcccaccggc |
| | | tggaaatgcttcgtttaccATTTTGCAGTGTGAGTttgggcTGCATGacTGCATGgCTGCATGaacacaT |
| | | ATTAATttccGGATGCCGTCCCGCCACGGGAAGGGACGAAGCACCAACACATGCTGC |
| | | CACGTGGATGGGTCCGAGAAACATGGCGCCAAGGCCAGACACAGACAGGTGACC |
| | | ACCTGCTGACCACCTGCAGTGTGGCTACGCCCTGGACAGCCAGAGCCACCCGCCT |
| | | AACACTGCCAATGCCGGTCCCAAGCCCGGATAAAAAGTGGAGGGGGGGGgaattctgc |
| | | agatatccatcacact |
| 49 | KCNQ1_ | agacccaagcttggtaccgagctcggatccgGCCACCATGGACTACAAAGACGATGACGACAAGcgc |
| | 3 | gtctccatctacagcacgcgccgcccggtgttggcgcgcacccacgtccagggccgcgtctacaacttcctcgagcgtcccaccggc |
| | | tggaaatgcttcgtttaccATTTTGCAGTGTGAGTttgggcTGCATGacTGCATGgtTGCATGaacacaT |
| | | ATTAATTCCAGAAACAGAAAGCAGGCTGGTGGCTGCCCCAGGGCGGGACTGCTGT |
| | | GGGCGTGGGGTTTTCTTTTGGGGTAATTAAACGTTCTGGATGTGGATGGTGGTGGT |
| | | GGCTACACAACTCTGAACATTCTAGAGCCACTTAGGGCCCTAGAGCCACCGCCTA |
| | | ACACTGCCAATGCCGGTCCCAAGCCCGGATAAAAAGTGGAGGGGGCGGgaattctgca |
| | | gatatccatcacact |
| 50 | KCNQ1_ | agacccaagcttggtaccgagctcggatccgGCCACCATGGACTACAAAGACGATGACGACAAGcgc |
| | 4 | gtctccatctacagcacgcgccgcccggtgttggcgcgcacccacgtccagggccgcgtctacaacttcctcgagcgtcccaccggc |
| | | @ggaaatgcttcgtttaccATTTTGCAGTGTGAGTttgggcTGCATGacTGCATGgTTGCATGaacacaT |
| | | ATTAATttcCCAAATGAACTGCAGTGTGTAATTACGTCTCTACAAAGCTGATTTTACA |
| | | AATCACTTCCAGAGAGCAGTCAACGCCCCCTGTCCAGGAGTTCAGGGCCCAGGG |
| | | CAGGGGTGTTGGTGGTCACAGCACAGAAGCCAGCCCGCCAGGTGGGGGCCGCCT |
| | | AACACTGCCAATGCCGGTGCCAAGCCCGGATAAAAAGTGGAGGGGGCGGgaattctgc |
| | | agatatccatcacact |
| 51 | KCNQ1_ | agacccaagcttagtaccgagctcggatccgGCCACCATGGACTACAAAGACGATGACGACAAGcgc |
| | 5 | gtctccatctacagcacgcgccgcccggtgttggcgcgcacccacgtccagggccgcgtctacaacttcctcgagcgtcccaccggc |
| | | tcgaaatgcttcgtttaccATTTTGCAGTGTGAGTttgggcTGCATGacTGCATGgTTGCATGaacacaT |
| | | ATTAATttccTCCTGACACCTGCACCAAGCGTCTGCTTCCCAGAGGAGCCTCCAGGC |
| | | GGCAGGCTGTGGGCCGCTCTCCTCCAGTTTCACTTCCTCCTTTCCAAGGCCCCGA |
| | | GGCCGAGCCACAGAACTCATGAAAGGGAAGGCGCACAGAGCGCCACCCCCGCCT |
| | | AACACTGCCAATGCCGGTCCCAAGCCCGGATAAAAAGTGGAGGGGGGGGgaattctgc |
| | | agatatccatcacact |
| 52 | SPTBN2_ | agacccaagcttggtaccgagctcggatcgGCCACCATGGACTACAAAGACGATGACGACAAGacc |
| | 1 | aagtgggtaaactcgcacctggcccgggtcacgtgccggggggggacctctacagcgacctccgggacggacgcaacctgctga |
| | | ggctcctcgagctgctctcggcCCAAATTCTGGTGAGTagggcTGCATGacTGCATGgtTGCATGaac |
| | | acaTATTAATtccCTGTGGAGGGACGGGGGCAAAATGGCACGGGACTGTGGGCTTCC |
| | | ACCTTCTTCCCCAGGCTTCACAGGGCCCAGCTTTGCACACCTTCCCCATGACCTAC |
| | | CTCAGGAACACAGACAGGCACAGCCCCAGGGCTGGAGCCAAAGCTGAGCTCCGC |
| | | CTAACACTGCCAATGCCGGTCCCAAGCCCGGATAAAAAGTGGAGGGGGCGGgaattc |
| | | tgcagatatccatcacact |
| 53 | SPTBN2_ | agacccaagcttggtaccgagctcggatccgGCCACCATGGACTACAAAGACGATGACGACAAGacc |
| | 2 | aagtgggtaaactcgcacctggcccgggtcacgtgccggggggggacctgtacagcgacctccgggacggacgcaacctgctga |
| | | ggctcctcgaggtgctctcgggCGAAATTCTGGTGAGTttgggcTGCATGacTGCATGgTTGCATGaac |
| | | acaTATTAATttccTACAGATGAGGCTTGGGCAGATTAAGATTGCCAAGGGAATTAATT |
| | | GCACCCAGAGTAGGTGGGAAAAGAAATGTTTTCAATGGGGCCACTGTTCCTGTCT |
| | | CCATGAAGCAATGCTGCTATGTAATCTAAGTGGCAAAGGCACTTGAAATGCCGCCT |
| | | AACACTGCCAATGCCGGTCCCAAGCCCGGATAAAAAGTGGAGGGGGGGGgaattctgc |
| | | agatatccatcacact |
| 54 | SPTBN2_ | agacccaagcttggtaccgagctcggatccgGCCACCATGGACTACAAAGACGATGACGACAAGacc |
| | 3 | aagtgggtaaactcgcacctggcccgggtcacgtgccggggggggacctgtacagcgacctccgggacggacgcaacctgctga |
| | | ggctcctcgaggtcctctcgggCGAAATTCTGGTGAGTttgggcTGCATGacTGCATGgtTGCATGaac |
| | | acaTATTAATUCCAAAGGCACTTGAAATGAACCCATCCTCTGAGCAGAGGGAGCCAC |
| | | TGCTTCCCGCCCTGTGCCGTCACTCTCTCTGAGGGCTGTTCTTCCAGCTGGTCCCC |
| | | TTGGACACTTTTCTAAGGCCCCCCCACTTCCCTTCATGACCACAGCTCACCCGCCT |
| | | AACACTGCCAATGCCGGTGCCAAGCCCGGATAAAAAGTGGAGGGGGGGGgaattctgc |
| | | agatatccatcacact |
| 55 | SPTBN2_ | agacccaagcttggtaccgagctcggatccgGCCACCATGGACTACAAAGACGATGACGACAAGacc |
| | 4 | Bagtgggtaaactcgcacctggcccgggtcacctgggggacctgtacagcgacctccgagaccgacgcaacctgctga |
| | | ggctcctcgaggtgctctcgggCGAAATTCTGGTGAGTttgggcTGCATGacTGCATGgTTGCATGaac |
| | | acaTATTAATaccTTCCCCAGCCTTCACAGGGCCCAGCTTTGCACACCTTGCCCATGA |
| | | CCTACCTCAGGAACACAGACAGGCACAGCCCCAGGGCTGGAGCCAAAGCTGAGC |
| | | TTACAGATGAGGCTTGGGCAGATTAAGATTCCCAAGGGAATTAATTGCACCCCGCC |
| | | TAACACTGCCAATGCCGGTGCCAAGCCGGGATAAAAAGTGGAGGGGGCGGgaattctg |
| | | cagatatccatcacact |
| 56 | SPTBN2_ | agacccaagcttggtaccgagctcggatccgGCCACCATGGACTACAAAGACGATGACGACAAGacc |
| | 5 | aagtgggtaaactcgcacctggcccgggtcacgtgccggggggggacctgtacagcgacctccgggacggacgcaacctgctga |
| | | ggctcctcgaggtgctctcgggCGAAATTCTGGTGAGTttgggcTGCATGacTGCATGgtTGCATGaac |
| | | acaTATTAATttccCTCAGGAACACAGACAGGCACAGCCCCAGGGCTGGAGCCAAAG |
| | | CTGACCTTACAGATGAGGCTTGGGCAGATTAAGATTCCCAAGGGAATTAATTGCAC |
| | | CCAGAGTAGGTGGGAAAAGAAATGTTTTCAATGGGGCCACTGTTCCTGTCTCCGC |
| | | CTAACACTGCCAATGCCGGTCCCAAGCCCGGATAAAAAGTGGAGGGGGCGGgaattc |
| | | tgcagatatccatcacact |
| 57 | MEF2C_ | agacccaagcttggtaccgagctcggatccgGCCACCATGGACTACAAAGACGATGACGACAAGatg |
| | 1 | gcgagaaaaaagattcagattacgaggattatggatgaagCAATCGACAGGTGAGTttgggcTGCATGacTG |
| | | CATGgTTGCATGaacacaTATTAATTTccAAAACAATGGTGTGTAGTTGTCAGAGCCCTGG |
| | | GCACTAACACATTCAGAAAGAAAGCAAATAAATCTAATATTAATTTCTGCTTATCAT |
| | | TTGAAAATGTGTGATTTCTAGCTTGCTGCTGAATGTGAGACTCTAAATGCAAGATT |
| | | TAATGCCGCCTAACACTGCCAATGCCGGTCCCAAGCCCGGATAAAAAGTGGAGGG |
| | | GGCGGgaattctgctatccatcacact |
| 58 | MEF2C_ | agacccaagcttggtaccgagctcggatcccGCCACCATGGACTACAAAGACGATGACGACAAGatg |
| | 2 | gggagaaaaaagattcagattacgaggattatggatgaacgCAATCGACAGGTGAGTttgggcTGCATGacTG |
| | | CATGgtTGCATGaacacaTATTAATaccTAGGGTATTTTAATGAAGGTGACCAGGGAACT |
| | | GGTCATGTTTCTTTGAGCAAGTTAAGATTTAAAAAAAAATTAATTTCAGCATGGTT |
| | | AGCATATTTCCCAATTAATAATTAGTTTGGAGCTTGTTTTTGACTATTTGAAGAGTG |
| | | GACATCCGCCTAACACTGCCAATGCCGGTCCCAAGCCCGGATAAAAAGTGGAGGG |
| | | GGCGGgaattctgcagatatccatcacact |
| 59 | MEF2C_ | agacccaagcttggtaccgagctcggatccgGCCACCATGGACTACAAAGACGATGACGACAAGag |
| | 3 | gggagaaaaaagattcagattacgaggattatggatgaacgCAATCGACAGGTGAGTttgggcTGCATGacTG |
| | | CATGgtTGCATGaacacaTATTAATttccGACATTAGGAACTTAGCATTTTAGATCTGTGTT |
| | | CAAACAAGTCTTAAAATTGTCTAGTCTCATGTCATTTAATTGAAAACACTTGAAGG |
| | | GATATGACTGTGATCTCAGACGATGTTGTATTCTGTTTTCTAGAAACCCAACAGGG |
| | | CACTGCCGCCTAACACTGCCAATGCCGGTCCCAAGCCCGGATAAAAAGTGGAGGG |
| | | GGCGGgaattctgcagatatccatcacact |
| 60 | MER2C_ | agacccaagcttcgtaccgctcggatccgGCCACCATGGACTACAAAGACGATGACGACAAGag |
| | 4 | gggagaaaaaagattcagattacgaggattatggatgaacgCAATCGACAGGTGAGTatgggcTGCATGacTG |
| | | CATGgtTGCATGaacacaTATTAATttccGGAAGTCCGTTAGCCATCATATGCTTAGGGTTT |
| | | TGGTTTCATTCTTCTGTGTATCTTTCTTCTTCTCTCAGGTCAGAAACTGTATTGTTAA |
| | | TGCCACCATTTTCTTCTTTTAATCTAATCTATTACCAAGACCCATAATTCCTTTCCTC |
| | | ACCGCCTAACACTGCCAATGCCGGTCCCAAGCCCGGATAAAAAGTGGAGGGGGC |
| | | CGgaattctgcagatatccatcacact |
| 61 | MEF2C_ | agacccaagcttggtaccgagctcggatccgGCCACCATGGACTACAAAGACGATGACGACAAGatg |
| | 5 | gggagaaaaaagattcagatcgaggattatggatgaacgCAATCGACAGGTGAGTagggcTGCATGacTG |
| | | CATGgtTGCATGaacacaTATTAATttccAAATATTTCTCAGGTTCACACTATTTCCCAGGC |
| | | AACTCTATCACTGATGTCCTTATCACTTCAATTTTGGACAGCCTAGCAGCTTCCTGT |
| | | CCTCGATGATCTCCCTGCCTGGAATCATTCCTTCCTCAATTTAACTCAAACTGCAAA |
| | | CCCCCGCCTAACACTGCCAATGCCGGTCCCAAGCCCGGATAAAAAGTGGAGGGG |
| | | CCGGgaattctgcaatccacact |
| 62 | ATP7B_ | agacccaagcttggtaccgagctcggatccgGCCACCATGGACTACAAAGACGATGACGACAAGaaa |
| | 1 | tatcggtgtctttggccgaagggactgcaacagttctttataatccctctgtaattagcccagaagaactcagagctgctatagaagacat |
| | | gggatttgaggctCTGTAGTGTCTGGTGAGTtgggcTGCATGacTGCATGgtTGCATGaacacaT |
| | | ATTAATttccGAGGTGGGGCAGTTAGTTCACATGTCACAATTTAATCACATGAAAATC |
| | | TGCTAAAGGCCTAGAATTGCCACTATAAGATTTTGATTTGGTTAAAATATTTAGGAC |
| | | AGTGGCTGCCAAAAATATTTACATAGGAAACACCCCCACTTTCCACCGCCTAACAC |
| | | TGCCAATGCCGGTCCCAAGCCCGGATAAAAAGTGGAGGGGGGGGgaattctgcagatatcc |
| | | atcacact |
| 63 | ATP7B_ | agacccaagcttggtaccgagctcggatccgGCCACCATGGACTACAAAGACGATGACGACAAGaza |
| | 2 | tatcggtgtctttggccgaagggactgcaacagttctttataatccctctgtaattagcccagaagaactcagagctgctatagaagacat |
| | | gggatttgaggctCTGTAGTGTCTGGTGAGTtgggcTGCATGacTGCATGgtTGCATGaacacaT |
| | | ATTAATttcCTCCCACCGAGCAGCCTCAGCCCCTGGGCTCCAGTAACTACCTTGTGC |
| | | CTCAGCCATGCTGGAGGACAGAGCTCCCTGCTGTTGTTAATCTCTGGGGTTCTTCC |
| | | CATTGCCTATTTGGATTTCCAGCTCTCTAATACCTTTGTAAATAGTCCGCCTAACAC |
| | | TGCCAATGCCGGTCCCAAGCCCGGATAAAAAGTGGAGGGGGCGGgaattctgcagatatcc |
| | | atcacact |
| 64 | ATP7B_ | agacccaagcttggtaccgagctcggatccgGCCACCATGGACTACAAAGACGATGACGACAAGaza |
| | 3 | tatcggtgtctttggccgaagggactgcaacagttctttataatccctctgtaattagcccagaagaactcagagctgctatagaagacat |
| | | gggatttgaggctCTGTAGTGTCTGGTGAGTagggcTGCATGacTGCATGgtTGCATGaacacaT |
| | | ATTAATttcCAGCCATCCTGGAGGACAGAGCTCCCTGCTGTTGTTAATCTCTGGGGT |
| | | TCTTCCCATTGCCTATTTGGATTTCCAGCTCTCTAATACCTTTGTAAATAGTTCTCTA |
| | | TATTAAATCCTTCCACTGAGCTACCCCGTAACTTCACCTTATCTCCGCCTAACACTG |
| | | CCAATGCCGGTCCCAAGCCCGGATAAAAAGTGGAGGGGGGGGgaattctgcagatatccatca |
| | | cact |
| 65 | ATP7B_ | agacccaagcttcgtaccgagctcggatccgGCCACCATGGACTACAAAGACGATGACGACAAGaaa |
| | 4 | tatcggtgtctttggccgaagggactgcaacagttctttataatccctctgtaattagcccagaagaactcagagctgctatagaagacat |
| | | gggatttgaggcttCTGTAGTGTCTGGTGAGTtaggcTGCATGacTGCATGgtTGCATGaacacaT |
| | | ATTAATtccTTCCCATTGCCTATTTGGATTTCCAGCTCTCTAATACCTTTGTAAATAGT |
| | | TCTCTATATTAAATCCTTCCACTGAGCTACCCCGTAACTTCACCTTATCTTTCATTTC |
| | | CTTCAGGAATACTTACTGCATGCGGGGTGCTATTGAGTCCAACCGCCTAACACTGC |
| | | CAATGCCGGTCCCAAGCCCGGATAAAAAGTGGAGGGGGGGGgaattctgcagatatccatcac |
| | | act |
| 66 | ATP7B_ | agacccaagcttggtaccgagctcggatccgGCCACCATGGACTACAAAGACGATGACGACAAGaaa |
| | 5 | tatcggtgtctttcgccgaagggactgcaacagttctttataatccctctgtaattagcccagaagaactcagagctgctatagaagacat |
| | | gggatttgaggchCTGTAGTGTCTGGTGAGTttgggcTGCATGacTGCATGgTTGCATGaacacaT |
| | | ATTAATtccTTGACCAAGTGGAGGGTGCTCTTCCAGCTCTTGAACAGGACCTAGAG |
| | | AGTTGGATGTATTAGATGGGCGTACGCAgTATGTGCCCAGTTGTATGATTGTGCGTT |
| | | TTCAAGGAAGGGAGTGTGGGTCGATTCGTTCAGTATCGACAgGGGGCCGCCTAAC |
| | | ACTGCCAATGCCGGTGCCAAGCCCGGATAAAAAGTGGAGGGGGCGGgaattctgcagata |
| | | tccatcacact |
| 67 | CBS_1 | agacccaagcttcgtaccgagctcggatccgGCCACCATGGACTACAAAGACGATGACGACAAGgg |
| | | ggtggatcccgaagggtccatcctcgcagagccggaggagctgaaccagacggagcagacaacctacgaggtggaagggatcgg |
| | | ctacgacttcatccccacggtgCTCGATAGAACGGTGAGTtgggCTGCATGacTGCATGgtTGCATG |
| | | aacacaTATTAATttccGACCAAGAGGGTGAACAGCCTCATGGTGGACCCCGTGTCTAG |
| | | GCGGTAGGACCCCAGAGGTCTCACCCCAGCCTCTCCCCCTGGGATCGGCACGTCT |
| | | GCAGACTTTCCAGTTCCAACACGTTTTGCAGACAGCAAAACTGGCCGCCAGCCCC |
| | | GCCTAACACTGCCAATGCCGGTCCCAAGCCCGGATAAAAAGTGGAGGGGGCGGga |
| | | attctgcagatatccatcacact |
| 68 | CBS_2 | agacccaagcttggtacgagctcggatccgGCCACCATGGACTACAAAGACGATGACGACAAGgg |
| | | ggtggatcccgaagggtccatcctcgcagagccggaggagctgaaccagacggagcagacaacctacgagctcgaagggatcgg |
| | | ctacgacttcatccccacggtcCTCGATAGAACGGTGAGTttgagcTGCATGacTGCATGgtTGCATG |
| | | aacacaTATTAATTTccACCCCAGAGGTCTCACCCCAGCCTCTCCCCCTGGGATCGGCAC |
| | | GTCTGCAGACTTTCCAGTTCCAACACGTTTTGCAGACAGCAAAACTGCCCGCCAG |
| | | CCCCACTGAGCATCCGTGTGACCCACACCGGCCCTTCGGTCTCTGCTCTCCCCCGC |
| | | CTAACACTGCCAATGCCGGTCCCAAGCCCGGATAAAAAGTGGAGGGGGGGGgaattc |
| | | tgcagatatccatcacact |
| 69 | CBS_3 | agacccaagcttggtaccgagctcggatccgGCCACCATGGACTACAAAGACGATGACGACAAGgg |
| | | ggtggatcccgaagcgtccatcctcgcagagccggaggagctgaaccagacggagcagacaacctacgaggtggaagggatcgg |
| | | ctacgacttcatccccacggtcCTCGATAGAACGGTGAGTttgggcTGCATGacTGCATGgtTGCATG |
| | | aacacaTATTAATttccGACTTTCCAGTTCCAACACGTTTTGCAGACAGCAAAACTGCCC |
| | | GCCAGCCCCACTGAGCATCCGTGTGACCCACACCGGCCCTTCGGTCTCTGCTCTC |
| | | CCTCAGGCCAAAGTCAGCTTTTTGGCTCTTAACAGACTTTTCCGGGGCCTTACCGC |
| | | CTAACACTGCCAATGCCGGTCCCAAGCCCGGATAAAAAGTGGAGGGGGGGGgaattc |
| | | tgcagatatccatcacact |
| 70 | CBS_4 | agacccaagcttggtaccgagctcggatccgGCCACCATGGACTACAAAGACGATGACGACAAGgg |
| | | ggtggatcccgaagcgtccatcctcgcagagccggaggagctgaaccagaccgagcagacaacctacgaggtggaagggatcgg |
| | | ctacgacttcatccccacggtcCTCGATAGAACGGTGAGTttgggcTGCATGacTGCATGgtTGCATG |
| | | aacacaTATTAATttccCCACTGAGCATCCGTGTGACCCACACCGGCCCTTCGGTCTCTG |
| | | CTCTCCCTCAGGCCAAAGTCAGCTTTTTGGCTCTTAACAGACTTTTCCGGGGCCTT |
| | | AATTTTCACACGTTTTCCCTGCTCCCTGCCTGTGCCACCTGGGCTACATCCCCGCC |
| | | TAACACTGCCAATGCCGGTCCCAAGCCCGGATAAAAAGTGGAGGGGGGGGgaattctg |
| | | cagatatccatcacact |
| 71 | CBS_5 | agacccaagcttggtaccgagctcggatccgGCCACCATGGACTACAAAGACGATGACGACAAGgg |
| | | ggtggatcccgaagggtccatcctcgcagagccggaggagctgaaccagacggagcagacaacctacgaggtggaagggatcgg |
| | | CtacgacttcatccccacggttCTCGATAGAACGGTGAGTtttgggcTGCATGacTGCATGgtTGCATG |
| | | aacacaTATTAATttccTTGACCAAGTGGAGGGTGCTCTTCCAGCTCTTGAACAGGACCT |
| | | AGAGAGTTGGATGTATTAGATGGGCGTACGCAgTATGTGCCCAGTTGTATGATTGTG |
| | | CGTTTTCAAGGAAGGGAGTGTGCGTCGATTCGTTCAGTATCGACAgGGGGCGGCC |
| | | TAACACTGCCAATGCCGGTCCCAAGCCCGGATAAAAAGTGGAGGGGGGGGgaattctg |
| | | cagatatccatcacact |
| 72 | HTT_Tw_ | agacccaagcttgGCCACCATGGACTACAAAGACGATGACGACAAGggc |
| | HDV | ccggctgtggctgaggagccCctCcaccgaccGTGAGTttgggcTGCATGacTGCATGgCTGCATGaacaca |
| | | TATTAATttcctccacttagttctacacctcattcaTcattgagtgtttctcgactactatgaataaacccttatactccatgttgc |
| | | gggcagaatggggatctggttcagggaagcacagggcacgagttcaccaatggctgtcaagctacgctgcCCGCCTAACA |
| | | CTGCCAATGCCGGTCCCAAGCCCGGATAAAAAGTGGAGGGGGGGGggccggcatggtcc |
| | | cagcctcctcgctggccatcattatgggacGCGGCCGCgaattctgcagtccata |
| | | cact |
| 73 | HTT_ | agacccaagcttggtaccgagctcggatccgGCCACCATGGACTACAAAGACGATGACGACAAGggc |
| | HDV_Tw | ccggctgtcgctgaggagccCctCcaccgaccGTGAGTttgggcTGCATGacTGCATGgTTGCATGaacaca |
| | | TATTAATttcctccacttagttctacacctcattcattcattcagtgagtgtttctcgactactatghaaaaccgttatactccatgttgc |
| | | gggcagaatggggatctggacagggaagcacagggcacgagttcaccaatggctgtcaagctacgctgcggccggcatggtccca. |
| | | gcctcctcgctggcgccggctcaacatgcttcggcatggcgaatgggacGCGGCCCCCCGCCTAACACTG |
| | | CCAATGCCGGTCCCAAGCCCGGATAAAAAGTGGAGGGGGCGGgaattctgcagatatccatca |
| | | cact |
| 74 | HTT_ | agacccaagcttggtaccgagctcggatccgGCCACCATGGACTACAAAGACGATGACGACAAGggc |
| | hyb1_ | ccggctgtcgctgaggagccCctCcaccgaccGTGAGTtgggcTGCATGacTGCATGgCTGCATGaacaca |
| | | TATTAATttccTTCATTCAGTGAGTGTTTCTCGACTACTATGAATAAACCGTTATACTC |
| | | CATGTTGCGGGCAGAATGGGGATCTGGACAGGGAAGCACAGGGCACGAGTTCAC |
| | | CAATGGCTGTCAAGCTACGCTGCTCACAGAAAAAACAGATGATGTTACCCGCCTA |
| | | ACACTGCCAATGCCGGTGCCAAGCCCGGATAAAAAGTGGAGGGGGGGGgaattctgca |
| | | gatatccatcacact |
| 75 | HTT_ | agacccaagcttggtaccgagctcggatccgGCCACCATGGACTACAAAGACGATGACGACAAGggc |
| | hyb_2 | ccgcctgtggctgaggagccCctCcaccgaccGTGAGTttgggcTGCATGacTGCATGgtTGCATGaacaca |
| | | TATTAATaccAATTAATATGTGTTGAATTACCCACTCCACTTAGTTCTACACCTCATTC |
| | | ATTCATTCAGTGAGTGTTTCTCGACTACTATGAATAAACCGTTATACTCCATGTTGC |
| | | GGGCAGAATGGGGATCTGGACAGGGAAGCACAGGGCACGAGTTCCCGCCTAACA |
| | | CTGCCAATGCCGGTGCCAAGCCCGGATAAAAAGTGGAGGGGGGGGgaattctgcagatatc |
| | | catcacact |
| 76 | HTT_ | agacccaagcttggtaccgagctcggatccgGCCACCATGGACTACAAAGACGATGACGACAAGggc |
| | hyb_3 | ccggctgtggctgaggagccCc:CcaccgaccGTGAGTttgggcTGCATGacTGCATGgTGCATGaacaca |
| | | TATTAATttccTCTACACCTCATTCATTCATTCAGTGAGTGTTTCTCGACTACTATGAAT |
| | | AAACCGTTATACTCCATGTTGCGGGCAGAATGGGGATCTGGACAGGGAAGCACAG |
| | | GGCACGAGTTCACCAATGGCTGTCAAGCTACGCTGCTCACAGAAAACCGCCTAAC |
| | | ACTGCCAATGCCGGTCCCAAGCCCGGATAAAAAGTGGAGGGGGGGGgaattctgcagata |
| | | tccatcacttct |
| 77 | HTT_ | agacccaagcttggtaccgagctcggatccgGCCACCATGGACTACAAAGACGATGACGACAAGggc |
| | hyb_4 | ccggctgtggctgaggagccCctCcaccgaccGTGAGTttgggcTGCATGacTGCATGgtTGCATGaacaca |
| | | TATTAATttccCACTCCACTTAGTTCTACACCTCATTCATTCATTCAGTGAGTGTTTCT |
| | | CGACTACTATGAATAAACCGTTATACTCCATGTTGCGGGCAGAATGGGGATCTGGA |
| | | CAGGGAAGCACAGGGCACGAGTTCACCAATGGCTGTCAAGCTACGCCCGCCTAA |
| | | CACTGCCAATGCCGGTCCCAAGCCCGGATAAAAAGTGGAGGGGGGGGgaattctgcaga |
| | | tatccatcacact |
| 78 | HTT_ | agacccaagcttagtaccgagctcggatccgGCCACCATGGACTACAAAGACGATGACGACAAGggc |
| | hyb_5 | ccggctgtggctgaggagccCctCcaccgaccGTGAGTttgggcTGCATGacTGCATGgTTGCATGaacaca |
| | | TATTAATttccACTTAGTTCTACACCTCATTCATTCATTCAGTGAGTGTTTCTCGACTA |
| | | CTATGAATAAACCGTTATACTCCATGTTGCGGGCAGAATGGGGATCTGGACAGGGA |
| | | AGCACAGGGCACGAGTTCACCAATGGCTGTCAAGCTACGCTGCTCACCGCCTAAC |
| | | ACTGCCAATGCCGGTCCCAAGCCCGGATAAAAAGTGGAGGGGGCGGgaattctgcagata |
| | | tccatcacact |
| 79 | MECP2_ | agacccaagcttggtaccgagctcggatccgGCCACCATGGACTACAAAGACGATGACGACAAGcat |
| | hyb_1 | catccgtgaccggggacccatgtatgatgaccccaccctgcctgaaggctggacacggaagcttaagcaaaggaaatctggccgct |
| | | ctgctgggaagtatgatgtgtacttaattaaGTGAGTttgggcTGCATGacTGCATGgtTGCATGaacacaTATT |
| | | AATttccCACAAGGAACAATTAGAGGCTCTCCATAGCAATGTCAGAGATAGGGCAGA |
| | | GCGGATGGTGGTGACAACGCTCTGACAAACGTTACTATTGAACGAGAGTCACACT |
| | | GCCTGGCTGCCCCTGAGGACCTGTCACCAAAGCCACTCACTCGTCCCGCCTAACA |
| | | CTGCCAATGCCGGTGCCAAGCCCGGATAAAAAGTGGAGGGGGCGGgaattctgcagatatc |
| | | ctttcacact |
| 80 | MECP2_ | agacccaagcttggtaccgagctcggatccgGCCACCATGGACTACAAAGACGATGACGACAAGcat |
| | hyb_2 | catccgtgaccggggacccatgtatgatgaccccacgcctgaaggctcgacacggaagcttaagcaaaaggaaatctggccgct |
| | | ctgctgggaagtatgatgtgtacttaattaaGTGAGTttgggcTGCATGacTGCATGgtTGCATGaacacaTATT |
| | | AATtTccGGAACAATTAGAGGCTCTCCATAGCAATGTCAGAGATAGGGCAGAGCGGA |
| | | TGGTGGTGACAACGCTCTGACAAACGTTACTATTGAACGAGAGTCACACTGCCTG |
| | | GCTGCCCCTGAGGACCTGTCACCAAAGCCACTCACTCGTCTGCCTCCGCCTAACA |
| | | CTGCCAATGCCGGTCCCAAGCCCGGATAAAAAGTGGAGGGGGCGGgaattctgcagatatc |
| | | catcacact |
| 81 | MECP2_ | agacccattgcttggtaccgagctcggatccgCCCACCATGGACTACAAAGACGATGACGACAAGcat |
| | hyb_3 | catccgtgaccggggacccatgtatgatgaccccaccctgcctgaaggctggacacggaagcttaagcaaaggaaatctggccgct |
| | | ctgctgggaagtatgatgtgtacttaattaaGTGAGTttgggcTGCATGacTGCATGgtTGCATGaacacaTATT |
| | | AATttccGAGGCTCTCCATAGCAATGTCAGAGATAGGGCAGAGGGGATGGTGGTGAC |
| | | AACGCTCTGACAAACGTTACTATTGAACGAGAGTCACACTGCCTGGCTGCCCCTG |
| | | AGGACCTGTCACCAAAGCCACTCACTGGTCTGCCTGCCGCTGACCCCGCCTAACA |
| | | CTGCCAATGCCGGTCCCAAGCCCGGATAAAAAGTGGAGGGGGCGGgaattctgcagatatc |
| | | catcacact |
| 82 | MECP2_ | agacccaagcttggtaccgagctcggatccgGCCACCATGGACTACAAAGACGATGACGACAAGcat |
| | hyb_4 | catccgtgaccggggacccatgtatgatgaccccaccctgcctgaaggctggacacggaagcttaagcaaaggaaatctggccgct |
| | | ctgctgggaagtatgatgtacttaattaaGTGAGTttgggcTGCATGacTGCATGatTGCATGaacacaTATT |
| | | AATttccAGGCCTCTCCAAAGTTCAGCAACCAAAGAGTCAGGCCTGGTACAGAAGG |
| | | GTTGTCAGGCTGAGGCCCATTCTGCTGGTTGGTGCCTGGGCCTGAGTCACCATCTA |
| | | GAACATTCCCAAGTTTGAACTCAGGTACGGTGCTCAGTCTCTCCACCGCCTAACA |
| | | CTGCCAATGCCGGTCCCAAGCCCGGATAAAAAGTGGAGGGGGGGGgaattctgcagatatc |
| | | catcacact |
| 83 | MECP2_ | agacccaagcttagtaccgagctcggatccgGCCACCATGGACTACAAAGACGATGACGACAAGct |
| | hyb_5 | catccgtgaccggggacccatgtatgatgaccccaccctgcctgaaggctggacacggaagcttaagcaaaggaaatctggccgct |
| | | ctgctgggaagtatgatgtgtacttaattaaGTGAGTttgggcTGCATGacTGCATGgTTGCATGaacacaTATT |
| | | AATttccCAGAGATAGGGCAGAGCGGATGGTGGTGACAACGCTCTGACAAACGTTA |
| | | CTATTGAACGAGAGTCACACTGCCTGGCTGCCCCTGAGGACCTGTCACCAAAGCC |
| | | ACTCACTCGTCTGCCTGCCGCTGACCCCCCCAGGCCTCTCCAAAGTCCGCCTAAC |
| | | ACTGCCAATGCCGGTCCCAAGCCGGGATAAAAAGTGGAGGGGGCGGgaattctgcagata |
| | | tccatcacact |
| 84 | MECP2_ | agacccaagcttggtaccgagctcggatccgGCCACCATGGACTACAAAGACGATGACGACAAGcat |
| | Tw_HDV | catccgtgaccggggacccatgtatgatgaccccaccctgcctgaaggctggacacggaagcttaagcaaaggaaatctggccgct |
| | | ctgctgggaagtatgatgtgtacttaattaaGTGAGTttgggcTGCATGacTGCATGgTGCATGaacacaTATT |
| | | AATttccAATTAGAGGCTCTCCATAGCAATGTCAGAGATAGGGCAGAGCGGATGGTG |
| | | GTGACAACGCTCTGACAAACGTTACTATTGAACGAGAGTCACACTGCCTGGCTGC |
| | | CCCTGAGGACCTGTCACCAAAGCCACTCACTCGTCTGCCTGCGGCCCGCCTAACA |
| | | CTGCCAATGCCGGTGCCAAGCCCGGATAAAAAGTGGAGGGGGCGGggccggcatggtcc |
| | | cagcctcctcgctggcgctgggcaacagcatggcgagggacGCGGCCGCgaattctgcagatatccatca |
| | | cact |
| 85 | MECP2_ | agacccaagcttggtaccgagctcggatccgGCCACCATGGACTACAAAGACGATGACGACAAGcat |
| | HDV_TW | catccgtgaccggggacccatgtatgatgaccccaccctgcctgaaggctggacacggaagcttaagcaaaggaaatctggccgct |
| | | ctgctgggaagtatgatgtgtacttaattaaGTGAGTttgggcTGCATGacTGCATGgTGCATGaacacaTATT |
| | | AATttccAATTAGAGGCTCTCCATAGCAATGTCAGAGATAGGGCAGAGCGGATGGTG |
| | | GTGACAACGCTCTGACAAACGTTACTATTGAACGAGAGTCACACTGCCTGGCTGC |
| | | CCCTGAGGACCTGTCACCAAAGCCACTCACTCGTCTGCCTGCCGCggccggcatggtccc |
| | | agcctcctcgctggcgccggctgggcaacatgcttcggcatggcgaatgggacGCGGCCGCCCGCCTAACACTG |
| | | CCAATGCCGGTCCCAAGCCCGGATAAAAAGTGGAGGGGGCGGgaattctgcagatatccatca |
| | | cact |
| 86 | HTT | agaccchagcttcgtaccgagctcggatccgGCCACCATGGACTACAAAGACGATGACGACAAGgcg |
| | AAV TW | accctggaaaagctgatgaaggccttcgagtccctcaagtccttccagcagAATcaAcagAATAACcagcaAAATcagA |
| | | ACcaAAACcaAcagAATcagcagAACcagccgccaccTccAccAccGccTccTccCccAcctcagcttcctcag |
| | | ccAccTccAcaggcacagccTctgctgcctcagccAcagccTcccccAccAccTcccccAccTccacccggcccggctgt |
| | | ggctgaggagccCctgcaccgaccGTGAGTtgggcTGCATCacTGCATGgtTGCATGaacacaTATTAA |
| | | Ttttcctccacttagttctacacctcattcattcattcagtgagtgtttctcgactactatgaataaaccgttatactccatgttgcgggcagaa |
| | | tagggatctggacagggaagcacagggcacgagttcaccaatggctgtcaagctacgctgcCCGCCTAACACTGCCA |
| | | ATGCCGGTCCCAAGCCCGGATAAAAAGTGGAGGGGGGGGgaattctgcacact |
| 87 | HTT AAV | agacccaagcttggtaccgagctcggtccgGCCACCATGGACTACAAAGACGATGACGACAAGgcg |
| | HDV | accctggaaaagctgatgaaggccttcgagtccctcaagtccttccagcagAATcaAcagAATAACcagcaAAATcagA |
| | | ACcaAAACcaAcagAATcagcagAACcagccgccaccTccAccAccGccTccTccCcctcagcttcctcag |
| | | ccAccTccAcaggcacagccTctgctgcctcagccAcagccTcccccAccAccTcccccAccTccacccggcccggctgt |
| | | ggctgaggagccCctgcaccgaccGTGAGTttgggcTGCATGacTGCATGgtTGCATGaxcacaTATTAA |
| | | Tttcctccacttagttctacacctcattcagtttctcgactactatgaataaaccgttatactccatgtccgggcagaa |
| | | tggggatctggacagcacgagttcaccaatggctgtcaagctacgctgcatggtcccagcctcctcg |
| | | ctggcgccgcctgctgcttcggcatggcaggacGCGGCCGCgaattctaatatccatcacact |
| 88 | HTT | agacccaagcttggtactcggatccgGCCACCATGGACTACAAAGACGATGACGACAAGgcg |
| | AAV NT | accctcgaaaagctTccctcagtccttccagcagAATcaAcagAATAACcagcaAAATcagA |
| | | ACcaAAACcagcagAACcagcccaccTccAccAccGccTccTccCccActcagcttcctcag |
| | | ccAccTccAcaggcacagccTctgctgcctcagccAcagccTcccccAccAccTcccccAccTccacccggcccggctgt |
| | | ggctgaggagccCctgcaccgaccGTGAGTttgggcTGCATGacTGCATGgTTGCATGaacacaTATTAA |
| | | TttccTTGACCAAGTGGAGGGTGCTCTTCCAGCTCTTGAACAGGACCTAGAGAGTTG |
| | | GATGTATTAGATGGGCGTACGCAgTATGTGCCCAGTTGTATGATTGTGCGTTTTCAA |
| | | GGAAGGGAGTGTGCGTCGATTCGTTCAGTATCGACAgGGGGCCGCCTAACACTGC |
| | | CAATGCCGGTCCCAAGCCCGGATAAAAAGTGGAGGGGGCGGgaattctgcagatatccatcac |
| | | Bct |
| 89 | MECP2_ | agacccaagcttggtaccgagctcggatccgGCCACCATGGACTACAAAGACGATGACGACAAGcat |
| | 5TS_cargo_ | catccgtgaccggggacccatgtatgatgaccccaccctgcctgaaggctggacacggaagcttaagcaaaggatatctggccgct |
| | 3 | ctgctgggaagtatgatgtgtacttaattaaGTGAGTttgggcTGCATGacTGCATGgTTGCATGaacacaTATT |
| | | AATttccGATAGAGGGCCTGCTACCTTGAGAACTTGCCACTCAGGGCAGCGACCTCT |
| | | GTACACGGTCATTTCAAGCACACCTGGTCTCAGTGTTCATTGTTTATGTTCCCCCC |
| | | GACCCCACCCTGGGCACATACATTTTCCTGCTCCATGAGGGATCCCGCCTAACACT |
| | | GCCAATGCCGGTCCCAAGCCCGGATAAAAAGTGGAGGGGGCGGgaattctgcagatatccat |
| | | cacact |
| 90 | MBCP2_ | agacccaagcttggtaccgagctcggatccgGCCACCATGGACTACAAAGACGATGACGACAAGcat |
| | 5TS_cargo_ | catccgtgaccggggttcccatgtatgatgaccccaccctgcctgaaggctggacacggaagcttaagcaaaggaaatctggccgct |
| | 4 | ctgctgggaagtatgatgtgtacttaattaaGTGAGTttgggcTGCATGacTGCATGgtTGCATGaacacaTATT |
| | | AATttccAGATAAAGACTTGAGGTGTGGAGAGGATAGAGGGCCTGCTACCTTGAGAA |
| | | CTTCCCACTCAGGGCAGCGACCTCTGTACACGGTCATTTCAAGCACACCTGGTCT |
| | | CAGTGTTCATTGTTTATGTTCCCCCCGACCCCACCCTGGGCACATCCGCCTAACAC |
| | | TGCCAATGCCGGTCCCAAGCCCGGATAAAAAGTGGAGGGGGGGGgaattctgcagatatcc |
| | | atcacact |
| 91 | MECP2_ | agacccaagcttggtaccgagctcggatccgGCCACCATGGACTACAAAGACGATGACGACAAGcat |
| | 5TS_cargo_ | catccgtgaccggggacccatgtatgatgaccccaccctgcctgaaggctggacacggaagcttaagcaaaggaaatctggccgct |
| | 5 | ctgctgggaagtatgatgtgtacttattttaaGTGAGTtgggcTGCATGacTGCATGgtTGCATGaacacaTATT |
| | | AATttccCTGCTTCCGCAGCTATTCCATCCCCAGATAAAGACTTGAGGTGTGGAGAG |
| | | GATAGAGGGCCTGCTACCTTGAGAACTTCCCACTCAGGGCAGCGACCTCTGTACA |
| | | CGGTCATTTCAAGCACACCTGGTCTCAGTGTTCATTGTTTATGTTCCGCCTAACACT |
| | | GCCAATGCCGGTCCCAAGCCCGGATAAAAAGTGGAGGGGGGGGgaattctgcagatatccat |
| | | cacact |
| 92 | MECP2_ | agacccaagcttggtaccgagctcggatccgGCCACCATGGACTACAAAGACGATGACGACAAGcat |
| | 5TS_cargo_ | catccgtgttccggggacccatgtatgatgaccccaccctgcctgaaggctggacacggaagcttaagcaaaggaaatctggccgct |
| | 6 | ctgctgggaagtatgatgtgtacttaattaaGTGAGTtttgggcTGCATGacTGCATGgTTGCATGaacacaTATT |
| | | AATttccTGAACCCCTAGCTCTGCAAGTTCCTCTGCTTCCGCAGCTATTGCATCGCCA |
| | | GATAAAGACTTGAGGTGTGGAGAGGATAGAGGGCCTGCTACCTTGAGAACTTGCC |
| | | ACTCAGGGCAGCGACCTCTGTACACGGTCATTTCAAGCACACCTCCGCCTAACAC |
| | | TGCCAATGCCGGTCCCAAGCCCGGATAAAAAGTGGAGGGGGCGGgaattctgcagatatcc |
| | | atcacact |
| 93 | MECP2_ | agacccaagcttggtaccgagctcggatccgGCCACCATGGACTACAAAGACGATGACGACAAGcat |
| | 5TS_cargo_ | catccgtgaccggggacccatgtatgatgaccccaccctgcctgaaggctggacacggaagcttaagcaaaggaaatctggccgct |
| | 21 | ctgctgggaagtatgatgtgtacttaattaaGTGAGTttgggcTGCATGacTGCATGgCTGCATGaacacaTATT |
| | | AATttccTATTGAACGAGAGTCACACTGCCTGGCTGCCCCTGAGGACCTGTCACCAA |
| | | AGCCACTCACTCGTCTGCCTGCCGCTGACCCCCCCAGGCCTCTCCAAAGTTCAGC |
| | | AACCAAAGAGTCAGGCCTGGTACAGAAGGGTTGTCAGGCTGAGGCCCGCCTAAC |
| | | ACTGCCAATGCCGGTCCCAAGCCCGGATAAAAAGTGGAGGGGGGGGgaattctgcagata |
| | | tccatcacact |
| 94 | MECP2_ | agacccaagcttggtaccgagctcggatccgGCCACCATGGACTACAAAGACGATGACGACAAGcat |
| | 5TS_cargo_ | catccgtgaccgcggacccatgtatgatgaccccaccctgcctgaaggctggacacggaagcttaagcaaaggaaatctggccgct |
| | 24 | ctgctgggaagtatgatgtgtacttaattaaGTGAGTttgggcTGCATGacTGCATGgtTGCATGaacacaTATT |
| | | AATttccAATTAGAGGCTCTCCATAGCAATGTCAGAGATAGGGCAGAGGGGATGGTG |
| | | GTGACAACGCTCTGACAAACGTTACTATTGAACGAGAGTCACACTGCCTGGCTGC |
| | | CCCTGAGGACCTGTCACCAAAGCCACTCACTCGTCTGCCTGCCGCCCGCCTAACA |
| | | CTGCCAATGCCGGTCCCAAGCCCGGATAAAAAGTGGAGGGGGGGGgaattctgcagatatc |
| | | Catcacact |
| 95 | HTT no | agacccaagcttggtaccgagctcggatccgGTCCACCATGGACTACAAAGACGATGACGACAAGggc |
| | twister | ccggctgtggctgaggagccCctCcaccgaccGTGAGTttgggcTGCATGacTGCATGgCTGCATGaacaca |
| | cargo | TATTAATttcctccacttagttctacacccattcattcattcagtgagtgttctcgactactatgaataaaccgttatactccatgttgc |
| | | gggcagaatggggatctggacggaagcacagggcacgagttcaccaatggctgtcaagctacgctgcgaattctgcagatatcc |
| | | atcacact |
| 96 | MECP2 | agacccaagcttggtaccgagctcggatccgGCCACCATGGACTACAAAGACGATGACGACAAGcat |
| | cargo 24 | catccgtgaccggggacccatgtatgatgaccccaccctgcctgaaggctggacacggaagcttaagcaaaggaaatctggccgct |
| | COMP14 | ctgctgggaagtatgatgtgtacttaattaaGTGAGTttgggcTGCATGacTGCATGgTTGCATGaacacaTATT |
| | | AATttccAATTAGAGGCTCTCCATAGCAATGTCAGAGATAGGGCAGAGCGGATGGTG |
| | | QTGACAACGCTCTGACAAACGTTACTATTGAACGAGAQTCACACTGCCTGGCTGC |
| | | CCCTGAGGACCTGTCACCAAAGCCACTCACTCGTCTGCCTGCCGCCCGCCTAACA |
| | | CTGCCAATGCCGGTCCCAAGCCCGGATAAAAAGTGGAGGGGGGGGAAAGGTTTT |
| | | TCTTTTCCTGAGAAATTTCTCAGGTTTTGCTTTTAAAAAAAAAGCAAAAgaattctgcag |
| | | atatccatcacttct |
| 97 | MECP2 | agacccaagcttggtaccgagctcggatccgGCCACCATGGACTACAAAGACGATGACGACAAGCHT |
| | cargo 24 | catccgtgaccggggttcccatgtatgatgaccccaccctgcctgaaggctggacacggaagcttaagcaaaggaaatctggccgct |
| | MALAT1 | ctgctgggaagtatgatgtgtacttaattaaGTGAGTttgggcTGCATGacTGCATGgtTGCATGaacacaTATT |
| | | AATttccAATTAGAGGCTCTCCATAGCAATGTCAGAGATAGGGCAGAGCGGATGGTG |
| | | GTGACAACGCTCTGACAAACGTTACTATTGAACGAGAGTCACACTGCCTGGCTGC |
| | | CCCTGAGGACCTGTCACCAAAGCCACTCACTCGTCTGCCTGCCGCCCGCCTAACA |
| | | CTGCCAATGCCGGTCCCAAGCCCGGATAAAAAGTGGAGGGGGGGGgaaggtttttcttttcct |
| | | gagaaaacaacacgtattgttttctcaggttttgctttttggccttttttctagcttaaaaagaattctgcagatatccat |
| | | cacact |
| 98 | MECP2 | agacccaagcttggtaccgagctcggatccgGCCACCATGGACTACAAAGACGATGACGACAAGcat |
| | cargo 24 | catccgtgaccggggacccatgtatgagaccccaccctgcctgaaggctggacacggaagcttaagcaaaggaaatctggccgct |
| | MENB | ctgctgggaagtatgatgtgtacttaattaaGTGAGTttgggcTGCATGacTGCATGgtTGCATGaacacaTATT |
| | | AATttccAATTAGAGGCTCTCCATAGCAATGTCAGAGATAGGGCAGAGCGGATGGTG |
| | | GTGACAACGCTCTGACAAACGTTACTATTGAACGAGAGTCACACTGCCTGGCTGC |
| | | CCCTGAGGACCTGTCACCAAAGCCACTCACTCGTCTGCCTGCCGCCCGCCTAACA |
| | | CTGCCAATGCCGGTCCCAAGCCCGGATAAAAAGTGGAGGGGGCGGAGGTGTTTCT |
| | | TTTACTGAGTGCAGCCCATGGCCGCACTCAGGTTTTGCTTTTCACCTTCCCATCTG |
| | | TGAAAGAGTGAGCAGGAAAAAGCAAAAgaattctgcagatatccatcacact |
| 99 | SMN1_ | agacccaagcttggtagGCCACCATGGACTACAAAGACGATGACGACAAGggc |
| | before | ccggctgtggctgaggagccCcCcaccgaccGTGAGTttgggcTGCATGacTGCATGgCTGCATGaacaca |
| | | TATTAATttcctccacttagttctacattcattcattcagtgagtgtttctcgactactatgaataaaccgttatactccatgttgc |
| | | gggcagaatggggatctggacagggaagcacagggcacgagttcaccaatggctgtcaagctacgctgcATAATTTTTGA |
| | | CCGCCTAACACTGCCAATGCCGGTCCCAAGCCCGGATAAAAAGTGGAGGGGGCG |
| 100 | SMN2_ | agacccaagcttggtaccgagctcggatccgGCCACCATGGACTACAAAGACGATGACGACAAGggc |
| | before | ccggctgtggctgaggagccCctCcaccgaccGTGAGTttgggcTGCATGacTGCATGgCTGCATGaacaca |
| | | TATTAATttcctccacttagttctacacctcattcattcattcagtgagtgtttctcgactactatgaataaaccgttatactccatgttgc |
| | | gggcagaatggcgatctggttcagggaagcacagggcacgagttcaccaatggctgtcaagctacgctccAATTTTTGACC |
| | | GCCTAACACTGCCAATGCCGGTCCCAAGCCCGGATAAAAAGTGGAGGGGGGGGga |
| | | attctgcagatatccatcacact |
| 101 | SMN3_ | agacccaagcttcgtaccgagctcggatccgGCCACCATGGACTACAAAGACGATGACGACAAGggc |
| | before | ccggctgtggctgaggagccCctCcaccgaccGTGAGTttgggcTGCATGacTGCATGgtTGCATGaacaca |
| | | TATTAATttcctccacttagttctacacctcattcattcattcagtgagtgtttctcgactactatgaataaaccgttatactccatgttgc |
| | | gggcagaatggggatctggacagggaagcacagggcacgagttcaccaatggctgtcaagctacgctgcATTTGTGGCC |
| | | GCCTAACACTGCCAATGCCGGTCCCAAGCCCGGATAAAAAGTGGAGGGGGGGGga |
| | | attctgcagatatccatcacact |
| 102 | SMNL_ | agacccaagcttcgtaccgagctcggatccgGCCACCATGGACTACAAAGACGATGACGACAAGggc |
| | after | ccggctgtggctgaggagccCcCCcaccgaccGTGAGTttgggcTGCATGacTGCATGgCTGCATGaacaca |
| | | TATTAATttcctccacttagttctacacctcattcattcattcagtgagtgtttctcgactactatgaataaacccttatactccatgttgc |
| | | gggcagaatggggatctggacagcgaagcacagggcacgagttcaccaatggctgtcaagctacgctgcCCGCCTAACA |
| | | CTGCCAATGCCGGTCCCAAGCCCGGATAAAAAGTGGAGGGGGCGGATAATTTTTG |
| | | Agaattctgcagcact |
| 103 | SMN2_ | agacccaagcttggtaccgagctcggatccgGCCACCATGGACTACAAAGACGATGACGACAAGggc |
| | after | ccggctgtggctgaggagccCctCcaccgaccGTGAGTttgggcTGCATGacTGCATGgtTGCATGaacaca |
| | | TATTAATttcctccacttagttctacacctcattcattcattcagtgagtgtttctcgactactatgaataaaccgttatactccatgttgc |
| | | gggcagaatggggatctcgacagggaagcacagggcacgagttcaccaatggctgtcaagctacgctgcCCGCCTAACA |
| | | CTGCCAATGCCGGTGCCAAGCCCGGATAAAAAGTGGAGGGGGGGGAATTTTTGAg |
| | | aattctgcagatatccatcacact |
| 104 | SMN3_ | agacccaagcttggtaccgagctcggatccgGCCACCATGGACTACAAAGACGATGACGACAAGggc |
| | after | ccggctgtggctgaggagccCctCcaccgaccGTGAGTtttgggcTGCATGacTGCATGgTTGCATGaacaca |
| | | TATTAATttcctccacttagttctacacctcattcattcattcagtgagtgtttctcgactactatgaataaaccgttatactccatgttgc |
| | | gggcagaatggggatctggacagggaagcacagggcacgagttcaccaatggctgtcaagctacgctgcCCGCCTAACA |
| | | CTGCCAATGCCGGTCCCAAGCCCGGATAAAAAGTGGAGGGGGGGGATTTGTGGgaa |
| | | tctgcagatatccatcacact |
| 105 | twister | CCCCCTAACACTGCCAATGCCGGTCCCAAGCCCGGATAAAAAGTGGAGGGGGCG |
| | | G |
| 106 | hammerhead | ctgatgagtccgtgaggacgaaacgagtaagctcgtcacaaaa |
| 107 | HDV | gcccggcatggtcccagcctcctcgctggcgccggctcgccaactttccttcggcatggcgaatcggacGCGGCCGC |
| 108 | Hairpin 1 | AAACAGAGAAGTCAACCAGAGAAACACACGTTGTGGTATATTACCTGGTA |
| 109 | Hairpin 2 | CAACAGCGAAGCGCGCCAGGGAAACACACCATGTGTGGTATATTATCTGGCA |
| 110 | Hairpin 3 | CAACAGCGAAGCGGAACGGCGAAACACACCTTGTGTGGTATATTACCCGTTG |
| 111 | Varkud | GGGAAAGCTTGCGAAGGGCGTCGTCGCCCCGAGCGGTAGTAAGCAGGGAACTCA |
| | Satellite | CCTCCAATTTCAGTACTGAAATTGTCGTAGCAGTTGACTACTGTTATGTGATTGGTA |
| | | GAGGCTAAGTGACGGTATTGGCGTAAGTCAGTATTGCAGCACAGCACAAGCCCGC |
| | | TTGCGAGAAT |
| 112 | glms | TAATTATAGCGCCCGAACTAAGCGCCCGGAAAAAGGCTTAGTTGACGAGGATGGA |
| | | GGTTATCGAATTTTCGGGGGGATGCCTCCCGGCTGAGTGTGCAGATCACAGCCGTA |
| | | AGGATTTCTTCAAACCAAGGGGGTGACTCCTTGAACAAAGAGAAATCACATGATC |
| | | T |
| 113 | twister | GGACCCGCAAGGCCGACGGCATCCGCCGCCGCTGGTGCAAGTCCAGCCGCCCCG |
| | sister | GGGGGGGCGCTCATGGGTAAAC |
| 114 | twister | GGACCCGCAAGGCCGACGGCATCCGCCGCCGCTGGTGCAAGTCCAGCCGCCCCA |
| | sister AT | TGGGGGGGGCGCTCATGGGTAAAC |
| | insert | |
| 115 | pistol | GGAGCCGTTCGGGCGGCTATAAACAGACCTCAGGCCCGAAGCGTGGCGGCGATC |
| | | CCCCGGTGGTA |
| 116 | hatchet | CATTCCTCAGAAAATGACAAACCTGTGGGGCGTAAGTAGATATGTACATATCTATG |
| | | ATCGTGCAGACGTTAAAATCAGGT |
| 137 | HOXA13_ | agacccaagcttggtaccgagctcggatccgGCCACCATGGACTACAAAGACGATGACGACAAGttg |
| | 1 | ggtcttcccatggaaagctaccagccctgggcgctgcccaacggctggaacggccaaatgtactgccccaaagagcaggcgcagc |
| | | ctccccacctctggaagtccACACTTCCTGGTGAGTtggccTGCATGaTGCATGgtTGCATGaacac |
| | | aTATTAATttcTACACAGGGATGGGACCCCAGCCAGGGCATAGGCGACAGCTCGATC |
| | | TGAGCCACCCGCCTTGCAGCGCCCGGCTGCTCTTTGCCACCCGCTGTACAATCCTG |
| | | TCTTCTGCTAAAGCCTAGAGGGTCAGTGGGGAAGGTAGTTAGTTCTGACCGCCTA |
| | | ACACTGCCAATGCCGGTCCCAAGCCCGGATAAAAAGTGGAGGGGGCGGgaattctgca |
| | | gatatccatcacact |
| 118 | HOXA13_ | agacccaagcttggtaccgagctcggatccgGCCACCATGGACTACAAAGACGATGACGACAAGag |
| | 2 | ggtcttcccatggaaagctaccagccctgggcgctgcccaacggctggaacggccaaatgtactgccccaaagagcaggcgcagc |
| | | ctccccacctctggaagtccACACTTCCTGGTGAGTttgggcTGCATGacTGCATGgtTGCATGaacac |
| | | aTATTAATttcCCACCCCCCTTGCAGCGCCCGGCTGCTCTTTGCCACCCGCTGTACA |
| | | ATCCTGTCTTCTGCTAAAGCCTAGAGGGTCAGTGGGGAAGGTAGTTAGTTCTGAA |
| | | CTGAAATGAAATCACCCAGGGCTCCAGTGACTTCCCCAACCCGGCCATCCCGCCT |
| | | AACACTGCCAATGCCGGTCCCAAGCCCGGATAAAAAGTGGAGGGGGGGGgaattctgc |
| | | agatatccatcacact |
| 119 | HOXA13_ | agacccaagcttggtaccgagctcggatccgGCCACCATGGACTACAAAGACGATGACGACAAGttg |
| | 3 | ggtcttcccatggaaagctaccagccctgggcgctgcccaacggctggaacggccaaatgtactgccccaaagagcaggcgcagc |
| | | ctccccacctctggaagtccACACTTCCTGGTGAGTagggcTGCATGacTGCATGgTTGCATGaacac |
| | | aTATTAATH@cTGTCTTCTGCTAAAGCCTAGAGGGTCAGTGGGGAAGGTAGTTAGTT |
| | | CTGAACTGAAATGAAATCACCCAGGGCTCCAGTGACTTCCCCAACCCGGCCATGC |
| | | TGCAGGAGCAGCGCGTAGGCAGCCTGGAGTGATAGCCTGGTCCAACGGCCCGCCT |
| | | AACACTGCCAATGCCGGTGCCAAGCCCGGATAAAAAGTGGAGGGGGGGGgaattctgc |
| | | agatatccatcacact |
| 120 | HOXA13_ | agacccaagcttagtaccgagctcagatccgGCCACCATGGACTACAAAGACGATGACGACAAGttg |
| | 4 | ggtcttcccatggaaagctaccagccctgggcgctgcccaacggctggaacggccazatgtactgccccaaagagcaggcgcagc |
| | | ctccccacctctggaagtccACACTTCCTGGTGAGTttgggcTGCATGacTGCATGgTTGCATGaacac |
| | | aTATTAATHccACTGAAATGAAATCACCCAGGGCTCCAGTGACTTCCCCAACCCGGC |
| | | CATCCTGCAGGAGCAGGGCGTAGGCAGCCTCGAGTGATAGCCTGGTCCAACGGCC |
| | | CACACCTTAGCGCCAGGCTCAAGGTACAACACTTCTGTGCCTGCCTCCTCCGCCT |
| | | AACACTGCCAATGCCGGTCCCAAGCCCGGATAAAAAGTGGAGGGGGGGGgaattctgc |
| | | agatatccatcacact |
| 121 | HOXA13_ | agacccaagcttggtaccgagctcggatccgGCCACCATGGACTACAAAGACGATGACGACAAGttg |
| | 5 | ggtcttcccatggaaagctaccagccctgggcgctgcccaacggctggaacggccaaatgtactgccccaaagagcaggcgcagc |
| | | ctccccacctctggaagtccACACTTCCTGGTGAGTtttgggcTGCATGacTGCATGgtTGCATGaacac |
| | | aTATTAATttccCTGCAGGAGCAGCGCGTAGGCAGCCTCGAGTGATAGCCTGGTCCAA |
| | | CCCCCCACACCTTAGCGCCAGGCTCAAGGTACAACACTTCTGTGCCTGCCTCCTT |
| | | TCTGGGTGCCCGTCCCAATACTCGAAGCTTCTACACTGAAGCCATTTTTCCGCCTA |
| | | ACACTGCCAATGCCGGTGCCAAGCCCGGATAAAAAGTGGAGGGGGCGGgaattctgca |
| | | gatatccatcacact |
| 122 | hTT | agacccaagcttggtaccgagctcggatcccGCCACCATGGACTACAAAGACGATGACGACAAGggc |
| | HDV | ccggctgtggctgaggagccCctCcaccgaccGTGAGTtttgggcTGCATGacTGCATGgCTGCATGaacaca |
| | Cargo | TATTAATttcctccacttagttctacacctcattcattcattcagtgagtgtttctcgactactatgaataxaccgttatactccatgttgc |
| | | gggcagaatggggatctgacgaagcacagggcacgagttcaccaatggctgtcaagctacgctgcggcatggtccca |
| | | ttgcttcggcatggcgaatgggacGCGGCCGCgaattctgcagatatccatcaca |
| | | 5 |
| 123 | HTT | agacccaagcttgcgagctcgtccgGCCACCATGGACTACAAAGACGATGACGACAAGctct |
| | MALAT1 | tagaagagaaggtcaattgggagttgaattaactagccctgttgctgcttctgaagagtttgaagatgaagaag |
| | 2 KB | aaagtcctgttaatttgacgaggattttgacagctttcaaagtggtgaggacactgaagactggttttggctttaccaatg |
| | | tgactgcacaccaaaaatggaaattttcaagacctggcatcaggctcctttctgtcaaggcacagacagcacacattgtcctggaagat |
| | | ggaactaagatgaaagcttactcctttggccatccatcctctgttgctggtgaagtggtttttaatactggcctgggagggtacccagaag |
| | | ctattactgaccctgacagattctcacaatggccaaccctattattgggaatggtggagctcctgatactactgctctgga |
| | | tgaactgggacttagcaaatatttggagtctaatggaatcaaggtttcaggtttgctggtgctggattatagtaaagactacaaccactgg |
| | | ctggctaccaagagtttagggcaatggctacaggaagaaaaggttcctgcaatttatggagtggacacaagaatgctgactaaaataat |
| | | tcgggataagggtacctgcttgggaagattgaatttgaaggtcagcctgtggattttgtggatccaaataaacagaatttgattgctgag |
| | | gtttcaaccaaggatagtgtacggcaatgaRcaaaagtggtagctgtagactgtgggattaaactacaatgtaatcc |
| | | gcctgctagtaaagcgaggagctgaagtgcacttagttccctggaaccatgatttcaccaagatggagtatgatgggattttgatcgcg |
| | | ggaggaccggggacagctcttgcagaaccactaattcagaatgtcagaaagattttggaggatcgcaaggagccattgtttg |
| | | gaatcagtacaggaaactaacaggattggctgctggtgccaaaacctacaagatgtccatggccaacagagggcagaatcagc |
| | | Ctgttttgaatatcacaaacaaacaggctttcattactgctcagaatcatggctatgccttggacaacaccctccctgctggctggaaacc |
| | | actttttgtgaatgtcaacgatcaaacaaatgaggggattatgcatgagagcaaacccttcttcgctgtgcagttccacccagaggtcac |
| | | cccggggccaatagacactagtacctgtttgattcctttttctcactgataaagaaaggaaaagctaccaccattacatcagtcttaccg |
| | | aagccagcactagttgcatctcgggttgaggtttccaaagtccttattctaggatcaggaggtctgtccattggtcaggctggagaatttg |
| | | attactcaggatctcaagctgtaaaagccatgaaggaagaaaatgtcaaaactgttctgatgaacccaaacattgcatcagtccagacc |
| | | aatgaggtgggcttaaagcaagcggatactgtctactttcttcccatcacccctcagtttgtcacagaggtcatcaaggcagaacagcca |
| | | gatgggttaattctgggcatgggtggccagacagctctgaactgtggagtggaactattcaagagaggtgtgctcaaggaatatggtgt |
| | | gaaagtcctgggaacttcagttgagtccattatggctacggaagacaggcagctgttttcagataaactaaatgagatcaatgaaaagat |
| | | tgctccaagttttgcagtggaatcgattgaggatgcactgaaggcagcagacaccattggctacccagtgatgatccgttccgcctatg |
| | | cactgggtgggttaggctcaggcatctgtcccaacagagagactttgatggacctcagcacaaaggcctttgctatgaccaaccaaatt |
| | | ctggtggagaagtcagtgacggcccggctgtggctgaggagccCctCcaccgaccGTGAGTttgggcTGCATGacTG |
| | | CATGgTTGCATGaacacaTATTAATttcctccacttagttctacacctcattcattcattcagtgagtgtttctcgactactatg |
| | | aataaaccgttatactccatgttgcgggcagaatggggatctggacagcgaagcacagggcacgagttcaccaatggctgtcaagct |
| | | acgctgcCCGCCTAACACTGCCAATGCCGGTCCCAAGCCCGGATAAAAAGTGGAGGG |
| | | GGCGGgaaggtttttcttttcctgagaaaacaacacgtattgttttctcaggttttgctttttggcctttttctagcttaaaaaaaaaaaaaa |
| | | caaaagaattctgcagatatccatcacact |
| 124 | HTT | agacccaagcttcgtaccgagctcggatccgGCCACCATGGACTACAAAGACGATGACGACAAGgga |
| | MALAT1 | tcaatccctgaccctagtttgccccttccaaatcaacttgagatcgctctaaggaaaattaaggagttacatcgaataattctagaaacac |
| | 5 KB | gggcaacttgcaaatcactagaagagaaactaaaagttgaaagaatctgctttaaggttagcagaacaaaatatactgtcaagagacaa |
| | | agtaatcaatgaactgaggcttcgattgcctgccactgcagaaagagaaaagctcatagctgagctaggcagaaaagagatggaacc |
| | | aaaatctcaccacacattgaaaattgctcatcaattccattgcaaacatgcaagcaaggttaaatcaaaaagaagaagtattaaagaagt |
| | | atcaacgtcttctagaaaaagccagagaggagcaaagagaaattgtgaagaaacatgaggaagaccttcatattcttcatcacagatta |
| | | gaactacaggctgatagttcactaaataaattcaaacaaacggcttgggatttaatgaaacagtctcccactccagttcctaccaacaag |
| | | cattttattcgtctggctgagatggaacagacagtagcagaacaagatgactctctttcctcactcttggtcaaactaaagaaagtatcac |
| | | aagatttggagagacaaagagaaatcactgaattaaaagtaaaagaatttgaaaatatcaaattacagcttcaagaaaaccatgaagat |
| | | gaagtgaaaaaagtaaaagcggaagtagaggatttaaagtatcttctggaccagtcacaaaaggagtcacagtgtttaaaatctgaact |
| | | tcaggctcaaaaagaatcaagagctccaacaactacaatgagaaatctagtagaacggctaaagagccaattagccttgaag |
| | | gagaaacaacagaaagcacttagtcgggcacttttagaactccgggcagaaatgacagcagctgctgaagaacgtattatttctgcaa |
| | | cttctcaaaaagaggccttgttcaacaaatcgttgatcgacatactagagagctaaagacacaagttgaagatttaaatgaaa |
| | | atcttttaaaattgattagaagcacttaaaacaagtaaaaacagagaaaactcactaactgataatttgaatgacttaaataatgaactgca |
| | | aaagaaacaaaaagcctataataaaatacttagagagaaagaggaaattgatcaagagaatgatgaactgaaaaggcaaattaaaag |
| | | actaaccagtcgattacagcgcacccctgacagataataaacaaagtctaattgaagaactccaaaggaaagttaaaaaactagag |
| | | aaccaattagagggaaaggtggaggaagtagacctaaaacctatgaaagaaaagaatgctaaagaagaattaattaggtgggaaga |
| | | aggtaaaaagtggcaagccaactatagaaggaattcgaaacaagttaaaagagaaagagggggaagtctttactttaacaaagcagtt |
| | | gaatactttgaaggatctttaaagccgataaagagaaacttactttgcagaggaaactaaaaacaactggcatgactgttgatca |
| | | ggttttgggaatacgagctttggagtcagaaaaagaattggaagaattaaaaaagagaaatcttgacttagaaaatgatatattgtatatg |
| | | agggcccaccaagctcttcctcgagattctgttgtagaagatttacatttacaaaatagatacctccaagaaaaacttcatgctttagaaaa |
| | | acagttttcaaaggatacatattctaagccttcaatttcaggaatagagtcagatgatcattgtcagagagaacaggagcttcagaagga |
| | | aaacttgaagttgtcatctgaaaatattgaactgaaatttcagctttgaacaagcaaataaagatttgccaagattaaagaatcaagtcaga |
| | | gatttgaaggaaatgtgtgaatttcttaagaaagaaaaagcagaagttcagcgtacttggccatgttagagagtctggtagaagtgg |
| | | aaagacaatcccagaactggaaaaaaccattggtttaatgaaaaaagtagtttgaaaagagaaaatgaacagttgaaaaaa |
| | | gcatcaggaatattgactagtgaaaaaatggctaatattgagcaggaazatgaaaaattgaaggctgaattagaaaaacttaxagctcat |
| | | cttgggcatcagttgagcatgcactatgaatccaagaccaaaggcacagaaaaaattattgctgaaaatgaaaggcttcgtaaagaact |
| | | taaaaaagaaactgatgctgcagagaaattacggatagcaaagaataatttagagatattaaatgagaagatgacagttcaactagaag |
| | | agactggtaagagattgcagtttgcagaaagcagaggtccacagcttgaaggtgctgacagtaagagctggaaatccattgtggttac |
| | | Bagaatgtatgaaaccaagttaaaagaattggaaactgatattgccaaaaaaaatcaaagcattactgaccttaaacagcttgtaaaaga |
| | | agcaacagagagagaacaaaaagttaacaaatacaatgaagaccttgaacaacagattaagattcttaaacatgttcctgaaggtgctg |
| | | agacagagcaaggccttaaacgggagcttcaagttcttagattagctaatcatcagctggataxagagaaagcagaattaatccatcag |
| | | atagaagctaacaaggaccaaagtggagctgaaagcaccatacctgatgctgatcaactaaaggaaaaaataaaagatctagagaca |
| | | cagctcaaaatgtcagatctagaaaagcagcatttgaaggaggaaataaagaagctgaaaaaagaactggaaaattttgatccttcattt |
| | | tttgaagaaattgaagatcttaagtataattacaaggaagaagtgaagaagaatattctcttagaagagaaggtaaaaaaactttcagaac |
| | | aattgggagttgaattaactagccctgttgctgcttctgaagagtttgaagatgaagaagaaagtcctgttaatttccccatttacatgacg |
| | | aggattttgacagctttcaaagtggtgaggacactgaagactggttttggctttaccaatgtgactgcacaccaaaaatggaaattttcaa |
| | | gacctggcatcaggctcctttctgtcaaggcacagacagcacacattgtcctggaagatggaactaagatgaaaggttactcctttggc |
| | | catccatcctctgttgctggtgaagtggtttttaatactggcctgggagggtacccagaagctattactgaccctgcctacaaaggacag |
| | | attctcacaatggccaaccctattattgggaatggtggagctcctgatactactgctctggatgaactgggacttagcaaatatttggagtc |
| | | taatggaatcaaggtttcaggtttgctcgtgctggattatagtaaagactacaaccactggctggctaccaagagtttagggcaatggct |
| | | acaggaagaaaaggttcctgcaatttatggagtggacacaagaatgctgactaxaataattcgggataagggtaccatgcttgggaag |
| | | attgaatttgaaggtcagcctgtggattttgtggatccaaataaacagaatttgattgctgaggtttcaaccaaggatgtcaaagtgtacgg |
| | | caaaggaaaccccacaaattgtagctgtagactgtgggattaaaaacaatgtaatccgcctgctagtaaagcgaggagctgaagt |
| | | gcacttagttccctggaaccatgatttcaccaagatggagtatgatgggattttgatcgcgggaagaccgcggaacccagctcttgcag |
| | | aaccactaattcagaatgtcagaaagattttggagagtgatcgcaaggagccattgtttggaatcagtacaggaaacttaataacaggat |
| | | tggctgctggtgccaaaacctacaagatgtccatggccaacagagggcagaatcagcctgttttgaatatcacaaacaaacaggctttc |
| | | attactgctcagaatcatggctatgccttggacaacaccctccctgctggctggaaaccactttttgtgaatgtcaacgatcttaacaaatg |
| | | aggggattatgcatgagagcaaacccttcttcgctgtgcagttccacccagaggtcaccccggggccaatagttcactgagtacctgttt |
| | | gattcctttttctcactgataaagaaaggaaaagctaccaccattacatcagtcttaccgaagccagcactagttgcatctcgggttgagg |
| | | tttccaaagtccttattctatcaggaggtctgtccattggtcaggctggagaatttgattactcaggatctcaagctgtaaaagccatg |
| | | aaggaagaaaatgtcaaaactgttctgatgaacccaaacattgcatcagtccagaccaatgagggggcttaaagcaagcggatactg |
| | | tctactttcttcccatcacccctcagtttgtcacagaggtcatcaaggcagaacagccagatgggttaattctgggcatgggggccaga |
| | | cagctctgaactgtggagtggaactattcaagagaggtgtgctcaaggaatatggtgtgaaagtcctgggaacttcagttgagtccatta |
| | | tggctacggaagacaggcagctgttttcagataaactaaatgagatcaatgaaaagattgctccaagttttgcagtggaatcgattgagg |
| | | atgcactgaaggcagcagacaccattggctacccagtgatgatccgttccgcctatgcactggggggttaggctcaggcatctgtccc |
| | | aacagagagactttgatggacctcagcacactaggcctttgctatgaccaaccaaattctggtggagaagtcagtgacggcccggctgt |
| | | ggctgaggagccCcCCcaccgaccGTGAGTagggcTGCATGacTGCATGgtTGCATGaacacaTATTA |
| | | ATttcctccacttagttctacacctcattcattcattcagtgagtgtttctcgactactatgaataaacccttatactccatgttgcgggcaga |
| | | atggggatctggacagggaagcacagggcacgagttcaccaatggctgtcaagctacgctgcCCGCCTAACACTGCC |
| | | AATGCCGGTCCCAAGCCCGGATAAAAAGTGGAGGGGGCGGgaaggtttttcttttcctgagaaaa |
| | | caacacgtattgttttctcaggttttgctttttggcctttttctagcttaaaaaaaaaaaaagcaaaagaattctgcagatatccatcacact |
| 125 | HTT | agacccaagcttggtaccgagctcggtccgGCCACCATGGACTACAAAGACGATGACGACAAGatct |
| | MALAT1 | caatgttcaacaaatcgttgatcgacatactagagagctaaagacacaagttgaagatttaaatgaaaatcttttaaaattgaaagaagca |
| | 4 KB | cttaaaacaagtaaaaacagagaaaactcactaactgataatttgaatgacttaaataatgaactgcaaaagaaaaaaaagcctataat |
| | | aaaatacttagagagaaagaggaaattgatcaagagaatgatgaactgaaaaggcaaattaaaagactaaccagtggattacagggc |
| | | aaacccctgacagataataaacaaagtctaattgaagaactccaaaggaaagttaaaaaactagagaaccaattagagggaaaggtg |
| | | gaggaagtagacctaaaacctatgaaagaaaagaatgctaaagaagaattaattagggggaagaaggtaaaaagtggcaagccaa |
| | | aatagaaggaattcgaaacaagttaaaagagaaagagggggaagtctttactttaacaaagcagttgaatactttgaaggatctttttgcc |
| | | aaagccgataaagagaaacttactttgcagaggaaactaaaaacaactggcatgactgttgatcaggttttgggaatacgagctttgga |
| | | gtcagaaaaagaattggaagaattaaaaaagagaaatcttgacttagaaaatgatatattgtatatgagggcccaccaagctcttcctcg |
| | | agattctgttgtagaagatttacatttacaaaatagatacctccaagaaaaacttcatgctttagaaaaacagttttcaaaggatacatattct |
| | | aagccttcaatttcaggaatagagtcagatgatcattgtcagagagaacaggagcttcagaaggaaaacttgaagttgtcatctgaaaat |
| | | attgaactgaaatttcagcttgaacaagcaaataaagatttgccaagattaaagaatcaagtcagagatttgaaggaaatgtgtgaatttct |
| | | taagaaagaaaaagcagaagttcagcggaaacttggccatgttagagggtctggtagaagtggaaagacaatcccagaactggaaa |
| | | Raaccattggtttaatgaaaaaagtagttgaaaaagtccagagagaaaatgaacagttgaaaaaagcatcaggaatattgactagtgaa |
| | | aaaatggctaatattgagcaggaaaatgaaaaattgaaggctgaattagaaaaacttaaagctcatcttgggcatcagttgagcatgcac |
| | | tatgaatccaagaccaaaggccagaaaaaattattcctgaaaatgaaaggcttcgtaaagaacttaaaaaagaaactgatgctgcag |
| | | agttaattacggatagcaaagaataatttagagatattaaatgagaagatgacagttcaactagaagagactggtaagagattgcagtttg |
| | | cagaaagcagaggtccacagcttgaaggtgctgacagtaagagctggaaatccattgtggttacaagaatgtatgaaaccaagttacta |
| | | agaattggaaactgatattgccaaaaaaaatcaaagcattactgaccttaaacagcttgtaaaagaagcaacagagagagaacaaaaa |
| | | gtttaacaaatacaatgaagaccttgaacaacagattaagattcttaaacatgttcctgaaggtgctgagacagagcaaggccttaaacg |
| | | ggagcttcaagttcttagttttagctaatcatcagctggataaagagaaagcagaattaatccatcagatagaagctaacaaggaccaaa |
| | | gtggagctgaaagcaccatacctgatgctgatcaactaaaggaaaaaataaaagatctagagacacagctcaaaatgtcagatctaga |
| | | aaagcagcatttgaaggaggaaataaagaagctgaaaaaagaactggaazattttgatccttcattttttgaagaaattgaagatcttaag |
| | | tataattacaaggaagaagtgaagaagaatattc.cctagaagagaaggtaaaaaaactttcaggcgagttgaattaactagc |
| | | cctgttgctgcttctgaagagtttgaagatgaagaagaaagtcctgttaatttccccatgacgagattttgacagctttcaaagtg |
| | | gtgaggacactgaagactggttttggctttaccaatgtgactgcacaccaaaaatggaaattttcaagacctggcatcaggctcctttctg |
| | | tcaaggcacagacagcacacattgtcctggaagatggaactaagatgaaaggttactcctttggccatccatcctctgttgctggtgaag |
| | | tggtttttaatactggcctgggagggtacccagaagctattactgaccctgcctacaaaggacagattctcacaatggccaaccctattat |
| | | tgggaatggtggagctcctgatactactgctctggatgaactgggacttagcaaatatttggagtctaatggaatcaaggtttcaggtttg |
| | | ctggtgctggattatagtaaagactacaaccactggctggctaccaagagtttagggcaatggctacaggaagaaaaggttcctgcaat |
| | | ttatggagtggacacaagaatgctgactaaaataattcgggataagggtaccatgcttgggaagattgaatttgaaggtcagcctgtgg |
| | | attttgtggatccaaatactacagaatttgattgctgaggtttcaaccaaggatgtcaaagtgtacggcaaaggaaaccccacaaaagtg |
| | | gtagctgtagactgtgggattaxaaacaatgtaatccgcctgctagtaaagcgaggagctgaagtgcacttagttccctggaaccatga |
| | | ttcaccaagatggagtatgatgggattttgatcgcgggaggaccggggaacccagctcttgcagaaccactaattcagaatgtcagaa |
| | | agattttggagagtgatcgcaaggagccattgttggaatcagtacaggaaacttaataacaggattggctgctggtgccaaaacctac |
| | | aagatgtccatggccaagagggcagaatcagcctgttttgaatatcacaaacaaacaggctttcattactgctcagaatcatggctat |
| | | gccttggacaacaccctccctgctggctggaaaccactttttgtgaatgtcaacgatcaaacaaatgaggggattatgcatgagagcaa |
| | | acccttcttcgctgtgcagttccacccagaggtcaccccggggccaatagacactgagtacctgtttgattcctttttctcactgataaaga |
| | | aaggaaaagctaccaccattacatcagtcttaccgaagccagcactagttgcatctcgggttgaggtttccaaagtccttattctaggatc |
| | | aggaggtctgtccattggtcaggctggagaatttgattactcaggatctcaagctgtaaaagccatgaaggaagaaaatgtcaaaactg |
| | | ttctgatgaacccaaacattgcatcagtccagaccaatgaggtgggcttaaagcaagcggatactgtctactttcttcccatcacccctca |
| | | gttttgtcacagaggtcatcaaggcagaacagccagatgggttaattctgggcatgggggccagacagctctgaactgtggagtggtta |
| | | ctattcaagagaggtgtgctctaggaatatggtgtgaaagtcctgggaacttcagttgagtccattatggctacggaagacaggcagct |
| | | gttttcagataaactaaatgagatcaatgaaaagattgctccaagttttgcagtggaatcgattgaggatgcactgaaggcagcagacac |
| | | cattggctacccagtgatgatccgttccgcctatgcactggggggttaggctcaggcatctgtcccaacagagagactttgatggacct |
| | | cagcacaaaggcctttgctatgaccaaccaaattctggtggagaagtcagtgacggcccggctgtggctgaggagccCctCcaccg |
| | | AccGTGAGTttgggcTGCATGacTGCATGgtTGCATGaacacaTATTAATttcctcchtcttagttctacacctc |
| | | attcattcattcagtgagtgtttctcgactactatgaataaaccgttatactccatgttgcgggcagaatggggatctggacagggaagca |
| | | cagggcacgagttcaccaatggctgtcaagctacgctccCCGCCTAACACTGCCAATGCCGGTGCCAAG |
| | | CCCGGATAAAAAGTGGAGGGGGGGGgaaggtttttcttttcctgagaaaacaacacgtattgttttctcaggttttg |
| | | cttaaaaaaaaaaaaagcaaaagaattctgcagatatccatcacact |
| 126 | HTT | agacccaagcttggtaccgagctcggatccgGCCACCATGGACTACAAAGACGATGACGACAAGaat |
| | MALAT1 | caatttggatgcacagaaggtggaacagatgttaagagatgaattagctgatagtgtgagcaaggcagtaagtgatgctgataggcaa |
| | 6 KB | cggattctagaattagagaagaatgaaatggaactaaaagttgaagtctcaaaactgagagagatttctgatattgccagaagacaagtt |
| | | gaaattttgaatgcacaacaacaatctagggacaaggaagtagagtccctcagaatgcaactgctagactatcaggcacagtctgatg |
| | | aaaagtcgctcattgccaagttgcaccaacataatgtctctcttcaactgagtgaggctactgctcttggtaagttggagtcaattactttct |
| | | aaactgcagaagatggaggcctacaacttgcgcttagagcagaaacttgatgaaaaagaacaggctctctattatgctcgtttggaggg |
| | | aagaaacagagcaaaaatctgcgccaaacaattcagtctctacgacgacagtttagtggagctttacccttggcacaacaggaaaag |
| | | ttctccaaaacaatgatatgacaaacttaagataatgcaagaaatgaaaaattctcaacaagaacatagaaatatggag |
| | | aacaaaacattggagatggaattaaaattaaagggcctggaagagttaataagcactttaaaggataccaaaggagcccactaaggtaa |
| | | tcaactggcatatgaaaatagaagaacttcgtcttcaagaacttaaactaaatcgggaattagtcaaggataaagattgaaataaaatattt |
| | | gaataacataatttctgaatatgaacgtacaatcagcagtcttgaagaagaaattgtgcaacagaacaagtttcatgaagaaagacaaat |
| | | ggcctgggatcaaagagaagttgacctggaacgccaactagacatttttgaccgtcagcaaaatgaaatactaaatgcggcacaaaag |
| | | tttgaagaagctacaggatcaatccctgaccctagtttgccccttccaaatcaacttgagatcgctctaaggaaaattaaggagaacattc |
| | | gaataattctagaaacacgacaacttgcaaatcactagaagagaaactaaaagagaaagaatctgctttaaggttagcagaacaaaa |
| | | tatactgtcaagagacaaagtaatcaatgaactgaggcttcgattgcctgccactgcagaaagagaaaagctcatagctgagctaggc |
| | | agaaaagagatggaaccaaaatctcaccacacattgaaaattgctcatcaaccattgcaaacatgcaagcaaggttaaatcaaaaag |
| | | aagaagtattaaagaagtatcaacgtcttctagaaaaagccagagaggagcaaagagaaattgtgaagaaacatgaggaagaccttc |
| | | atattcttcatcacagattagaactacaggctgatagttcactaaataaattcaaacaaacggcttgggatttaatgaaacagtctcccact |
| | | ccagttcctaccaacaagcattttattcgtctggctgagatggaacagacagtagcagaacaagatgactctctttcctcactcttggtca |
| | | aactaaagaaagagagacaaagagaaatcactgaattaaaagtaaaagaatttgaaaatatcaaattacagcttc |
| | | aagaaaaccatgaagatgaagtgaaaaaagtaactagcggaagtagaggatttaaagtatcttctggaccagtcacaaaaggagtcac |
| | | agtgtttaazatctgaacttcaggctcaaaaagaagcaaattcaagagctccaacaactacaatgagaaatctagtagaacggctaaag |
| | | agccaattagccttgaaggagaaacaacagaaagcacttagtcgggcacttttagaactccgggcagaaatgacagcagctgctgaa |
| | | gaacgtattatttctgcaacttctcaaaaagaggcccatctcaatgttcaacaaatcgttgatcgacatactagagagctaaagacacaag |
| | | tttgaagatttaaatgaaaatcttttaaaattgaaagaagcacttaaaacaagtaaaaacagagaaaactcactaactgataatttgaatgac |
| | | ttaaataatgaactgcaaaagaaacaaaaagcctataataaaatacttagagagaaagaggaaattgatcaagagaatgatgaactgaa |
| | | aaggcaaattaaaagactaaccagtggattacagggcaaacccctgacagataataaacaaagtctaattgaagaactccaaaggaa |
| | | agttaaaaaactagagaaccaattagagggaaaggtggaggaagtagacctaaaacctatgaaagaaaagaatgctaaagaagaatt |
| | | aattaggtgggaagaaggtaaaaagtggcaagccaaaatagaaggaattcgaaacaagttaaaagagaaagagggggaagtcttta |
| | | ctttaacaaagcagttgaatactttgaaggatctttttgccaaagccgataaagagaaacttactttgcagaggaaactaaaaacaactgg |
| | | catgactgttgatcaggttttgggaatacgagctttggagtcagaaaaagaattggaagaattaaaaaagagaaatcttgacttagaaaa |
| | | tgatatattgtatatgagcgcccaccaagctcttcctcgagattctgttgtagaagatttacatttacaaaatagatacctccaagaaaaact |
| | | tcatgctttagaaaaacagttttcaaaggatacatattctaagccttcaatttcaggaatagagtcagatgatcattgtcagagagaacagg |
| | | agcttcagaaggaaaacttgaagttgtcatctgaaaatattgaactgaaatttcagcttgaacaagcaaataaagatttgccaagattaaa |
| | | gaatcaagtcagagatttgaaggaaatgtgtgaatttcttaagaaagaaaaagcagaagttcagcggaaacttggccatgttagagggt |
| | | ctggtagaagtggaaagacaatcccagaactggaaaaaaccattggtttaatgaaaaaagtagttgaaaaagtccagagagaaaatga |
| | | acagttgaaaaaagctcaggaatattgactagtgaaaaaatggctaatattgagcaggaaaatgaaaaattgaaggctgaattagaaa |
| | | aacttaaagctcatcttgggcatcagttgagcatgcactatgaatccaagaccaaaggcacagaaaaaattattgctgaaaatgaaagg |
| | | cttcgtaaagaacttaaaaaagaaactgatgctgcagagaaattacggatagcaaagaataatttagagatattaaatgagaagatgac |
| | | agttcaactagaagagactggtaagagattgcagtttgcagaaagcagaggtccacagcttgaaggtgctgacagtaagagctggaa |
| | | atccattgtcgttacaagaatgtatgaaaccaagttaaaagaattggaaactgatattgccaaaaaaaatcaaagcattactgaccttaaa |
| | | cagcttgtaaaagaagcaacagagagagaacaaaaagttaacaaatacaatgaagaccttgaacaacagattaagattcttaaacatgt |
| | | tcctgaaggtgctgagacagagcaaggccttaaacgggagcttcaagttcttagattagctaatcatcagctggataaagagaaagca |
| | | gaattaatccatcagatagaagctaacaaggttccaaagtggagctgaaagcaccatacctgatgctgatcaactaaaggaaaaaataa |
| | | aagatctagagacacagctcaaaatgtcagatctagaaaagcagcatttgaaggaggaaataaagaagctgaaaaaagaactggaaa |
| | | attttgatccttcattttttgaagaaattgaagatcttaagtataattacaaggaagaagtgaagaagaatattctcttagaagagaaggtaa |
| | | aaaaactttcagaacaattgggagttgaattaactagccctgttgctgcttctgaagagtttgaagatgaagaagaaagtcctgttaatttc |
| | | cccatttacatgacgaggattttgacagctttcaaagtgatgaggacactgaagactggttttggctttaccaatgtgactgcacaccacta |
| | | aatggaaattttcaaggcatcaggctcctttctgtcaaggcacagacagcacacattgtcctggaagatggaactaagatgaaa |
| | | ggttactcctttggccatccatcctctgttgctggtgaagtggtttttaatactggcctgggagggtacccagaagctattactgaccctgc |
| | | ctacaaaggacagatttggccaaccctattattgggaatggtggagctcctgatactactgctctggatgaactgggacttag |
| | | caaatatttggagtctaatggaatcaaggtttcaggtttgctggtgctggattatagtaaagactacaaccactggctggctaccaagagt |
| | | ttagggcaatggctacaggaagaaaaggttcctgcaatttatggagtggacacaagaatgctgactaaaataattcgggataagggtac |
| | | catgcttggaagattgaatttgaaggctcagcctgtggattttgtggatccaaataaacagaatttgattgctgagggtttcaaccaaggatg |
| | | tcaaagtgtacggcacacattaagtggtagctgtagactgtgggattaaaaacaatgtaatccgcctgctagtaaagcg |
| | | aggagctgaagtgcacttagttccctggaaccatgatttcaccaagatggagtatgatgggattttgatcgcgggaggaccggggaac |
| | | ccagctcttgcagttaccactaattcagaatgtcagaaagattttggagagtgatcgcaaggagccattgtttggaatcagtacaggaaa |
| | | cttaataacaggattggctgctggtgccaaaacctacaagatgtccatggccaacagagggcagaatcagcctgttttgaatatcacaa |
| | | acaaacaggctttcattactgctcagaatcatggctatgccttggacaacaccctccctgctggctggaaaccactttttgtgaatgtcaa |
| | | cgatcaaacaaatgaggggattatgcatgagagcaaacccttcttcgctgtgcagttccacccagaggtcaccccggggccaataga |
| | | cactgagtacctgtttgattcctttttctcactgataaagaaaggaaaagctaccaccattacatcagtcttaccgaagccagcactagttg |
| | | catctcgggttgaggtttccaaagtccttattctaggatcaggaggtctgtccattggtcaggctggagaatttgattactcaggatctcaa |
| | | gctgtaaaagccatgaaggaagaaaatgtcaaaactgttctgatgaacccaaacattgcatcagtccagaccaatgttggtgggcttaa |
| | | agcaagcggatactgtctactttcttcccatcacccctcagttgtcacagaggtcatcaaggcagaacagccagtttgggttaattctgg |
| | | gcatgggtggccagacagctctgaactgtggagtggaactattcaagagaggtgtgctcaaggaatatggtgtgaaagtcctgggaa |
| | | cttcagttgagtccattatggctacggaagacaggcagctgttttcagataaactaaatgagatcaatgaaaagattgctccaagtttgc |
| | | agtggaatcgattgaggatgcactgaaggcagcagacaccattggctacccagtgatgatccgttccgcctatgcactggggggtta |
| | | ggctcaggcatctgtcccaacagagagactttgatggacctcagcacaaaggcctttgctatgaccaaccaaattctggtggagaagtc |
| | | agtgacggcccggctgtagctgaggagccCctCcaccgaccGTGAGTttgggcTGCATGacTGCATGgITGCA |
| | | TOaacacaTATTAATttcctccacttagttctacacctcattcattcattcagtgagtgtttctcgactactatgaataaaccgttatact |
| | | ccatgttgcgggcagaatggggatctggacagcgaagcacagggcacgagttcaccaatggctgtcaagctacgctgcCCOCC |
| | | TAACACTGCCAATGCCGGTOCCAAGCCOGGATAAAAAGTGGAGGGGGCGCgaaggttt |
| | | tcttttcctgagaaaacaacacgtattgttttctcaggttttgctttttggcctttttctagcttaaaaaaaaaagcaaaagaattctgcag |
| | | atatccatcacact |
| 127 | HTT | agacccaagcttggtaccgagctcggatccgGCCACCATGGACTACAAAGACGATGACGACAAGgaa |
| | MALAT1 | tagttgaggctctgaagaggttaaaagattatgaatcgggagtatatggtttagaagatgctgtcaaattgtaaaaacc |
| | 8 KB | aaattttaaataagagatcgagagattgaaatattaacaaaggaaatcaataaacttgaattgaagatcagcttgatgaaaatga |
| | | ggcacttagagagcgtgtgggccttgaaccaaagachatgattgatttaactgaatttagaaatagcaaacacttaaaacagcagcagt |
| | | acagagctgaaaaccagattcttttgaaagagattgaaagtctagaggaagaacgacttgatctgaaaaaaaaaattcgtcaaatggct |
| | | caagactagaggttaaaagaagtgcaacttcaggattaaccactgaggacctgaacctaactgaaaacatttctcaaggagatagaataa |
| | | gtgaaagaaaattggatttattgagcctcaaaaatatgagtgaagcacaatcaaagaatgaatttctttcaagagaactaattgaaaaaga |
| | | aagagatttagaaaggagtaggacagtgatagccaaatttcagaataaattaaaagaattagttgaagaaaataagcaacttgaagaag |
| | | gtatgaaagaaatattgcaagcaattaaggaaatgcagaaagatcctgatgttaaaggaggagaaacatctctaattatccctagccttg |
| | | aaagactagttaatgctatagaatcaaagaatgcagaaggaatctttgatgcgagtctgcatttgaaagcccaagttgatcagcttaccg |
| | | gaagaaatgaagaattaagacaggagctcagggaatctcggaaagaggctataaattattcacagcagttggcaaaagctaatttaaa |
| | | gatagaccatcttgaaaaagaaactatgtcttttacgacaatcagaaggatcaaatgttgtttttaaaggaattgacttacctgatgggatag |
| | | caccatctagtcccagtatcattaattctcagaatgaatatttaatacatttgttacaggaactagaaaataaagaaaaaaagttaaagaatt |
| | | tagaagattctcttgaagattacaacagactaatttgctgtaattcgtcatcaacaaagtttgttgtataaagaatacctaagtgaaaaggag |
| | | acctggaaaacagaatctaaacaataaaagaggaaaagagaaaacttgaggatcaagtccaacaagatgctataaaagtaaaagaa |
| | | tataataatttgctcaatgtcttcagatggattcggatgaaatgaaaaaaatacttgcagaaaatagtaggaaaattactgttttgcaagtg |
| | | aatgaaaaatcacttataaggcaatatacaaccttagtagaattggagcgacaacttagaaaagaaaatgagaagcaaaagaatgaatt |
| | | gtttgtcaatggaggctgaagtttgtgaaaaaattgggtgtttgcaaagatttaaggaaatggccattttcaagattgcagctctccaaaaa |
| | | gtttgtagataatagtgtttctttgtctgaactagaactggctaataaacagtacaatgaactgactgctaagtacagggacatcttgcaaaa |
| | | agataatatgcttgttcaaagaacaagtaacttggaacacctggagtgtgaaaacatctccttaaaagaacaagtggagtctataaataa |
| | | agaactggagattaccaaggaaaaacttcacactattgaacaagcctgggaacaggaaactaaattaggtaatgaatctagcatggata |
| | | aggcaaagaaatcaataaccaacagtgacattgtttccatttcaaaaaaaataactatgctggaaatgaaggaattaaatgaaaggcag |
| | | cgggctgaacattgtcaaaaaatgtatgaacacttacggacttcgttaaagcaaatggaggaacgtaattttgaattggaaaccaaattttg |
| | | ctgagcttaccaaaatcaatttggatgcacagaaggtggaacagatgttaagagatgaattagctgatagtgtgagcaaggcagtaagt |
| | | gatgctgataggcaacggattctagaattagagaagaatgaaatggaactaaaagttgaagtgtcaaaactgagagagatttctgatatt |
| | | gccagaagacaagttgaaattttgaatgcacaacaacaatctagggacaaggaagtagagtccctcagaatgcaactgctagactatc |
| | | aggcacagtctgatgaaaagtcgctcattgccaagttgcaccaacataatgtctctcttcaactgagtgaggctactgctcttggtaagtt |
| | | ggagtcaattacatctaaactgcagaagatggaggcctacaacttgcgcttagagcagaaacttgatgaaaaagaacaggctctctatt |
| | | atgctcgtttggagggaagaaacagagcaaaacatctgcgccaaacaattcagtctctacgacgacagtttagtggagctttacccttg |
| | | gcacaacaggaaaagttctccaaaacaatgattcaactacaaaatgacaaacttaagataatgcaagaaatgaaaaattctcaacaaga |
| | | acatagaaatatggagaacaaaacattggagatggaattaaaattaaagggcctggaagagttaataagcactttaaaggataccaaa |
| | | ggagcccaaaaggtaatcaactggcatatgaaaatagaagaacttcgtcttcaagaacttaaactaaatcgggaattagtcaaggataa |
| | | agaagactataaaatatttgaataacataatttctgaatatgaacgtacaatcagcagtcttgaagaagaaattgtgcaacagaacaagttt |
| | | catgaagttaagacaaatggcctgggatcaaagagaagttgacctggaacgccaactagacatttttgaccgtcagcaaaatgaaatac |
| | | taaatgcggcacaaaagtttgaagaagctacaggatcaatccctgaccctagtttgccccttccaaatcaacttgagatcgctctaagga |
| | | aaattaaggagaacattcgaataattctagaaacacgggcaacttgcaaatcactagaagagaaactaaaagagaaagaatctgcttta |
| | | agcttagcagaacaaaatatactgtcaagagacaaagtaatcaatgaactgaggcttcgattgcctgccactgcagaaagagaaaagc |
| | | tcatagctgagctaggcagaaaagagatggaaccaaaatctcaccacacattgaaaattgctcatcaaaccattgcaaacatgcaagc |
| | | aaggttaaatcaaaaagaagaagtattaaagaagtatcaacgtcttctagaaaaagccagagaggagcaaagagaaattgtgaagaa |
| | | acatgaggaagaccttcatattcttcatcacagattagaactacaggctgatagttcactaaataaattcaaacaaacggcttgggatttaa |
| | | tgaaacagtctcccactccagttcctaccaacaagcattttattcgtctcgctgagatggaacagacagtagcagaacaagatgactctc |
| | | tttcctcactcttggtcaaactaaagaaagtatcacaagatttggagagacaaagagaaatcactgaattaaaagtaaaagaatttgaaaa |
| | | tatcaaattacagcttcaagaaaaccatgaagatgaagtgaaaaaagtaaaagcggaagtagaggatttaaagtatcttctggaccagt |
| | | cacaaaaggagtcacagtgtttaaaatctgaacttcaggctcaaaaagaagcactattcaagagctccaacaactacaatgagaaatcta |
| | | gtagaacggctaaagagccaattagccttgaaggagaaacaacagaaagcacttagtcgggcacttttagaactccgggcagaaatg |
| | | acagcagctgctgaagaacgtattatttctgcaacttctcaaaaagaggcccatctcaatgttcaacaaatcgttgatcgacatactagag |
| | | agctaaagacacaagttgaagatttaaatgaaaatcttttaaaattgaaagaagcacttaaaacaagtaaaaacagagaaaactcactaa |
| | | ctgataatttgaatgacttaaataatgaactccaaaagaaacaaaaagcctataataaaatacttagagagaaagaggaaattgatcaag |
| | | agaatgatgaactgaaaaggcaaattaaaagactaaccagtggattacagggcaaacccctgacagataataaacaaagtctaattga |
| | | agaactccaaaggaaagttaaaaaactagagaaccaattagagggaaaggggaggaagtagacctaaaacctatgaaagaaaaga |
| | | atgctaaagaagaattaattaggtgggaagaaggtaaaaagtggcaagccaaaatagaaggaattcgaaacaagttaaaagagaaag |
| | | agggggaagtctttactttaacaaagcagttgaatactttgaaggatctttttgccaaagccgataaagagaaacttactttgcagaggaa |
| | | actaaaaacaactggcatgactgttgtcaggttttgggaatacgagctttggagtcagaaaaagaattggaagaattaaaaaagagaa |
| | | atcttgacttagaaaatgatatattgtatatgagggcccaccaagctcttcctcgagattctgttgtagaagatttacatttacaaaatagata |
| | | cctccaagaaaaacttcacagttttcaaaggatacatattctaagccttcaatttcaggaatagagtcagatgatcattg |
| | | tcagagagaacaggagcttcgaaggaaaacttgaagttgtcatctgaaaatattgaactgaaatttcagcttgaacaagcaaataaag |
| | | atttgccaagattaaagaatcaagtcagagatttgaaggaaatgtgtgaatttcttaagaaagaaaaagcagaagttcagcggaaacttg |
| | | gccatgttagagggtctgtagaagtggaaagacaatcccagaactggaaaaaaccattggtttaatgaaaaaagtagttgaaaaagt |
| | | ccagagagaaaatgaacagttgaaaaaagcatcaggaatattgactagtgaaaaaatcgctaatattgagcaggaaaatgaaaaattg |
| | | Baggctgaattagaaaaacttaaagctcatcttgggcatcagttgagcatgcactatgaatccaagaccaaaggcacagaaaaaattatt |
| | | gctgaaaatgaaaggcttcgtaaagattcttaaaaaagaaactgatgctgcagagaaattacggatagcaaagaataatttagagatatt |
| | | aaatgagaagatgacagttcactctagaagagactggtaagagattgcagttttgcagaaagcagaggtccacagcttgaaggtgctga |
| | | cagtaagagctggaaatccattgtggttacaagaatgtatgaaaccaagttaaaagaattggaaactgatattgccaaaaaaaatcaaa |
| | | gcattactgaccttaaacagcttgtaaaagaagcaacagagagagaacaaaaagttaacaaatacaatgaagaccttgaacaacagat |
| | | taagattcttaaacatgttcctgaaggtgctgagacagagcaaggccttaaacgggagcttcaagttcttagattagctaatcatcagctg |
| | | gataaagagaaagcagaattaatccatcagatagaagctaacaaggaccaaagtggagctgaaagcaccatacctgatgctgatcaa |
| | | ctaaaggaaaaaataaaagatctagagacacagctcaaaatgtcagatctagaaaagcagcatttgaaggaggaaataaagaagctg |
| | | aaaaaagaactcgaaaattttgatccttcattttttgaagaaattgaagatcttaagtataattacaaggaagaagtgaagaagaatattctc |
| | | ttagaagagaaggtaaaaaaactttcagaacaattgggagttgaattaactagccctgttgctgcttctgaagagtttgaagatgaagaa |
| | | gaaagtcctgttaatttccccatttacatgacgaggattttgacagctttcaaagtggtgaggacactgaagactggttttggctttaccaat |
| | | gtgactgcacaccaaaaatggaaattttcaagacctggcatcaggctcctttctgtcaaggcacagacagcacacattgtcctggaaga |
| | | tggaactaagatgaaaggttactcctttggccatccatcctctgttgctggtgaagtggtttttaatactggcctgggagggtacccagaa |
| | | gctattactgaccctgcctacaaaggacagattctcacaatggccaaccctattattgggaatggtggagctcctgatactactgctctgg |
| | | atgaactgggacttagcaaatatttggagtctaatggaatcaaggtttcaggtttgctggtgctggattatagtaaagactacaaccactg |
| | | gctggctaccaagagtttaacaatggctacaggaagaaaaggttcctgcaatttatggagtggacacaagaatgctgactaaaata |
| | | attcgggataagggtaccatgcttgggaagattgaatttgaaggtcagcctgtggattttgtggatccaaataaacagaatttgattgctg |
| | | aggtttcaaccaaggatgtcaaagtgtacggcaaaggaaaccccacaaaagtggtagctgtagactgtgggattaaaaacaatgtaat |
| | | ccgcctgctagtaaagcgaggagctgaagtgcacttagttccctggaaccatgatttcaccaagatggagtatgatgggattttgatcgc |
| | | gggaggaccgcggaacccagctcttgagaacactaattcagaatgtcagaaagattttggagagtgatcgcaaggagccattgttt |
| | | ggaatcagtacaggaaacttaataacaggattggctgctggtgccaaaacctacaagatgtccatggccaacagagggcagaatcag |
| | | cctgttttgaatatcacaaacaaacaggctttcattactgctcagaatcatggctatgccttggacaacaccctccctgctggctggaaac |
| | | cactttttgtgaatgtcaacgatcaaacaaatgaggggattatgcatgagagcaaacccttcttcgctgtgcagttccacccagaggtca |
| | | ccccggggccaatagacactgagtacctgtttgattcctttttctcactgataaagaaaggaaaagctaccaccattacatcagtcttacc |
| | | gaagccagcactagttgcatctcgggttgaggtttccaaagtccttattctaggatcaggaggtctgtccattggtcaggctggagaattt |
| | | gattactcaggatctcaagctgtaaaagccatgaaggaagaaaatgtcaaaactgttctgatgaacccaaacattgcatcagtccagac |
| | | caatgaggtgggcttaaagcaagcggatactgtctactttcttcccatcacccctcagtttgtcacagaggtcatcttaggcagttacagcc |
| | | agatgggttaattctgggcatgggtggccagacagctctgaactgtggagtggaactattcaagagaggtgtgctcaaggaatatggt |
| | | gtgaaagtcctgggaacttcagttgagtccattatggctacggaagacaggcagctgttttcagataaactaaatgagatcaatgaaaag. |
| | | attgctccaagttttgcagtggaatcgattgaggatgcactgaaggcagcagacaccattggctacccagtgatgatccgttccgcctat |
| | | gcactggggggttaggctcaggcatctgtcccaacagagagactttgatggacctcagcacaaaggcctttgctatgaccaaccaaa |
| | | ttctggtggagaagtcagtgacggcccggctgtggctgaggagccCctCcaccgaccGTGAGTttgggcTGCATGacT |
| | | GCATOgtTOCATGaacacaTATTAATttcctccacttagttctacacctcattcattcattcagtgagtgtttctcgactactat |
| | | gaataaaccgttatactccatgttgcgggcagaatggggatctggacagggaagcacagggcacgagttcaccaatggctgtcaagc |
| | | tacgctgcCCGCCTAACACTGCCAATGCCGGTCCCAAGCCCGGATAAAAAGTGGAGGG |
| | | GGCGGgaaggtttttcttttcctgagaaaacaacacgtattgttttctcaggttttgctttttggcctttttctagcttaaaaaaaaaaaag |
| | | caaaagaattctacaccatcacact |
| 128 | HTT | agacccaagcttggtaccgagctcggatccgGCCACCATGGACTACAAAGACGATGACGACAAGGC |
| | MALAT1 | CATGAAGATCGAGTGCCGCATCACCGGCACCCTGAACGGCGTGGAGTTCGAGCTG |
| | 10 KB | GTGGGGGGCGGAGAGGGCACCCCCGAGCAGGGCCGCATGACCAACAAGATGAA |
| | | GAGCACCAAAGGCGCCCTGACCTTCAGCCCCTACCTGCTGAGCCACGTGATGGGC |
| | | TACGGCTTCTACCACTTCGGCACCTACCCCAGCGGCTACGAGAACCCCTTCCTGCA |
| | | CGCCATCAACAACGGCGGCTACACCAACACCCGCATCGAGAAGTACGAGGACGG |
| | | CGGCGTGCTGCACGTGAGCTTCAGCTACCGCTACGAGGCCGGCCGCGTGATCGGC |
| | | GACTTCAAGGTGGTGGGCACCGGCTTCCCCGAGGACAGCGTGATCTTCACCGACA |
| | | AGATCATCCGCAGCAACGCCACCGTGGAGCACCTGCACCCCATGGGCGATAACGT |
| | | GCTGGTGGGCAGCTTCGCCCGCACCTTCAGCCTGCGCGACGGCGGCTACTACAGC |
| | | TTCGTGGTGGACAGCCACATGCACTTCAAGAGCOCCATCCACCCCAGCATOCTOC |
| | | AGAACGGGGGCCCCATGTTCGCCTTCCGCCGCGTGGAGGAGCTGCACAGCAACA |
| | | CCGAGCTGGGCATCGTGGAGTACCAGCACGCCTTCAAGACCCCCATCOCCTTCGC |
| | | CAGATCTCGAGCTCGAagtcgacatgccacctaatataaactggaaagaaataatgaaagttgacccagatgacctgc |
| | | cccgtcaagaagaactggcagataatttattgatttccttatccaaggtggaagtaaatgagctaaaaagtgaaaagcaagaaaatgtga |
| | | tacaccttttcagaattactcagtcactaatgaagatgaaagctcaagaagtggagctggctttggaagaagtagaaaaagctggagaa |
| | | gaacaagcaactatttgaaaatcaattaaaaactaaagtaatgaaactggaaaatgaactggagatggctcagcagtctgcaggtggac |
| | | gagatactcggtttttacgtaatgaaatttgccaacttgaaaaacaattagaacaaaaagatagagaattggaggacatggaaaaggag |
| | | ttggagaaagagaagaaagttaatgagcaattggctcttcgaaatgaggaggcagaaaatgaaaacagcaaattaagaagagagaa |
| | | caaacgtctaaagaaaaagaatgaacaactttgtcaggatattattgactaccagazacaaatagattcacagaaagaaacacttttatca |
| | | agaagaggggaagacagtgactaccgatcacagttgtctaaaaaaaactatgagcttatccaatatcttgatgaaattcagactttaaca |
| | | gaagctaatgagaaaattgaagttcagaatcagaaatgagaaaaaatttagaagagtctgtacaggaaatggagaagatgactgatg |
| | | aatataatagaatgaaagctattgtgcatcagacagataatgtaatagatcagttaaaaaaagaaaacgatcattatcaacttcaagtgca |
| | | ggagcttacagatcttctgaaatcaaaaaatgaagaagatgatccaattatggtagctgtcaatgcaaaagtagaagaatggaagctaat |
| | | tttgtcttctaaagatgatgaaattattgagtatcagcaaatgttacataacctaagggagaaacttaagaatgctcagcttgatgctgataa |
| | | aagtaatgttatggctctacagcagggtattcaggaacgagacagtcaaattaagatgctcaccgaacaagtagaacaatatacaaaa |
| | | gaaatggaaaagaatacttgtattattgaagatttgaaaaatgagctccaaagaaacaaaggtgcttcaaccctttctcaacagactcata |
| | | tgaaaattcagtcaacgttagacattttaaaagagaaaactaaagaggctgagagaacagctgaactggctgaggctgatgctaggga |
| | | aaaggataaagaattagttgaggctctgaagaggttaaaagattatgaatcgggagtatatggtttagaagatgctgtcgttgaaataaa |
| | | gaattgtaaaaaccaaattaaaataagagatcgagagattgaaatattaacaaaggaaatcaataaacttgaattgaagatcagtgatttc |
| | | cttgatgaaaatgaggcacttaagagcptgtgggccttgaaccaaagacaatgattgatttaactgaatttagaaatagcaaacactta |
| | | aaacagcagcagtacagagctgaaaaccagattcttttgaaagagattgaaagtctagaggaagaacgacttgatctgaaaaaaaaaa |
| | | ttcgtcaaatggctcaagaaagaggaaaaagaagtgcaacttcaggattaaccactgaggacctgaacctaactgaaaacatttctcaa |
| | | ggagatagaataagtgaaagaaaattggatttattgagcctcaaaaatatgagtgaagcacaatcaaagaatgaatttctttcaagagaa |
| | | ctaattgaaaaagaaagagatttagaaaggagtaggacagtgatagccaaatttcagaataaattaaaagaattagttgaagaaaataa |
| | | gcaacttgaagaaggtatgaaagaaatattgcaagcaattaaggaaatgcagaaagatcctgatgttaaaggaggagaaacatctcta |
| | | attatccctagccttgaaagactagttaatgctatagaatcaaagaatgcagaaggaatctttgatgcgagtctgcatttgaaagcccaag |
| | | ttgatcagcttaccggaagaaatgaagaattaagacaggagctcagggaatctcggaaagaggctataaattattcacagcagttggc |
| | | aaaagctaatttaaagatagaccatcttgaaaaagaaactagtcttttacgacaatcagaaggatcaaatgttgtttttaaaggaattgactt |
| | | acctgatgggatagcaccatctagtgccagtatcattaattctcagaatgaatatttaatacatttgttacaggaactagaaaataaagaaa |
| | | aaaagttaaagaatttagaagattctcttgaagattacaacagaaaatttgctgtaattcgtcatcaacaaagtttgttgtataaagaatacct |
| | | aagtgaaaaggagacctggaaaacagaatctaaaacaataaaagaggaaaagagaaaacttgaggatcaagtccaacaagatgcta |
| | | taaaagtaaaagaatataataatttgctcaatgctcttcagatggattcggatgaaatgaaaaaaatacttgcagaaaatagtaggaaaatt |
| | | actgttttgcaagtgaatgaaaaatcacttataaggcaatatacaaccttagtagaattggagcgacaacttagaaaagaaaatgagaag |
| | | caaaagaatgaattgttgtcaatggaggctgaagtttgtgaaaaaattgggtgtttgcaaagatttaaggaaatggccattttcaagattgc |
| | | agctctccaaaaagttgtagataatagtgtttctttgtctgaactagaactggctaataaacagtacaatgaactgactgctaagtacaggg |
| | | acatcttgcaaaaagataatatgcttgttcaaagaacaagtaacttggaacacctggagtgtgaaaacatctccttaaaagaacaagtgg |
| | | agtctataaataaagaactggagattaccaaggaaaaacttcacactattgaacaagcctgggaacaggaaactaaattaggtaatgaa |
| | | tctagcatggataaggcaaagaaatcaataaccaacagtgacattgtttccatttcaaaaaaaataactatgctggaaatgaaggaattaa |
| | | atgaaaggcagcgggctgaacattgtcaaaaaatgtatgaacacttacggacttcgttaaagcaaatggaggaacgtaattttgaattgg |
| | | aaaccaaatttgctgagcttaccaaaatcaatttggatgcacagaaggtggaacagatgttaagagatgaattagctgatagtgtgagca |
| | | ttggcagtaagtgatgctgataggcaacggattctagaattagagaagaatgaaatggaactaaaagttgaagtgtcaaaactgagaga |
| | | gatttctgatattgccagaagacaagttgaaattttgaatgcacaacaacaatctagggacaaggaagtagagtccctcagaatgcaact |
| | | gctagactatcaggcacagtctgatgaaaagtcgctcattgccaagttgcaccaacataatgtctctcttcaactgagtgaggctactgct |
| | | cttggtaagttggagtcaattacatctaaactgcagaagatggaggcctacaacttgcgcttagagcagaaacttgatgaaaaagaaca |
| | | ggctctctattatgctaagaaacagagcaaaacatctgcgccaaacaattcagtctctacgacgacagtttagtggag |
| | | Ctttacccttggcacaacaggaaaagttctccaaaacaatgattcaactacaaaatgacaaacttaagataatgcaagaaatgaaaaatt |
| | | Ctcaacaagaacatagatatggagaacaaaacattggagatggaattaaaattaaagggcctggaagagttaataagcactttaaag |
| | | gataccaaaggagcccaaaagtaatcaactggcatatgaaaatagaagaacttcgtcttcaagaacttaaactaaatcgggaattagt |
| | | caaggataaagaagaaataaaatatttgttataacataatttctgaatatgaacgtacaatcagcagtcttgaagaagaaattgtgcaaca |
| | | gaacaagtttcatgaagaaagacaaatggcctgggatcaaagagaagttgacctggaacgccaactagacatttttgaccgtcagcaa |
| | | gctctaaggaaaattaaggagaacattcgaataattctagaaacacgggcaacttgcaaatcactagaagagaaactaaaagagaaag |
| | | ctaggcagaaaagagatggaaccaaaatctcaccacacattgaaaattgctcatcaaaccattgcaaa |
| | | catgcaagcaaggttgaagaagtattaaagaagtatcaacgtcttctagaaaaagccagagaggagcaaagagaaat |
| | | tgtgaagaaacatgaggaagaccttcatattcttcatcacagattagaactacaggctgatagttcactaaataaattcaaacaaacggct |
| | | tgggatttaatgattactcccactccagttcctaccaacaagcattttattcgtctggctgagatggaacagacagtagcagaacaa |
| | | gatgactctctttcctcggtcaaactaaagaaagtatcacaagatttggagagacaaagagaaatcactgaattaaaagtaaaa |
| | | gaatttgaaaatatcatagcttcaagaactaccatgaagatgaagtgaaaaaagtaaaagcggaagtagaggatttaaagtatctt |
| | | ctggaccagtcacaagagtcacagtgtttaaaatctgaacttcaggctcaaaaagaagcaaattcaagagctccaacaactacaat |
| | | gagaaatctagtagaacggctaaagagccaattagccttgaaggagaaacaacagaaagcacttagtcgggcacttttagaactccg |
| | | ggcagaaatgacagcagctgctgaagaacgtattatttctgcaacttctcaaaaagaggcccatctcaatgttcaacaaatcgttgatcg |
| | | acatactagagagctaaagacacaagttgaagatttaaatgaaaatcttttaaaattgaaagaagcacttaaaacaagtaaaaacagaga |
| | | aaactcactaactgataatttgaatgacttaaataatgaactgcaaaagaaacaaaaagcctataataaaatacttagagagaaagagga |
| | | aattgatcaagagaatgatgaactgaaaaggcaaattaaaagactaaccagtggattacagggcaaacccctgacagataataaacaa |
| | | agtctaattgaagaactccaaaggaaagttaaaaaactagagaaccaattagagggaaaggtggaggaagtagacctaaaacctatg |
| | | aaagaaaagaatgctaaagaagaattaattaggtgggaagaaggtaaaaagtggcaagccaaaatagaaggaattcgaaacaagtta |
| | | aaagagaaagaggagtctttactttaacaaagcagttgaatactttgaaggatctttttgccaaagccgataaagagaaacttactt |
| | | tgcagaggttaactaaaaacaactggcatgactgttgatcaggttttgggaatacgagctttggagtcagaaaaagaattggaagaatta |
| | | aaaaagagaaatcttgacttagaaaatgatatattgtatatgagggcccaccaagctcttcctcgagattctgttgtagaagatttacattta |
| | | caactatagatacctccaagaaaaacttcatgctttagaaaaacagtttcaaaggatacatattctaagccttcaatttcaggaatagagtc |
| | | agatgatcattgtcagagagaacaggagcttcagaaggaaaachtgaagtgtcatctgaaaatattgaactgaaatttcagcttgaaca |
| | | agcaaataaagatttgccaagattaaagaatcaagtcagagatttgaaggaaatgtgtgaatttcttaagaaagaaaaagcagaagttca |
| | | gcggaaacttggccatgttagagggtctggtagaagtggaaagacaatcccagaactggaaaaaaccattggtttaatgaaaaaagta |
| | | gttgaaaaagtccagagagaaaatgaacagttgaaaaaagcatcaggaatattgactagtgaaaaaatggctaatattgagcaggaaa |
| | | atgaaaaattgaagcctgaattagactaaacttaaagctcatcttgggcatcagttgagcatgcactatgaatccaagaccaaaggcaca |
| | | gaaaaaattattgctgaaaatgaaaggcttcgtaaagaacttaaaaaagaaactgatgctgcagagaaattacggatagcaaagaataa |
| | | tttagagatattaaatgagaagatgacagttcaactagaagagactggtaagagattgcagtttgcagaaagcagaggtccacagcttg |
| | | aaggtgctgacagtaagagctggaatccattgtggttacaagaatgtatgaaaccaagttaaaagaattggaaactgatattgccaaa |
| | | aaaaatcaaagcattactgaccttaaacagcttgtaaaagaagcaacagagagagaacaaaaagttaacaaatacaatgaagaccttg |
| | | aacaacagattaagattcttaaacatgttcctgaaggtgctgagacagagcaaggccttaaacgggagcttcaagttcttagattagcta |
| | | atcatcagctggataaagagaaagcagaattaatccatcagatagaagctaacaaggaccaaagtggagctgaaagcaccatacctg |
| | | atgctgatcaactaaaggaaaaaataaaagatctagagacacagctcaaaatgtcagatctagaaaagcagcatttgaacgaggaaat |
| | | aaagaagctgaaaaaagaactggaaaattttgatccttcattttttgaagaaattgaagatcttaagtataattacaaggaagaagtgaag |
| | | aagaatattctcttagaagagaaggtaaaaaaactttcagaacaattgggagttgaattaactagccctgttgctgcttctgaagagtttga |
| | | agatgaagaagaaagtcctcttaatttccccatttacatgacgaggattttgacagctttcaaagtggtgaggacactgaagactggtttt |
| | | ggctttaccaatgtgactgcacaccaaaaatggttaattttcaagacctggcatcaggctcctttctgtcaaggcacagacagcacacatt |
| | | gtcctggaagatggaactaagatgaaaggttactcctttggccatccatcctctgttgctggtgaagtggtttttaatactggcctgggag |
| | | ggtacccagaagctattactgaccctgcctacaaaggacagattctcacaatggccaaccctattattgggaatggtggagctcctgata |
| | | ctactgctctggatgaactgggacttagcaaatatttggagtctaatggaatcaaggtttcaggtttgctggtgctggattatagtaaagac |
| | | tacaaccactggctggctaccaagagtttagggcaatggctacaggaagaaaaggttcctgcaatttatggagtggacacaagaatgc |
| | | tgactaaaataattcgggataagggtaccatgcttgggaagattgaatttgaaggtcagcctgtggattttgtggatccaaataaacagaa |
| | | tttgattgctgaggtttcaaccaaggatgtcaaagtgtacggcaaaggaaaccccacaaaagtggtagctgtagactgtgggattaaaa |
| | | acaatgtaatccgcctgctagtaaagcgaggagctgaagtgcacttagttccctggaaccatgatttcaccaagatggagtatgatggg |
| | | attttgatcgcgggaggaccggggaacccagctcttgcagaaccactaattcagaatgtcagaaagattttggagagtgatcgcaagg |
| | | agccattgtttggaatcagtacaggaaacttaataacaggattggctgctggtgccaaaacctacaagatgtccatggccaacagagg |
| | | gcagaatcagcctgttttgaatatcacaaacaaacaggctttcattactgctcagaatcatggctatgccttggacaacaccctccctgct |
| | | ggctggaaaccactttttgtgaatgtcaacgatcaaacaaatgaggggattatgcatgagagcaaacccttcttcgctgtgcagttccac |
| | | ccagaggtcaccccggggccaatagacactgagtacctgtttgattcctttttctcactgataaagaaaggaaaagctaccaccattaca |
| | | tcagtcttaccgaagccagcactagttgcatctcgggttgaggtttccaaagtccttattctaggatcaggaggtctgtccattggtcagg |
| | | ctggagaatttgattactcaggatctcaagctgtaaaagccatgaaggttagaaaatgtcaaaactgttctgatgaacccttaacattgcat |
| | | cagtccagttccaatgaggtgggcttatagcaagcggatactgtctactttcttcccatcacccctcagtttgtcacagaggtcatcaagg |
| | | cagaacagccagatgggttaattctgggcatgggggccagacagctctgaactgtggagtggaactattcaagagaggtgtgctcaa |
| | | ggttatatgctgtgaaagtcctgggaacttcagttgagtccattatggctacggaagacaggcagctgttttcagataaactaaatgagat |
| | | caatgaaaagattgctccaagttttgcagtggaatcgattgaggatgcactgaaggcagcagacaccattggctacccagtgatgatcc |
| | | gttccgcctatgcactgggtgcttagctcaggcatctgtcccaacagagagactttgatggacctcagcacaaaggcctttgctatg |
| | | accaaccaaattctggtggagaagtcagtgacggcccggctgtggctgaggagccCctCcaccgaccGTGAGTttgggcTG |
| | | CATGacTGCATGgtTGCATGaacacaTATTAATttcctccacttagttctacacctcattcattcattcagtgagtgtttc |
| | | gctgtcaagctacgctgcCCGCCTAACACTOCCAATGCCGOTCCCAAGCCCGGATAAAAAGT |
| | | GGAGGGGGCGGgaaggtttttcttttcctgagaaaacaacacgtttttgttttctcaggttttgctttttggcctttttctagcttaaaa |
| | | aaaaaaaaagcaaaagttattctgcagatatccatcacact |

The foregoing written specification is considered to be sufficient to enable one skilled in the art to practice the invention. The present invention is not to be limited in scope by examples provided, since the examples are intended as a single illustration of one aspect of the invention and other functionally equivalent embodiments are within the scope of the invention. Various modifications of the invention in addition to those shown and described herein will become apparent to those skilled in the art from the foregoing description and fall within the scope of the appended claims. The advantages and objects of the invention are not necessarily encompassed by each embodiment of the invention.

### REFERENCES

1. Komor, A. C., Kim, Y. B., Packer, M. S., Zuris, J. A. & Liu, D. R. Programmable editing of a target base in genomic DNA without double-stranded DNA cleavage. Nature 533, 420-424 (2016).
2. Nishida, K. et al. Targeted nucleotide editing using hybrid prokaryotic and vertebrate adaptive immune systems. Science 353. (2016).
3. Gaudelli, N. M. et al. Programmable base editing of A-T to G-C in genomic DNA without DNA cleavage. Nature 551, 464-471 (2017).
4. Anzalone, A. V. et al. Search-and-replace genome editing without double-strand breaks or donor DNA. Nature 576, 149-157 (2019).
5. Yarnall, M. T. N. et al. Drag-and-drop genome insertion of large sequences without double-strand DNA cleavage using CRISPR-directed integrases. Nat. Biotechnol. 41, 500-512 (2023).
6. Anzalone, A. V. et al. Programmable deletion, replacement, integration and inversion of large DNA sequences with twin prime editing. Nat. Biotechnol. 40, 731-740 (2022).
7. Lampe, G. D. et al. Targeted DNA integration in human cells without double-strand breaks using CRISPR-associated transposases. Nat. Biotechnol. (2023) doi:10.1038/s41587-023-01748-1.
8. Zuo, E. et al. Cytosine base editor generates substantial off-target single-nucleotide variants in mouse embryos. Science 364.289-292 (2019).
9. Fiunara, M. et al. Genotoxic effects of base and prime editing in human hematopoietic stem cells. Nat. Biotechnol. (2023) doi:10.1038/s41587-023-01915-4.
10. Vallecillo-Viejo, I. C., Liscovitch-Braucr, N., Montiel-Gonzalez, M. F., Eisenberg, E. & Rosenthal, J. J. C. Abundant off-target edits from site-directed RNA editing can be reduced by nuclear localization of the editing enzyme. RNA Biol. 15, 104-114 (2018).
11. Katrekar, D. et al. Efficient in vitro and in vivo RNA editing via recruitment of endogenous ADARs using circular guide RNAs. Nat. Biotechnol. 40, 938-945 (2022).
12. Ruchika & Nakamura, T. Understanding RNA editing and its use in gene editing. Gene Genome Ed. 3-4, 100021 (2022).
13. Cox, D. B. T. et al. RNA editing with CRISPR-Cas13. Science 358, 1019-1027 (2017).
14. Abudayyeh, O. O. et al. A cytosine deaminase for programmable single-base RNA editing. Science 365, 382-386 (2019).
15. Merkle, T. et al. Precise RNA editing by recruiting endogenous ADARs with antisense oligonucleotides. Nat. Biotechnol. (2019) doi:10.1038/s41587-019-0013-6.
16. Vogel, P. et al. Efficient and precise editing of endogenous transcripts with SNAP-tagged ADARs. Nat. Methods 15, 535-538 (2018).
17. Fukuda, M. et al. Construction of a guide-RNA for site-directed RNA mutagenesis utilising intracellular A-to-I RNA editing. Sci. Rep. 7, 41478 (2017).
18. Wettengel, J., Reautschnig. P., Geisler, S., Kahle, P. J. & Stafforst, T. Harnessing human ADAR2 for RNA repair—Recoding a PINK1 mutation rescues mitophagy. Nucleic Acids Res. 45, 2797-2808 (2017).
19. Montiel-GonzAlez, M. F., Vallecillo-Viejo, I. C. & Rosenthal, J. J. C. An efficient system for selectively altering genetic information within mRNAs. Nucleic Acids Res. 44, e157 (2016).
20. Vogel, P., Schneider, M. F., Wettengel, J. & Stafforst, T. Improving site-directed RNA editing in vitro and in cell culture by chemical modification of the guideRNA. Angew. Chen. Int. Ed Engl. 53, 6267-6271 (2014).
21. Montiel-Gonzalez, M. F., Vallecillo-Viejo, I., Yudowski, G. A. & Rosenthal, J. J. C. Correction of mutations within the cystic fibrosis transmembrane conductance regulator by site-directed RNA editing. Proc. Natl. Acad. Sci. U.S.A. 110, 18285-18290 (2013).
22. Rees, H. A. & Liu, D. R. Base editing: precision chemistry on the genome and transcriptome of living cells. Nat. Rev. Genet. 19, 770-788 (2018).
23. Berger. A. et al. mRNA trans-splicing in gene therapy for genetic diseases. Wiley Interdiscip. Rev. RNA 7, 487-498 (2016).
24. Puttaraju, M., Jamison, S. F., Mansfield, S. G., Garcia-Blanco, M. A. & Mitchell, L. G. Spliceosome-mediated RNA trans-splicing as a tool for gene therapy. Nat. Biotechnol. 17, 246-252 (1999).
25. Liu, X. et al. Partial correction of endogenous DeltaF508 CFTR in human cystic fibrosis airway epithelia by spliceosome-mediated RNA trans-splicing. Nat. Biotechnol. 20, 47-52(2002).
26. Wang, J. et al. Trans-splicing into highly abundant albumin transcripts for production of therapeutic proteins in vivo. Mol. Ther. 17, 343-351 (2009).
27. Coady, T. H. & Lorson, C. L. Trans-splicing-mediated improvement in a severe mouse model of spinal muscular atrophy. J. Neurosci. 30, 126-130 (2010).
28. Coady. T. H., Shababi, M., Tullis, G. E. & Lorson. C. L. Restoration of SMN function: delivery of a trans-splicing RNA re-directs SMN2 pre-mRNA splicing. Mol. Ther. 15, 1471-1478 (2007).
29. Berger, A. et al. Repair of rhodopsin mRNA by spliceosome-mediated RNA trans-splicing: a new approach for autosomal dominant retinitis pigmentosa. Mol. Ther. 23, 918-930 (2015).
30. Rindt, H. et al. Replacement of huntingtin exon 1 by trans-splicing. Cell. Mol. Life Sci. 69, 4191-4204(2012).
31. Antonarakis, S. E. & Cooper, D. N. Human Gene Mutation: Mechanisms and Consequences, in Vogel and Motulsky's Human Genetics (eds. Speicher, M. R., Motulsky, A. G. & Antonarakis, S. E.) 319-363 (Springer Berlin Heidelberg, 2010). doi:10.1007/978-3-540-37654-5_12.

What is claimed is:

1. A non-naturally occurring, engineered composition comprising:
a trans-splicing template polynucleotide comprising
(a) an insertion sequence;
(b) a 5′ splicing motif sequence;
(c) optionally, a linker sequence;
(d) a hybridization sequence; and
(e) a nucleic acid sequence encoding a ribozyme.

2. The composition of claim 1, wherein (a)-(e) are arranged 5′ to 3′.

3. The composition of claim 1, wherein the ribozyme is capable of cleaving RNA.

4. The composition of claim 1, wherein the ribozyme is capable of cleaving DNA.

5. The composition of claim 1, wherein the ribozyme is a self-cleaving ribozyme.

6. The composition of claim 1, wherein the ribozyme is a naturally occurring ribozyme or a synthetic ribozyme.

7. The composition of claim 1, wherein the ribozyme is selected from Twister ribozyme, Hammerhead (HH) ribozyme, Hepatitis Delta Virus (HDV) ribozyme, Hairpin 1 ribozyme, Hairpin 2 ribozyme, Hairpin 3 ribozyme, Varkud Satellite ribozyme, gImS ribozyme, twister sister ribozyme, pistol ribozyme, and hatchet ribozyme.

8. The composition of claim 1, wherein the trans-splicing template polynucleotide comprises a sequence that is at least 90% identical to one of SEQ ID NOs: 1-7 9-104, or 117-128.

9. The composition of claim 1, wherein the trans-splicing template polynucleotide comprises a sequence of one of SEQ ID NOs: 1-7, 9-104, or 117-128.

10. The composition of claim 1, wherein the insertion sequence is
(i) less than 1-2 kilobases;
(ii) about 1-2 kilobases; or
(iii) greater than 1-2 kilobases.

11. The composition of claim 1, wherein the 5′ splicing motif is GURAGU.

12. The composition of claim 1, wherein the linker is about 14 bp to 100 bp.

13. The composition of claim 1, wherein the hybridization sequence is about 50 bp to 400 bp.

14. A cell comprising the trans-splicing template polynucleotide of claim 1.

15. The cell of claim 14, wherein the cell is a prokaryotic cell or a eukaryotic cell.

16. The cell of claim 15, wherein the eukaryotic cell is a mammalian cell or a plant cell.

17. A method of editing a target RNA sequence in a cell comprising administering to the cell an effective amount of the trans-splicing template polynucleotide of claim 1.

18. The method of claim 17, wherein the delivering to the cell is by a viral vector, optionally wherein the viral vector is Adeno-associated viral (AAV) vector, a virus, optionally wherein the virus is an Adenovirus, a lentivirus, a herpes simplex virus; and/or a lipid nanoparticle.

19. A method of editing a target RNA sequence via 3′ trans-splicing in a cell, the method comprising delivering to the cell a non-naturally occurring, engineered trans-splicing template polynucleotide comprising:
(a) a nucleic acid sequence encoding a ribozyme;
(b) a hybridization sequence;
(c) a 3′ splicing motif sequence;
(d) optionally, a linker sequence; and
(e) an insertion sequence.

20. A method of editing a target RNA sequence in a cell, the method comprising delivering to the cell
(i) a trans-splicing template polynucleotide comprising a nucleic acid sequence encoding a ribozyme, wherein the trans-splicing template polynucleotide hybridizes to at least a portion of the target RNA sequence;
(ii) a polynucleotide encoding a Cas7-11 enzyme; and
(iii) a polynucleotide encoding a Cas7-11 guide RNA sequence;
causing cleavage and insertion steps to achieve editing of the target RNA sequence via simultaneous 5′ and 3′ trans-splicing.
